(19) United States
(12) Patent Application Publication
(10) Pub. No.: US 2023/0389416 A1
(43) Pub. Date: Nov. 30, 2023

(54) AMINE-CONTAINING COMPOUND, LIGHT-EMITTING DEVICE INCLUDING THE SAME, ELECTRONIC APPARATUS INCLUDING THE LIGHT-EMITTING DEVICE, AND ELECTRONIC DEVICE INCLUDING THE ELECTRONIC APPARATUS

(71) Applicant: Samsung Display Co., Ltd., Yongin-si (KR)

(72) Inventors: Eunjae Jeong, Yongin-si (KR); Dongjun Kim, Yongin-si (KR); Beomjoon Kim, Yongin-si (KR); Chaeyeong Kim, Yongin-si (KR); Sanghyun Han, Yongin-si (KR)

(73) Assignee: Samsung Display Co., Ltd., Yongin-si, Gyeonggi-do (KR)

(21) Appl. No.: 18/105,481

(22) Filed: Feb. 3, 2023

(30) Foreign Application Priority Data
May 25, 2022 (KR) 10-2022-0064239

(51) Int. Cl. H10K 85/60; H01L 33/50; H10K 50/11; H10K 50/15

(52) U.S. Cl. CPC H10K 85/633; H01L 33/508; H10K 50/11; H10K 50/156

(57) ABSTRACT

Embodiments provide a light-emitting device that includes: a first electrode; a second electrode facing the first electrode; an interlayer between the first electrode and the second electrode and including an emission layer; and an amine-containing compound represented by Formula 1, an amine-containing compound represented by Formula 2, an amine-containing compound represented by Formula 3, or any combination thereof, and Formulae 1 to 3 are described in the specification:

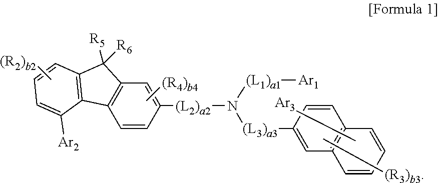

### CROSS-REFERENCE TO RELATED APPLICATION(S)

This application claims priority to and benefits of Korean Patent Application No. 10-2022-0064239 under 35 U.S.C. § 119, filed on May 25, 2022, in the Korean Intellectual Property Office, the entire contents of which are incorporated herein by reference.

### BACKGROUND

### 1. Technical Field

Embodiments relate to an amine-containing compound, a light-emitting device including the same, an electronic apparatus including the light-emitting device, and an electronic device including the electronic apparatus.

### 2. Description of the Related Art

An organic light-emitting device may have wide viewing angles, excellent contrast, and a short response time as compared to an inorganic light-emitting device. An organic light-emitting device may include a first electrode, a hole transport region, an emission layer, an electron transport region, and a second electrode. Holes provided from the first electrode may move toward the emission layer through the hole transport region. Electrons provided from the second electrode may move toward the emission layer through the electron transport region. Carriers such as the holes and the electrons may combine in the emission layer. Excitons may be generated by the combination of the carriers. Light is generated as the excitons transition from an excited state to a ground state.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

Embodiments include an amine-containing compound having improved hole transport characteristics and a light-emitting device including the amine-containing compound and thus having a low driving voltage, high luminance, high luminescence efficiency, and long lifespan. Embodiments include a high-quality electronic apparatus and an electronic device including the light-emitting device.

Additional aspects will be set forth in part in the description which follows and, in part, will be apparent from the description, or may be learned by practice of the embodiments of the disclosure.

According to embodiments, provided is an amine-containing compound which may be represented by one of Formulae 1 to 3.

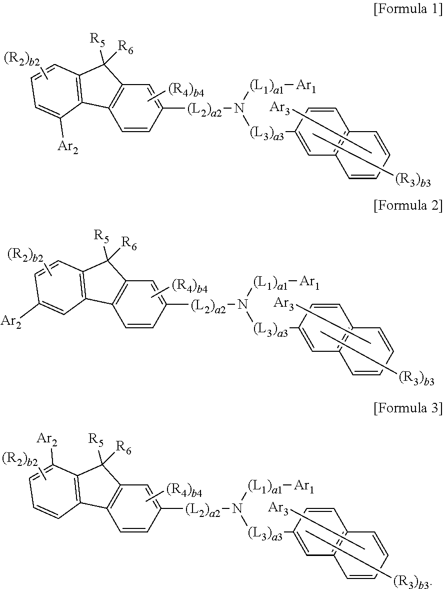

In Formulae 1 to 3,
L₁ to L₃ and Ar₁ to Ar₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, except that each of L₁ and Ar₁ may not be a fluorene group unsubstituted or substituted with at least one R₁₀ₐ,
a1 to a3 may each independently be an integer from 0 to 5; wherein when a1 is 0, a group represented by *-(L₁)ₐ₁-*′ may be a single bond, when a2 is 0, a group represented by *-(L₂)ₐ₂-*′ may be a single bond, when a3 is 0, a group represented by *-(L₃)ₐ₃-*′ may be a single bond, when a1 is 2 to 5, multiple L₁(s) may be identical to or different from each other, when a2 is 2 to 5, multiple L₂(s) may be identical to or different from each other, and when a3 is 2 to 5, multiple L₃(s) may be identical to or different from each other,
R₂ to R₆ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₅ and R₆ may optionally be bound to each other to form a C₃-C₆₀ carbocyclic group unsubstituted or substituted with at least one R₁₀ₐ or a C₁-C₆ heterocyclic group unsubstituted or substituted with at least one R₁₀ₐ,
b2 and b4 may each independently be an integer from 0 to 3,
b3 may be an integer from 0 to 6,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
and *′ each indicate a binding site to an adjacent atom.

In an embodiment, the amine-containing compound may not include a carbazole group and a fluorene group.

In an embodiment, R₁₀ₐ may be deuterium, a C₁-C₂₀ alkyl group, a cyclohexyl group, a cycloheptyl group, a cyclooctyl group, an adamantanyl group, a norbornanyl group, a phenyl group, a naphthyl group, a phenanthrenyl group, a dibenzofuranyl group, or a dibenzothiophenyl group.

In an embodiment, L₁ to L₃ and Ar₁ to Ar₃ may each independently be a cyclohexane group, a cycloheptane group, a cyclooctane group, an adamantane group, a norbornane group, a benzene group, a naphthalene group, a phenanthrene group, a dibenzofuran group, or a dibenzothiophene group, each unsubstituted or substituted with deuterium, a C₁-C₂₀ alkyl group, a cyclohexyl group, a cycloheptyl group, a cyclooctyl group, an adamantanyl group, a norbornanyl group, a phenyl group, a naphthyl group, a phenanthrenyl group, a dibenzofuranyl group, a dibenzothiophenyl group, or any combination thereof.

In an embodiment, L₁ to L₃ may each independently be a group represented by one of Formulae 4-1 to 4-3, which are explained below.

In an embodiment, Ar₁ to Ar₃ may each independently be a group represented by one of Formulae 5-1 to 5-7, which are explained below.

In an embodiment, Ar₁ to Ar₃ may each independently be a group represented by one of Formulae 6-1 to 6-12, which are explained below.

In an embodiment, in Formulae 1 to 3, a group represented by

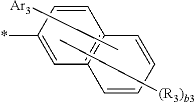

may be a group represented by one of Formulae A-1 to A-3, which are explained below.

In an embodiment, R₅ and R₆ may be linked to each other to form a cyclopentane group or a cyclohexane group, each unsubstituted or substituted with at least one R₁₀ₐ.

In an embodiment, the amine-containing compound may be one of Compounds 1 to 217, which are explained below.

According to embodiments, provided is a light-emitting device which may include: a first electrode; a second electrode facing the first electrode; an interlayer between the first electrode and the second electrode and including an emission layer; and an amine-containing compound represented by Formula 1, an amine-containing compound represented by Formula 2, an amine-containing compound represented by Formula 3, or any combination thereof, wherein Formulae 1 to 3 are explained herein.

In an embodiment, the interlayer may further include a hole transport region between the first electrode and the emission layer, and an electron transport region between the emission layer and the second electrode; the hole transport region may include a hole injection layer, a hole transport layer, an emission auxiliary layer, an electron blocking layer, or any combination thereof; and the electron transport region may include a buffer layer, a hole blocking layer, an electron control layer, an electron transport layer, an electron injection layer, or any combination thereof.

In an embodiment, the interlayer may include the amine-containing compound represented by Formula 1, the amine-containing compound represented by Formula 2, the amine-containing compound represented by Formula 3, or any combination thereof.

In an embodiment, the hole transport region may include the amine-containing compound represented by Formula 1, the amine-containing compound represented by Formula 2, the amine-containing compound represented by Formula 3, or any combination thereof.

In an embodiment, the hole transport layer may include the amine-containing compound represented by Formula 1, the amine-containing compound represented by Formula 2, the amine-containing compound represented by Formula 3, or any combination thereof; and the hole transport layer may directly contact the emission layer.

In an embodiment, the light-emitting device may further include a capping layer outside the first electrode, wherein the capping layer may include the amine-containing compound represented by Formula 1, the amine-containing compound represented by Formula 2, the amine-containing compound represented by Formula 3, or any combination thereof.

In an embodiment, the light-emitting device may further include a first capping layer outside the first electrode, and a second capping layer outside the second electrode, wherein the first capping layer or the second capping layer may include the amine-containing compound represented by Formula 1, the amine-containing compound represented by Formula 2, the amine-containing compound represented by Formula 3, or any combination thereof.

According to embodiments, provided is an electronic apparatus which may include the light-emitting device.

In an embodiment, the electronic apparatus may further include: a thin-film transistor electrically connected to the light-emitting device; and a color filter, a color conversion layer, a touch screen layer, a polarizing layer, or any combination thereof.

According to embodiments, provided is an electronic device which may include the electronic apparatus, wherein the electronic device may be a flat panel display, a curved display, a computer monitor, a medical monitor, a television, a billboard, an indoor light, an outdoor light, a signal light, a head-up display, a fully transparent display, a partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall with multiple displays tiled together, a theater screen, a stadium screen, a phototherapy device, or a signboard.

It is to be understood that the embodiments above are described in a generic and explanatory sense only and not for the purpose of limitation, and the disclosure is not limited to the embodiments described above.

### BRIEF DESCRIPTION OF THE DRAWINGS

The above and other aspects and features of the disclosure will be more apparent by describing in detail embodiments thereof with reference to the accompanying drawings, in which:

FIG. 1 is a schematic cross-sectional view of a light-emitting device according to an embodiment;

FIG. 2 is a schematic cross-sectional view of an electronic apparatus according to an embodiment;

FIG. 3 is a schematic cross-sectional view of an electronic apparatus according to another embodiment;

FIG. 4 is a schematic perspective view of an electronic device including a light-emitting device according to an embodiment;

FIG. 5 is a schematic perspective view of the exterior of a vehicle as an electronic device including a light-emitting device according to an embodiment; and

FIGS. 6A to 6C are each a schematic diagram illustrating the interior of a vehicle, according to embodiments.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like numbers refer to like elements throughout.

In the description, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the description, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group consisting of” for the purpose of its meaning and interpretation. For example, “at least one of A, B, and C” may be understood to mean A only, B only, C only, or any combination of two or more of A, B, and C, such as ABC, ACC, BC, or CC. When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element.

Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

According to embodiments, a light-emitting device may include: a first electrode; a second electrode facing the first electrode; an interlayer between the first electrode and the second electrode and including an emission layer; and an amine-containing compound represented by Formula 1, an amine-containing compound represented by Formula 2, an amine-containing compound represented by Formula 3, or any combination thereof. Formulae 1 to 3 will be explained below.

In an embodiment, the first electrode may be an anode, and the second electrode may be a cathode. In an embodiment, the emission layer may include a dopant and a host, and may emit light. Details of the dopant and the host are described below.

The term “interlayer” as used herein may refer to a single layer and/or multiple layers between the first electrode and the second electrode of a light-emitting device.

In an embodiment, the interlayer may further include a hole transport region between the first electrode and the emission layer, and an electron transport region between the emission layer and the second electrode. In an embodiment, the hole transport region may include a hole injection layer, a hole transport layer, an emission auxiliary layer, an electron blocking layer, or any combination thereof. In an embodiment, the electron transport region may include a buffer layer, a hole blocking layer, an electron control layer, an electron transport layer, an electron injection layer, or any combination thereof.

For example, the hole transport region may include a hole injection layer disposed on the first electrode and a hole transport layer located between the hole injection layer and the emission layer. The hole injection layer may have a single-layered structure or a multi-layered structure. The hole transport layer may have a single-layered structure or a multi-layered structure. For example, the hole transport layer may include a first hole transport layer, a second hole transport layer, and a third hole transport layer, sequentially disposed on the hole injection layer.

For example, the electron transport region may include an electron transport layer disposed on the emission layer and an electron injection layer located between the electron transport layer and the second electrode.

In an embodiment, the interlayer may include the amine-containing compound. For example, the interlayer may include the amine-containing compound represented by Formula 1, the amine-containing compound represented by Formula 2, the amine-containing compound represented by Formula 3, or any combination thereof.

In an embodiment, the hole transport region may include the amine-containing compound. For example, the hole transport region may include the amine-containing compound represented by Formula 1, the amine-containing compound represented by Formula 2, the amine-containing compound represented by Formula 3, or any combination thereof.

In an embodiment, the hole transport layer may include the amine-containing compound, and the hole transport layer may directly contact the emission layer. For example, the hole transport layer may include the amine-containing compound represented by Formula 1, the amine-containing compound represented by Formula 2, the amine-containing compound represented by Formula 3, or any combination thereof. For example, the hole transport layer may include a first hole transport layer, a second hole transport layer, and a third hole transport layer, and the third hole transport layer may include the amine-containing compound. In another example, the first hole transport layer and the third hole transport layer may each include the amine-containing compound. In yet another example, the first to third hole transport layers may each include the amine-containing compound.

In an embodiment, the light-emitting device may further include a capping layer outside the first electrode, and the capping layer may include the amine-containing compound.

In an embodiment, the light-emitting device may further include a first capping layer outside the first electrode, and a second capping layer outside the second electrode, and the first capping layer or the second capping layer may include the amine-containing compound. For example, the first capping layer or the second capping layer may include the amine-containing compound represented by Formula 1, the amine-containing compound represented by Formula 2, the amine-containing compound represented by Formula 3, or any combination thereof. For example, the amine-containing compound may be included in the first capping layer among the first capping layer, the first electrode, and the interlayer that are sequentially arranged. In another example, the amine-containing compound may be included in the second capping layer among the interlayer, the second electrode, and the second capping layer that are sequentially arranged. The first capping layer and the second capping layer may each include the amine-containing compound.

According to embodiments, an electronic apparatus may include the light-emitting device.

In an embodiment, the electronic apparatus may further include: a thin-film transistor electrically connected to the light-emitting device; and a color filter, a color conversion layer, a touch screen layer, a polarization layer, or any combination thereof. For example, the electronic apparatus may include the light-emitting device, the thin-film transistor, and the color filter. In another example, the electronic apparatus may include the light-emitting device, the thin-film transistor, the color filter, and the color conversion layer.

According to embodiments, an electronic device may include the electronic apparatus, and the electronic device may be a flat panel display, a curved display, a computer monitor, a medical monitor, a TV, a billboard, an indoor light, an outdoor light, a signal light, a head-up display, a fully transparent display, a partially transparent display, a flexible display, a rollable display, a foldable display, a stretchable display, a laser printer, a phone, a cell phone, a tablet, a phablet, a personal digital assistant (PDA), a wearable device, a laptop computer, a digital camera, a camcorder, a viewfinder, a micro display, a 3D display, a virtual reality display, an augmented reality display, a vehicle, a video wall including multiple displays tiled together, a theater screen, a stadium screen, a phototherapy device, or a signboard.

In embodiments, an amine-containing compound may be represented by one of Formulae 1 to 3:

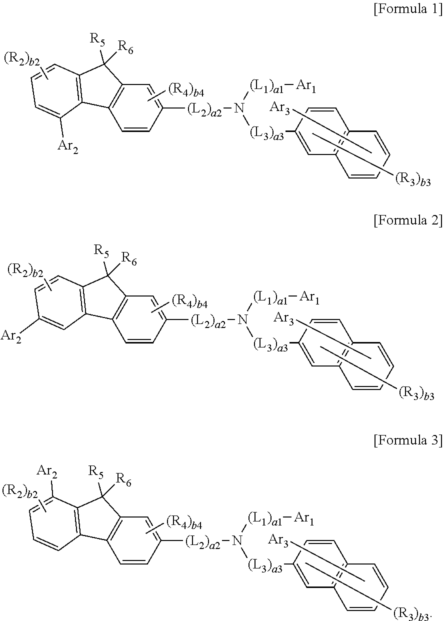

In Formulae 1 to 3,
L₁ to L₃ and Ar₁ to Ar₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, except that each of L₁ and Ar₁ may not be fluorene group unsubstituted or substituted with at least one R₁₀ₐ,
a1 to a3 may each independently be an integer from 0 to 5, wherein when a1 is 0, a group represented by *-(L₁)ₐ₁-*′ may be a single bond, when a2 is 0, a group represented by *-(L₂)ₐ₂-*′ may be a single bond, when a3 is 0, a group represented by *-(L₃)ₐ₃-*′ may be a single bond, when a1 is 2 to 5, multiple L₁(s) may be identical to or different from each other, when a2 is 2 to 5, multiple L₂(s) may be identical to or different from each other, and when a3 is 2 to 5, multiple L₃(s) may be identical to or different from each other,
R₂ to R₆ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₅ and R₆ may optionally be bound to each other to form a C₃-C₆₀ carbocyclic group unsubstituted or substituted with at least one R₁₀ₐ or a C₁-C₆₀ heterocyclic group unsubstituted or substituted with at least one R₁₀ₐ,
b2 and b4 may each independently be an integer from 0 to 3,
b3 may be an integer from 0 to 6,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each indicate a binding site to an adjacent atom.

In an embodiment, the amine-containing compound may not include a carbazole group and a fluorene group.

In an embodiment, R₁₀ₐ may be deuterium, a C₁-C₂₀ alkyl group, a cyclohexyl group, a cycloheptyl group, a cyclooctyl group, an adamantanyl group, a norbornanyl group, a phenyl group, a naphthyl group, a phenanthrenyl group, a dibenzofuranyl group, or a dibenzothiophenyl group.

In an embodiment, L₁ to L₃ and Ar₁ to Ar₃ may each independently be a cyclohexane group, a cycloheptane group, a cyclooctane group, an adamantane group, a norbornane group, a benzene group, a naphthalene group, a phenanthrene group, a dibenzofuran group, or a dibenzothiophene group, each unsubstituted or substituted with deuterium, a C₁-C₂₀ alkyl group, a cyclohexyl group, a cycloheptyl group, a cyclooctyl group, an adamantanyl group, a norbornanyl group, a phenyl group, a naphthyl group, a phenanthrenyl group, a dibenzofuranyl group, a dibenzothiophenyl group, or any combination thereof.

In an embodiment, L₁ to L₃ may each independently be a group represented by one of Formulae 4-1 to 4-3:

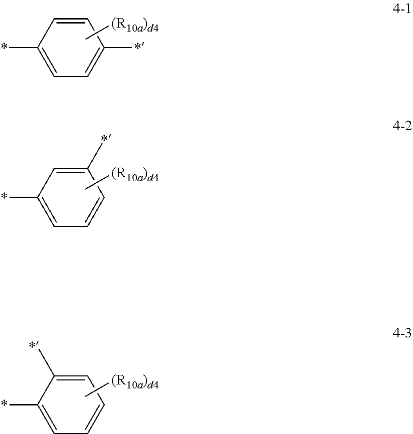

In Formulae 4-1 to 4-3
R₁₀ₐ is the same as described herein,
d4 may be an integer from 0 to 4, and
* and *′ each indicate a binding site to an adjacent atom.

In an embodiment, Ar₁ to Ar₃ may each independently be a group represented by one of Formulae 5-1 to 5-7:

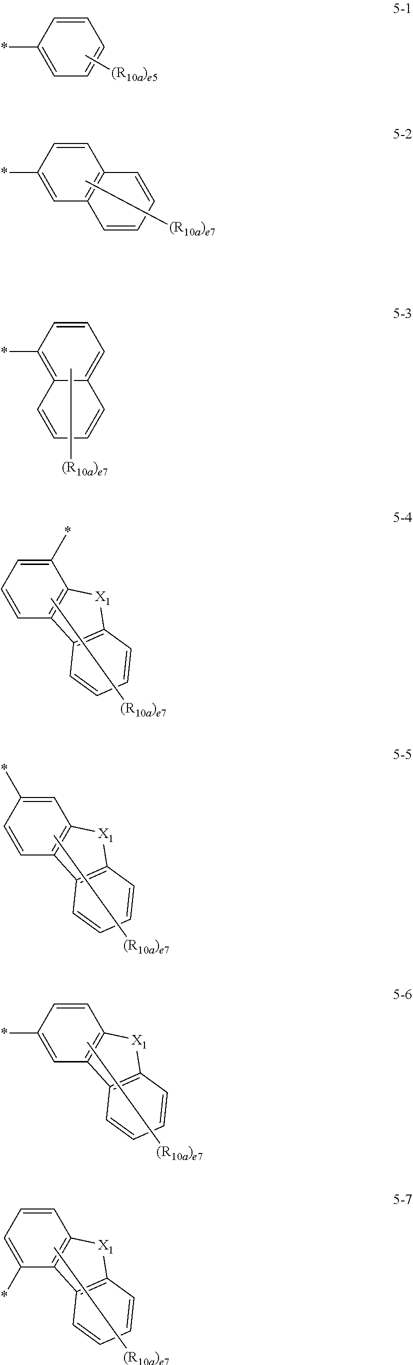

In Formulae 5-1 to 5-7,
R₁₀ₐ is the same as described herein,
X₁ may be O or S,
e5 may be an integer from 0 to 5,
e7 may be an integer from 0 to 7, and
* indicates a binding site to an adjacent atom.

In an embodiment, Ar₁ to Ar₃ may each independently be a group represented by one of Formulae 6-1 to 6-12:

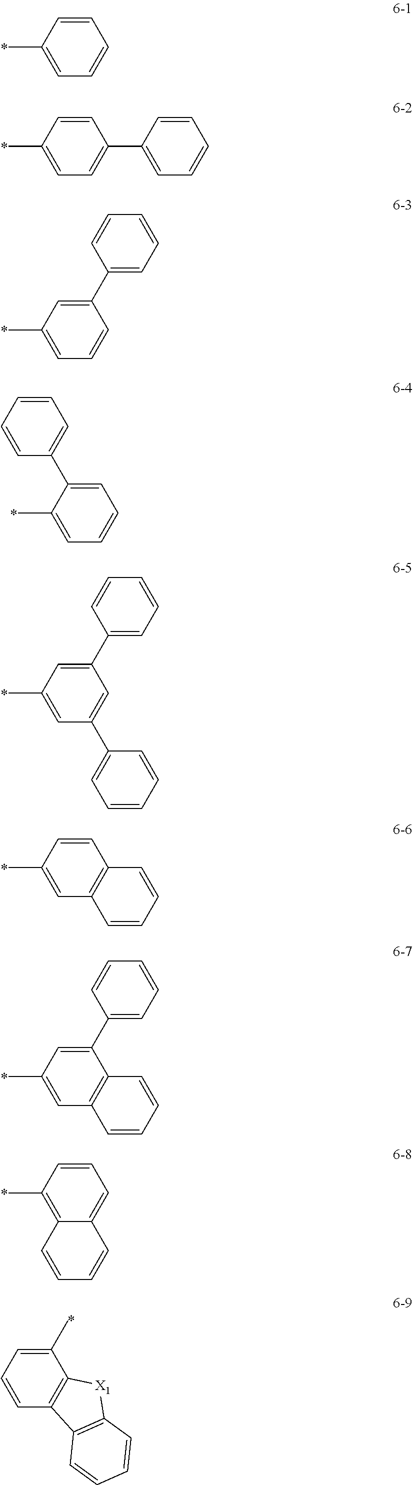

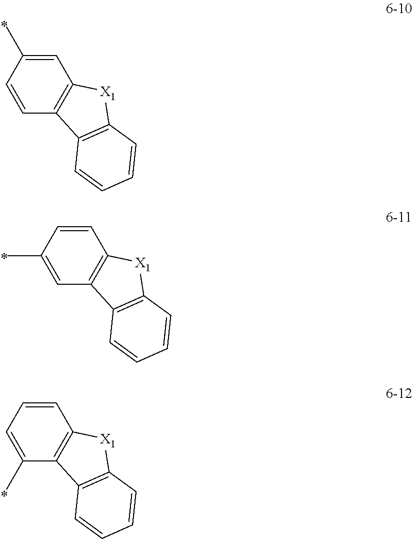

In Formulae 6-1 to 6-7,
X₁ may be O or S, and
* indicates a binding site to an adjacent atom.

In an embodiment, in Formulae 1 to 3, a group represented by

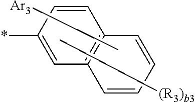

may be a group represented by one of Formulae A-1 to A-3:

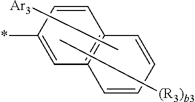

In Formulae A-1 to A-3,
Ar₃, R₃, and b3 are each the same as described herein and
* indicates a binding site to an adjacent atom.

In an embodiment, R₅ and R₆ may be linked to each other to form a cyclopentane group or a cyclohexane group, each unsubstituted or substituted with at least one R₁₀ₐ.

In an embodiment, the amine-containing compound may be one of Compounds 1 to 217:

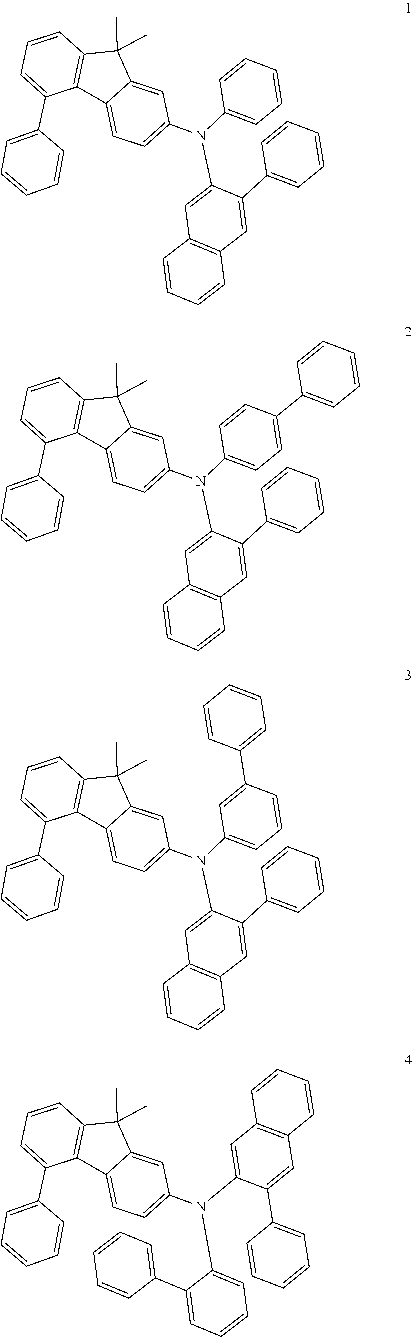

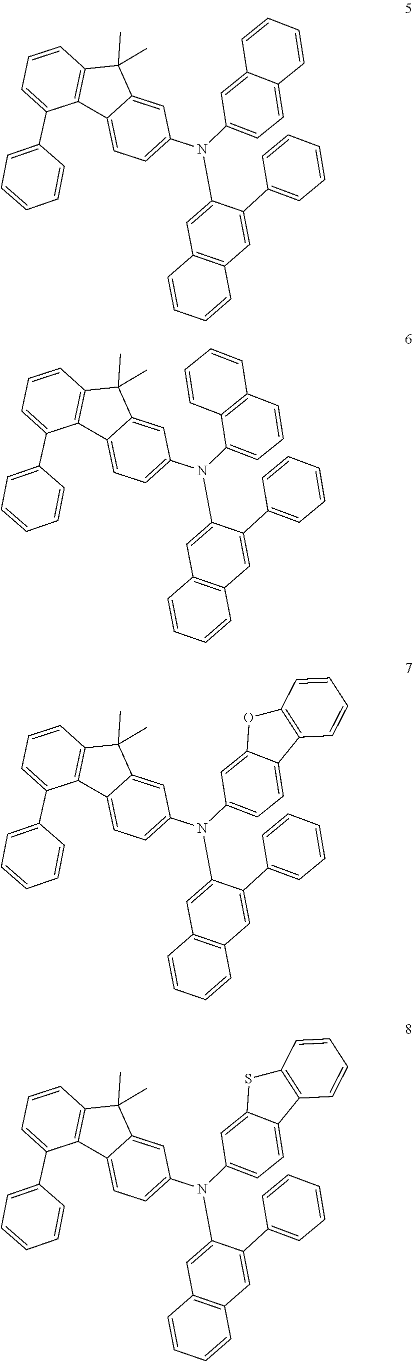

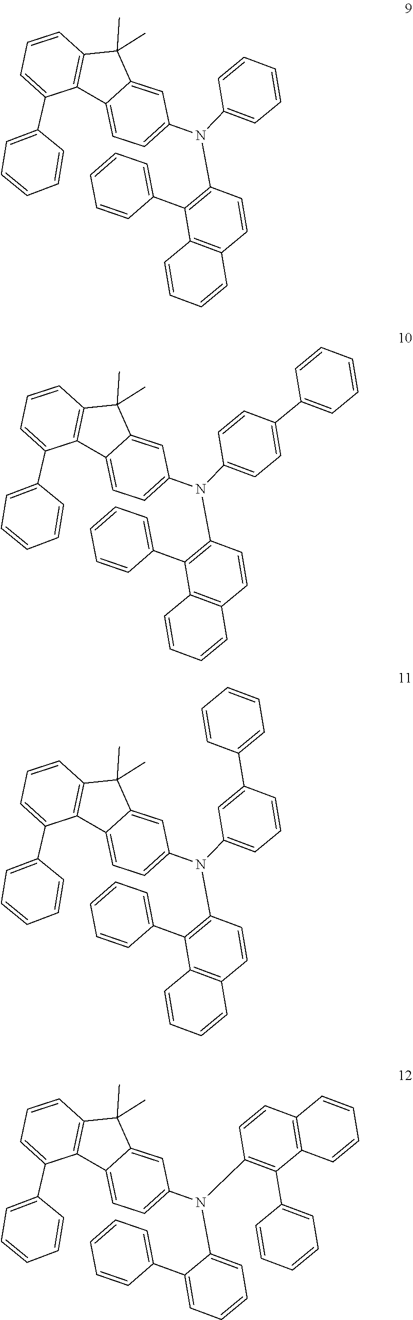

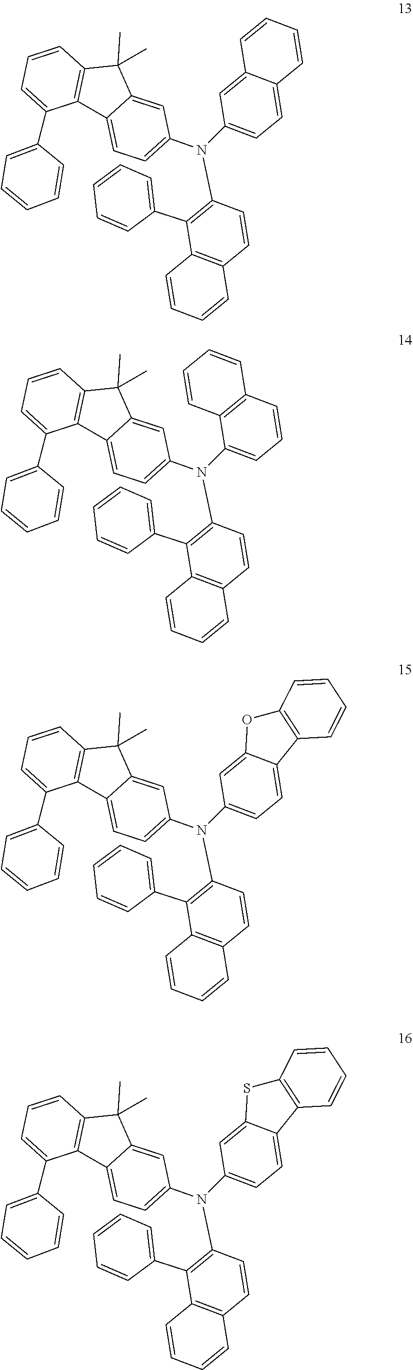

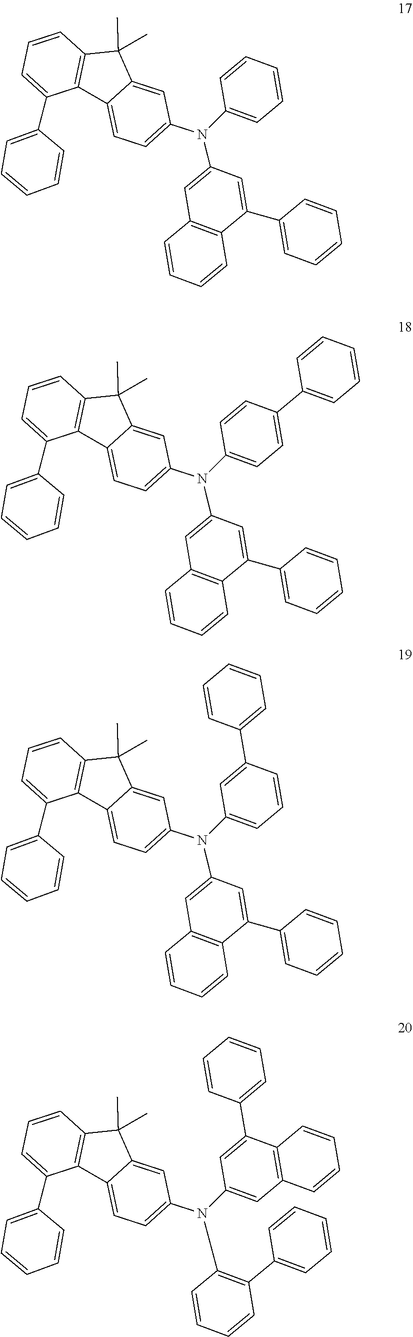

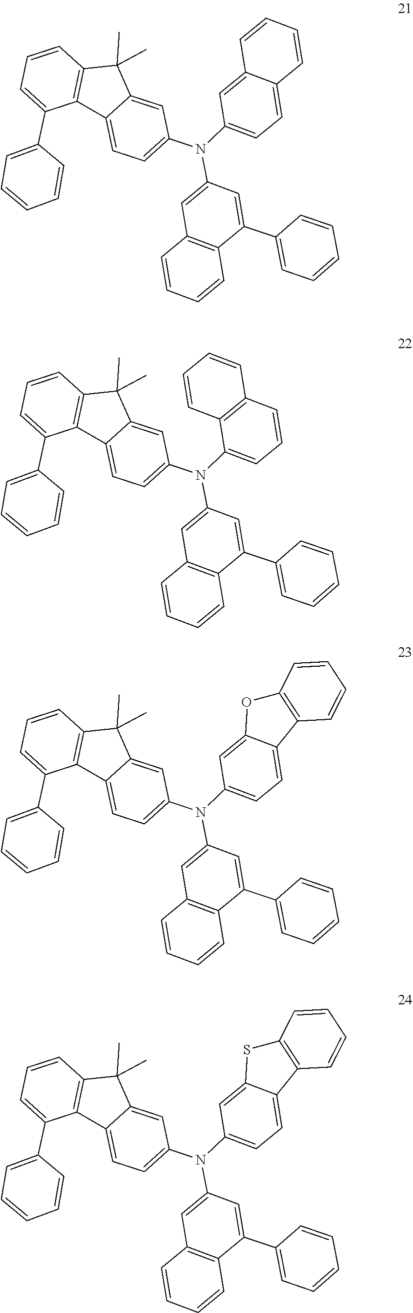 [SMILES: CC1(C)c2cc(N(c3ccc4ccccc4c3)c3cc(-c4ccccc4)c4ccccc4c3)ccc2-c2c(-c3ccccc3)cccc21 ; CC1(C)c2cc(N(c3cc(-c4ccccc4)c4ccccc4c3)c3cccc4ccccc34)ccc2-c2c(-c3ccccc3)cccc21 ; CC1(C)c2cc(N(c3cc(-c4ccccc4)c4ccccc4c3)c3ccc4c(c3)oc3ccccc34)ccc2-c2c(-c3ccccc3)cccc21 ; CC1(C)c2cc(N(c3cc(-c4ccccc4)c4ccccc4c3)c3ccc4c(c3)sc3ccccc34)ccc2-c2c(-c3ccccc3)cccc21]

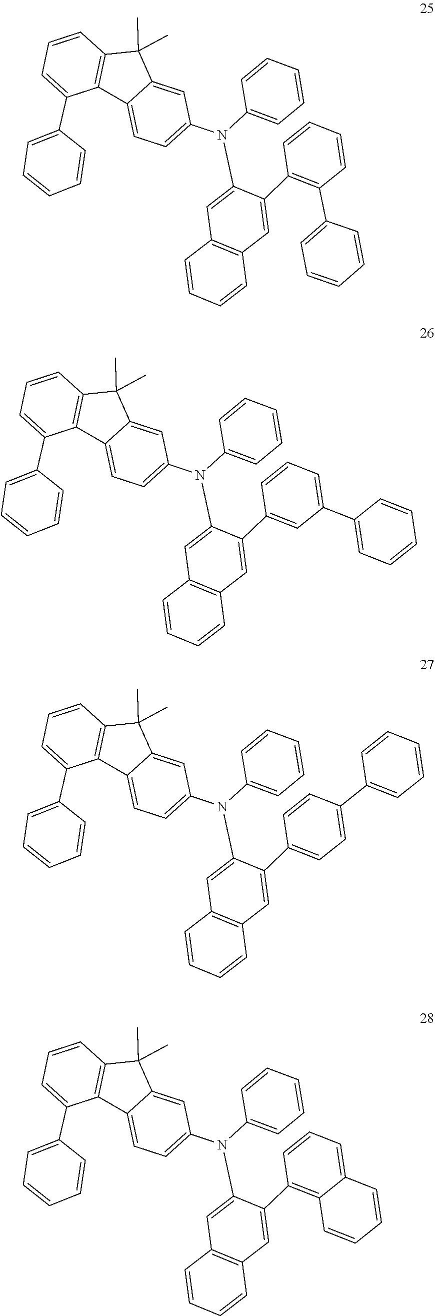 [SMILES: CC1(C)c2cc(N(c3ccccc3)c3cc4ccccc4cc3-c3ccccc3-c3ccccc3)ccc2-c2c(-c3ccccc3)cccc21 ; CC1(C)c2cc(N(c3ccccc3)c3cc4ccccc4cc3-c3cccc(-c4ccccc4)c3)ccc2-c2c(-c3ccccc3)cccc21 ; CC1(C)c2cc(N(c3ccccc3)c3cc4ccccc4cc3-c3ccc(-c4ccccc4)cc3)ccc2-c2c(-c3ccccc3)cccc21 ; CC1(C)c2cc(N(c3ccccc3)c3cc4ccccc4cc3-c3cccc4ccccc34)ccc2-c2c(-c3ccccc3)cccc21]

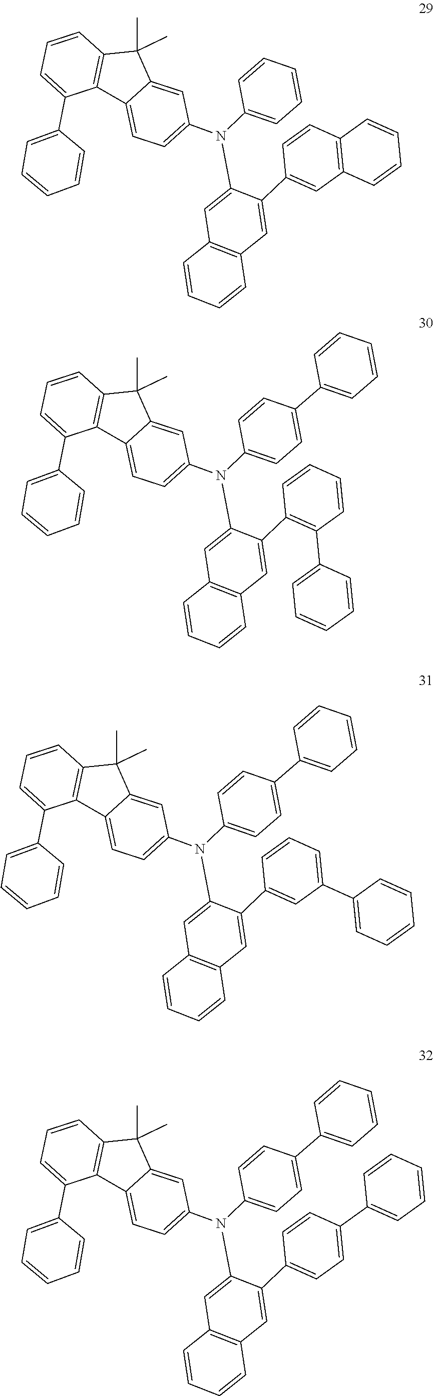 [SMILES: CC1(C)c2cc(N(c3ccccc3)c3cc4ccccc4cc3-c3ccc4ccccc4c3)ccc2-c2c(-c3ccccc3)cccc21 ; CC1(C)c2cc(N(c3ccc(-c4ccccc4)cc3)c3cc4ccccc4cc3-c3ccccc3-c3ccccc3)ccc2-c2c(-c3ccccc3)cccc21 ; CC1(C)c2cc(N(c3ccc(-c4ccccc4)cc3)c3cc4ccccc4cc3-c3cccc(-c4ccccc4)c3)ccc2-c2c(-c3ccccc3)cccc21 ; CC1(C)c2cc(N(c3ccc(-c4ccccc4)cc3)c3cc4ccccc4cc3-c3ccc(-c4ccccc4)cc3)ccc2-c2c(-c3ccccc3)cccc21]

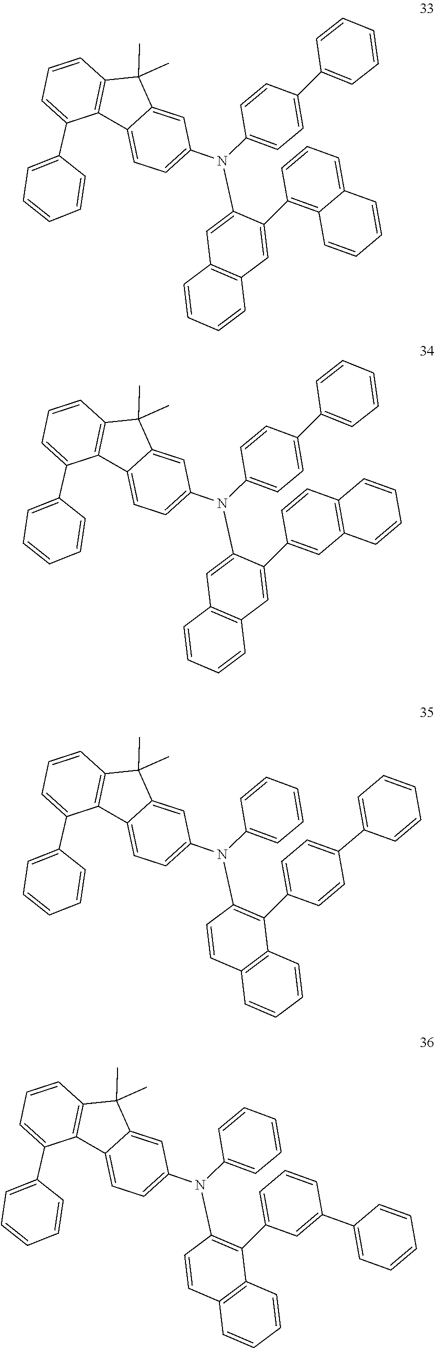 [SMILES: CC1(C)c2cc(N(c3ccc(-c4ccccc4)cc3)c3cc4ccccc4cc3-c3cccc4ccccc34)ccc2-c2c(-c3ccccc3)cccc21 ; CC1(C)c2cc(N(c3ccc(-c4ccccc4)cc3)c3cc4ccccc4cc3-c3ccc4ccccc4c3)ccc2-c2c(-c3ccccc3)cccc21 ; CC1(C)c2cc(N(c3ccccc3)c3ccc4ccccc4c3-c3ccc(-c4ccccc4)cc3)ccc2-c2c(-c3ccccc3)cccc21 ; CC1(C)c2cc(N(c3ccccc3)c3ccc4ccccc4c3-c3cccc(-c4ccccc4)c3)ccc2-c2c(-c3ccccc3)cccc21]

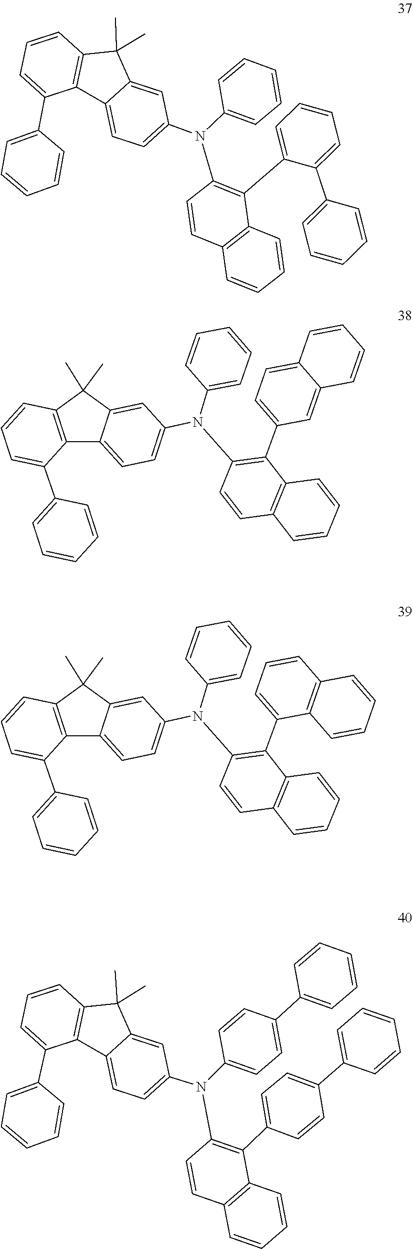

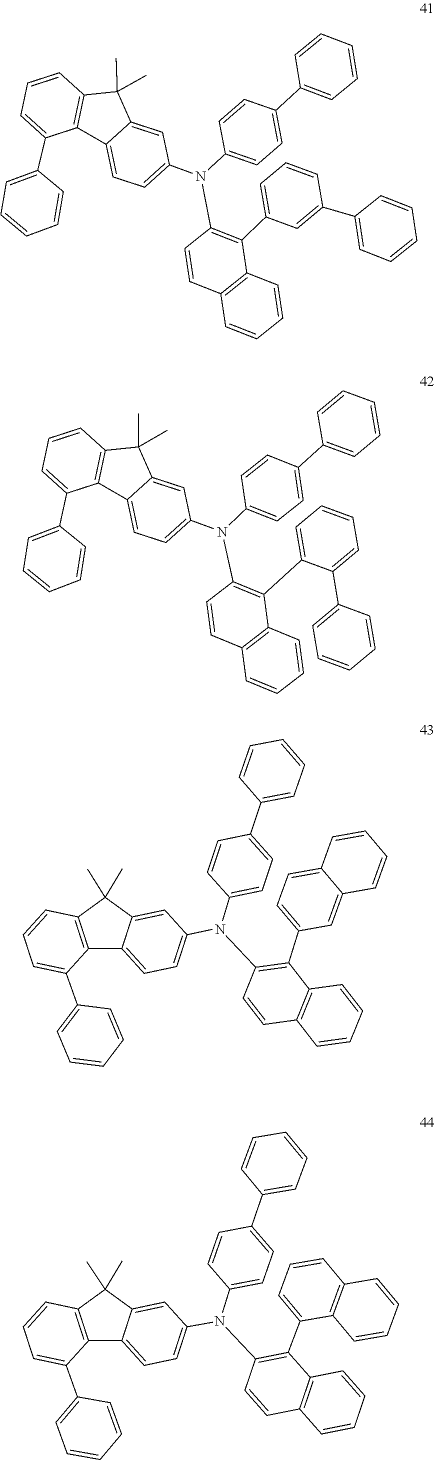

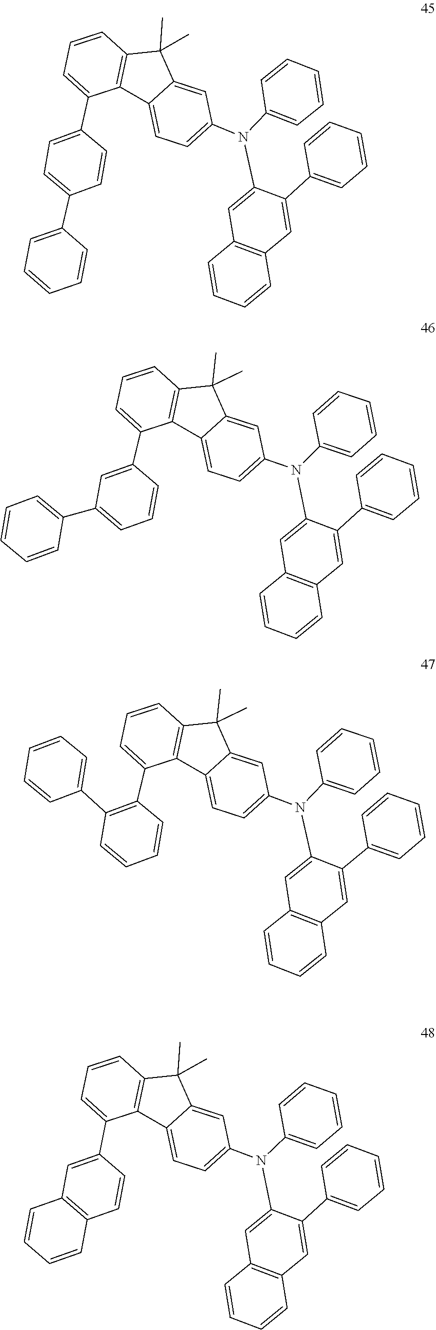 [SMILES: CC1(C)c2cc(N(c3ccccc3)c3cc4ccccc4cc3-c3ccccc3)ccc2-c2c(-c3ccc(-c4ccccc4)cc3)cccc21 ; CC1(C)c2cc(N(c3ccccc3)c3cc4ccccc4cc3-c3ccccc3)ccc2-c2c(-c3cccc(-c4ccccc4)c3)cccc21 ; CC1(C)c2cc(N(c3ccccc3)c3cc4ccccc4cc3-c3ccccc3)ccc2-c2c(-c3ccccc3-c3ccccc3)cccc21 ; CC1(C)c2cc(N(c3ccccc3)c3cc4ccccc4cc3-c3ccccc3)ccc2-c2c(-c3ccc4ccccc4c3)cccc21]

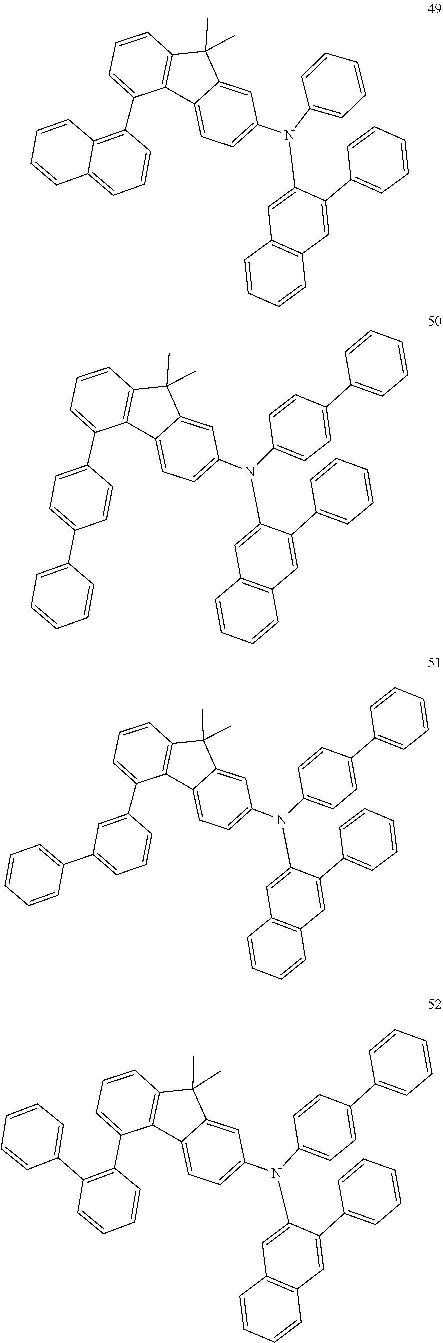 [SMILES: CC1(C)c2cc(N(c3ccccc3)c3cc4ccccc4cc3-c3ccccc3)ccc2-c2c(-c3cccc4ccccc34)cccc21 ; CC1(C)c2cc(N(c3ccc(-c4ccccc4)cc3)c3cc4ccccc4cc3-c3ccccc3)ccc2-c2c(-c3ccc(-c4ccccc4)cc3)cccc21 ; CC1(C)c2cc(N(c3ccc(-c4ccccc4)cc3)c3cc4ccccc4cc3-c3ccccc3)ccc2-c2c(-c3cccc(-c4ccccc4)c3)cccc21 ; CC1(C)c2cc(N(c3ccc(-c4ccccc4)cc3)c3cc4ccccc4cc3-c3ccccc3)ccc2-c2c(-c3ccccc3-c3ccccc3)cccc21]

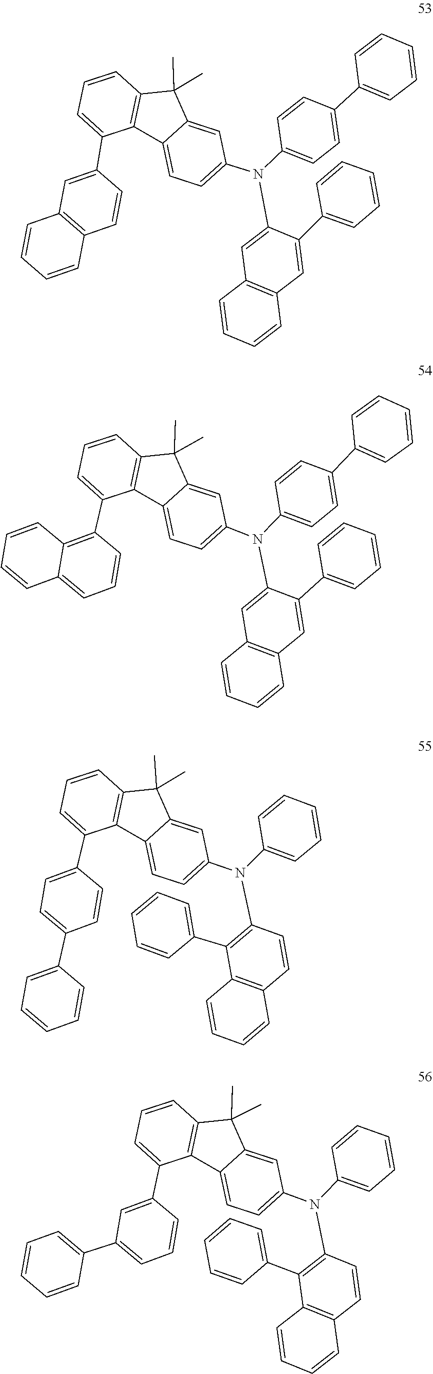

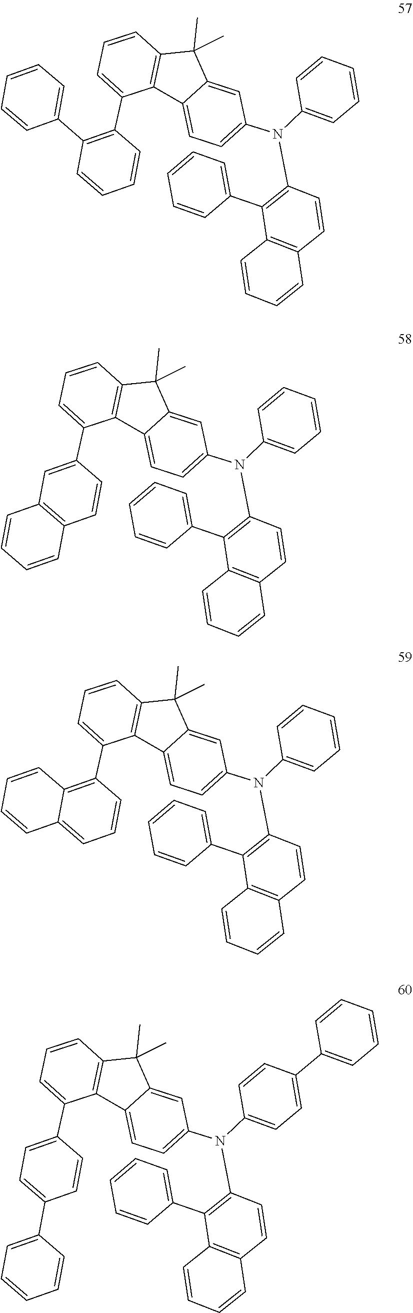 [SMILES: CC1(C)c2cc(N(c3ccccc3)c3ccc4ccccc4c3-c3ccccc3)ccc2-c2c(-c3ccccc3-c3ccccc3)cccc21 ; CC1(C)c2cc(N(c3ccccc3)c3ccc4ccccc4c3-c3ccccc3)ccc2-c2c(-c3ccc4ccccc4c3)cccc21 ; CC1(C)c2cc(N(c3ccccc3)c3ccc4ccccc4c3-c3ccccc3)ccc2-c2c(-c3cccc4ccccc34)cccc21 ; CC1(C)c2cc(N(c3ccc(-c4ccccc4)cc3)c3ccc4ccccc4c3-c3ccccc3)ccc2-c2c(-c3ccc(-c4ccccc4)cc3)cccc21]

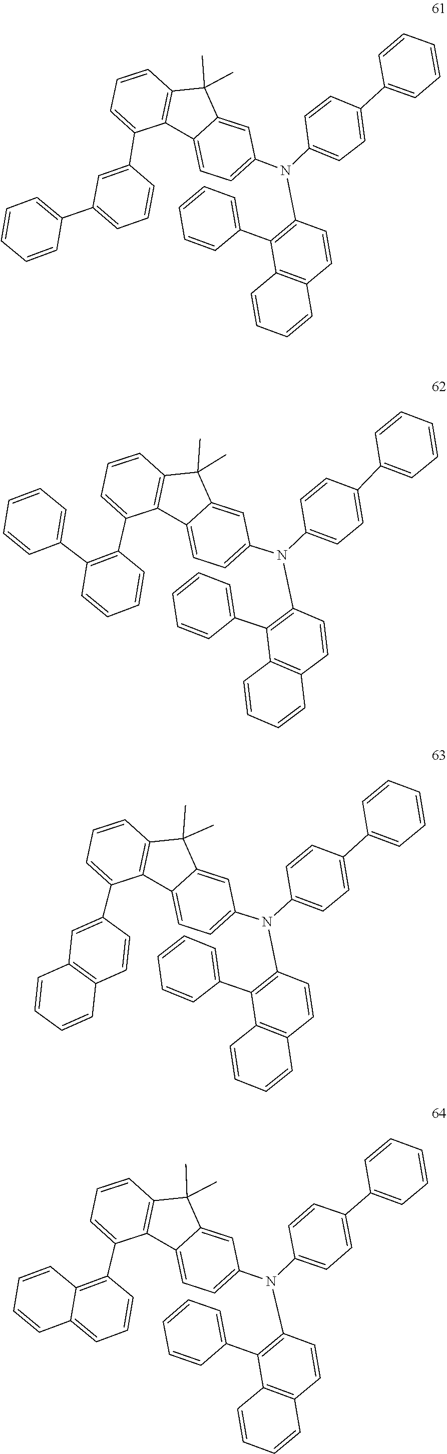 [SMILES: CC1(C)c2cc(N(c3ccc(-c4ccccc4)cc3)c3ccc4ccccc4c3-c3ccccc3)ccc2-c2c(-c3cccc(-c4ccccc4)c3)cccc21 ; CC1(C)c2cc(N(c3ccc(-c4ccccc4)cc3)c3ccc4ccccc4c3-c3ccccc3)ccc2-c2c(-c3ccccc3-c3ccccc3)cccc21 ; CC1(C)c2cc(N(c3ccc(-c4ccccc4)cc3)c3ccc4ccccc4c3-c3ccccc3)ccc2-c2c(-c3ccc4ccccc4c3)cccc21 ; CC1(C)c2cc(N(c3ccc(-c4ccccc4)cc3)c3ccc4ccccc4c3-c3ccccc3)ccc2-c2c(-c3cccc4ccccc34)cccc21]

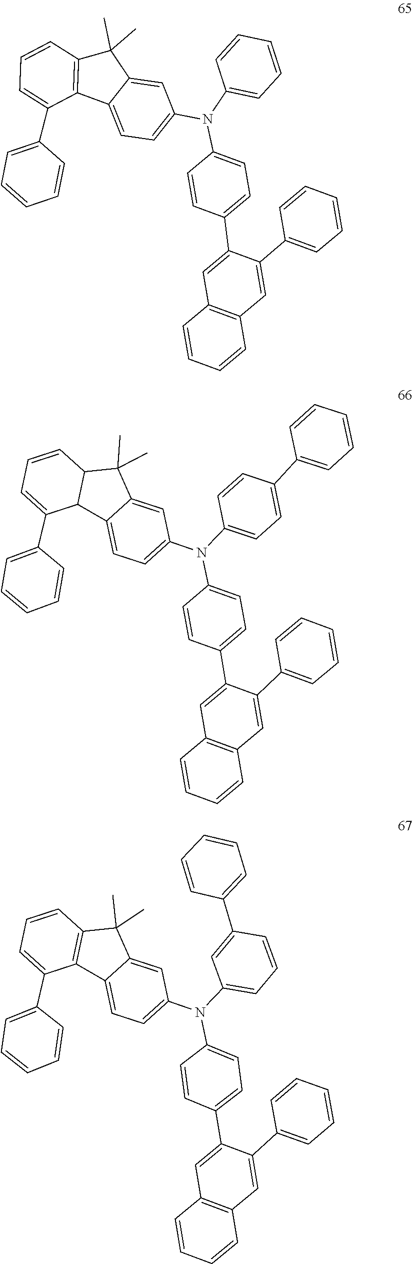

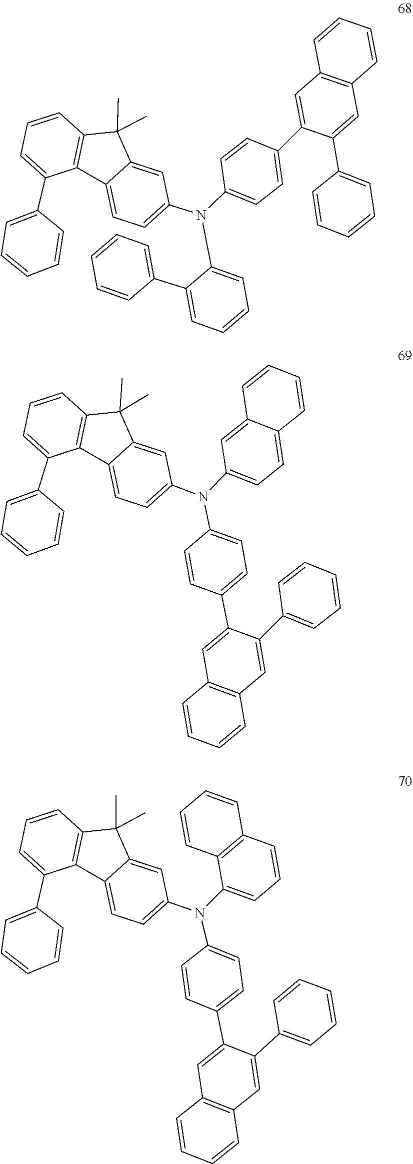

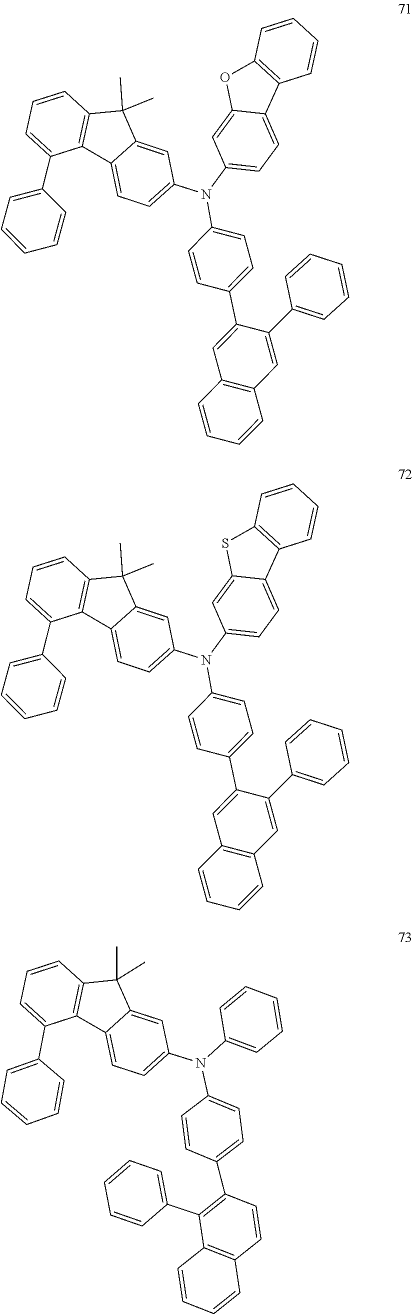 [SMILES: CC1(C)c2cc(N(c3ccc(-c4cc5ccccc5cc4-c4ccccc4)cc3)c3ccc4c(c3)oc3ccccc34)ccc2-c2c(-c3ccccc3)cccc21 ; CC1(C)c2cc(N(c3ccc(-c4cc5ccccc5cc4-c4ccccc4)cc3)c3ccc4c(c3)sc3ccccc34)ccc2-c2c(-c3ccccc3)cccc21 ; CC1(C)c2cc(N(c3ccccc3)c3ccc(-c4ccc5ccccc5c4-c4ccccc4)cc3)ccc2-c2c(-c3ccccc3)cccc21]

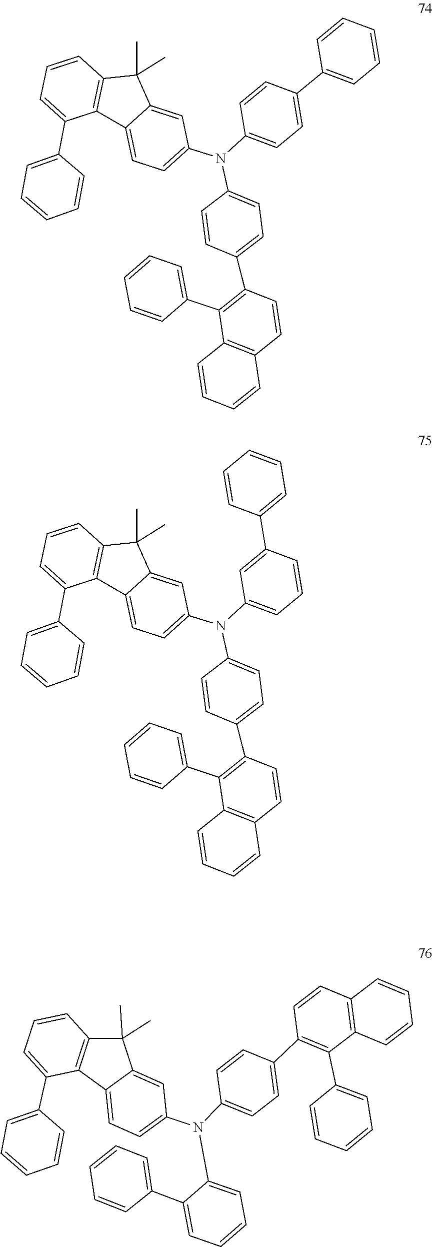

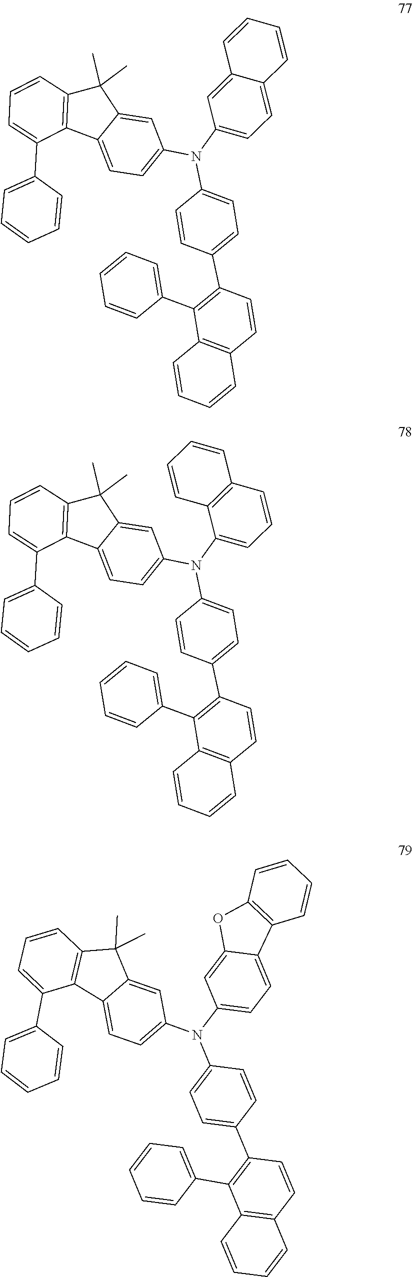

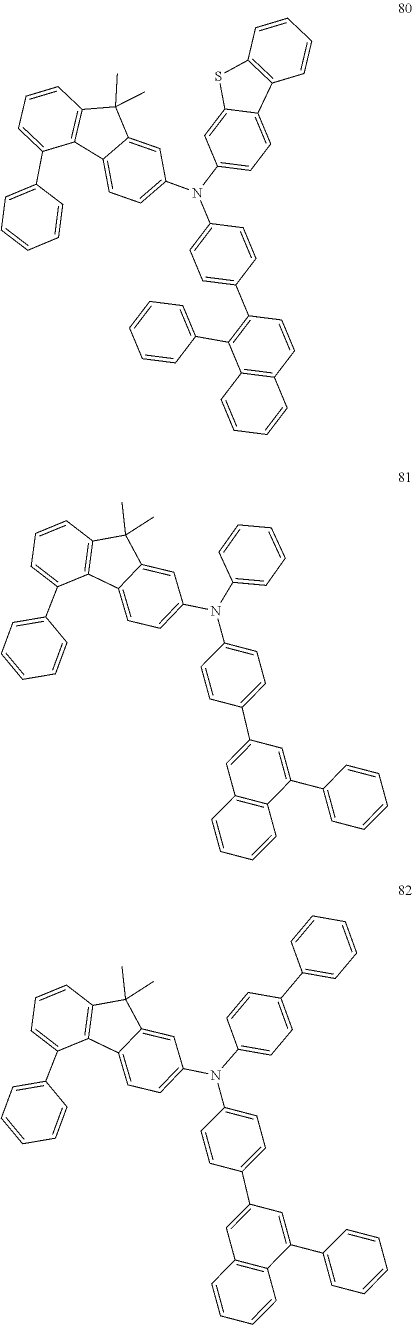 [SMILES: CC1(C)c2cc(N(c3ccc(-c4ccc5ccccc5c4-c4ccccc4)cc3)c3ccc4c(c3)sc3ccccc34)ccc2-c2c(-c3ccccc3)cccc21 ; CC1(C)c2cc(N(c3ccccc3)c3ccc(-c4cc(-c5ccccc5)c5ccccc5c4)cc3)ccc2-c2c(-c3ccccc3)cccc21 ; CC1(C)c2cc(N(c3ccc(-c4ccccc4)cc3)c3ccc(-c4cc(-c5ccccc5)c5ccccc5c4)cc3)ccc2-c2c(-c3ccccc3)cccc21]

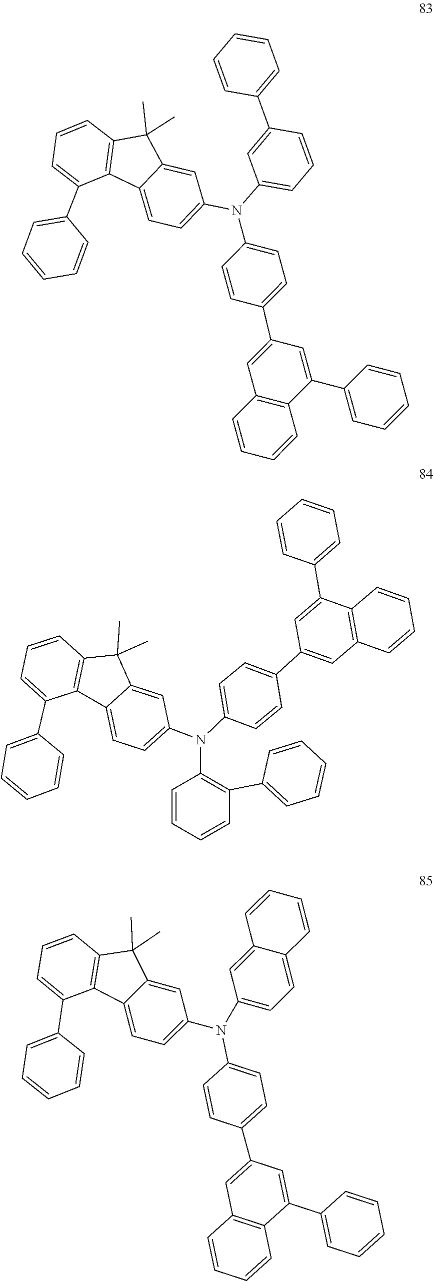 [SMILES: CC1(C)c2cc(N(c3ccc(-c4cc(-c5ccccc5)c5ccccc5c4)cc3)c3cccc(-c4ccccc4)c3)ccc2-c2c(-c3ccccc3)cccc21 ; CC1(C)c2cc(N(c3ccc(-c4cc(-c5ccccc5)c5ccccc5c4)cc3)c3ccccc3-c3ccccc3)ccc2-c2c(-c3ccccc3)cccc21 ; CC1(C)c2cc(N(c3ccc(-c4cc(-c5ccccc5)c5ccccc5c4)cc3)c3ccc4ccccc4c3)ccc2-c2c(-c3ccccc3)cccc21]

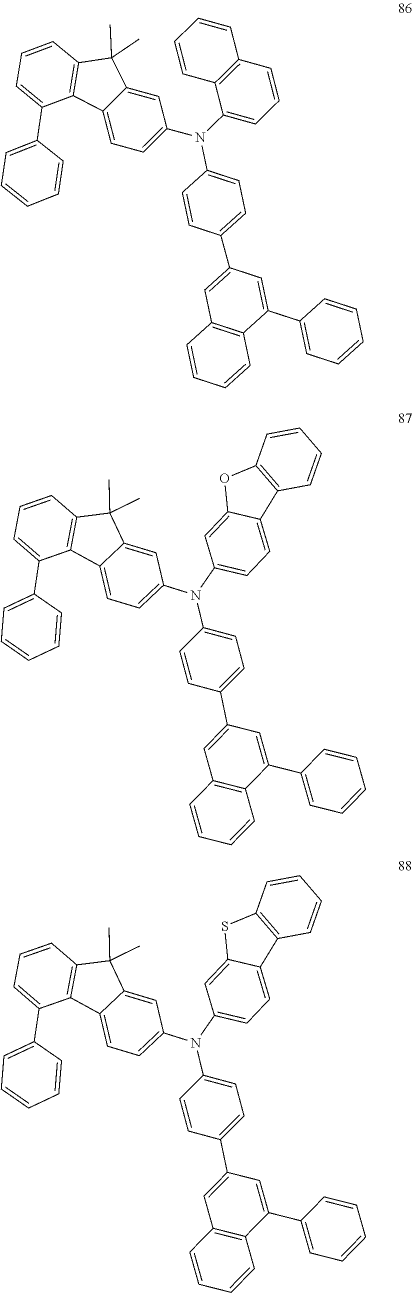 [SMILES: CC1(C)c2cc(N(c3ccc(-c4cc(-c5ccccc5)c5ccccc5c4)cc3)c3cccc4ccccc34)ccc2-c2c(-c3ccccc3)cccc21 ; CC1(C)c2cc(N(c3ccc(-c4cc(-c5ccccc5)c5ccccc5c4)cc3)c3ccc4c(c3)oc3ccccc34)ccc2-c2c(-c3ccccc3)cccc21 ; CC1(C)c2cc(N(c3ccc(-c4cc(-c5ccccc5)c5ccccc5c4)cc3)c3ccc4c(c3)sc3ccccc34)ccc2-c2c(-c3ccccc3)cccc21]

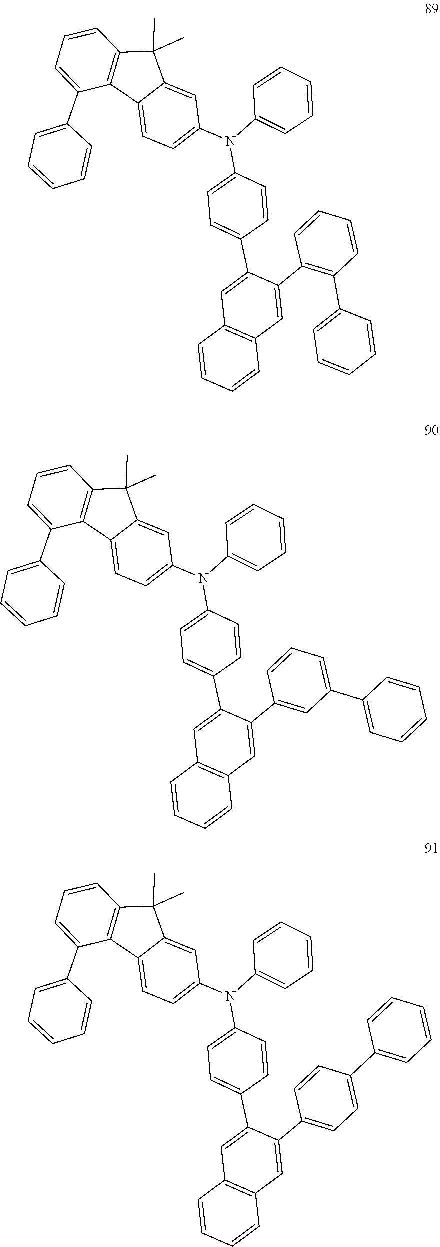 [SMILES: CC1(C)c2cc(N(c3ccccc3)c3ccc(-c4cc5ccccc5cc4-c4ccccc4-c4ccccc4)cc3)ccc2-c2c(-c3ccccc3)cccc21 ; CC1(C)c2cc(N(c3ccccc3)c3ccc(-c4cc5ccccc5cc4-c4cccc(-c5ccccc5)c4)cc3)ccc2-c2c(-c3ccccc3)cccc21 ; CC1(C)c2cc(N(c3ccccc3)c3ccc(-c4cc5ccccc5cc4-c4ccc(-c5ccccc5)cc4)cc3)ccc2-c2c(-c3ccccc3)cccc21]

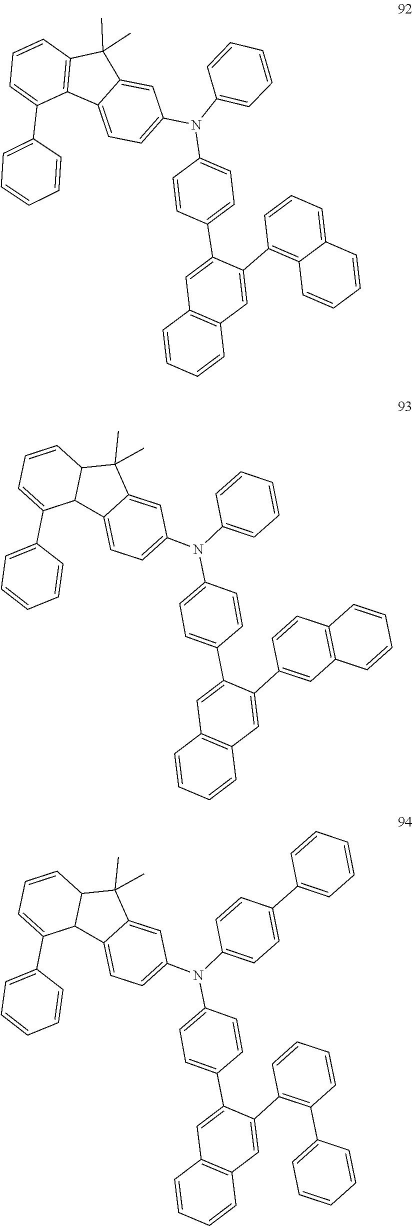 [SMILES: CC1(C)c2cc(N(c3ccccc3)c3ccc(-c4cc5ccccc5cc4-c4cccc5ccccc45)cc3)ccc2-c2c(-c3ccccc3)cccc21 ; CC1(C)c2cc(N(c3ccccc3)c3ccc(-c4cc5ccccc5cc4-c4ccc5ccccc5c4)cc3)ccc2C2C(c3ccccc3)=CC=CC21 ; CC1(C)c2cc(N(c3ccc(-c4ccccc4)cc3)c3ccc(-c4cc5ccccc5cc4-c4ccccc4-c4ccccc4)cc3)ccc2C2C(c3ccccc3)=CC=CC21]

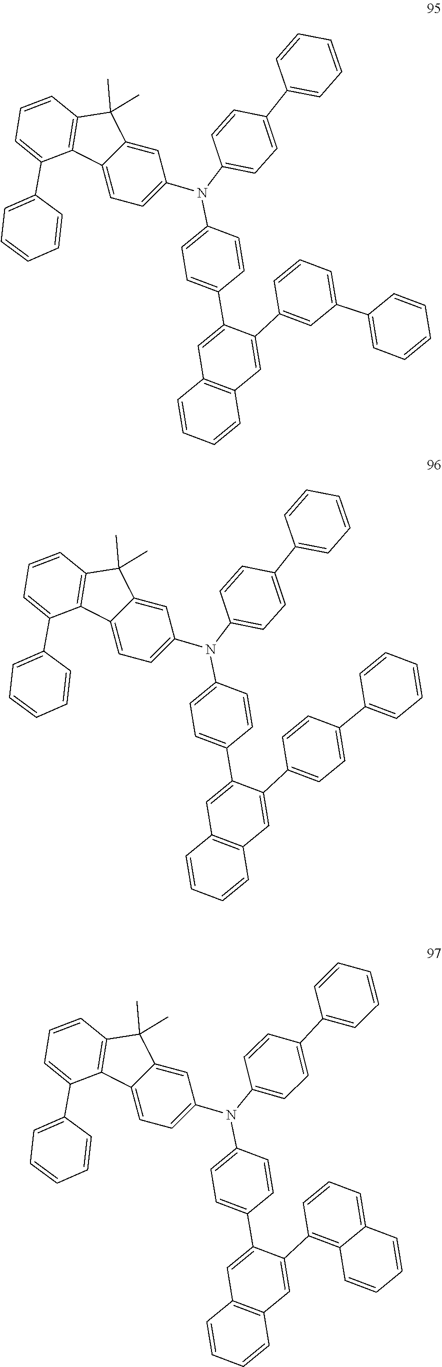 [SMILES: CC1(C)c2cc(N(c3ccc(-c4ccccc4)cc3)c3ccc(-c4cc5ccccc5cc4-c4cccc(-c5ccccc5)c4)cc3)ccc2-c2c(-c3ccccc3)cccc21 ; CC1(C)c2cc(N(c3ccc(-c4ccccc4)cc3)c3ccc(-c4cc5ccccc5cc4-c4ccc(-c5ccccc5)cc4)cc3)ccc2-c2c(-c3ccccc3)cccc21 ; CC1(C)c2cc(N(c3ccc(-c4ccccc4)cc3)c3ccc(-c4cc5ccccc5cc4-c4cccc5ccccc45)cc3)ccc2-c2c(-c3ccccc3)cccc21]

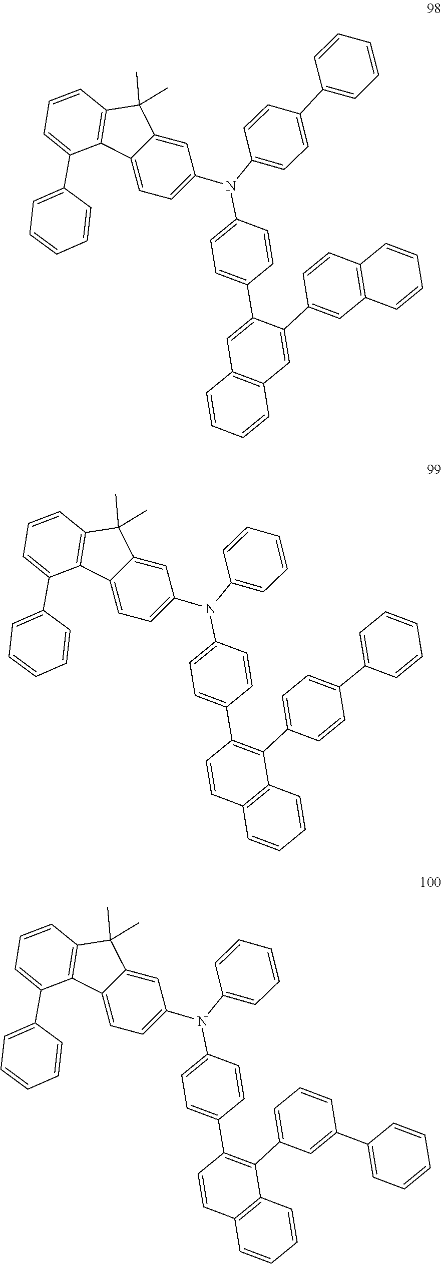 [SMILES: CC1(C)c2cc(N(c3ccc(-c4ccccc4)cc3)c3ccc(-c4cc5ccccc5cc4-c4ccc5ccccc5c4)cc3)ccc2-c2c(-c3ccccc3)cccc21 ; CC1(C)c2cc(N(c3ccccc3)c3ccc(-c4ccc5ccccc5c4-c4ccc(-c5ccccc5)cc4)cc3)ccc2-c2c(-c3ccccc3)cccc21 ; CC1(C)c2cc(N(c3ccccc3)c3ccc(-c4ccc5ccccc5c4-c4cccc(-c5ccccc5)c4)cc3)ccc2-c2c(-c3ccccc3)cccc21]

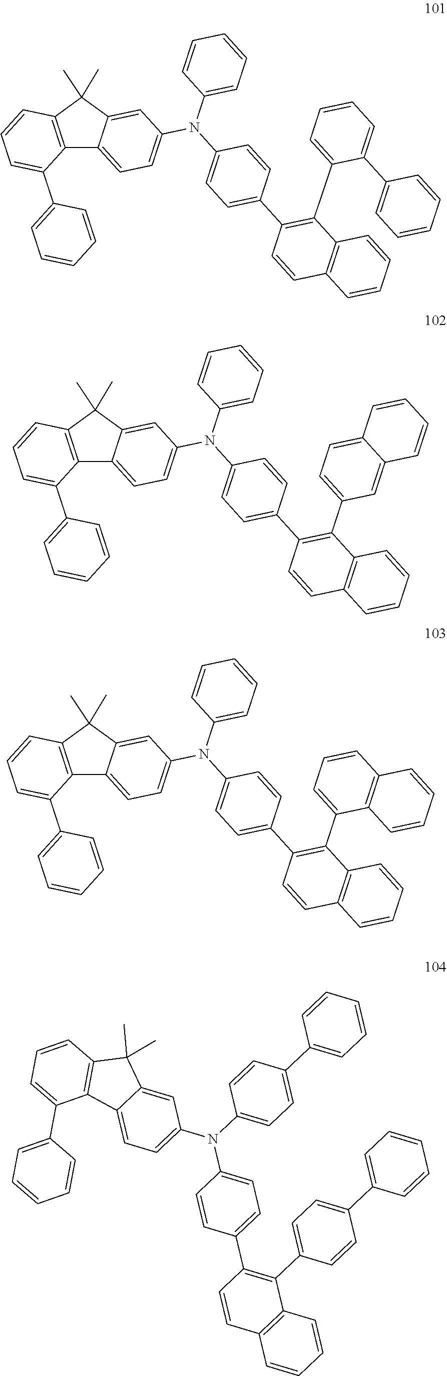 [SMILES: CC1(C)c2cc(N(c3ccccc3)c3ccc(-c4ccc5ccccc5c4-c4ccccc4-c4ccccc4)cc3)ccc2-c2c(-c3ccccc3)cccc21 ; CC1(C)c2cc(N(c3ccccc3)c3ccc(-c4ccc5ccccc5c4-c4ccc5ccccc5c4)cc3)ccc2-c2c(-c3ccccc3)cccc21 ; CC1(C)c2cc(N(c3ccccc3)c3ccc(-c4ccc5ccccc5c4-c4cccc5ccccc45)cc3)ccc2-c2c(-c3ccccc3)cccc21 ; CC1(C)c2cc(N(c3ccc(-c4ccccc4)cc3)c3ccc(-c4ccc5ccccc5c4-c4ccc(-c5ccccc5)cc4)cc3)ccc2-c2c(-c3ccccc3)cccc21]

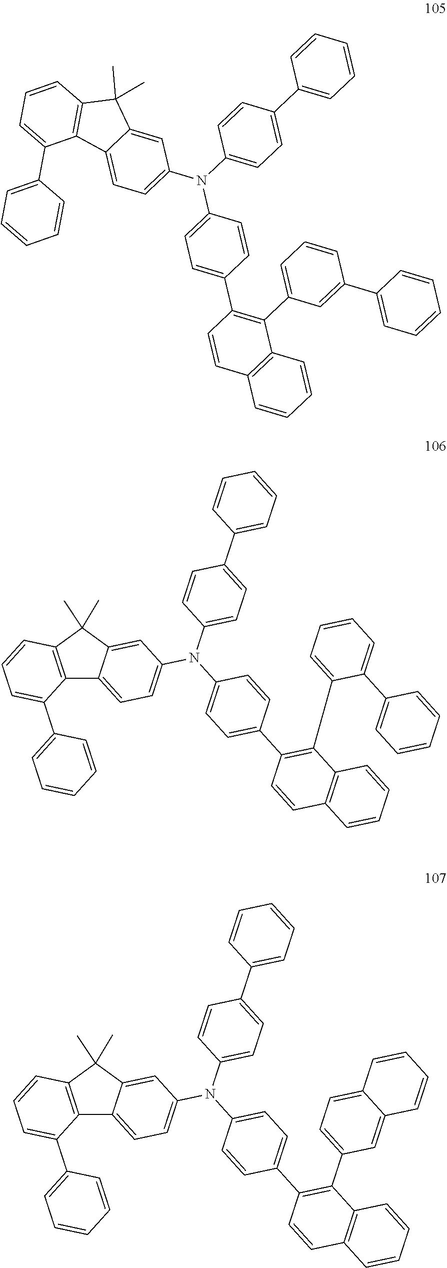 [SMILES: CC1(C)c2cc(N(c3ccc(-c4ccccc4)cc3)c3ccc(-c4ccc5ccccc5c4-c4cccc(-c5ccccc5)c4)cc3)ccc2-c2c(-c3ccccc3)cccc21 ; CC1(C)c2cc(N(c3ccc(-c4ccccc4)cc3)c3ccc(-c4ccc5ccccc5c4-c4ccccc4-c4ccccc4)cc3)ccc2-c2c(-c3ccccc3)cccc21 ; CC1(C)c2cc(N(c3ccc(-c4ccccc4)cc3)c3ccc(-c4ccc5ccccc5c4-c4ccc5ccccc5c4)cc3)ccc2-c2c(-c3ccccc3)cccc21]

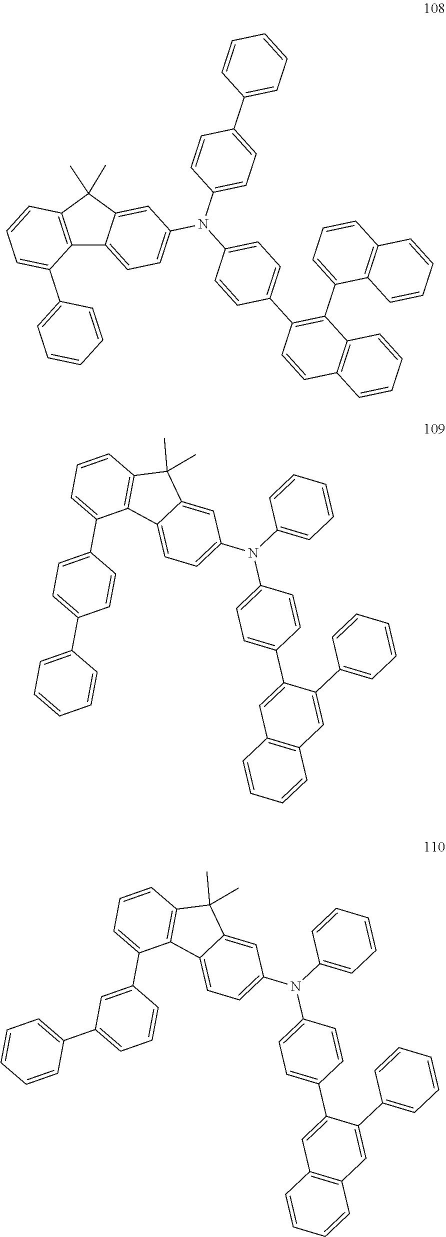 [SMILES: CC1(C)c2cc(N(c3ccc(-c4ccccc4)cc3)c3ccc(-c4ccc5ccccc5c4-c4cccc5ccccc45)cc3)ccc2-c2c(-c3ccccc3)cccc21 ; CC1(C)c2cc(N(c3ccccc3)c3ccc(-c4cc5ccccc5cc4-c4ccccc4)cc3)ccc2-c2c(-c3ccc(-c4ccccc4)cc3)cccc21 ; CC1(C)c2cc(N(c3ccccc3)c3ccc(-c4cc5ccccc5cc4-c4ccccc4)cc3)ccc2-c2c(-c3cccc(-c4ccccc4)c3)cccc21]

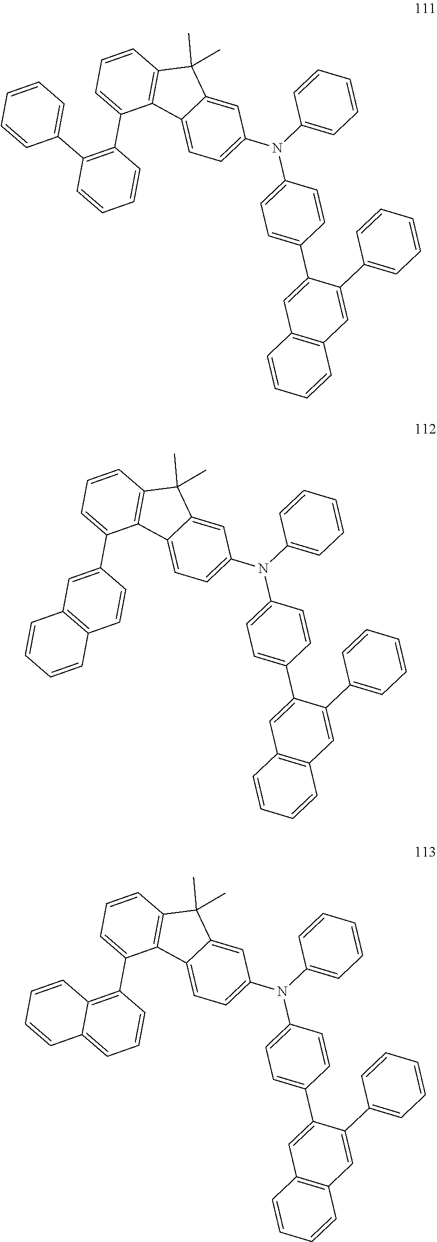 [SMILES: CC1(C)c2cc(N(c3ccccc3)c3ccc(-c4cc5ccccc5cc4-c4ccccc4)cc3)ccc2-c2c(-c3ccccc3-c3ccccc3)cccc21 ; CC1(C)c2cc(N(c3ccccc3)c3ccc(-c4cc5ccccc5cc4-c4ccccc4)cc3)ccc2-c2c(-c3ccc4ccccc4c3)cccc21 ; CC1(C)c2cc(N(c3ccccc3)c3ccc(-c4cc5ccccc5cc4-c4ccccc4)cc3)ccc2-c2c(-c3cccc4ccccc34)cccc21]

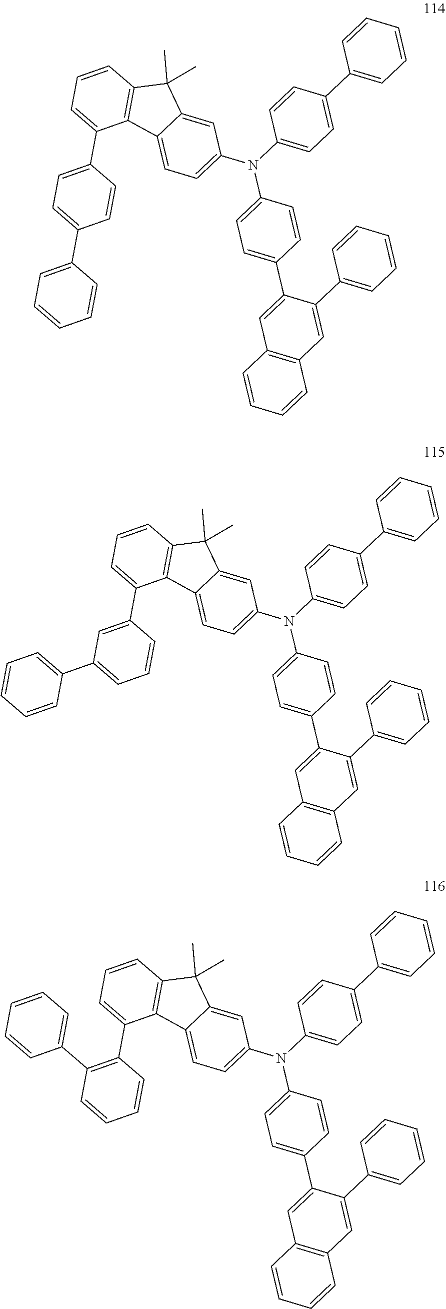 [SMILES: CC1(C)c2cc(N(c3ccc(-c4ccccc4)cc3)c3ccc(-c4cc5ccccc5cc4-c4ccccc4)cc3)ccc2-c2c(-c3ccc(-c4ccccc4)cc3)cccc21 ; CC1(C)c2cc(N(c3ccc(-c4ccccc4)cc3)c3ccc(-c4cc5ccccc5cc4-c4ccccc4)cc3)ccc2-c2c(-c3cccc(-c4ccccc4)c3)cccc21 ; CC1(C)c2cc(N(c3ccc(-c4ccccc4)cc3)c3ccc(-c4cc5ccccc5cc4-c4ccccc4)cc3)ccc2-c2c(-c3ccccc3-c3ccccc3)cccc21]

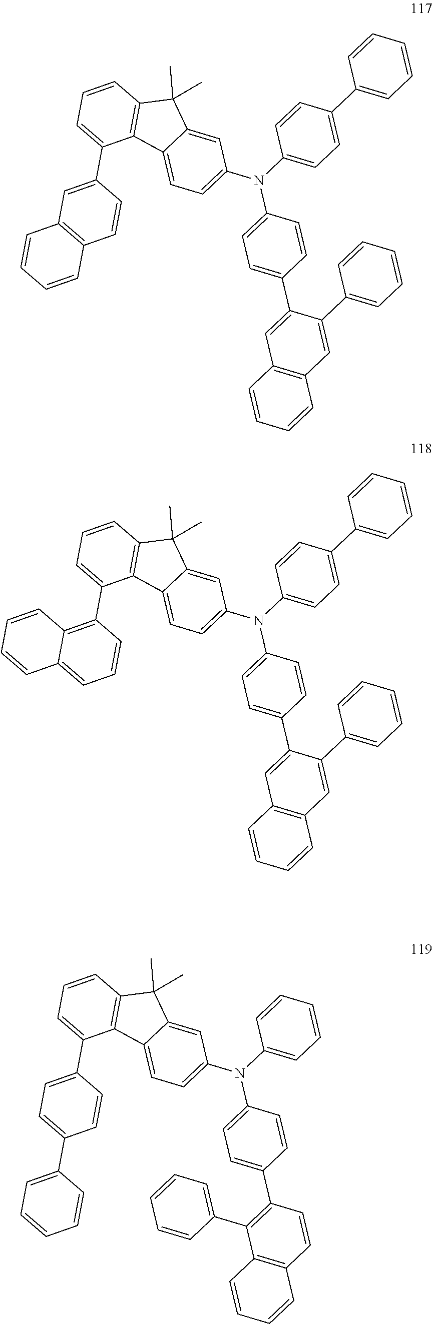 [SMILES: CC1(C)c2cc(N(c3ccc(-c4ccccc4)cc3)c3ccc(-c4cc5ccccc5cc4-c4ccccc4)cc3)ccc2-c2c(-c3ccc4ccccc4c3)cccc21 ; CC1(C)c2cc(N(c3ccc(-c4ccccc4)cc3)c3ccc(-c4cc5ccccc5cc4-c4ccccc4)cc3)ccc2-c2c(-c3cccc4ccccc34)cccc21 ; CC1(C)c2cc(N(c3ccccc3)c3ccc(-c4ccc5ccccc5c4-c4ccccc4)cc3)ccc2-c2c(-c3ccc(-c4ccccc4)cc3)cccc21]

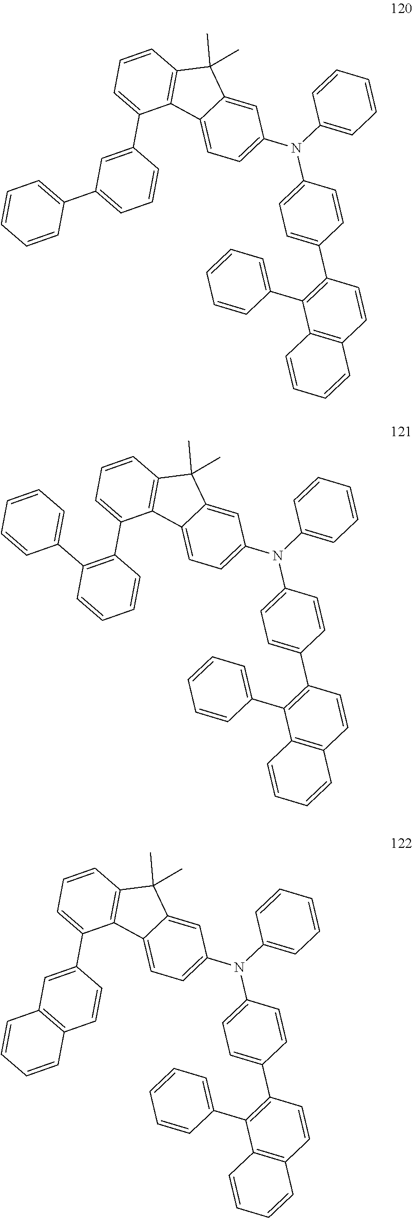 [SMILES: CC1(C)c2cc(N(c3ccccc3)c3ccc(-c4ccc5ccccc5c4-c4ccccc4)cc3)ccc2-c2c(-c3cccc(-c4ccccc4)c3)cccc21 ; CC1(C)c2cc(N(c3ccccc3)c3ccc(-c4ccc5ccccc5c4-c4ccccc4)cc3)ccc2-c2c(-c3ccccc3-c3ccccc3)cccc21 ; CC1(C)c2cc(N(c3ccccc3)c3ccc(-c4ccc5ccccc5c4-c4ccccc4)cc3)ccc2-c2c(-c3ccc4ccccc4c3)cccc21]

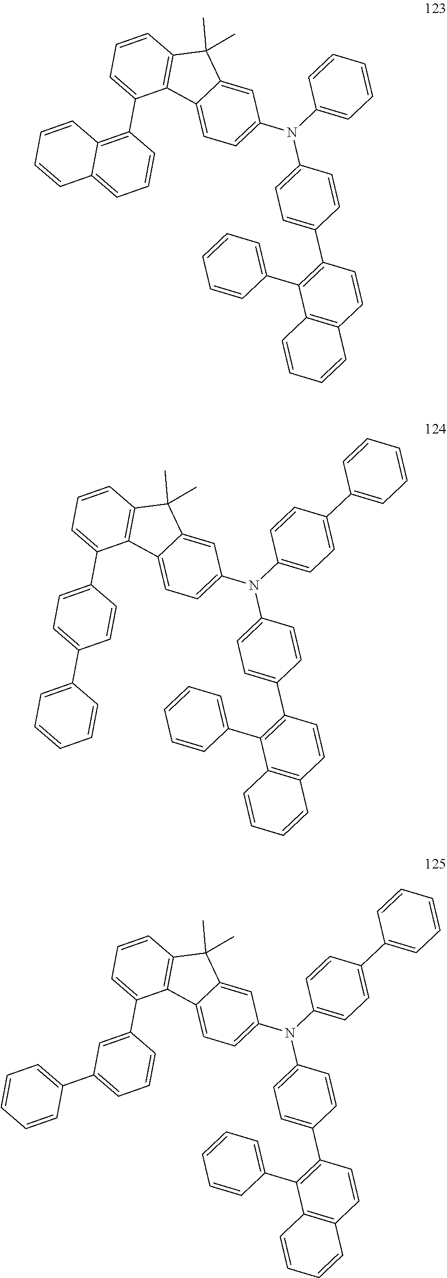 [SMILES: CC1(C)c2cc(N(c3ccccc3)c3ccc(-c4ccc5ccccc5c4-c4ccccc4)cc3)ccc2-c2c(-c3cccc4ccccc34)cccc21 ; CC1(C)c2cc(N(c3ccc(-c4ccccc4)cc3)c3ccc(-c4ccc5ccccc5c4-c4ccccc4)cc3)ccc2-c2c(-c3ccc(-c4ccccc4)cc3)cccc21 ; CC1(C)c2cc(N(c3ccc(-c4ccccc4)cc3)c3ccc(-c4ccc5ccccc5c4-c4ccccc4)cc3)ccc2-c2c(-c3cccc(-c4ccccc4)c3)cccc21]

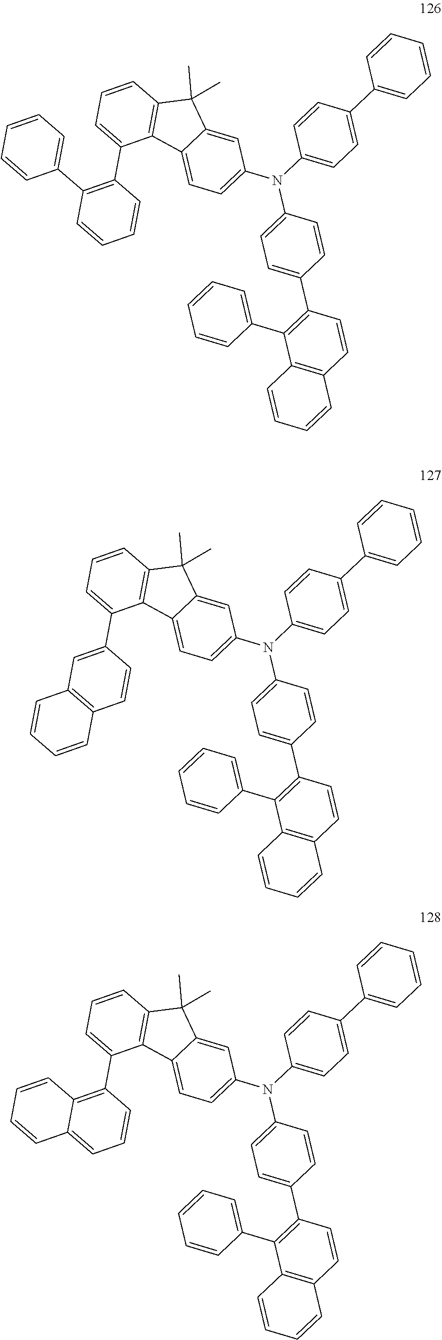 [SMILES: CC1(C)c2cc(N(c3ccc(-c4ccccc4)cc3)c3ccc(-c4ccc5ccccc5c4-c4ccccc4)cc3)ccc2-c2c(-c3ccccc3-c3ccccc3)cccc21 ; CC1(C)c2cc(N(c3ccc(-c4ccccc4)cc3)c3ccc(-c4ccc5ccccc5c4-c4ccccc4)cc3)ccc2-c2c(-c3ccc4ccccc4c3)cccc21 ; CC1(C)c2cc(N(c3ccc(-c4ccccc4)cc3)c3ccc(-c4ccc5ccccc5c4-c4ccccc4)cc3)ccc2-c2c(-c3cccc4ccccc34)cccc21]

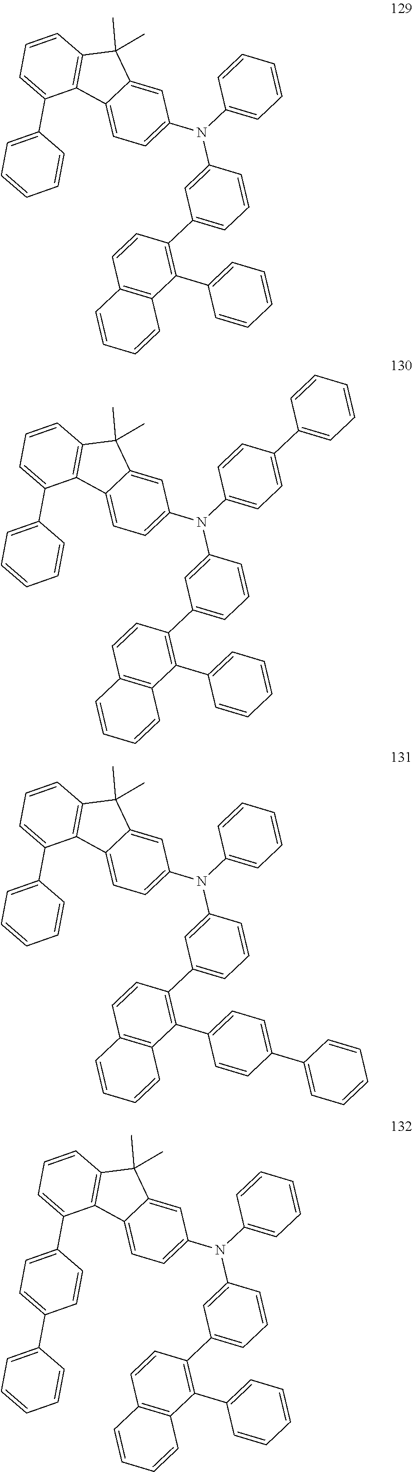

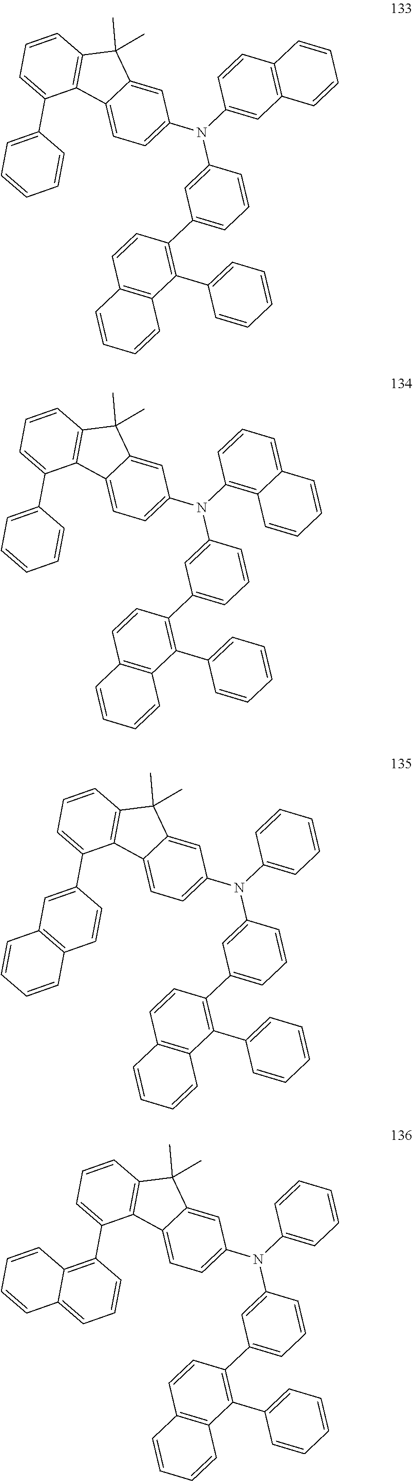

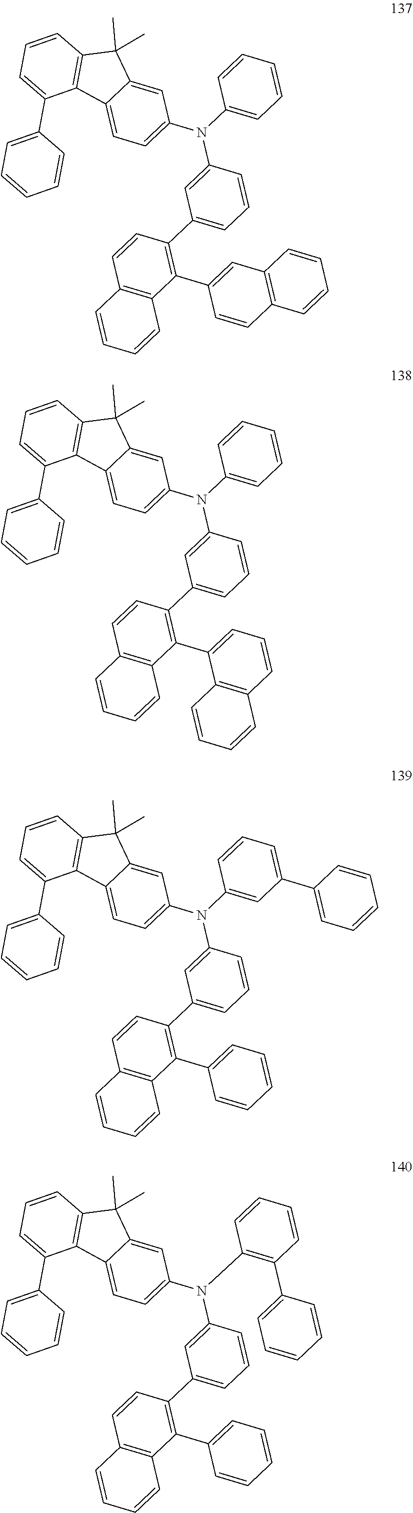

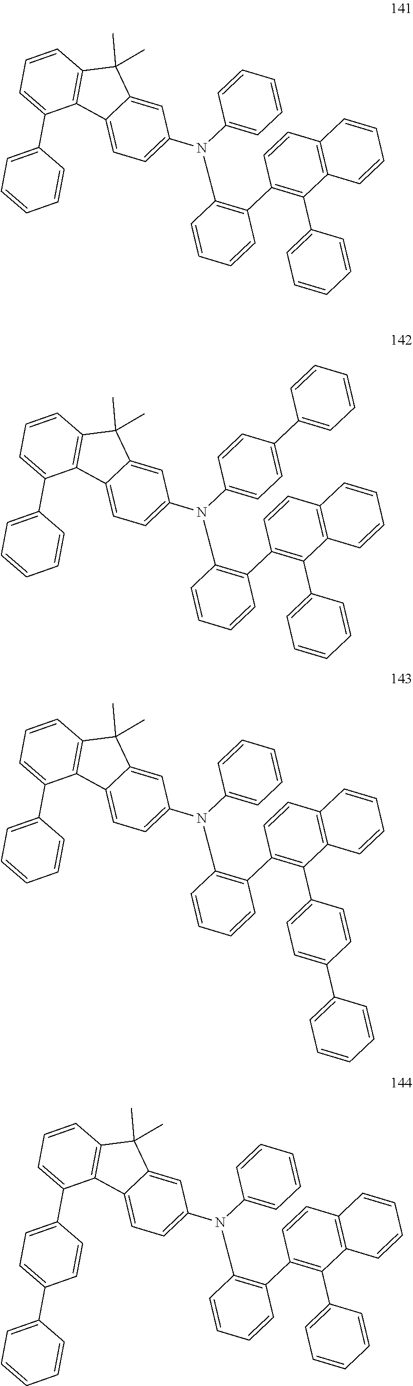 [SMILES: CC1(C)c2cc(N(c3ccccc3)c3ccccc3-c3ccc4ccccc4c3-c3ccccc3)ccc2-c2c(-c3ccccc3)cccc21 ; CC1(C)c2cc(N(c3ccc(-c4ccccc4)cc3)c3ccccc3-c3ccc4ccccc4c3-c3ccccc3)ccc2-c2c(-c3ccccc3)cccc21 ; CC1(C)c2cc(N(c3ccccc3)c3ccccc3-c3ccc4ccccc4c3-c3ccc(-c4ccccc4)cc3)ccc2-c2c(-c3ccccc3)cccc21 ; CC1(C)c2cc(N(c3ccccc3)c3ccccc3-c3ccc4ccccc4c3-c3ccccc3)ccc2-c2c(-c3ccc(-c4ccccc4)cc3)cccc21]

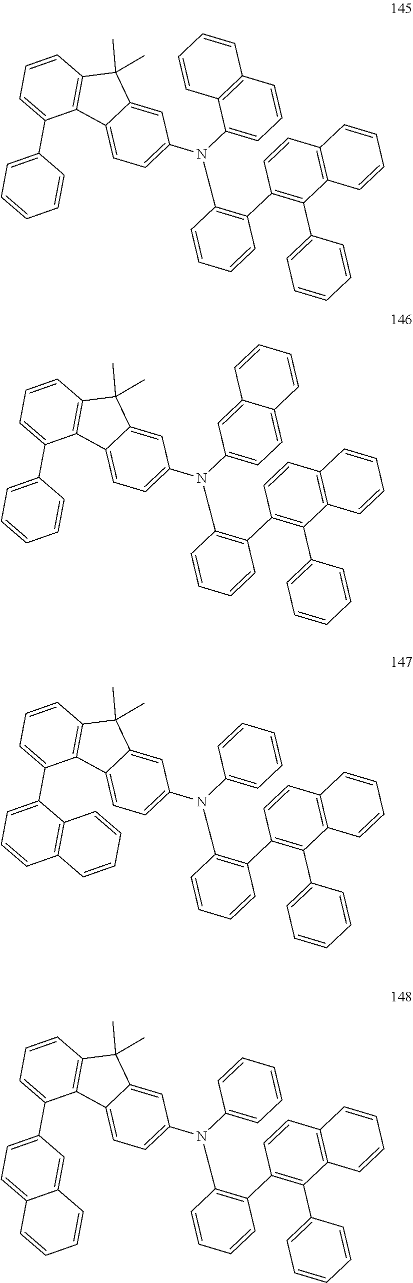 [SMILES: CC1(C)c2cc(N(c3ccccc3-c3ccc4ccccc4c3-c3ccccc3)c3cccc4ccccc34)ccc2-c2c(-c3ccccc3)cccc21 ; CC1(C)c2cc(N(c3ccc4ccccc4c3)c3ccccc3-c3ccc4ccccc4c3-c3ccccc3)ccc2-c2c(-c3ccccc3)cccc21 ; CC1(C)c2cc(N(c3ccccc3)c3ccccc3-c3ccc4ccccc4c3-c3ccccc3)ccc2-c2c(-c3cccc4ccccc34)cccc21 ; CC1(C)c2cc(N(c3ccccc3)c3ccccc3-c3ccc4ccccc4c3-c3ccccc3)ccc2-c2c(-c3ccc4ccccc4c3)cccc21]

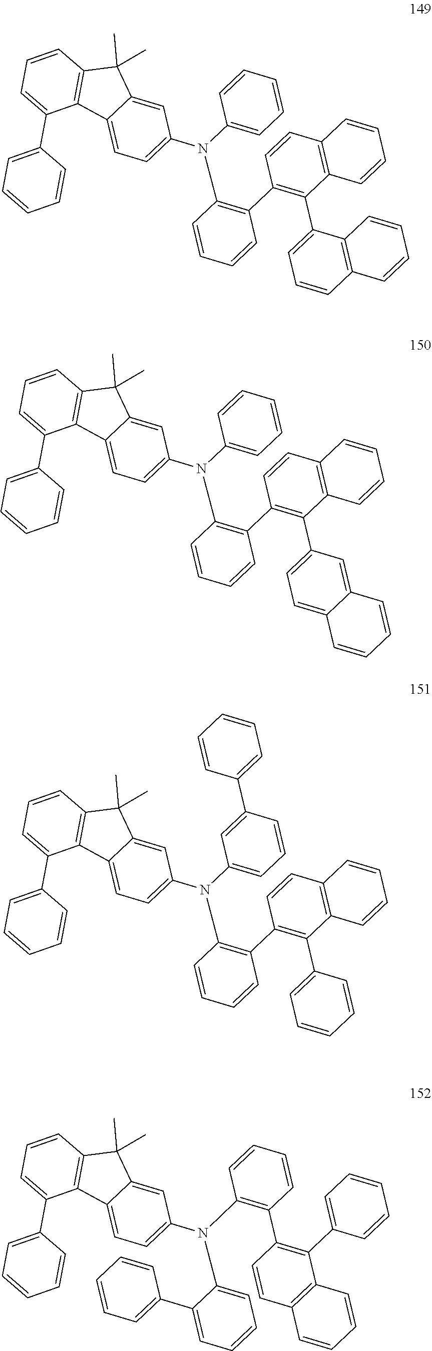 [SMILES: CC1(C)c2cc(N(c3ccccc3)c3ccccc3-c3ccc4ccccc4c3-c3cccc4ccccc34)ccc2-c2c(-c3ccccc3)cccc21 ; CC1(C)c2cc(N(c3ccccc3)c3ccccc3-c3ccc4ccccc4c3-c3ccc4ccccc4c3)ccc2-c2c(-c3ccccc3)cccc21 ; CC1(C)c2cc(N(c3cccc(-c4ccccc4)c3)c3ccccc3-c3ccc4ccccc4c3-c3ccccc3)ccc2-c2c(-c3ccccc3)cccc21 ; CC1(C)c2cc(N(c3ccccc3-c3ccccc3)c3ccccc3-c3ccc4ccccc4c3-c3ccccc3)ccc2-c2c(-c3ccccc3)cccc21]

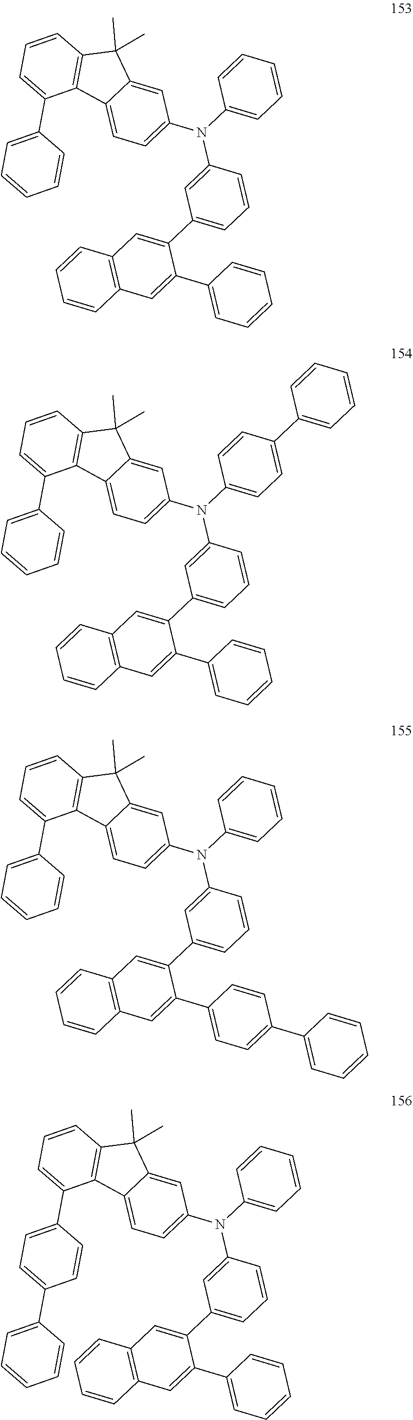 [SMILES: CC1(C)c2cc(N(c3ccccc3)c3cccc(-c4cc5ccccc5cc4-c4ccccc4)c3)ccc2-c2c(-c3ccccc3)cccc21 ; CC1(C)c2cc(N(c3ccc(-c4ccccc4)cc3)c3cccc(-c4cc5ccccc5cc4-c4ccccc4)c3)ccc2-c2c(-c3ccccc3)cccc21 ; CC1(C)c2cc(N(c3ccccc3)c3cccc(-c4cc5ccccc5cc4-c4ccc(-c5ccccc5)cc4)c3)ccc2-c2c(-c3ccccc3)cccc21 ; CC1(C)c2cc(N(c3ccccc3)c3cccc(-c4cc5ccccc5cc4-c4ccccc4)c3)ccc2-c2c(-c3ccc(-c4ccccc4)cc3)cccc21]

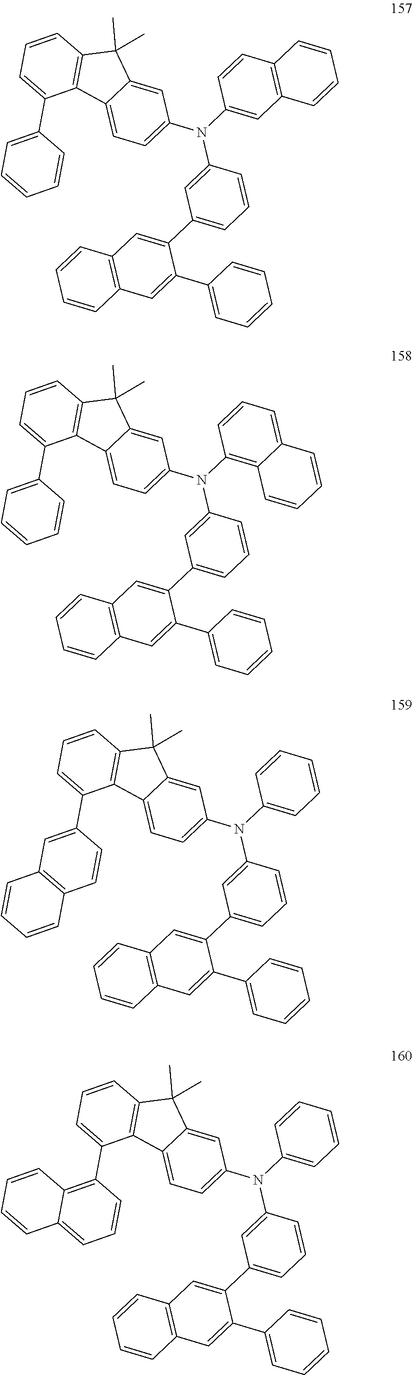 [SMILES: CC1(C)c2cc(N(c3cccc(-c4cc5ccccc5cc4-c4ccccc4)c3)c3ccc4ccccc4c3)ccc2-c2c(-c3ccccc3)cccc21 ; CC1(C)c2cc(N(c3cccc(-c4cc5ccccc5cc4-c4ccccc4)c3)c3cccc4ccccc34)ccc2-c2c(-c3ccccc3)cccc21 ; CC1(C)c2cc(N(c3ccccc3)c3cccc(-c4cc5ccccc5cc4-c4ccccc4)c3)ccc2-c2c(-c3ccc4ccccc4c3)cccc21 ; CC1(C)c2cc(N(c3ccccc3)c3cccc(-c4cc5ccccc5cc4-c4ccccc4)c3)ccc2-c2c(-c3cccc4ccccc34)cccc21]

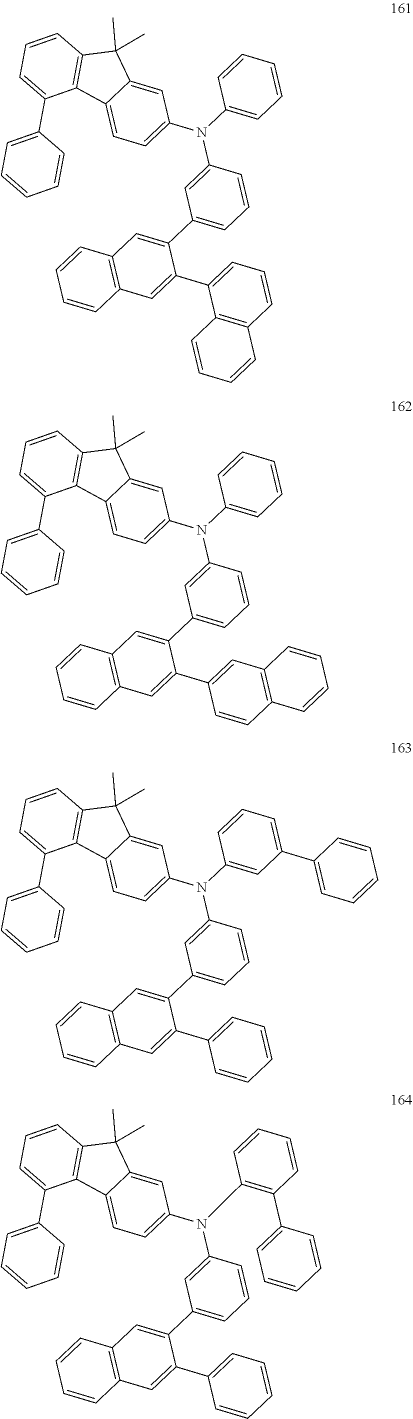

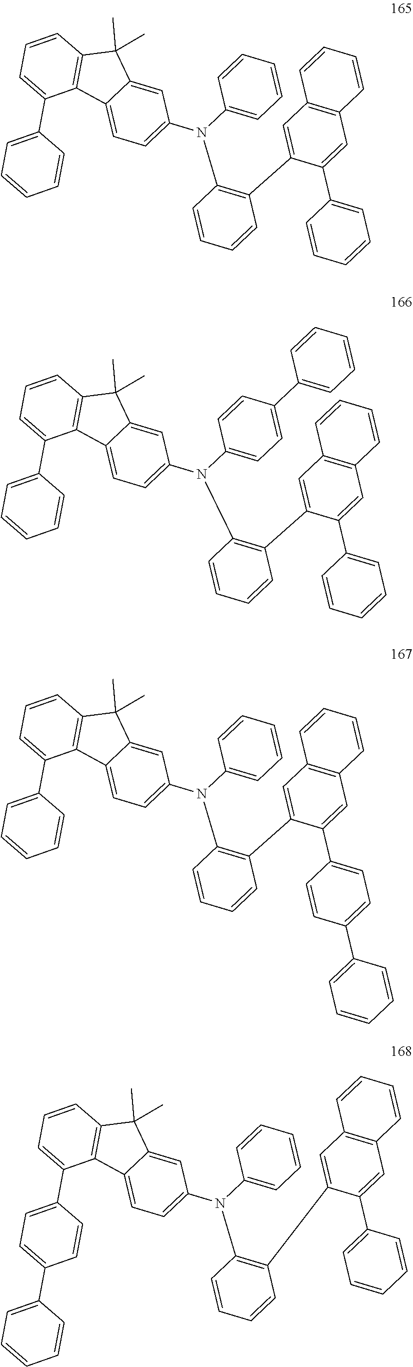 [SMILES: CC1(C)c2cc(N(c3ccccc3)c3ccccc3-c3cc4ccccc4cc3-c3ccccc3)ccc2-c2c(-c3ccccc3)cccc21 ; CC1(C)c2cc(N(c3ccc(-c4ccccc4)cc3)c3ccccc3-c3cc4ccccc4cc3-c3ccccc3)ccc2-c2c(-c3ccccc3)cccc21 ; CC1(C)c2cc(N(c3ccccc3)c3ccccc3-c3cc4ccccc4cc3-c3ccc(-c4ccccc4)cc3)ccc2-c2c(-c3ccccc3)cccc21 ; CC1(C)c2cc(N(c3ccccc3)c3ccccc3-c3cc4ccccc4cc3-c3ccccc3)ccc2-c2c(-c3ccc(-c4ccccc4)cc3)cccc21]

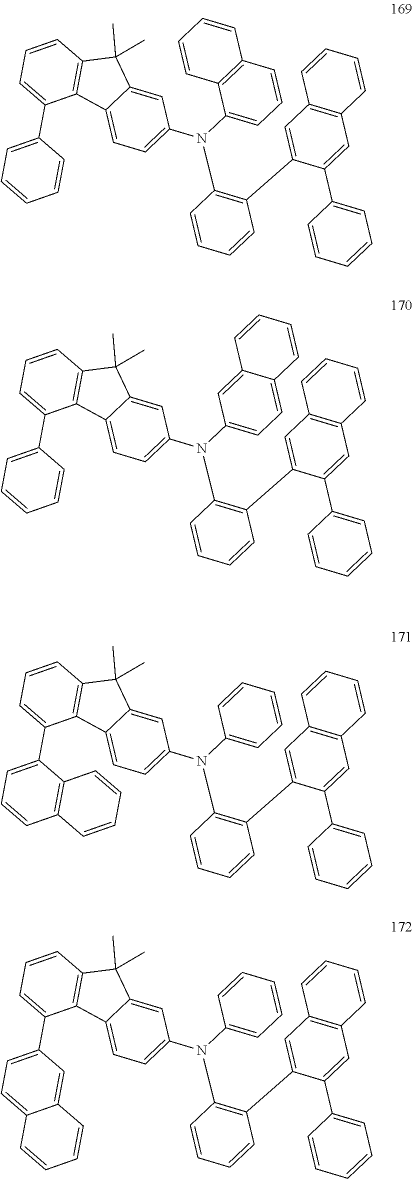 [SMILES: CC1(C)c2cc(N(c3ccccc3-c3cc4ccccc4cc3-c3ccccc3)c3cccc4ccccc34)ccc2-c2c(-c3ccccc3)cccc21 ; CC1(C)c2cc(N(c3ccc4ccccc4c3)c3ccccc3-c3cc4ccccc4cc3-c3ccccc3)ccc2-c2c(-c3ccccc3)cccc21 ; CC1(C)c2cc(N(c3ccccc3)c3ccccc3-c3cc4ccccc4cc3-c3ccccc3)ccc2-c2c(-c3cccc4ccccc34)cccc21 ; CC1(C)c2cc(N(c3ccccc3)c3ccccc3-c3cc4ccccc4cc3-c3ccccc3)ccc2-c2c(-c3ccc4ccccc4c3)cccc21]

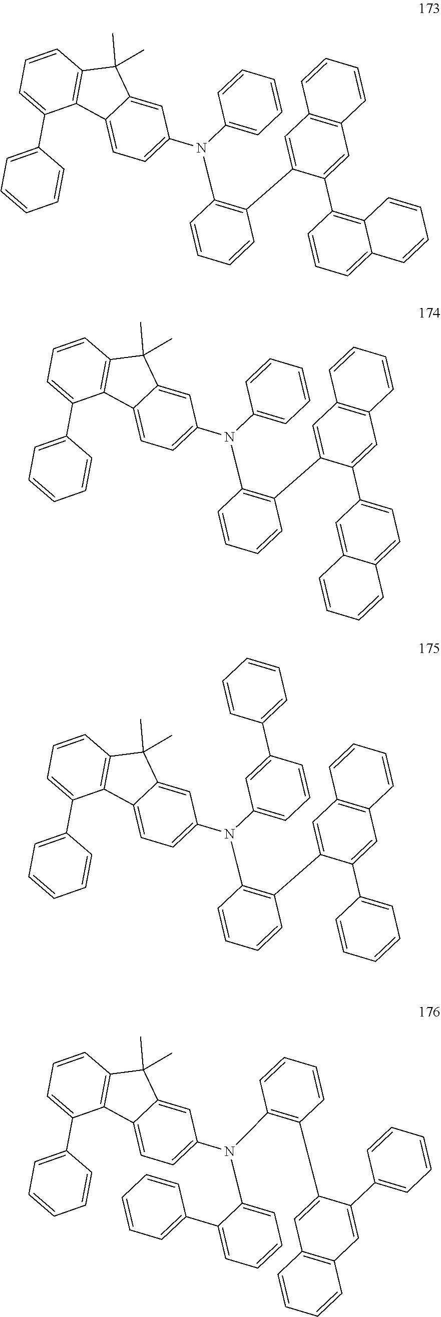 [SMILES: CC1(C)c2cc(N(c3ccccc3)c3ccccc3-c3cc4ccccc4cc3-c3cccc4ccccc34)ccc2-c2c(-c3ccccc3)cccc21 ; CC1(C)c2cc(N(c3ccccc3)c3ccccc3-c3cc4ccccc4cc3-c3ccc4ccccc4c3)ccc2-c2c(-c3ccccc3)cccc21 ; CC1(C)c2cc(N(c3cccc(-c4ccccc4)c3)c3ccccc3-c3cc4ccccc4cc3-c3ccccc3)ccc2-c2c(-c3ccccc3)cccc21 ; CC1(C)c2cc(N(c3ccccc3-c3ccccc3)c3ccccc3-c3cc4ccccc4cc3-c3ccccc3)ccc2-c2c(-c3ccccc3)cccc21]

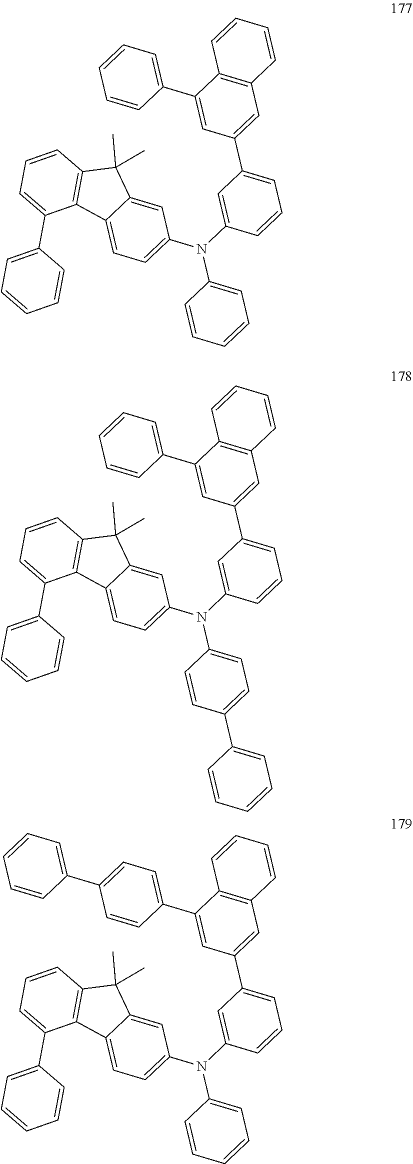

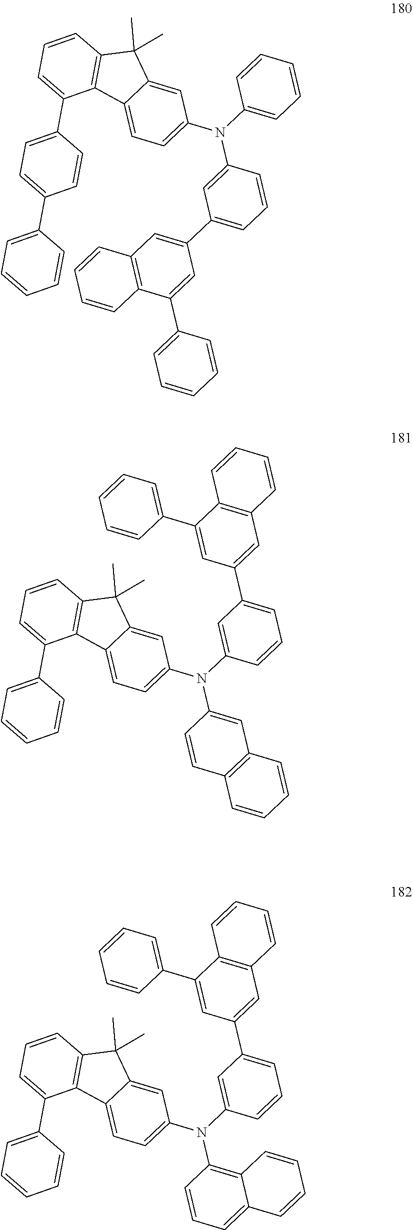 [SMILES: CC1(C)c2cc(N(c3ccccc3)c3cccc(-c4cc(-c5ccccc5)c5ccccc5c4)c3)ccc2-c2c(-c3ccc(-c4ccccc4)cc3)cccc21 ; CC1(C)c2cc(N(c3cccc(-c4cc(-c5ccccc5)c5ccccc5c4)c3)c3ccc4ccccc4c3)ccc2-c2c(-c3ccccc3)cccc21 ; CC1(C)c2cc(N(c3cccc(-c4cc(-c5ccccc5)c5ccccc5c4)c3)c3cccc4ccccc34)ccc2-c2c(-c3ccccc3)cccc21]

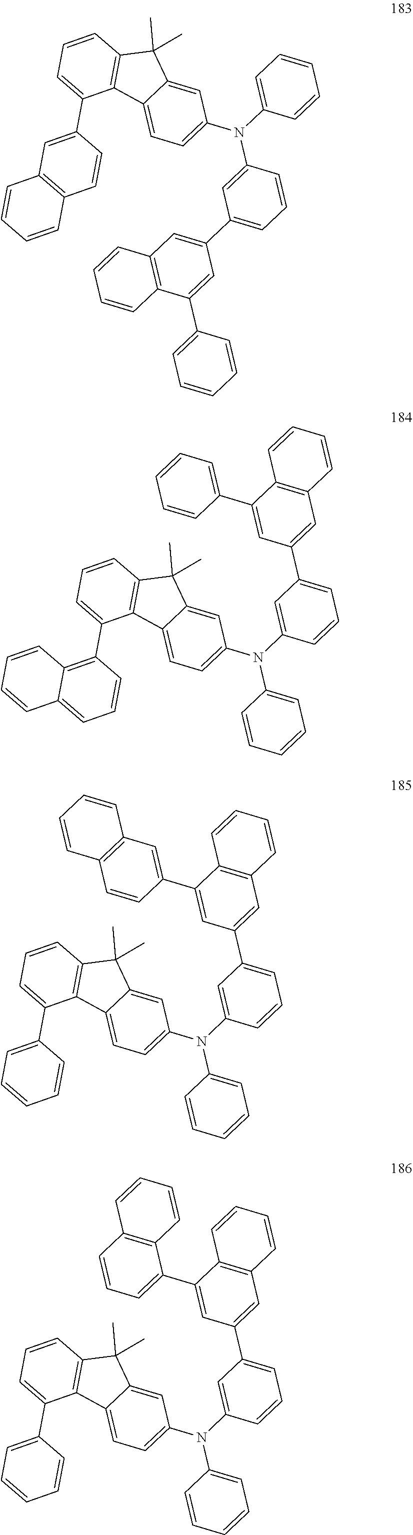 [SMILES: CC1(C)c2cc(N(c3ccccc3)c3cccc(-c4cc(-c5ccccc5)c5ccccc5c4)c3)ccc2-c2c(-c3ccc4ccccc4c3)cccc21 ; CC1(C)c2cc(N(c3ccccc3)c3cccc(-c4cc(-c5ccccc5)c5ccccc5c4)c3)ccc2-c2c(-c3cccc4ccccc34)cccc21 ; CC1(C)c2cc(N(c3ccccc3)c3cccc(-c4cc(-c5ccc6ccccc6c5)c5ccccc5c4)c3)ccc2-c2c(-c3ccccc3)cccc21 ; CC1(C)c2cc(N(c3ccccc3)c3cccc(-c4cc(-c5cccc6ccccc56)c5ccccc5c4)c3)ccc2-c2c(-c3ccccc3)cccc21]

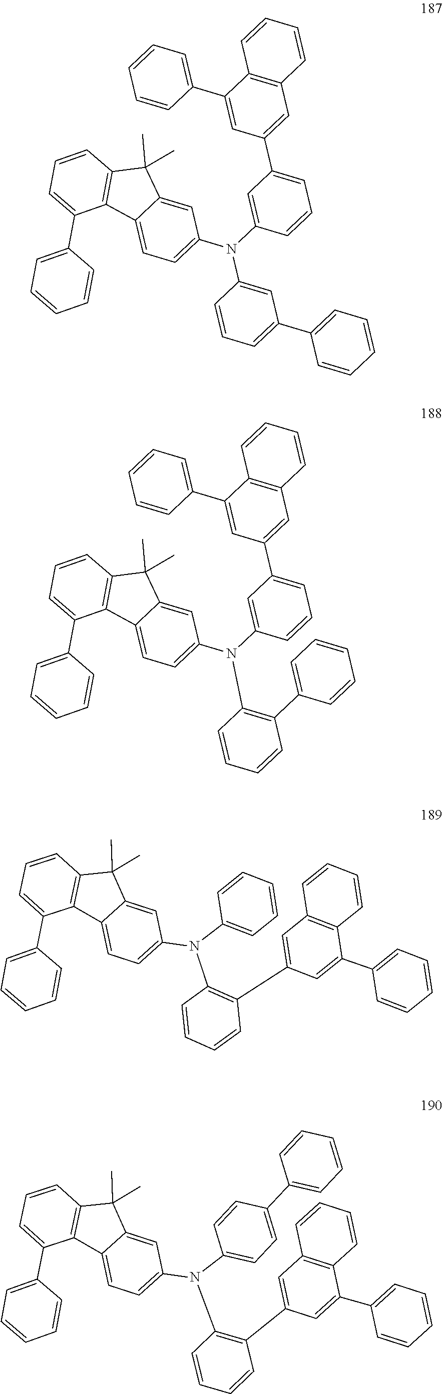 [SMILES: CC1(C)c2cc(N(c3cccc(-c4ccccc4)c3)c3cccc(-c4cc(-c5ccccc5)c5ccccc5c4)c3)ccc2-c2c(-c3ccccc3)cccc21 ; CC1(C)c2cc(N(c3cccc(-c4cc(-c5ccccc5)c5ccccc5c4)c3)c3ccccc3-c3ccccc3)ccc2-c2c(-c3ccccc3)cccc21 ; CC1(C)c2cc(N(c3ccccc3)c3ccccc3-c3cc(-c4ccccc4)c4ccccc4c3)ccc2-c2c(-c3ccccc3)cccc21 ; CC1(C)c2cc(N(c3ccc(-c4ccccc4)cc3)c3ccccc3-c3cc(-c4ccccc4)c4ccccc4c3)ccc2-c2c(-c3ccccc3)cccc21]

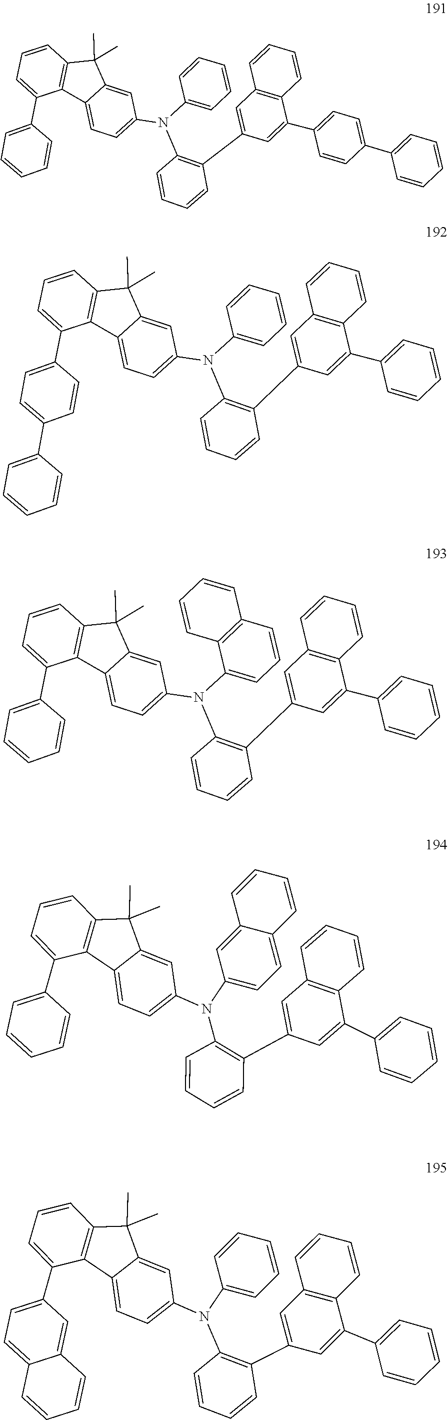 [SMILES: CC1(C)c2cc(N(c3ccccc3)c3ccccc3-c3cc(-c4ccc(-c5ccccc5)cc4)c4ccccc4c3)ccc2-c2c(-c3ccccc3)cccc21 ; CC1(C)c2cc(N(c3ccccc3)c3ccccc3-c3cc(-c4ccccc4)c4ccccc4c3)ccc2-c2c(-c3ccc(-c4ccccc4)cc3)cccc21 ; CC1(C)c2cc(N(c3ccccc3-c3cc(-c4ccccc4)c4ccccc4c3)c3cccc4ccccc34)ccc2-c2c(-c3ccccc3)cccc21 ; CC1(C)c2cc(N(c3ccc4ccccc4c3)c3ccccc3-c3cc(-c4ccccc4)c4ccccc4c3)ccc2-c2c(-c3ccccc3)cccc21 ; CC1(C)c2cc(N(c3ccccc3)c3ccccc3-c3cc(-c4ccccc4)c4ccccc4c3)ccc2-c2c(-c3ccc4ccccc4c3)cccc21]

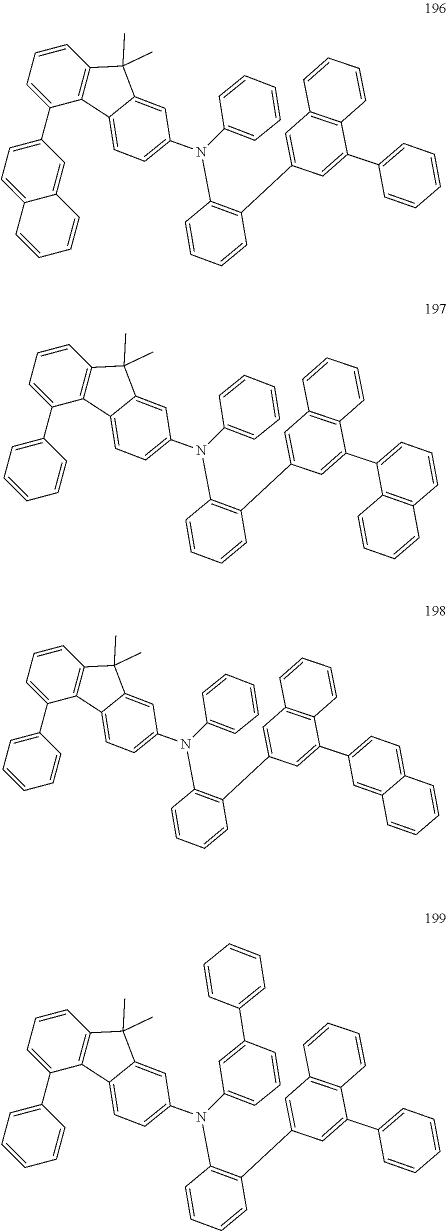 [SMILES: CC1(C)c2cc(N(c3ccccc3)c3ccccc3-c3cc(-c4ccccc4)c4ccccc4c3)ccc2-c2c(-c3ccc4ccccc4c3)cccc21 ; CC1(C)c2cc(N(c3ccccc3)c3ccccc3-c3cc(-c4cccc5ccccc45)c4ccccc4c3)ccc2-c2c(-c3ccccc3)cccc21 ; CC1(C)c2cc(N(c3ccccc3)c3ccccc3-c3cc(-c4ccc5ccccc5c4)c4ccccc4c3)ccc2-c2c(-c3ccccc3)cccc21 ; CC1(C)c2cc(N(c3cccc(-c4ccccc4)c3)c3ccccc3-c3cc(-c4ccccc4)c4ccccc4c3)ccc2-c2c(-c3ccccc3)cccc21]

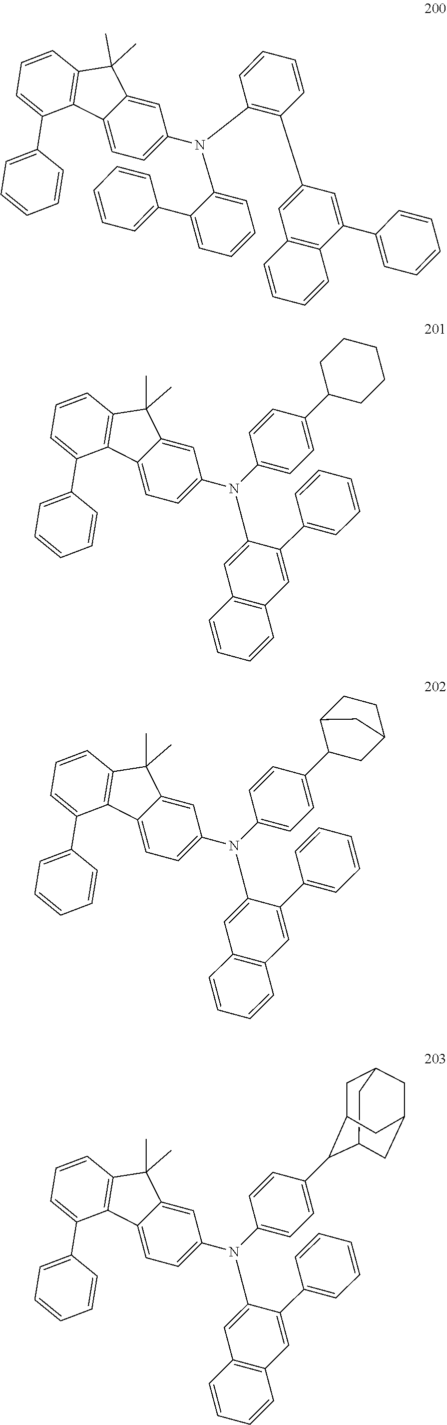 [SMILES: CC1(C)c2cc(N(c3ccccc3-c3ccccc3)c3ccccc3-c3cc(-c4ccccc4)c4ccccc4c3)ccc2-c2c(-c3ccccc3)cccc21 ; CC1(C)c2cc(N(c3ccc(C4CCCCC4)cc3)c3cc4ccccc4cc3-c3ccccc3)ccc2-c2c(-c3ccccc3)cccc21 ; CC1(C)c2cc(N(c3ccc(C4CC5CCC4C5)cc3)c3cc4ccccc4cc3-c3ccccc3)ccc2-c2c(-c3ccccc3)cccc21 ; CC1(C)c2cc(N(c3ccc(C4C5CC6CC(C5)CC4C6)cc3)c3cc4ccccc4cc3-c3ccccc3)ccc2-c2c(-c3ccccc3)cccc21]

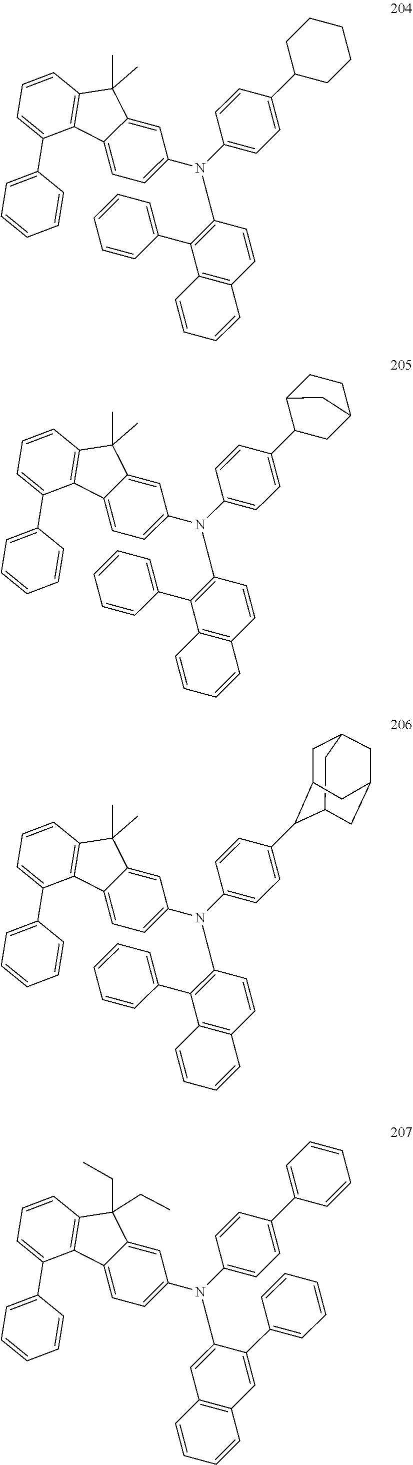 [SMILES: CC1(C)c2cc(N(c3ccc(C4CCCCC4)cc3)c3ccc4ccccc4c3-c3ccccc3)ccc2-c2c(-c3ccccc3)cccc21 ; CC1(C)c2cc(N(c3ccc(C4CC5CCC4C5)cc3)c3ccc4ccccc4c3-c3ccccc3)ccc2-c2c(-c3ccccc3)cccc21 ; CC1(C)c2cc(N(c3ccc(C4C5CC6CC(C5)CC4C6)cc3)c3ccc4ccccc4c3-c3ccccc3)ccc2-c2c(-c3ccccc3)cccc21 ; CCC1(CC)c2cc(N(c3ccc(-c4ccccc4)cc3)c3cc4ccccc4cc3-c3ccccc3)ccc2-c2c(-c3ccccc3)cccc21]

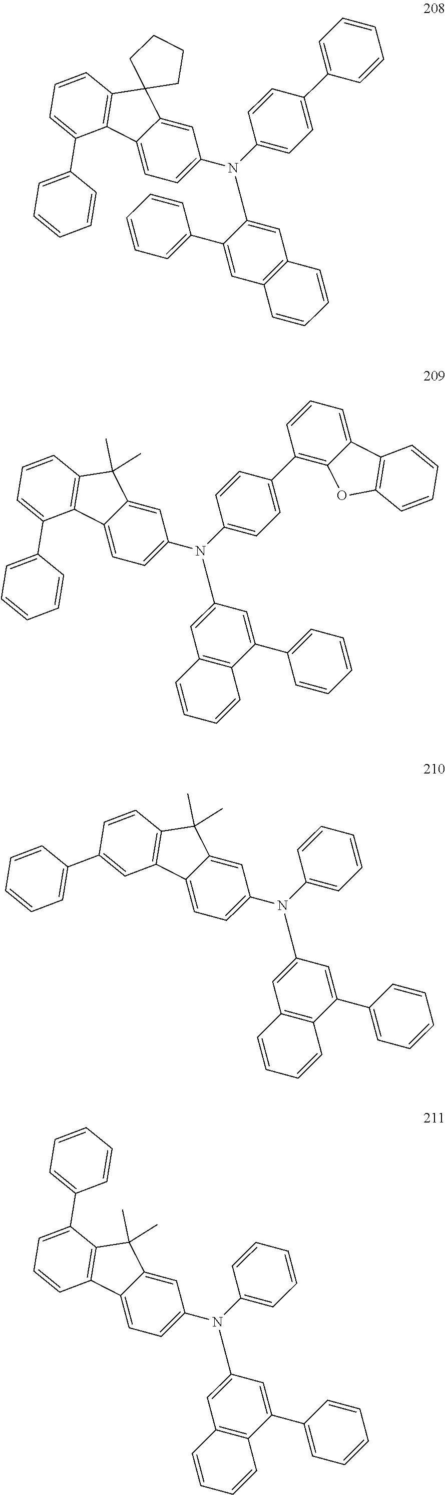 [SMILES: c1ccc(-c2ccc(N(c3ccc4c(c3)C3(CCCC3)c3cccc(-c5ccccc5)c3-4)c3cc4ccccc4cc3-c3ccccc3)cc2)cc1 ; CC1(C)c2cc(N(c3ccc(-c4cccc5c4oc4ccccc45)cc3)c3cc(-c4ccccc4)c4ccccc4c3)ccc2-c2c(-c3ccccc3)cccc21 ; CC1(C)c2ccc(-c3ccccc3)cc2-c2ccc(N(c3ccccc3)c3cc(-c4ccccc4)c4ccccc4c3)cc21 ; CC1(C)c2cc(N(c3ccccc3)c3cc(-c4ccccc4)c4ccccc4c3)ccc2-c2cccc(-c3ccccc3)c21]

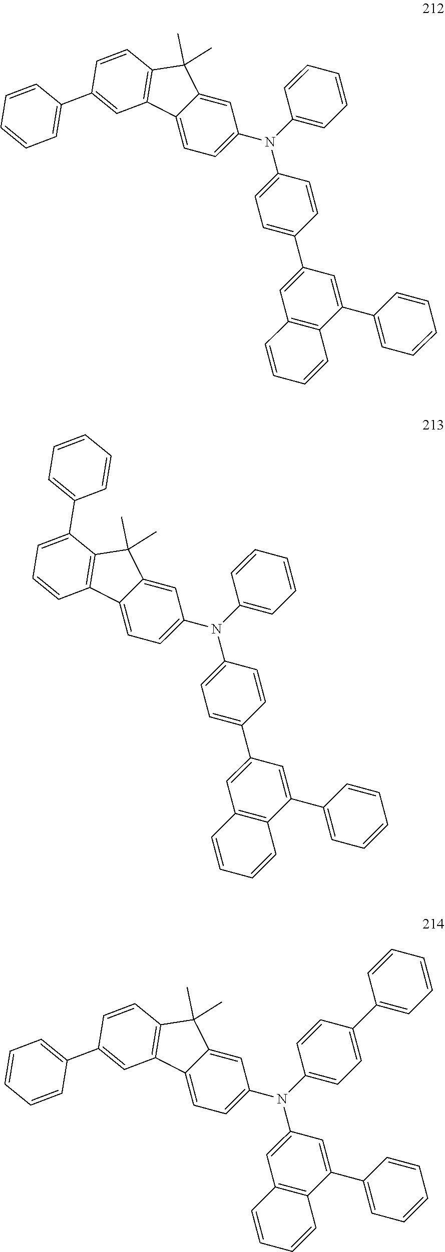 [SMILES: CC1(C)c2ccc(-c3ccccc3)cc2-c2ccc(N(c3ccccc3)c3ccc(-c4cc(-c5ccccc5)c5ccccc5c4)cc3)cc21 ; CC1(C)c2cc(N(c3ccccc3)c3ccc(-c4cc(-c5ccccc5)c5ccccc5c4)cc3)ccc2-c2cccc(-c3ccccc3)c21 ; CC1(C)c2ccc(-c3ccccc3)cc2-c2ccc(N(c3ccc(-c4ccccc4)cc3)c3cc(-c4ccccc4)c4ccccc4c3)cc21]

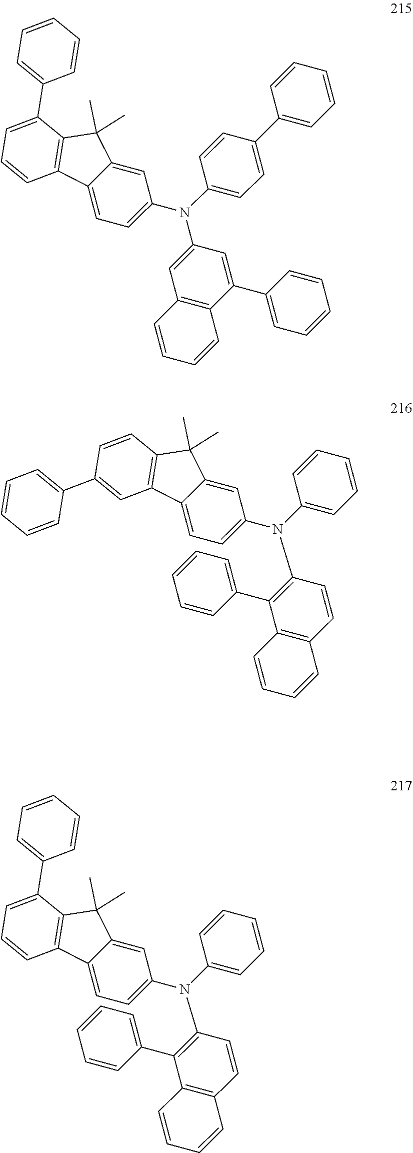 [SMILES: CC1(C)c2cc(N(c3ccc(-c4ccccc4)cc3)c3cc(-c4ccccc4)c4ccccc4c3)ccc2-c2cccc(-c3ccccc3)c21 ; CC1(C)c2ccc(-c3ccccc3)cc2-c2ccc(N(c3ccccc3)c3ccc4ccccc4c3-c3ccccc3)cc21 ; CC1(C)c2cc(N(c3ccccc3)c3ccc4ccccc4c3-c3ccccc3)ccc2-c2cccc(-c3ccccc3)c21]

In the amine-containing compound represented by one of Formulae 1 to 3, Ar₁ to Ar₃ may be a C₃-C₆₀ carbocyclic group unsubstituted or substituted with at least one R₁₀ₐ or a C₁-C₆₀ heterocyclic group unsubstituted or substituted with at least one R₁₀ₐ. In an embodiment, L₁ and Ar₁ may each not be a fluorene group unsubstituted or substituted with at least one R₁₀ₐ. The amine group may be linked to a carbon at a 2-position of a fluorene moiety, and may be linked to a carbon at a 2-position of a naphthalene moiety. Accordingly, the amine-containing compound may have an appropriate energy level for a hole transport layer, and due to an increase in a π-conjugation, holes generated in the amine may be effectively stabilized. Therefore, the amine-containing compound represented by one of Formulae 1 to 3 may have excellent hole transport characteristics, and the light-emitting device including the amine-containing compound represented by one of Formulae 1 to 3 may have a low driving voltage, high luminance, high luminescence efficiency, and long lifespan.

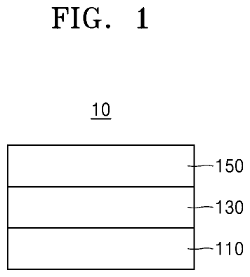

### DESCRIPTION OF FIG. 1

FIG. 1 is a schematic cross-sectional view of a light-emitting device 10 according to an embodiment. The light-emitting device 10 may include a first electrode 110, an interlayer 130, and a second electrode 150.

Hereinafter, a structure of the light-emitting device 10 according to an embodiment and a method of manufacturing the light-emitting device 10 will be described in connection with FIG. 1.

[First Electrode 110]

In FIG. 1, a substrate may be further included under the first electrode 110 and/or on the second electrode 150. In an embodiment, the substrate may be a glass substrate or a plastic substrate. In embodiments, the substrate may be a flexible substrate. For example, a flexible substrate may include plastics with excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by applying a material for forming the first electrode 110 onto a substrate by using a deposition or sputtering method. When the first electrode 110 is an anode, a material for forming the first electrode 110 may be a high work function material to facilitate the injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. When the first electrode 110 is a semi-transmissive electrode or a reflective electrode, a material for forming the first electrode 110 may include magnesium (Mg), silver (Ag), aluminum (AI), aluminum-lithium (Al—Li), calcium (Ca), magnesium-indium (Mg—In), magnesium-silver (Mg—Ag), or any combination thereof.

The first electrode 110 may have a structure consisting of a single layer or a structure including multiple layers. For example, the first electrode 110 may have a three-layered structure of ITO/Ag/ITO.

[Interlayer 130]

The interlayer 130 may be disposed on the first electrode 110. The interlayer 130 may include an emission layer.

The interlayer 130 may further include a hole transport region between the first electrode 110 and the emission layer and an electron transport region between the emission layer and the second electrode 150.

The interlayer 130 may further include, in addition to various organic materials, a metal-containing compound such as an organometallic compound, an inorganic material such as quantum dots, or the like.

The interlayer 130 may include two or more emitting units stacked between the first electrode 110 and the second electrode 150, and at least one charge generation layer located between the two or more emitting units. When the interlayer 130 includes the two or more emitting units and the at least one charge generation layer as described above, the light-emitting device 10 may be a tandem light-emitting device.

[Hole Transport Region in Interlayer 130]

The hole transport region may have a structure consisting of a layer consisting of a single material, ii) a structure consisting of a layer including different materials, or a structure including multiple layers including different materials.

The hole transport region may include a hole injection layer, a hole transport layer, an emission auxiliary layer, an electron blocking layer, or any combination thereof.

In embodiments, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein the layers of each structure may be stacked from the first electrode 110 in its respective stated order, but the structure of the hole transport region is not limited thereto.

The hole transport region may include a compound represented by Formula 201, a compound represented by Formula 202, or any combination thereof:

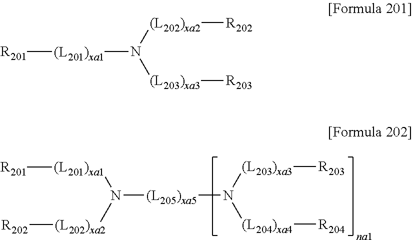

In Formulae 201 and 202,
L₂₀₁ to L₂₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ, or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (for example, a carbazole group or the like) unsubstituted or substituted with at least one R₁₀ₐ (for example, see Compound HT16),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and
na1 may be an integer from 1 to 4.

In embodiments, the compound represented by Formula 201 and the compound represented by Formula 202 may each independently include at least one of groups represented by Formulae CY201 to CY217:

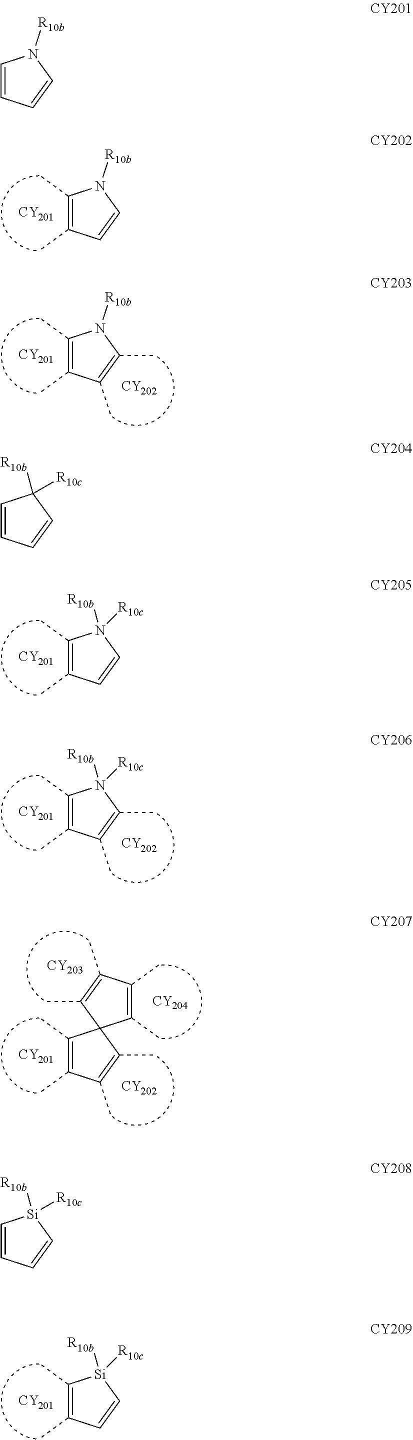

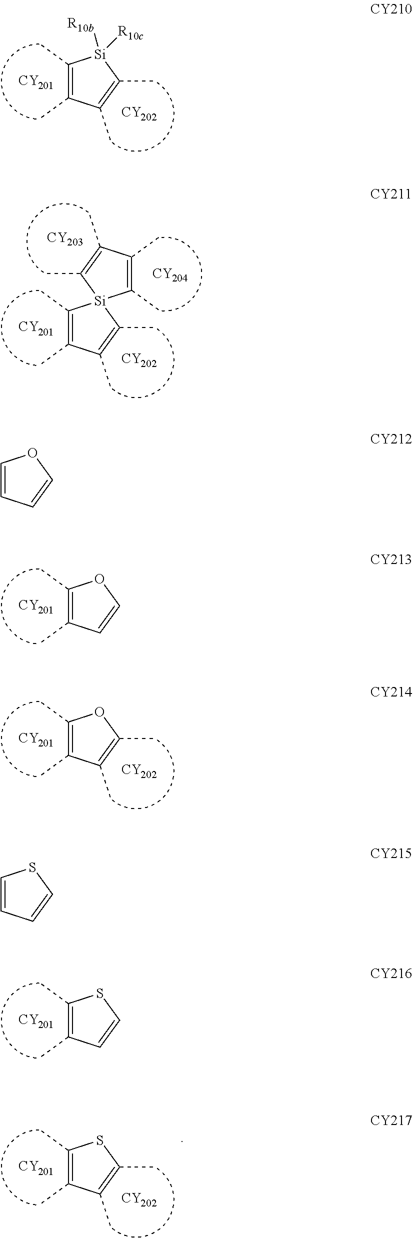

In Formulae CY201 to CY217, R_{10b} and R_{10c} may each independently be the same as described with respect to R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described herein.

In an embodiment, in Formulae CY201 to CY217, ring CY₂₀₁ to ring CY₂₀₄ may each independently be a benzene group, a naphthalene group, a phenanthrene group, or an anthracene group.

In embodiments, the compound represented by Formula 201 and the compound represented by Formula 202 may each independently include at least one of groups represented by Formulae CY201 to CY203.

In embodiments, the compound represented by Formula 201 may include at least one of groups represented by Formulae CY201 to CY203 and at least one of groups represented by Formulae CY204 to CY217.

In embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In embodiments, the compound represented by Formula 201 and the compound represented by Formula 202 may each not include a group represented by one of Formulae CY201 to CY203.

In embodiments, the compound represented by Formula 201 and the compound represented by Formula 202 may each not include a group represented by one of Formulae CY201 to CY203, and may each independently include at least one of groups represented by Formulae CY204 to CY217.

In embodiments, the compound represented by Formula 201 and the compound represented by Formula 202 may each not include a group represented by one of Formulae CY201 to CY217.

In an embodiment, the hole transport region may include one of Compounds HT1 to HT46, m-MTDATA, TDATA, 2-TNATA, NPB(NPD), p-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

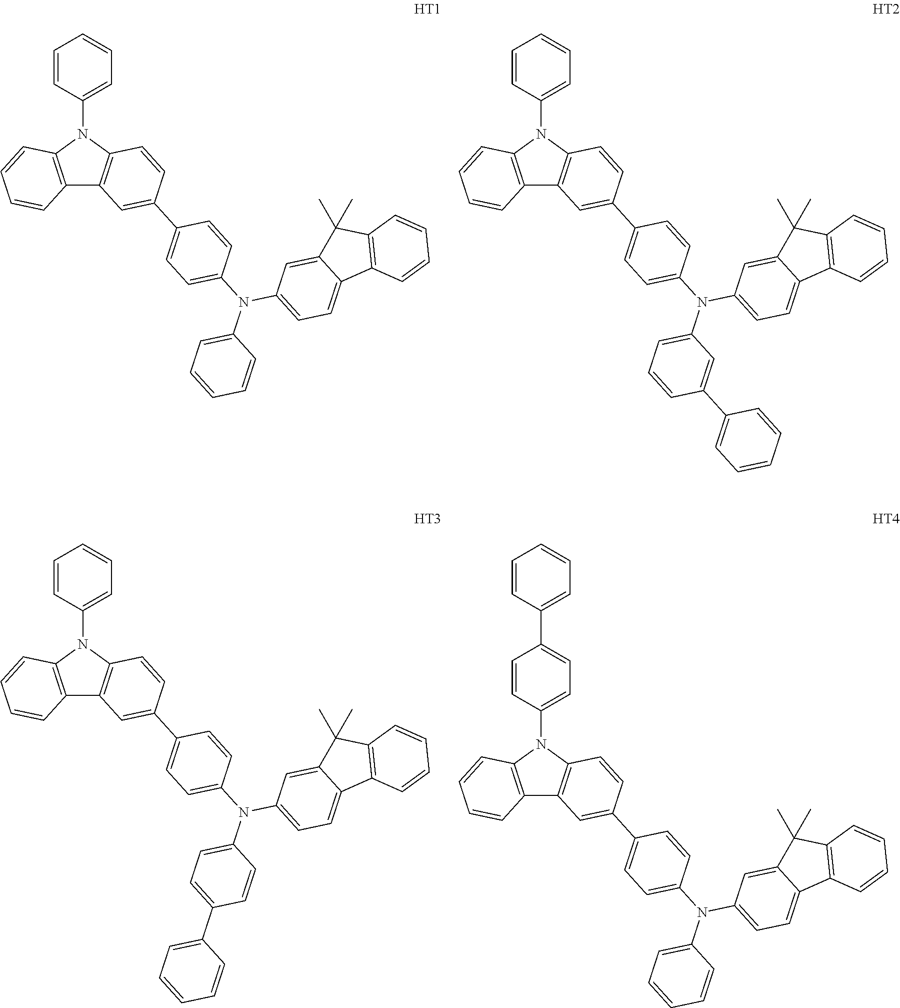

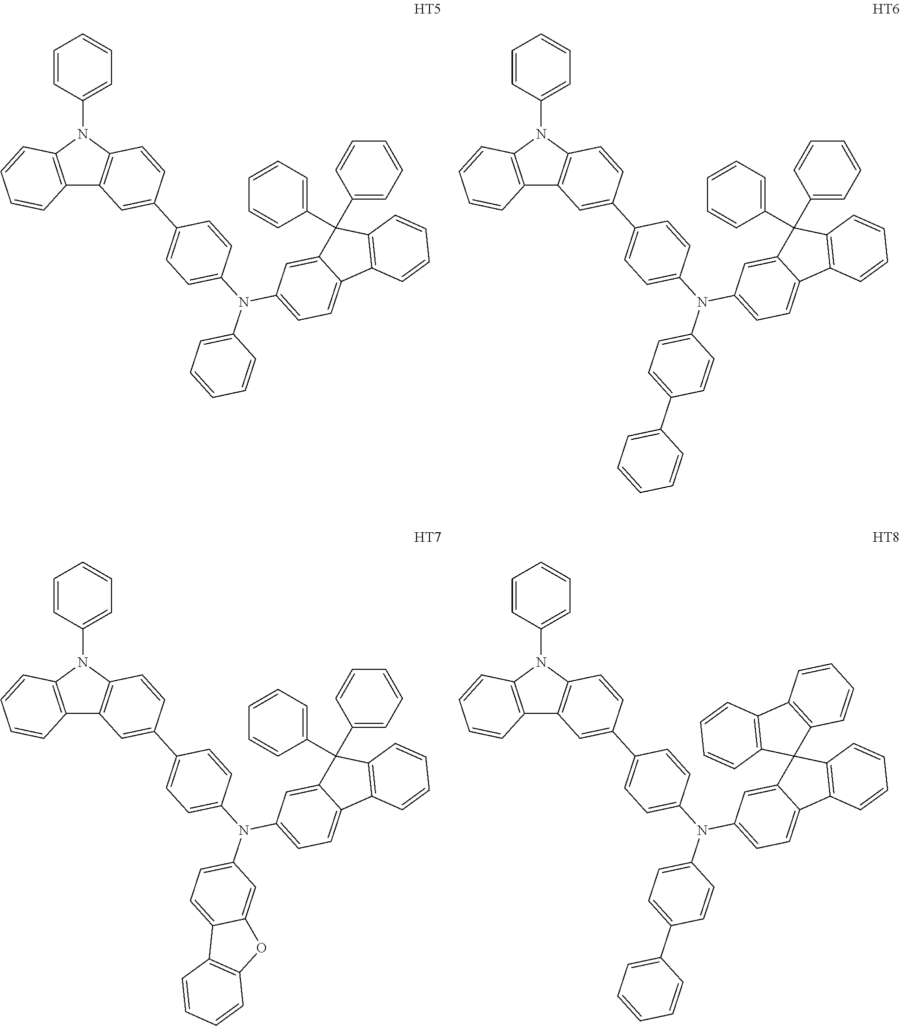 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

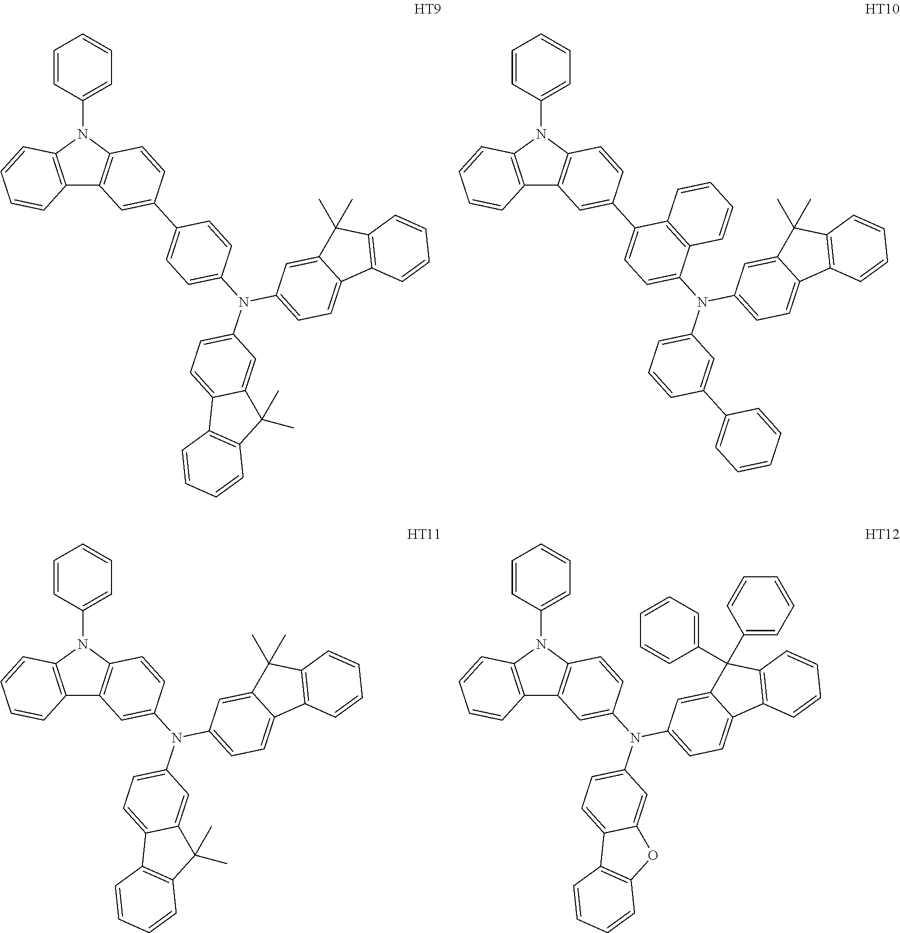 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

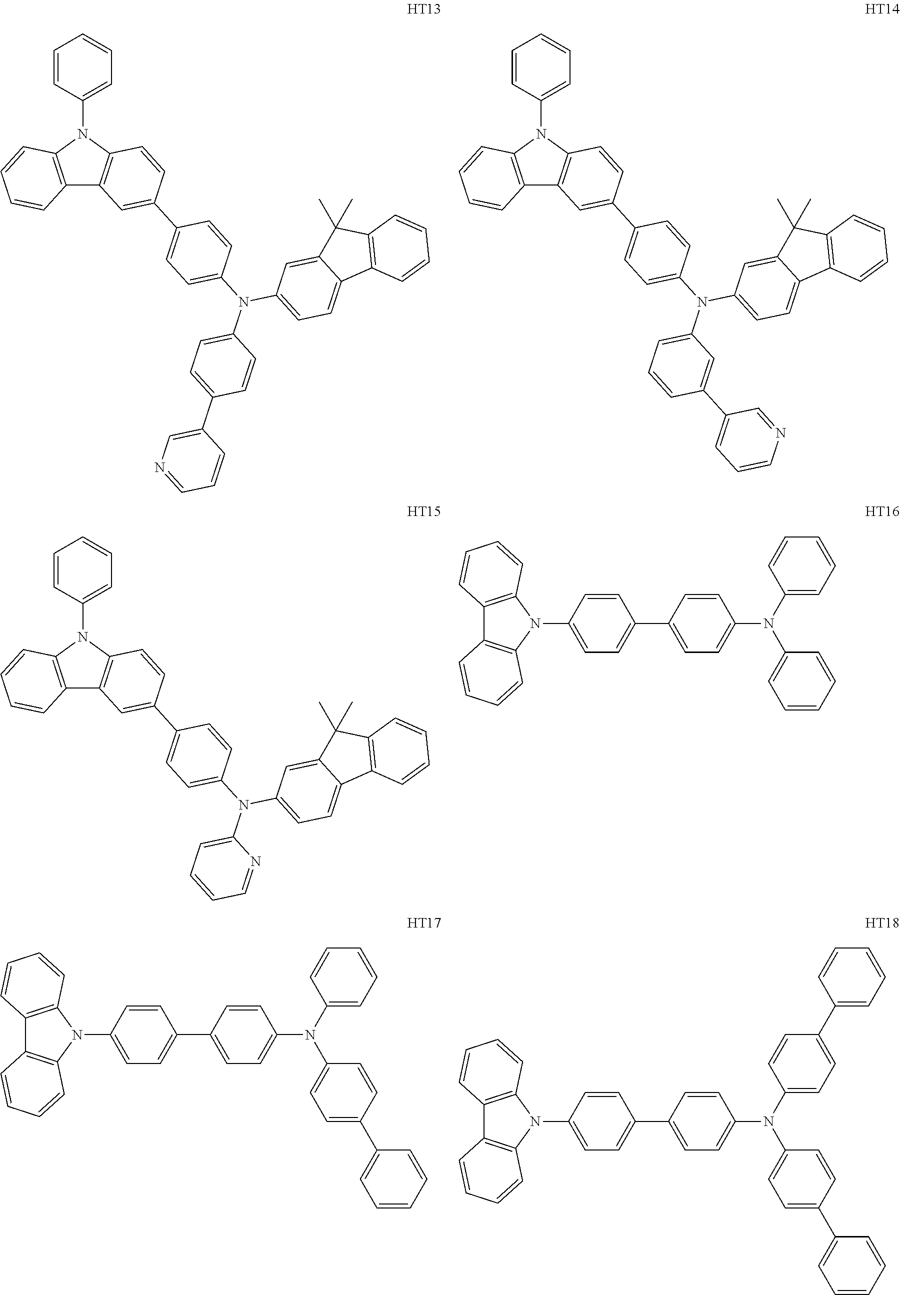

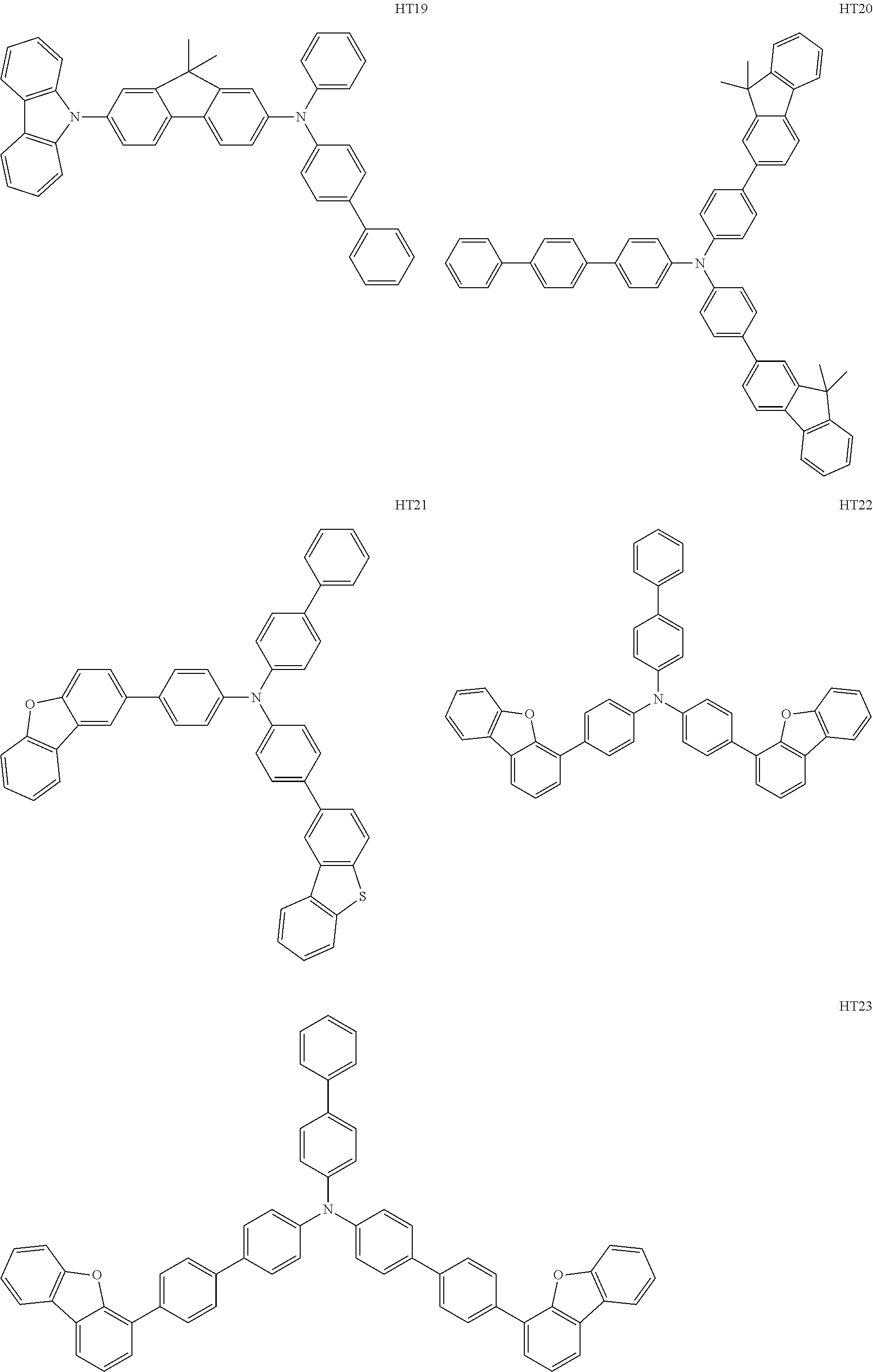

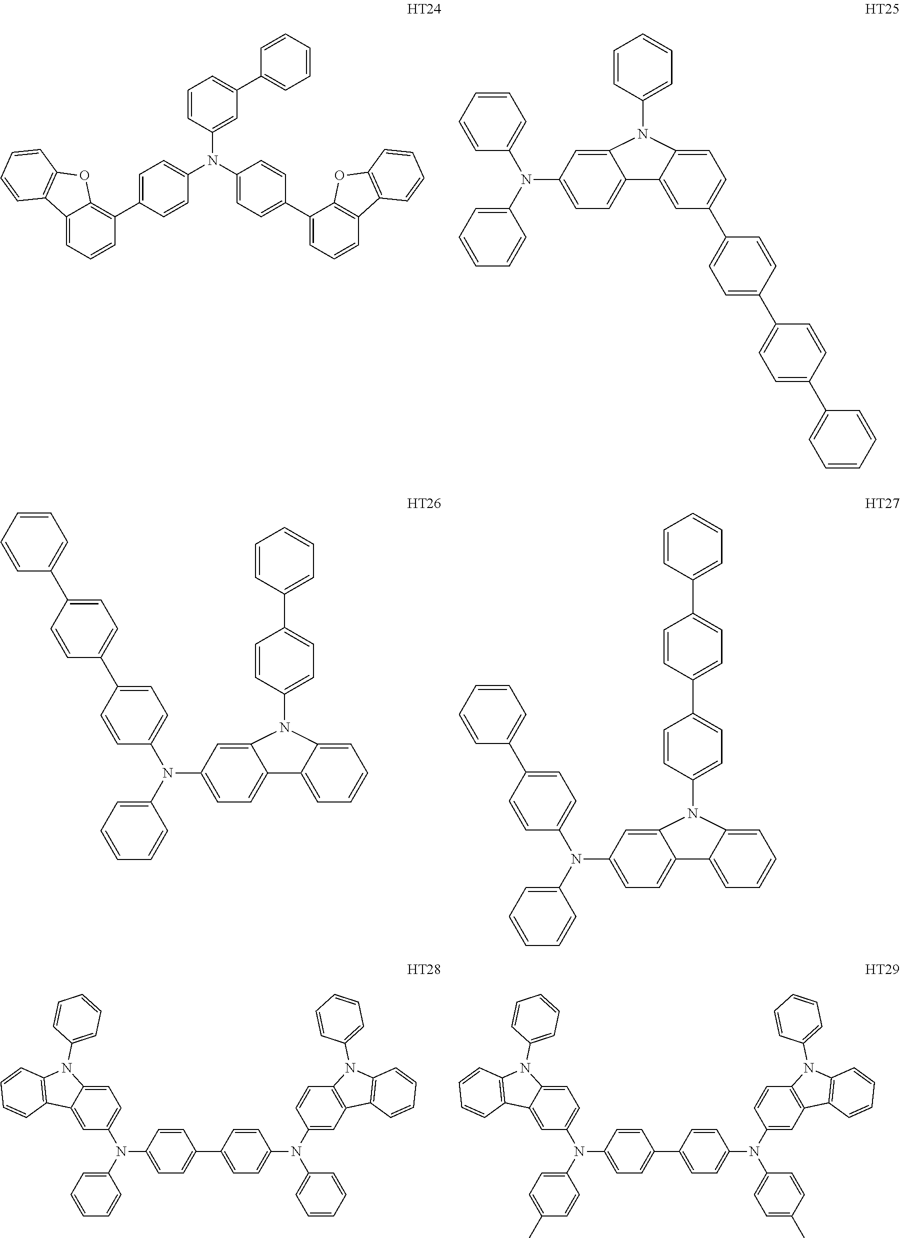

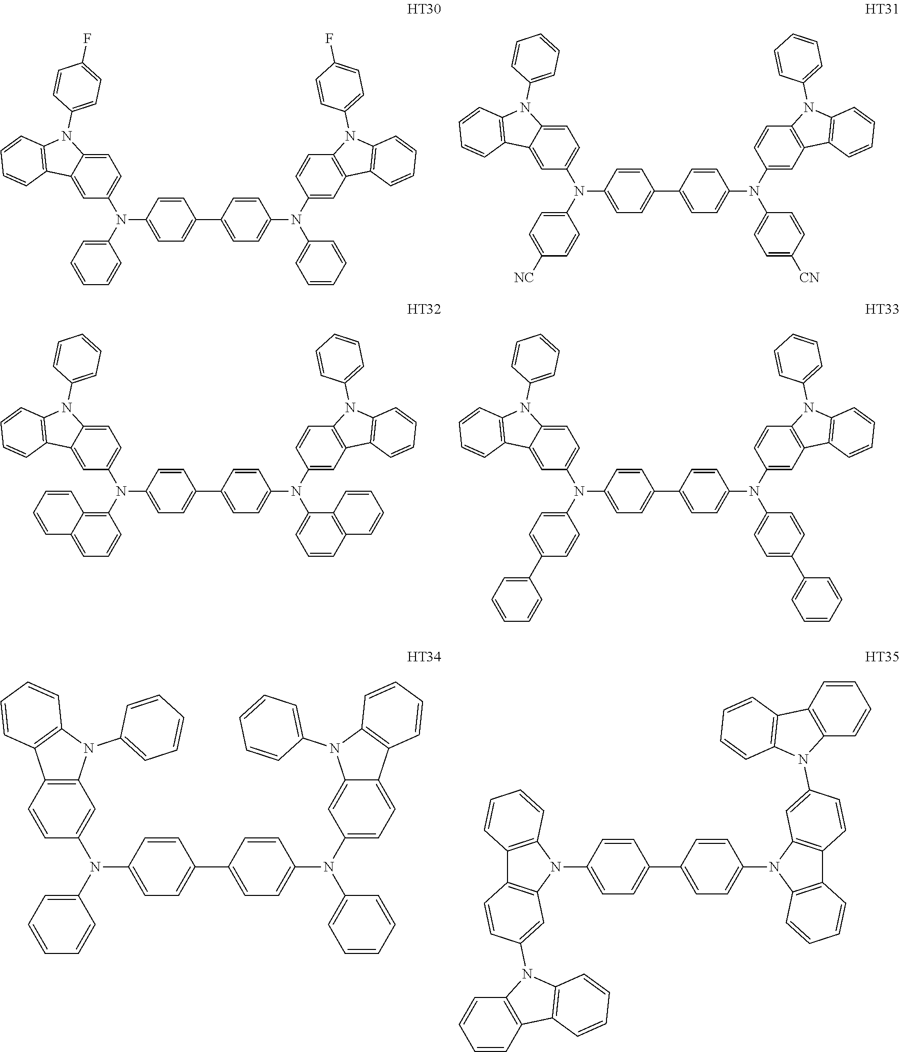

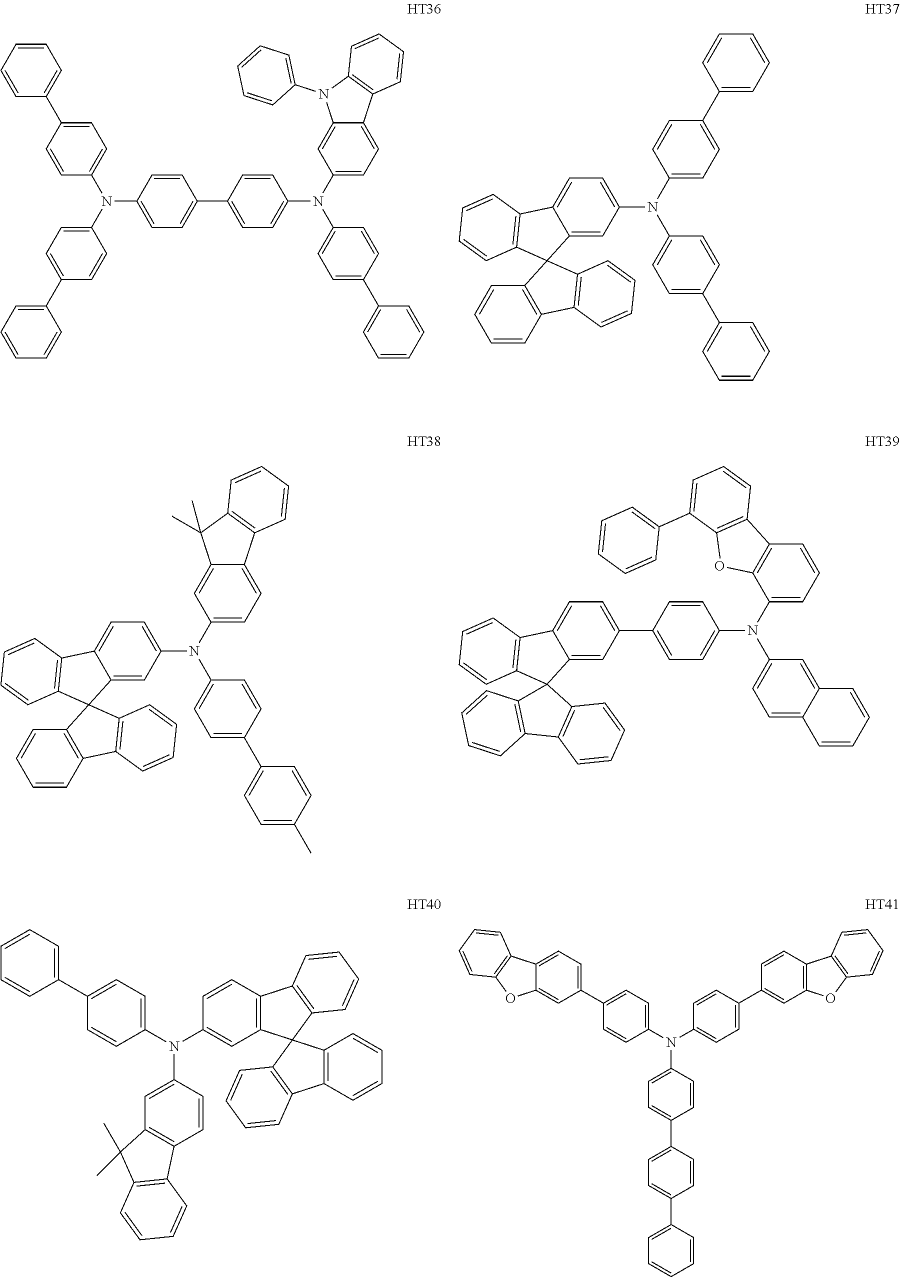

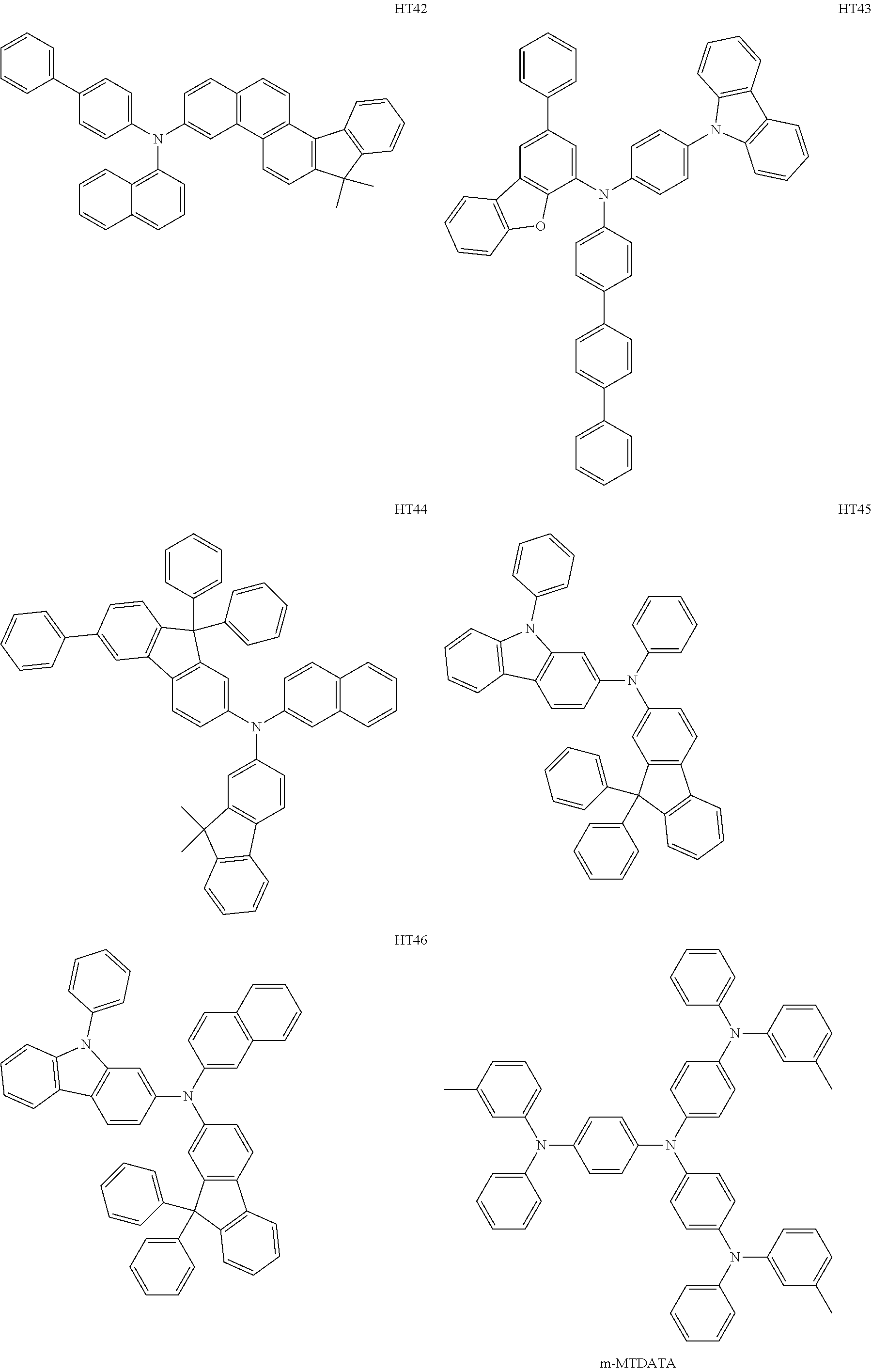

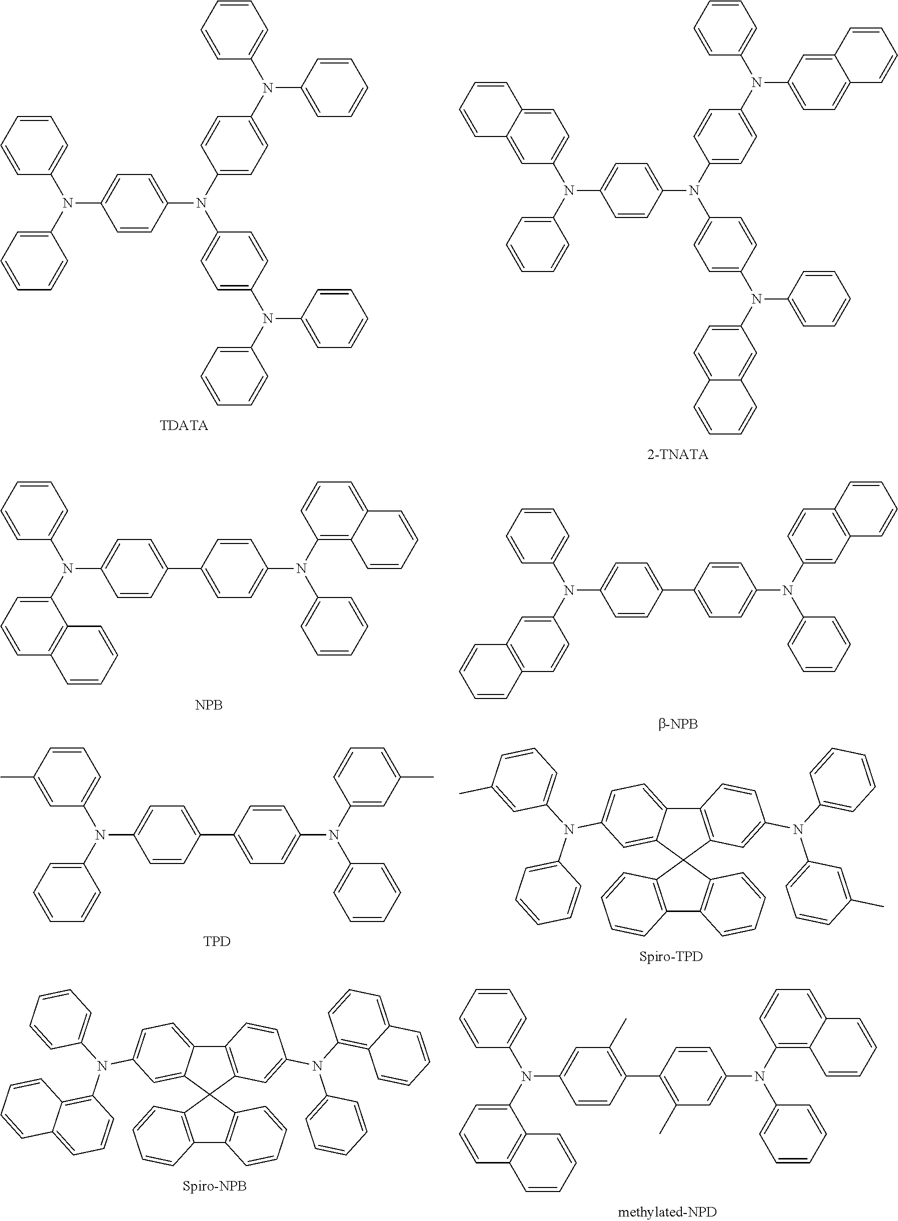

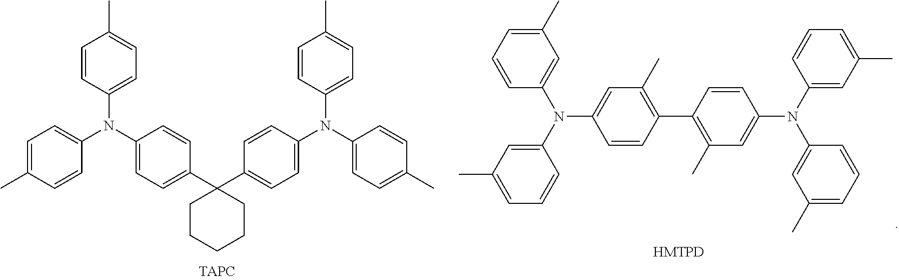

A thickness of the hole transport region may be in a range of about 50 Å to about 10,000 Å. For example, the thickness of the hole transport region may be in a range of about 100 Å to about 4,000 Å. When the hole transport region includes a hole injection layer, a hole transport layer, or any combination thereof, a thickness of the hole injection layer may be in a range of about 100 Å to about 9,000 Å, and a thickness of the hole transport layer may be in a range of about 50 Å to about 2,000 Å. For example, the thickness of the hole injection layer may be in a range of about 100 Å to about 1,000 Å. For example, the thickness of the hole transport layer may be in a range of about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may compensate for an optical resonance distance according to a wavelength of light emitted from the emission layer, and thus, light emitting efficiency may be improved. The electron blocking layer may prevent electron leakage from the emission layer to the hole transport region. Materials that may be included in the hole transport region may be included in the emission auxiliary layer and the electron blocking layer.

[p-Dopant]

The hole transport region may further include, in addition to these materials, a charge generation material for the improvement of conductive properties. The charge generation material may be uniformly or non-uniformly dispersed in the hole transport region (for example, in the form of a single layer consisting of a charge generation material).

The charge generation material may be, for example, a p-dopant.

For example, a lowest unoccupied molecular orbital (LUMO) energy level of the p-dopant may be equal to or less than about −3.5 eV.

In embodiments, the p-dopant may include a quinone derivative, a cyano group-containing compound, a compound including element EL1 and element EL2, or any combination thereof.

Examples of the quinone derivative may include TCNQ, F4-TCNQ, etc.

Examples of the cyano group-containing compound may include HAT-CN, and a compound represented by Formula 221:

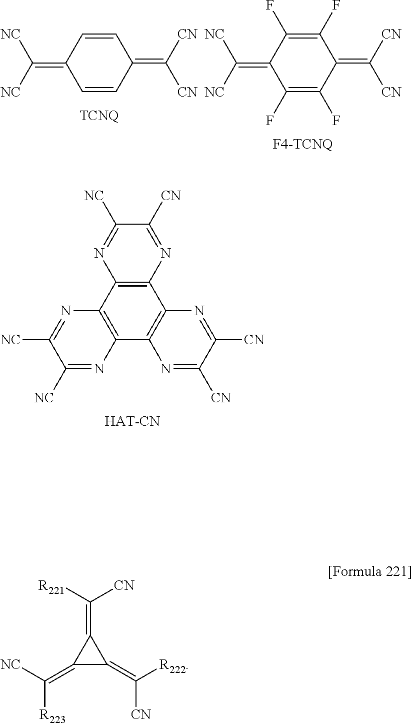

In Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with: a cyano group; —F; —Cl; —Br; -A; a C₁-C₂₀ alkyl group substituted with a cyano group, —F, —Cl, —Br, —I, or any combination thereof; or any combination thereof.

In the compound including element EL1 and element EL2, element EL1 may be a metal, a metalloid, or any combination thereof, and element EL2 may be a non-metal, a metalloid, or any combination thereof.

Examples of the metal may include: an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Examples of the metalloid may include silicon (Si), antimony (Sb), and tellurium (Te).

Examples of the non-metal may include oxygen (O) and a halogen (for example, F, Cl, Br, I, etc.).

Examples of the compound including element EL1 and element EL2 may include a metal oxide, a metal halide (for example, a metal fluoride, a metal chloride, a metal bromide, or a metal iodide), a metalloid halide (for example, a metalloid fluoride, a metalloid chloride, a metalloid bromide, or a metalloid iodide), a metal telluride, or any combination thereof.

Examples of the metal oxide may include tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), and rhenium oxide (for example, ReO₃, etc.).

Examples of the metal halide may include an alkali metal halide, an alkaline earth metal halide, a transition metal halide, a post-transition metal halide, and a lanthanide metal halide.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, and CsI.

Examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, and BaI₂.

Examples of the transition metal halide may include a titanium halide (for example, TiF₄, TiCl₄, TiBr₄, TiI₄, etc.), a zirconium halide (for example, ZrF₄, ZrCl₄, ZrBr₄, ZrI₄, etc.), a hafnium halide (for example, HfF₄, HfCl₄, HfBr₄, HfI₄, etc.), a vanadium halide (for example, VF₃, VCI₃, VBr₃, VI₃, etc.), a niobium halide (for example, NbF₃, NbCl₃, NbBr₃, NbI₃, etc.), a tantalum halide (for example, TaF₃, TaCI₃, TaBr₃, TaI₃, etc.), a chromium halide (for example, CrF₃, CrCl₃, CrBr₃, CrI₃, etc.), a molybdenum halide (for example, MoF₃, MoCI₃, MoBr₃, MoI₃, etc.), a tungsten halide (for example, WF₃, WCl₃, WBr₃, WI₃, etc.), a manganese halide (for example, MnF₂, MnCl₂, MnBr₂, MnI₂, etc.), a technetium halide (for example, TcF₂, TcCl₂, TcBr₂, TcI₂, etc.), a rhenium halide (for example, ReF₂, ReCl₂, ReBr₂, ReI₂, etc.), an iron halide (for example, FeF₂, FeCl₂, FeBr₂, FeI₂, etc.), a ruthenium halide (for example, RuF₂, RuCl₂, RuBr₂, RuI₂, etc.), an osmium halide (for example, OsF₂, OSCl₂, OsBr₂, OSI₂, etc.), a cobalt halide (for example, CoF₂, CoCl₂, CoBr₂, CoI₂, etc.), a rhodium halide (for example, RhF₂, RhCl₂, RhBr₂, RhI₂, etc.), an iridium halide (for example, IrF₂, IrCl₂, IrBr₂, IrI₂, etc.), a nickel halide (for example, NiF₂, NiCl₂, NiBr₂, Nil₂, etc.), a palladium halide (for example, PdF₂, PdCl₂, PdBr₂, PdI₂, etc.), a platinum halide (for example, PtF₂, PtCl₂, PtBr₂, PtI₂, etc.), a copper halide (for example, CuF, CuCl, CuBr, CuI, etc.), a silver halide (for example, AgF, AgCl, AgBr, AgI, etc.), and a gold halide (for example, AuF, AuCl, AuBr, AuI, etc.).

Examples of the post-transition metal halide may include a zinc halide (for example, ZnF₂, ZnCl₂, ZnBr₂, ZnI₂, etc.), an indium halide (for example, InI₃, etc.), and a tin halide (for example, SnI₂, etc.).

Examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and the like.

Examples of the metalloid halide may include an antimony halide (for example, SbCl₅, etc.).

Examples of the metal telluride may include an alkali metal telluride (for example, Li₂Te, Na₂Te, K₂Te, Rb₂Te, Cs₂Te, etc.), an alkaline earth metal telluride (for example, BeTe, MgTe, CaTe, SrTe, BaTe, etc.), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (for example, ZnTe, etc.), and a lanthanide metal telluride (for example, LaTe, CeTe, PrTe, NdTe, PmTe, EuTe, GdTe, TbTe, DyTe, HoTe, ErTe, TmTe, YbTe, LuTe, etc.).

[Emission Layer in Interlayer 130]

When the light-emitting device 10 is a full-color light-emitting device, the emission layer may be patterned into a red emission layer, a green emission layer, and/or a blue emission layer, according to a subpixel. In embodiments, the emission layer may have a stacked structure of two or more layers of a red emission layer, a green emission layer, and a blue emission layer, in which the two or more layers may contact each other or may be separated from each other to emit white light. In embodiments, the emission layer may include two or more materials of a red light-emitting material, a green light-emitting material, and a blue light-emitting material, in which the two or more materials are mixed with each other in a single layer to emit white light.

The emission layer may include a host and a dopant. The dopant may include a phosphorescent dopant, a fluorescent dopant, or any combination thereof.

An amount of the dopant in the emission layer may be in a range of about 0.01 parts by weight to about 15 parts by weight, based on 100 parts by weight of the host.

In embodiments, the emission layer may include a quantum dot.

In embodiments, the emission layer may include a delayed fluorescence material. The delayed fluorescence material may serve as a host or as a dopant in the emission layer.

A thickness of the emission layer may be in a range of about 100 Å to about 1,000 Å. For example, the thickness of the emission layer may be in a range of about 200 Å to about 600 Å. When the thickness of the emission layer is within these ranges, excellent light-emission characteristics may be obtained without a substantial increase in driving voltage.

[Host]

In embodiments, the host may include a compound represented by Formula 301:

[Ar₃₀₁]_{xb11}-[(L₃₀₁)_{xb1}-R₃₀₁]_{xb21} [Formula 301]

In Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each independently be the same as described herein with respect to Q₁.

In an embodiment, in Formula 301, when xb11 is 2 or more, two or more of Ar₃₀₁(s) may be linked to each other via a single bond.

In embodiments, the host may include a compound represented by Formula 301-1, a compound represented by Formula 301-2, or any combination thereof:

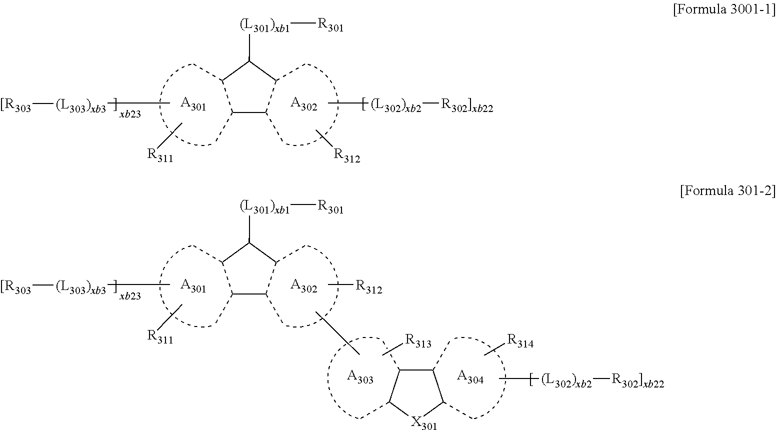

In Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be the same as described herein,
L₃₀₂ to L₃₀₄ may each independently be the same as described herein with respect to with L₃₀₁,
xb2 to xb4 may each independently be the same as described herein with respect to xb1, and
R₃₀₂ to R₃₀₅ and R₃₁₁ to R₃₁₄ may each independently be the same as described herein with respect to R₃₀₁.

In embodiments, the host may include an alkali earth metal complex, a post-transition metal complex, or any combination thereof. For example, the host may include a Be complex (for example, Compound H55), an Mg complex, a Zn complex, or any combination thereof.

In embodiments, the host may include one of Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

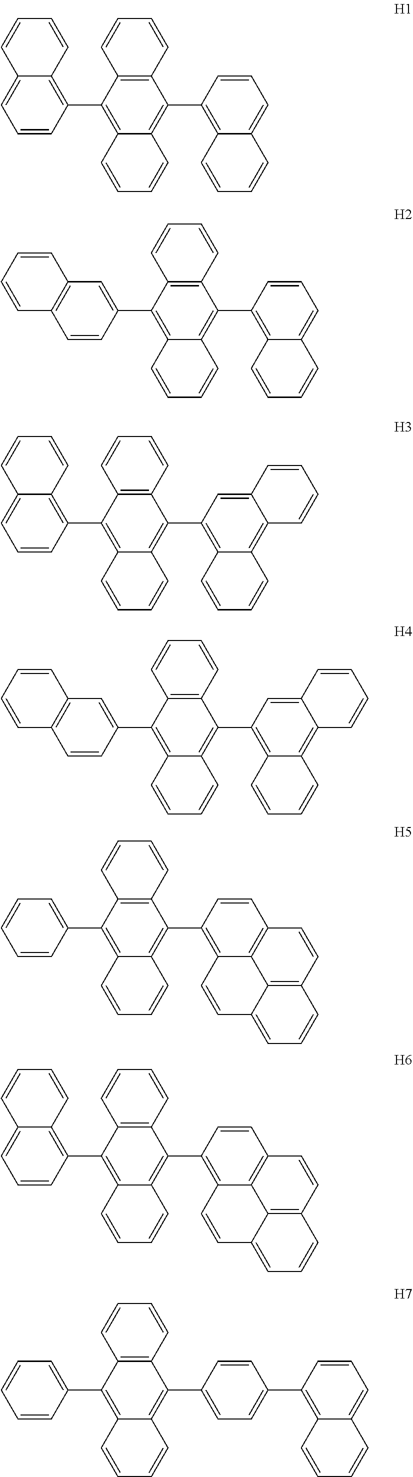

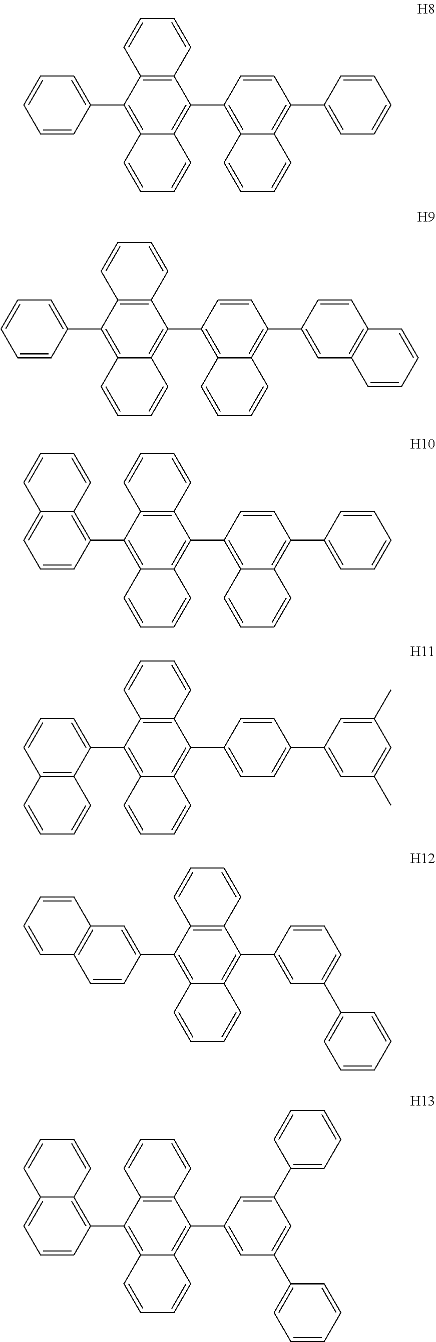

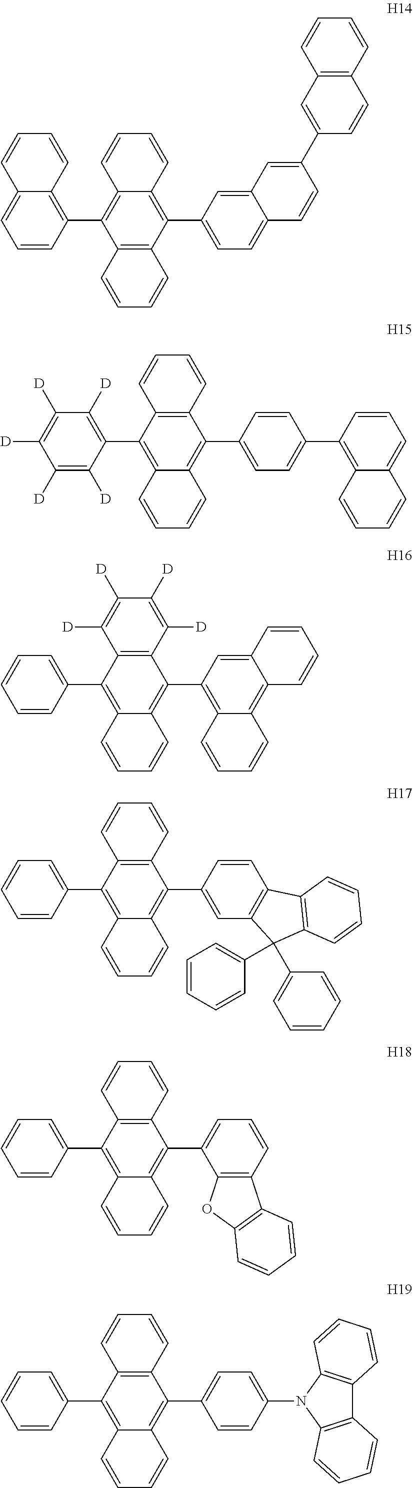

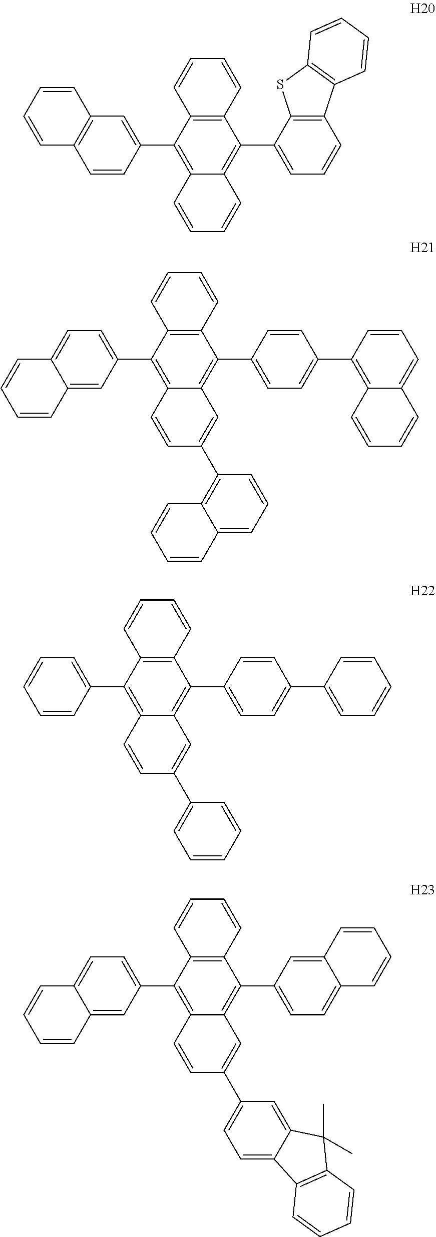

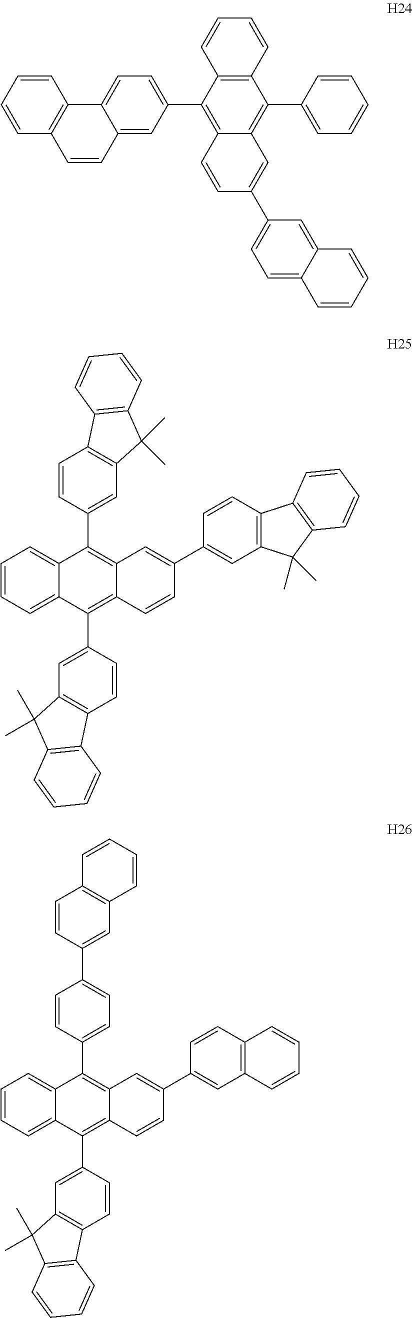

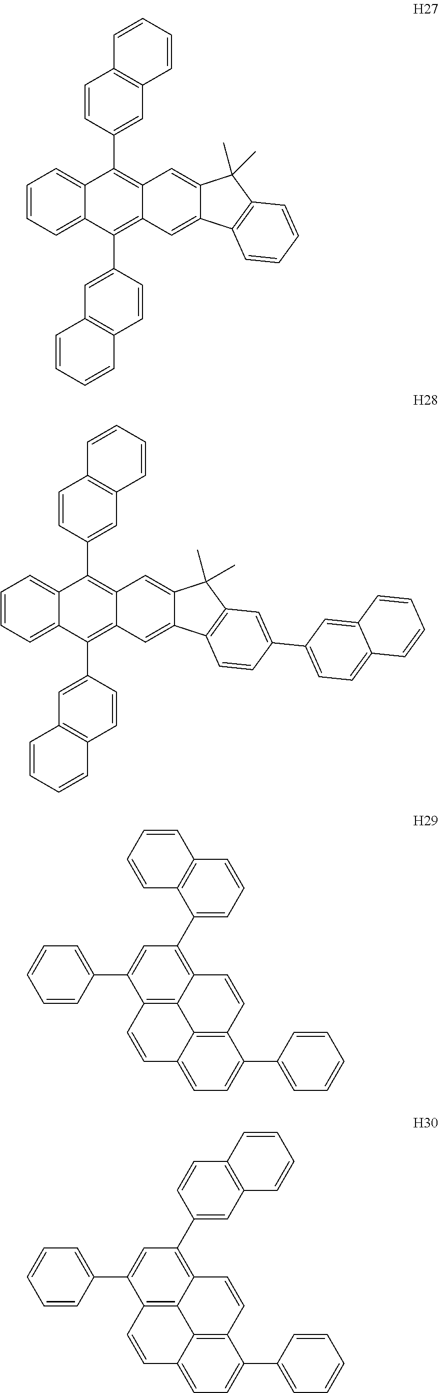

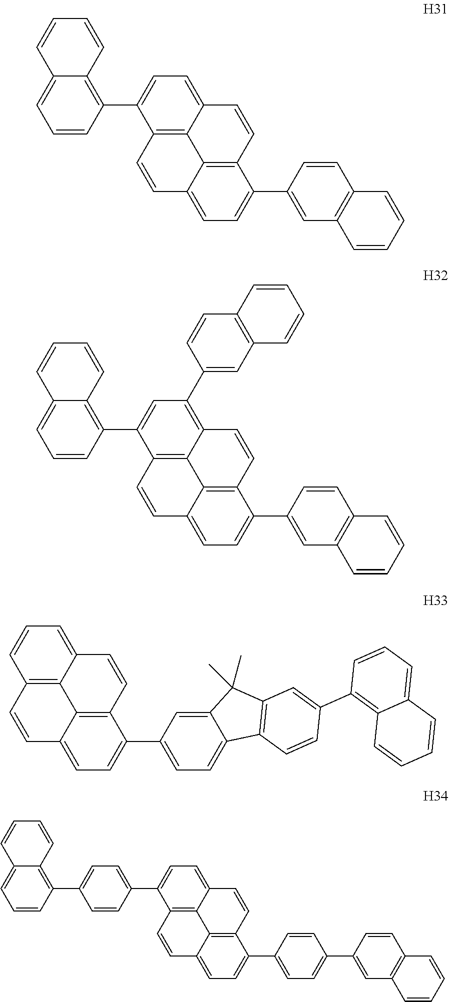

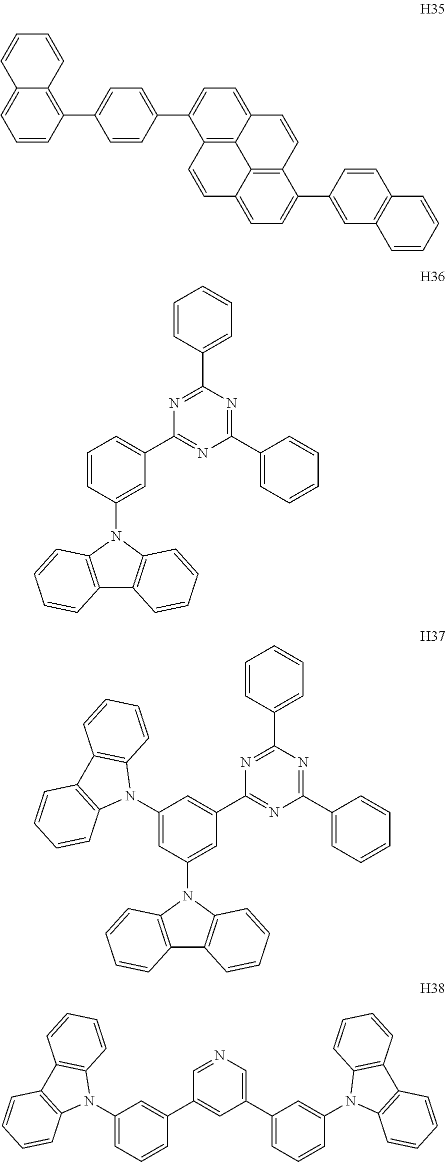

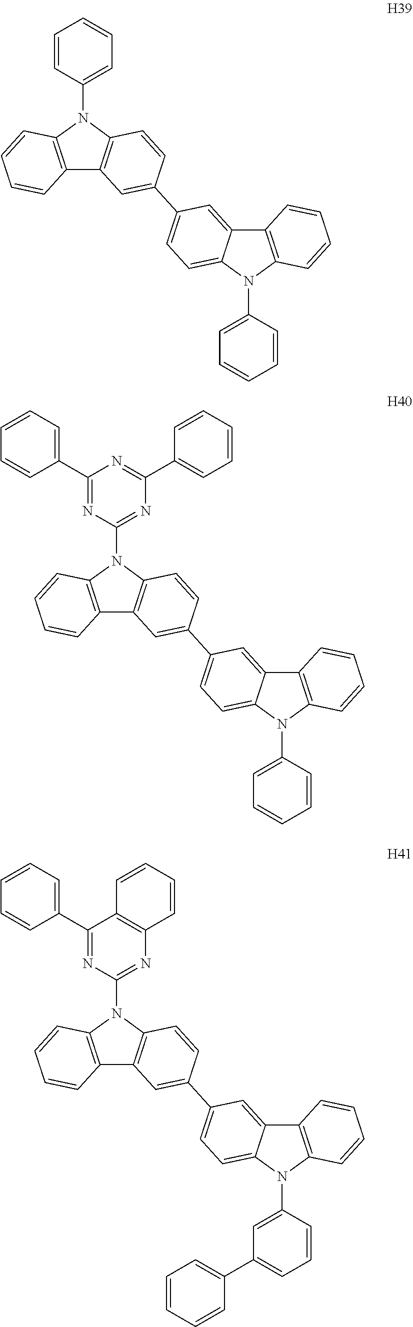 [SMILES: c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1]

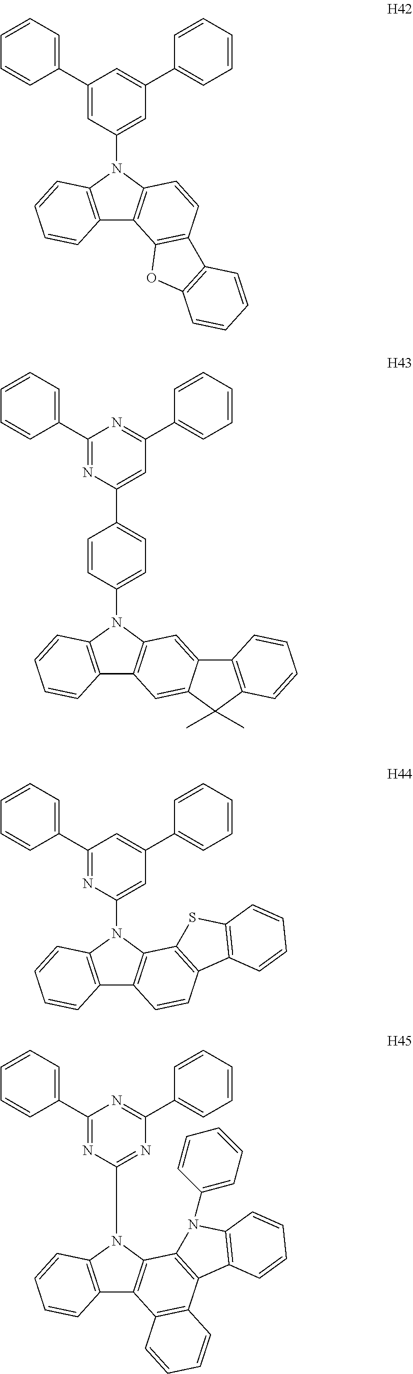 [SMILES: c1ccc(-c2cc(-c3ccccc3)cc(-n3c4ccccc4c4c5oc6ccccc6c5ccc43)c2)cc1 ; CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1]

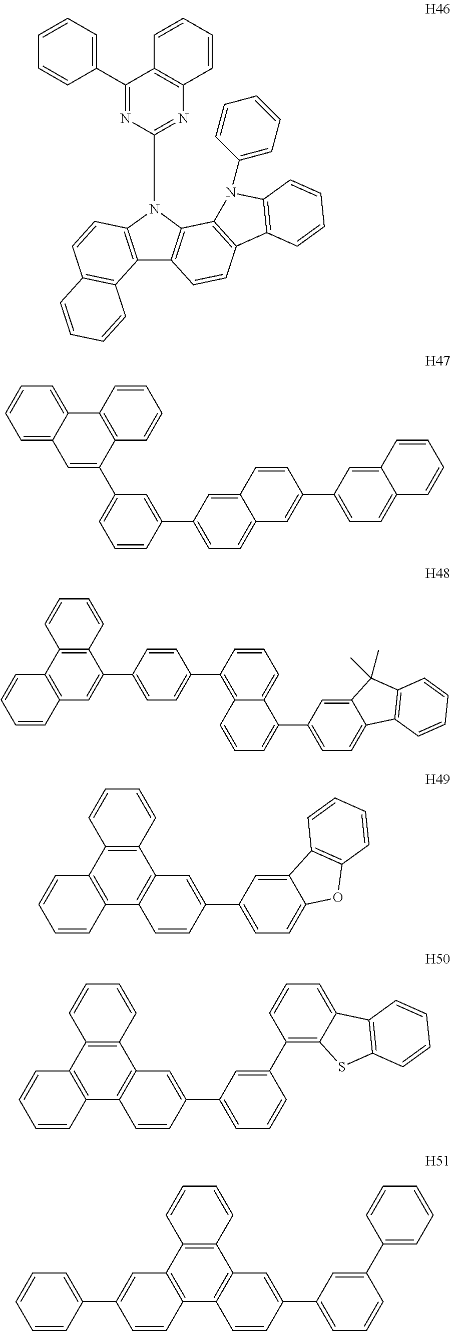

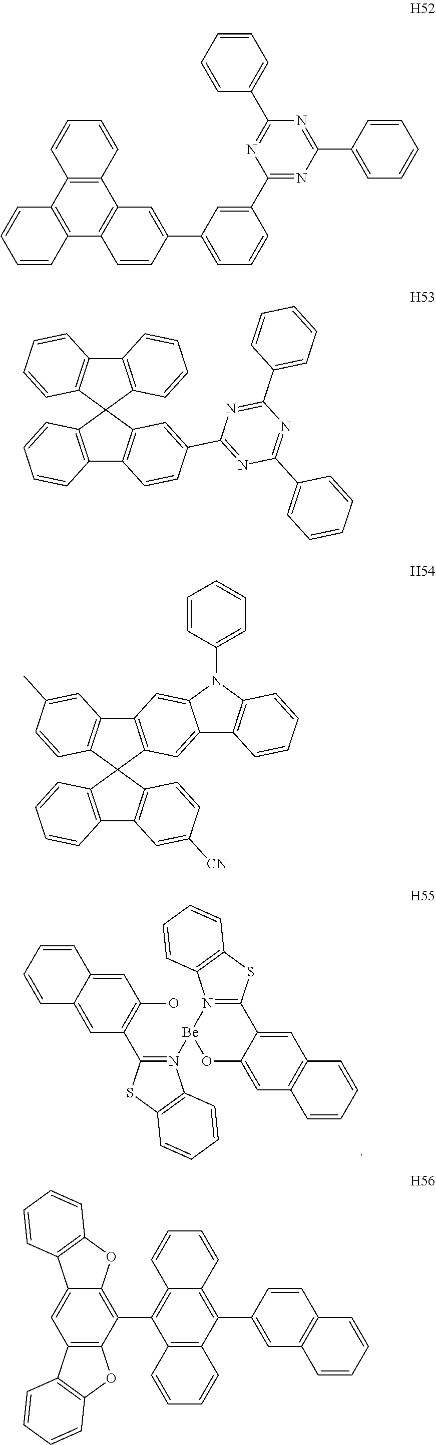

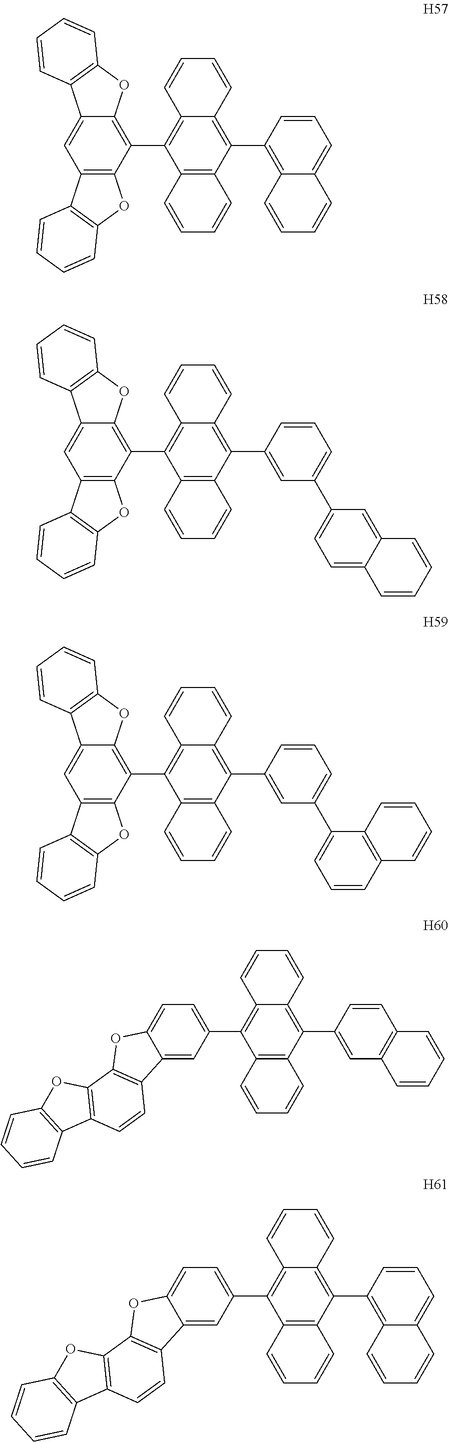 [SMILES: c1ccc2c(-c3c4ccccc4c(-c4c5oc6ccccc6c5cc5c4oc4ccccc45)c4ccccc34)cccc2c1 ; c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1]

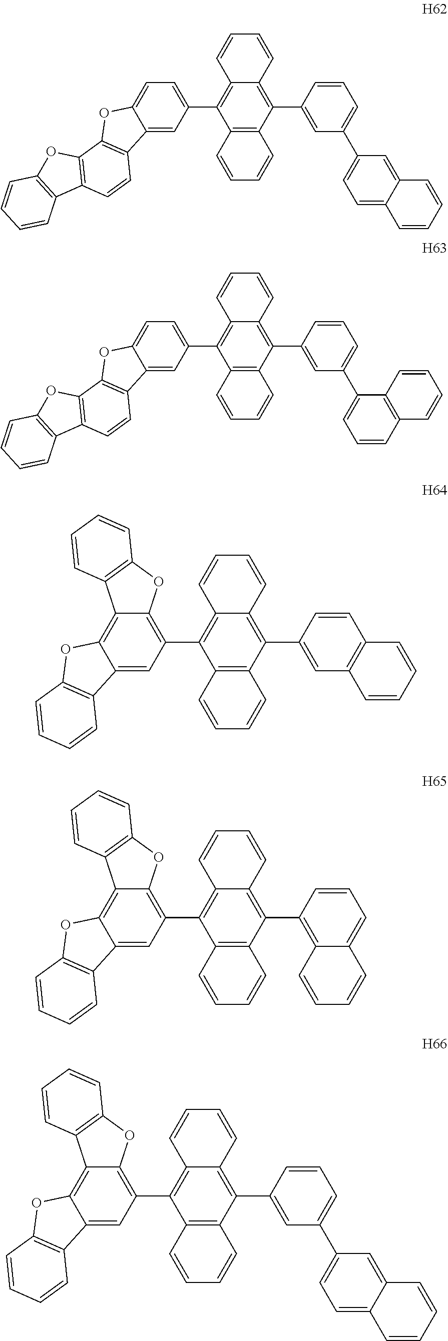

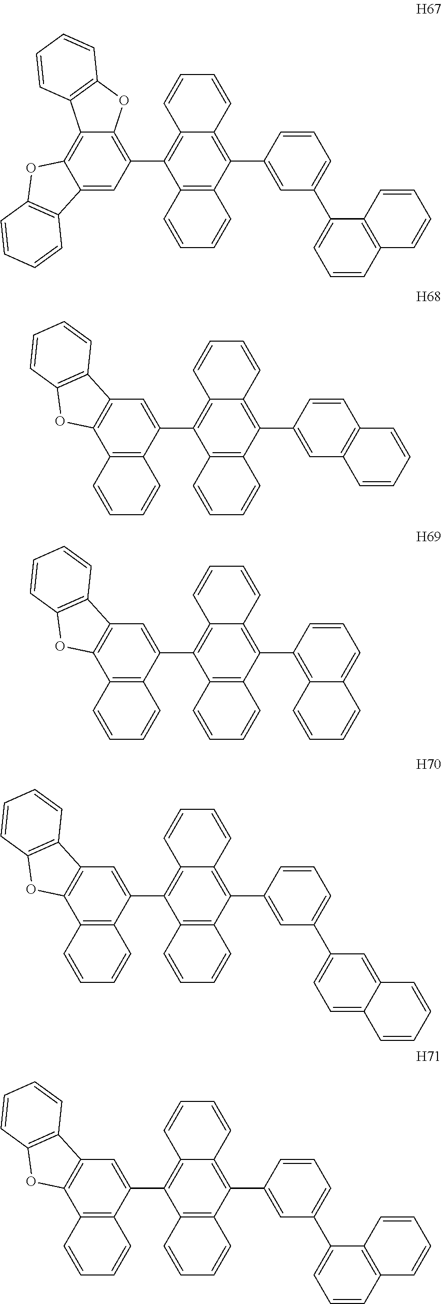 [SMILES: c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1]

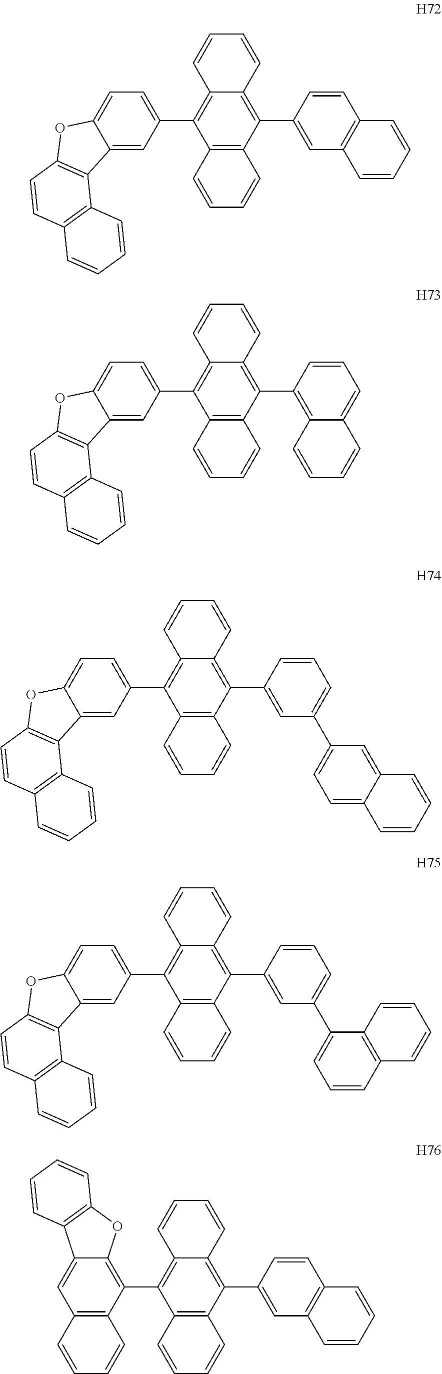

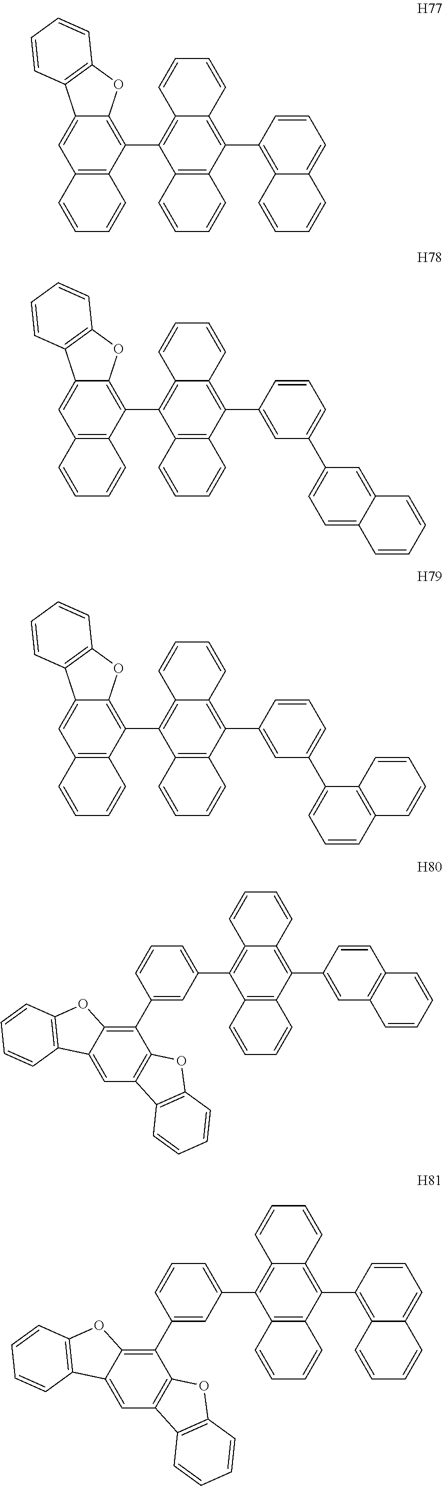 [SMILES: c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1]

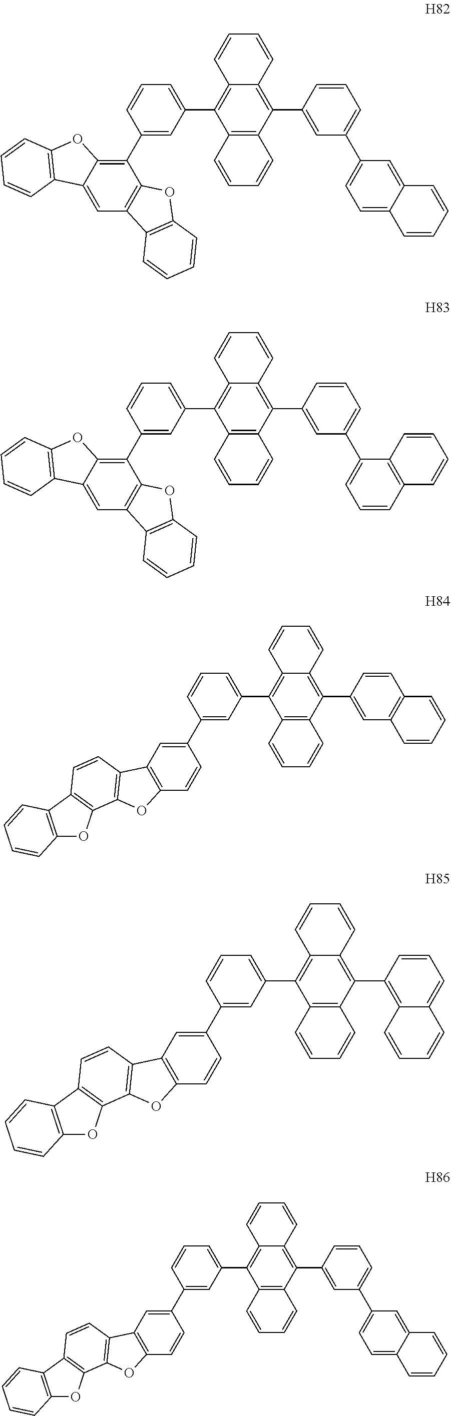 [SMILES: c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1]

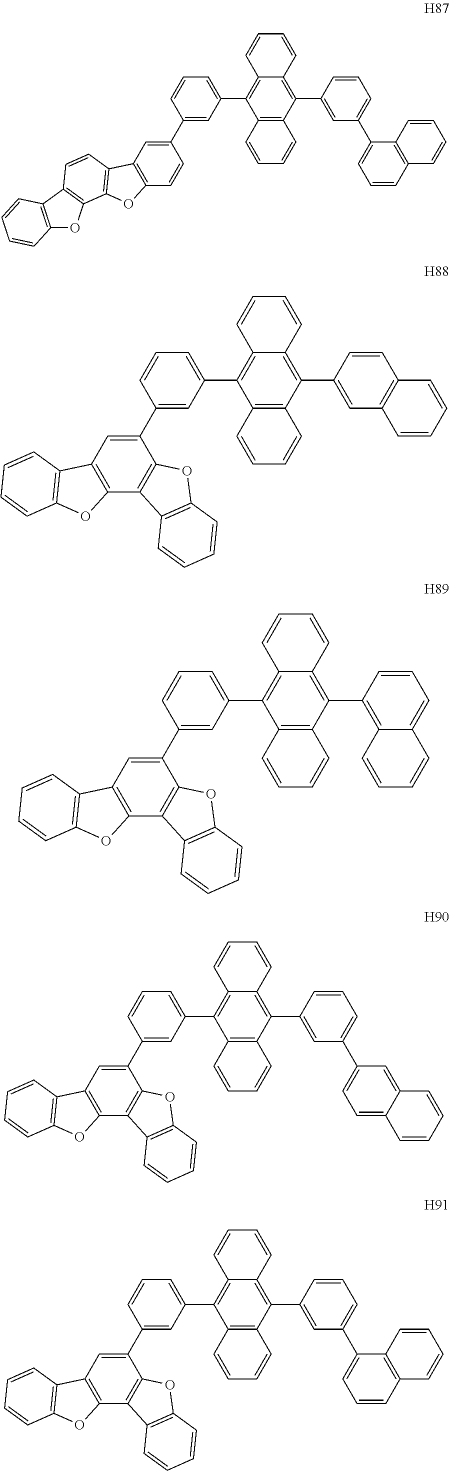 [SMILES: c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1]

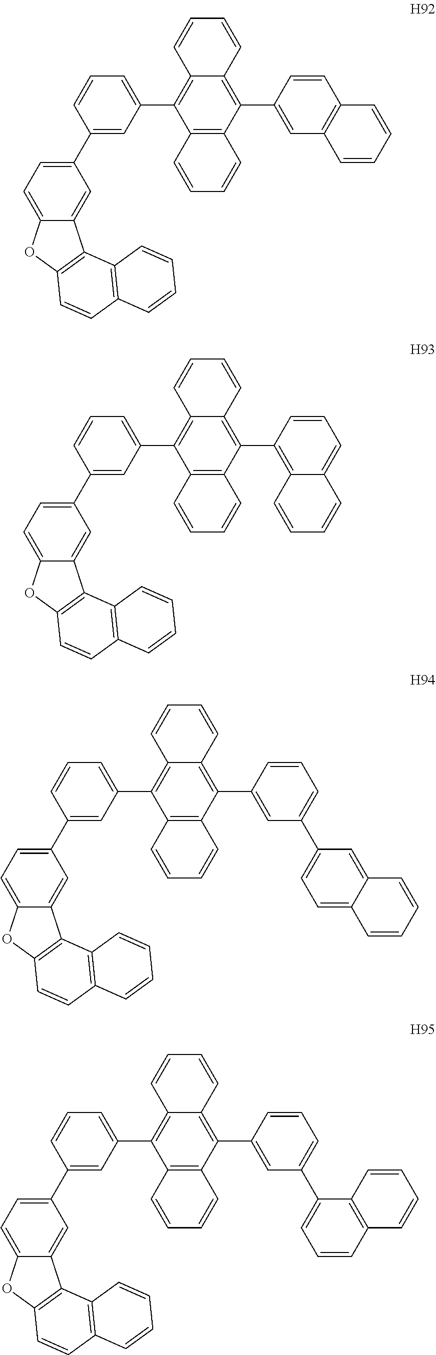 [SMILES: c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1]

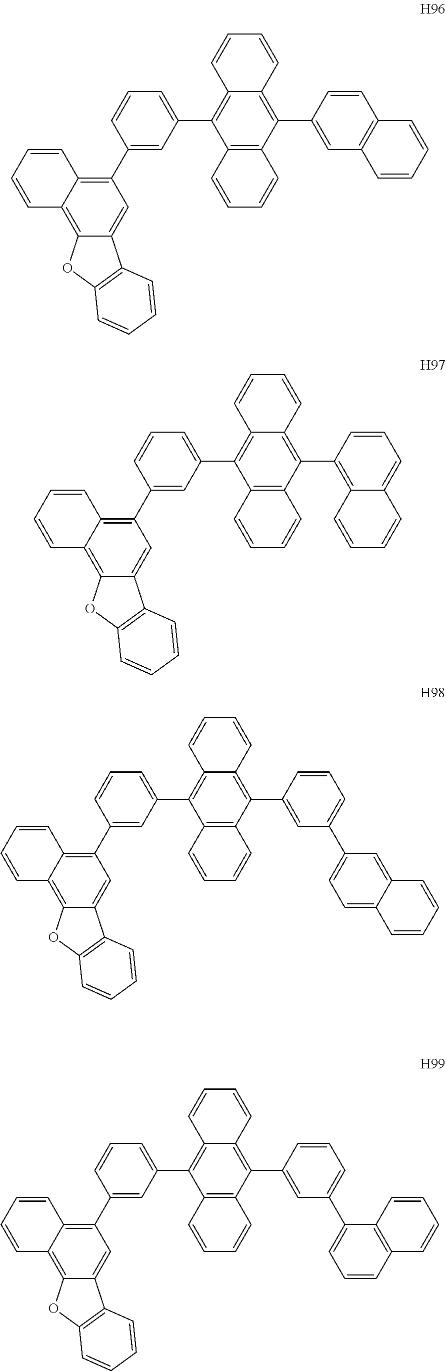 [SMILES: c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1]

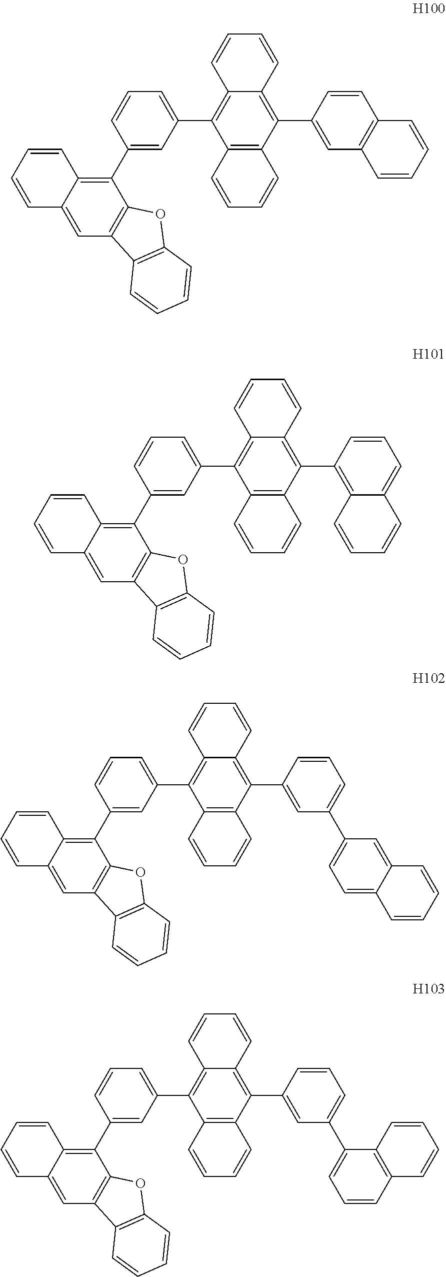 [SMILES: c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1]

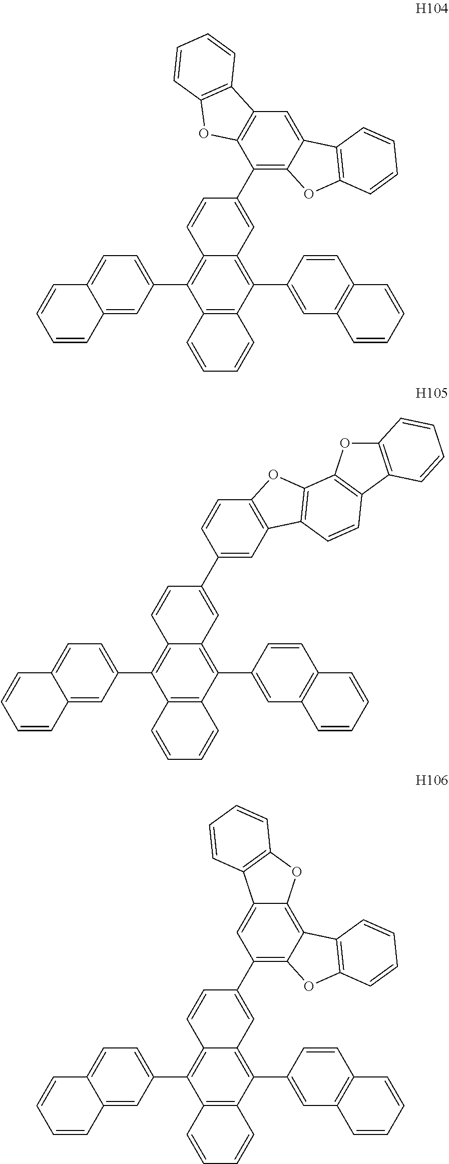 [SMILES: c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1]

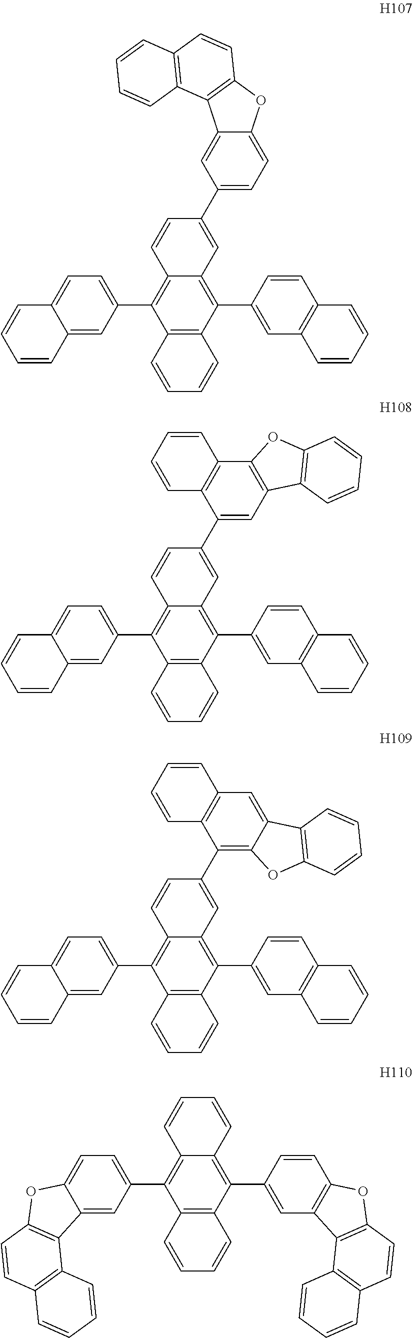

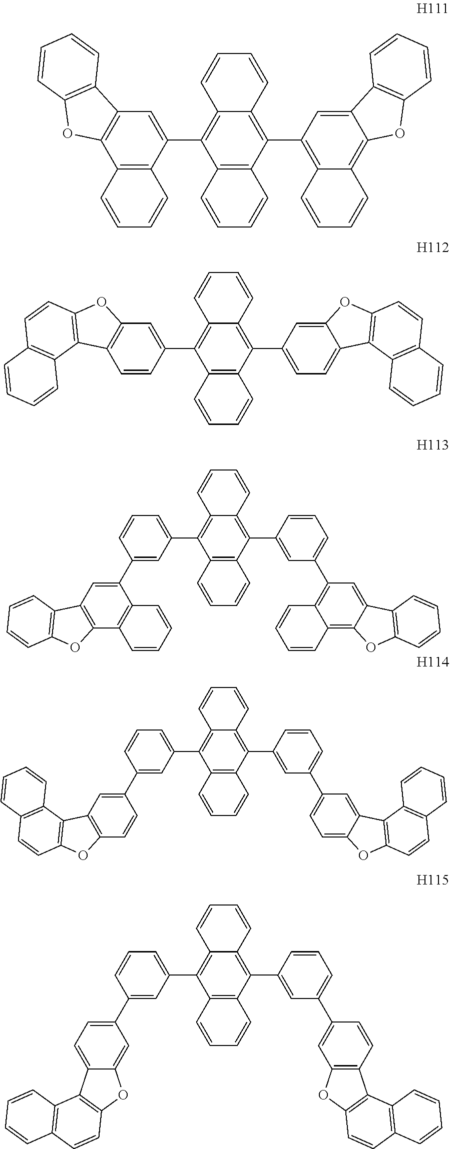 [SMILES: c1ccc2c(c1)oc1c3ccccc3c(-c3c4ccccc4c(-c4cc5c6ccccc6oc5c5ccccc45)c4ccccc34)cc21 ; c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1]

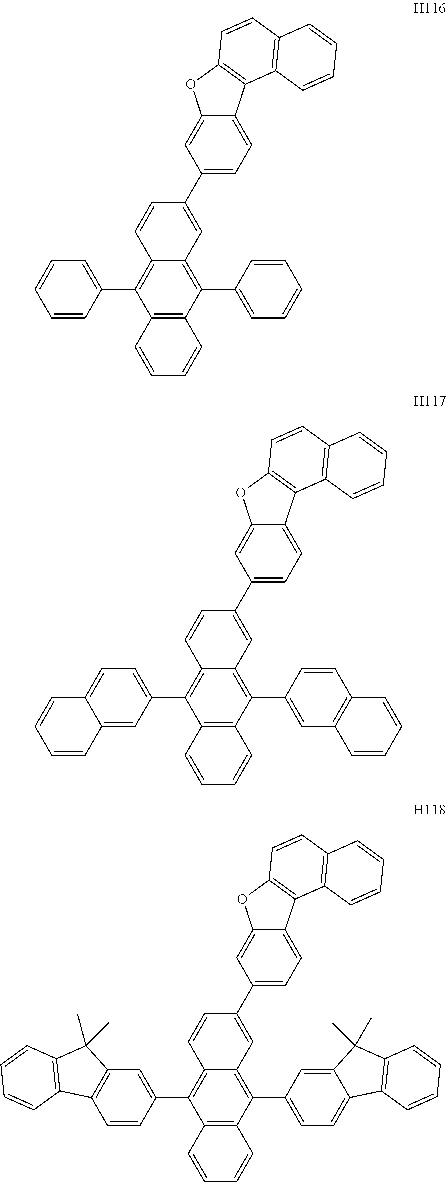 [SMILES: c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

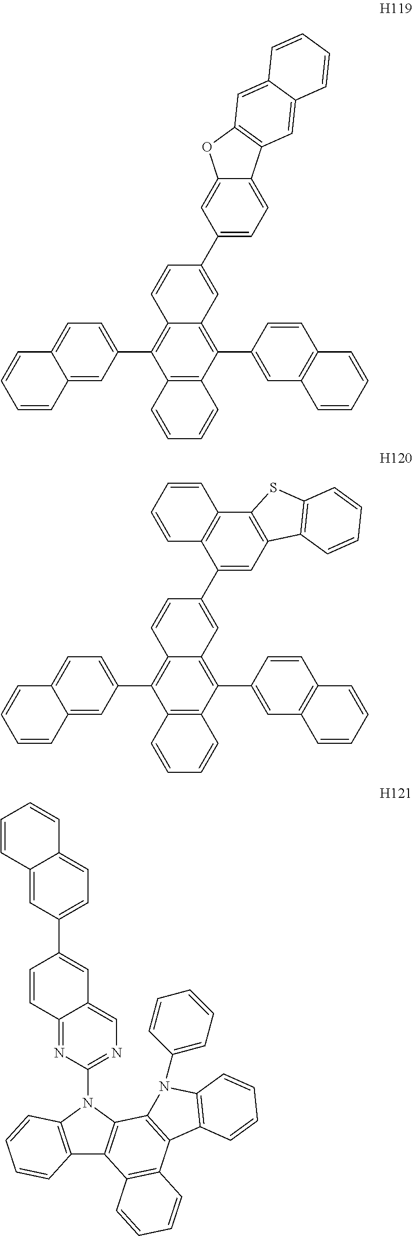

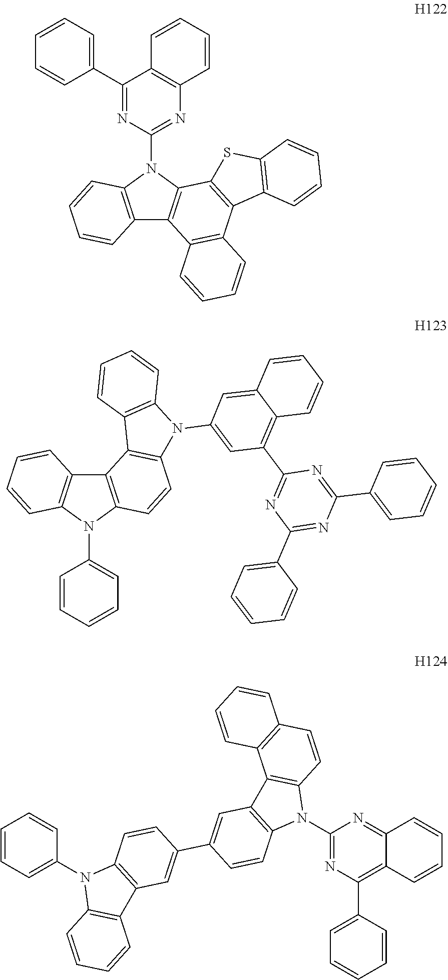 [SMILES: c1ccc(-c2nc(-n3c4ccccc4c4c5ccccc5c5c6ccccc6sc5c43)nc3ccccc23)cc1 ; c1ccc(-c2nc(-c3ccccc3)nc(-c3cc(-n4c5ccccc5c5c6c7ccccc7n(-c7ccccc7)c6ccc54)cc4ccccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

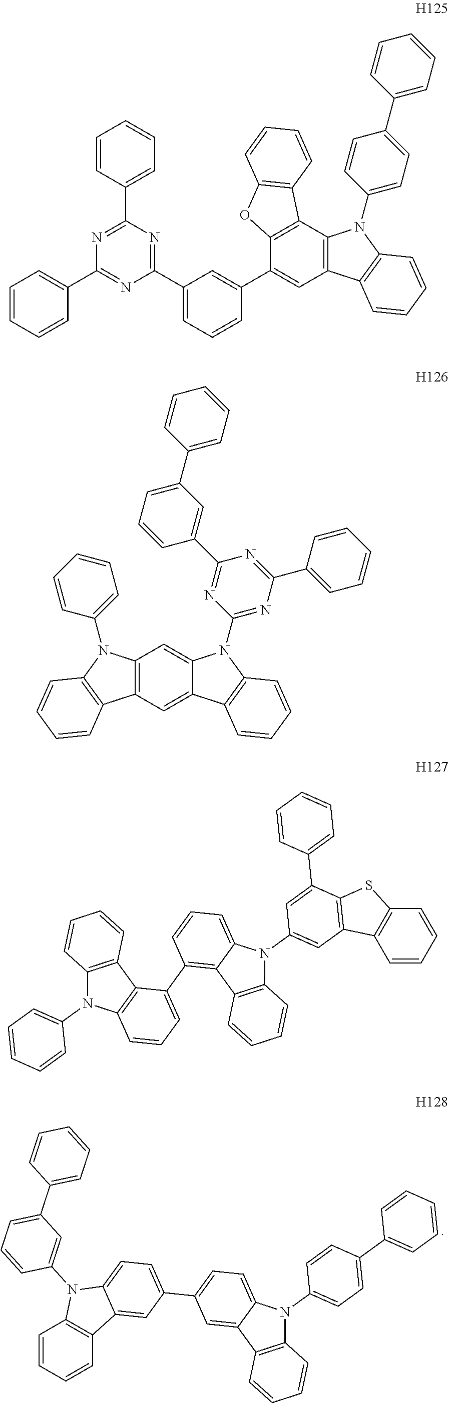 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

[Phosphorescent Dopant]

In embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

In embodiments, the phosphorescent dopant may include an organometallic compound represented by Formula 401:

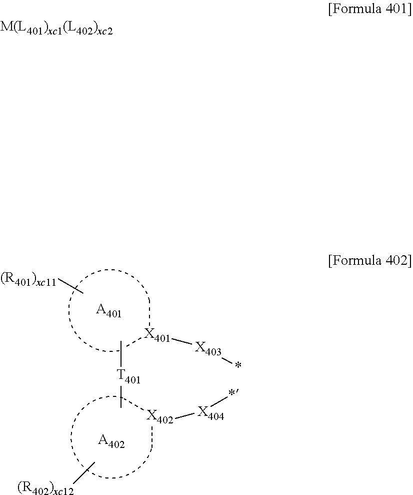

In Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein when xc1 is two or more, two or more of L₄₀₁(s) may be identical to or different from each other,
L₄₀₂ may be an organic ligand, and xc2 may be 0, 1, 2, 3, or 4, wherein when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *═C═*′,
X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each independently be the same as described herein with respect to Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each independently be the same as described herein with respect to Q₁,
xc11 and xc12 may each independently be an integer from 0 to 10, and
and *′ in Formula 402 each indicate a binding site to M in Formula 401.

In an embodiment, in Formula 402, X₄₀₁ may be nitrogen and X₄₀₂ may be carbon, or X₄₀₁ and X₄₀₂ may each be nitrogen.

In embodiments, in Formula 401, when xc1 is 2 or more, two ring A₄₀₁(s) in two or more of L₄₀₁(s) may optionally be linked to each other via T₄₀₂, which is a linking group, and two ring A₄₀₂(s) may optionally be linked to each other via T₄₀₃, which is a linking group (see Compounds PD1 to PD4 and PD7). T₄₀₂ and T₄₀₃ may each independently be the same as described herein with respect to T₄₀₁.

In Formula 401, L₄₀₂ may be an organic ligand. In an embodiment, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, etc.), or any combination thereof.

The phosphorescent dopant may include, for example, one of Compounds PD1 to PD39, or any combination thereof:

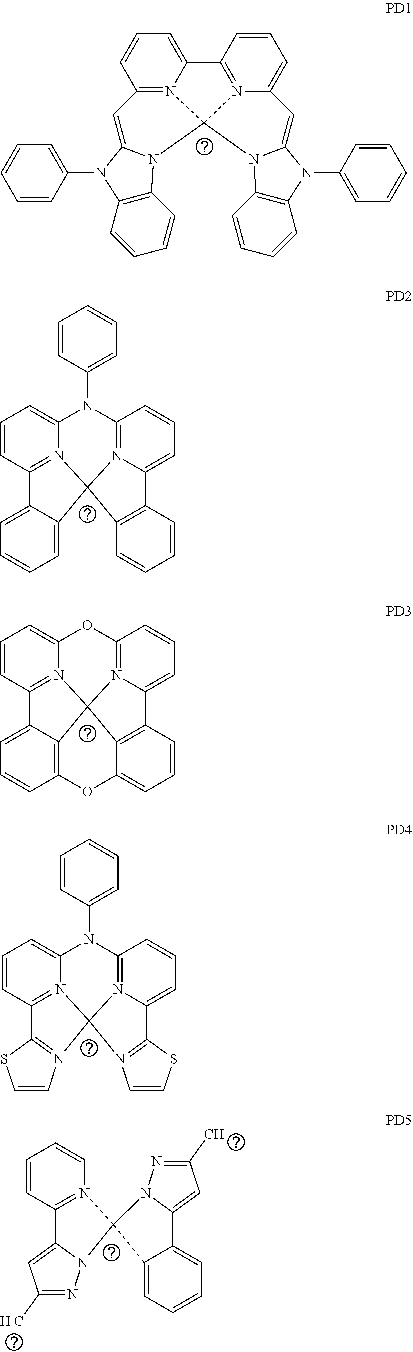

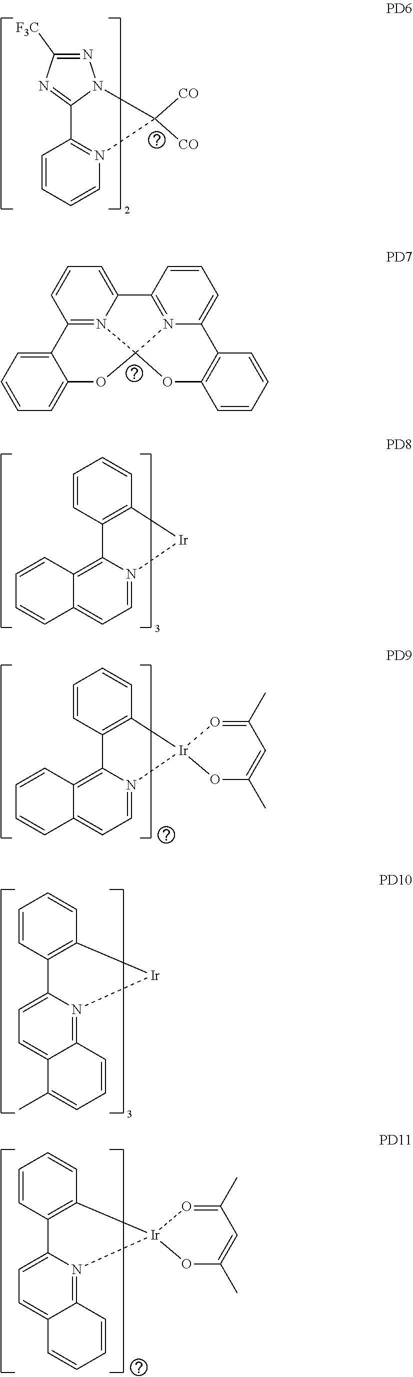

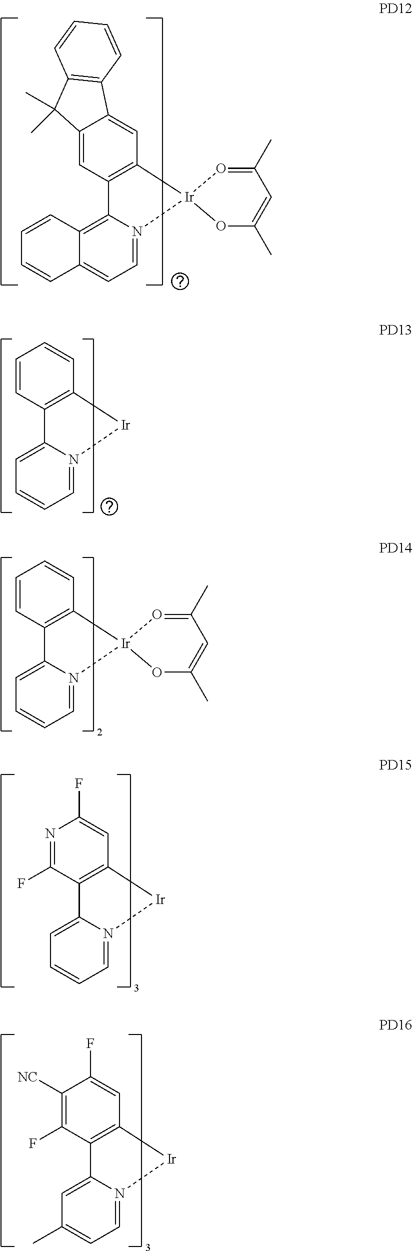

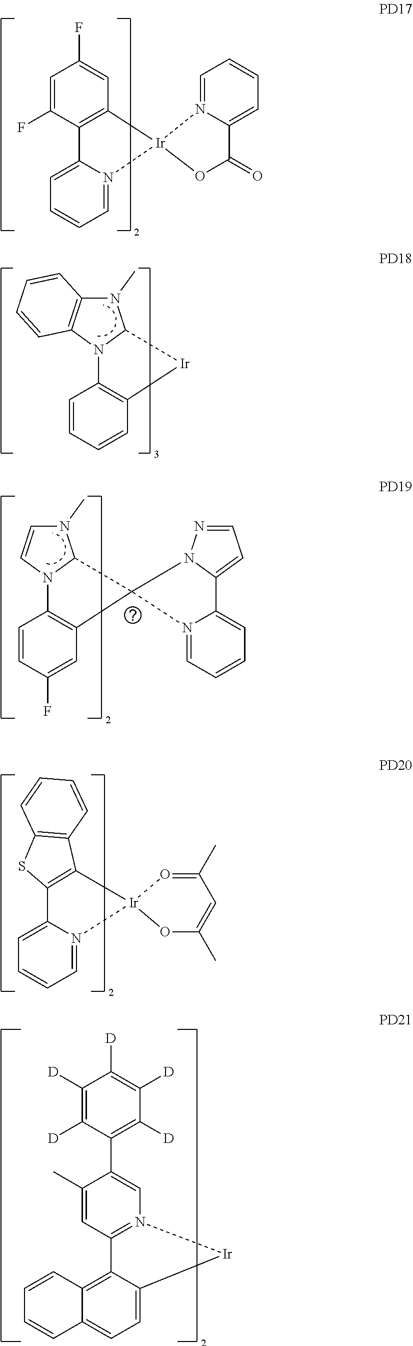

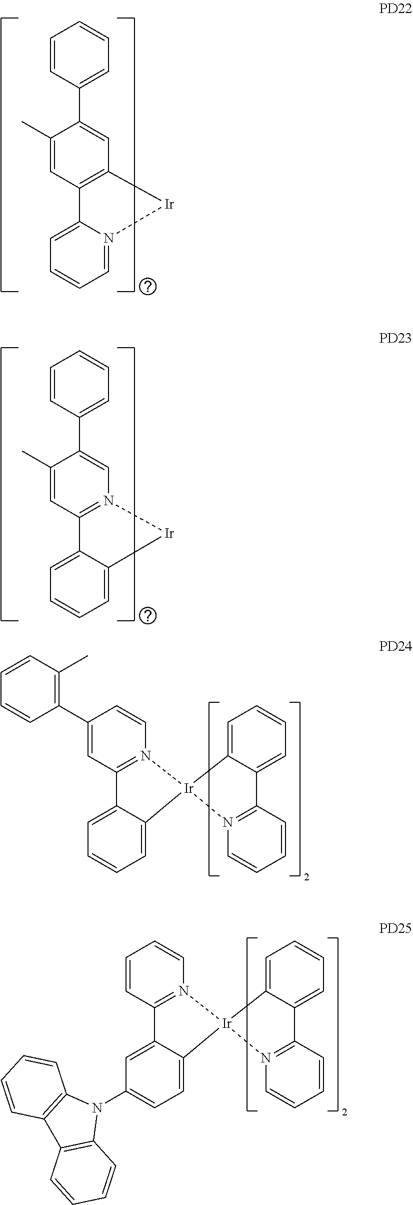

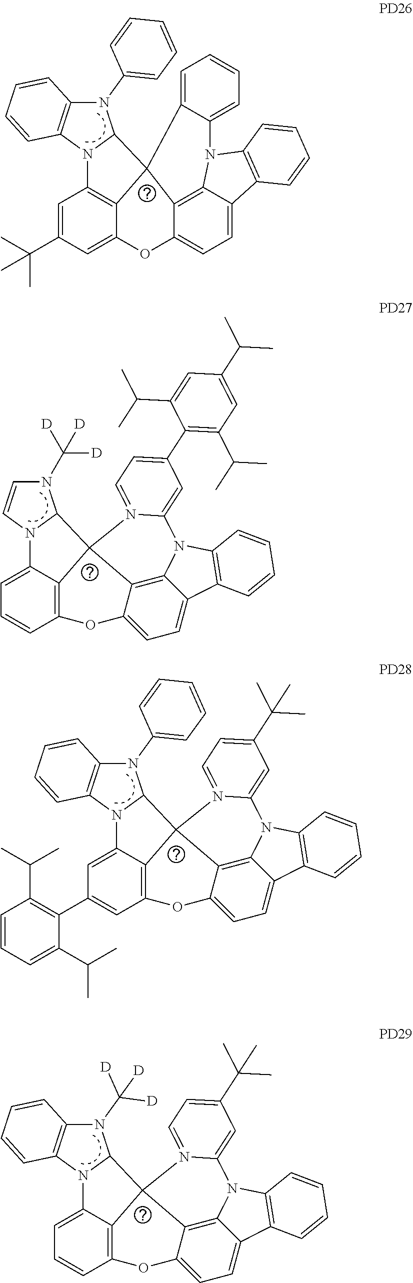

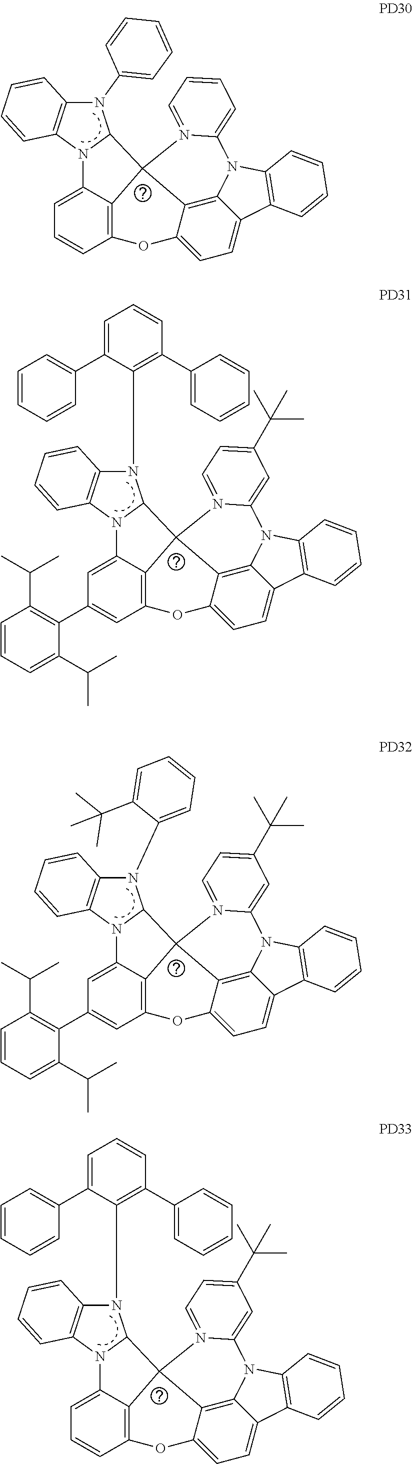

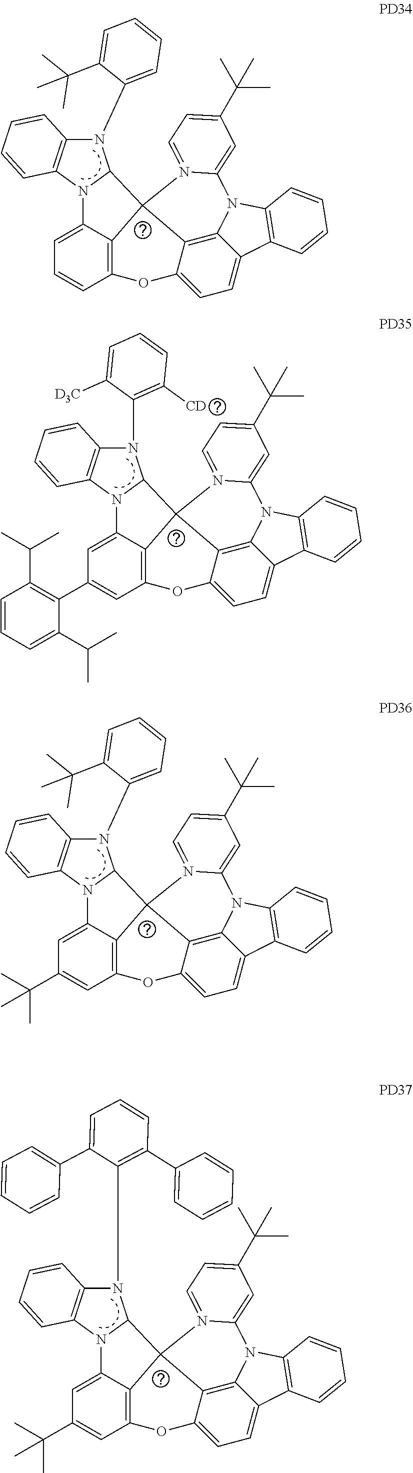

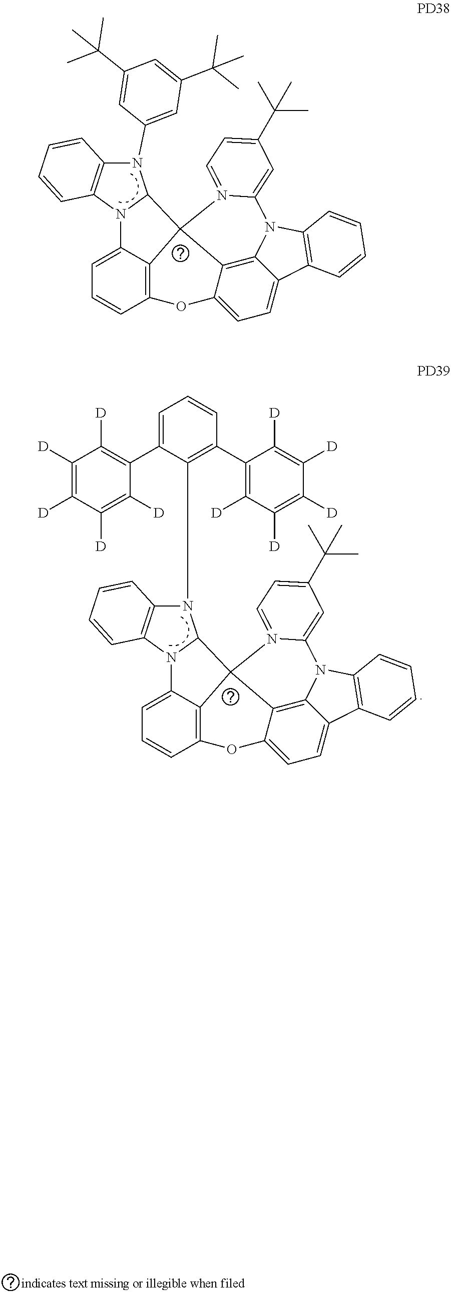

[Fluorescent Dopant]

The fluorescent dopant may include an amine group-containing compound, a styryl group-containing compound, or any combination thereof.

In an embodiment, the fluorescent dopant may include a compound represented by Formula 501:

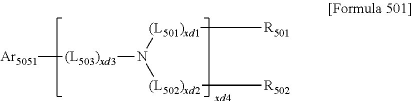

In Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

In an embodiment, in Formula 501, Ar₅₀₁ may be a condensed cyclic group (for example, an anthracene group, a chrysene group, or a pyrene group) in which three or more monocyclic groups are condensed together.

In embodiments, in Formula 501, xd4 may be 2.

In an embodiment, the fluorescent dopant may include one of Compounds FD1 to FD37, DPVBi, DPAVBi, or any combination thereof:

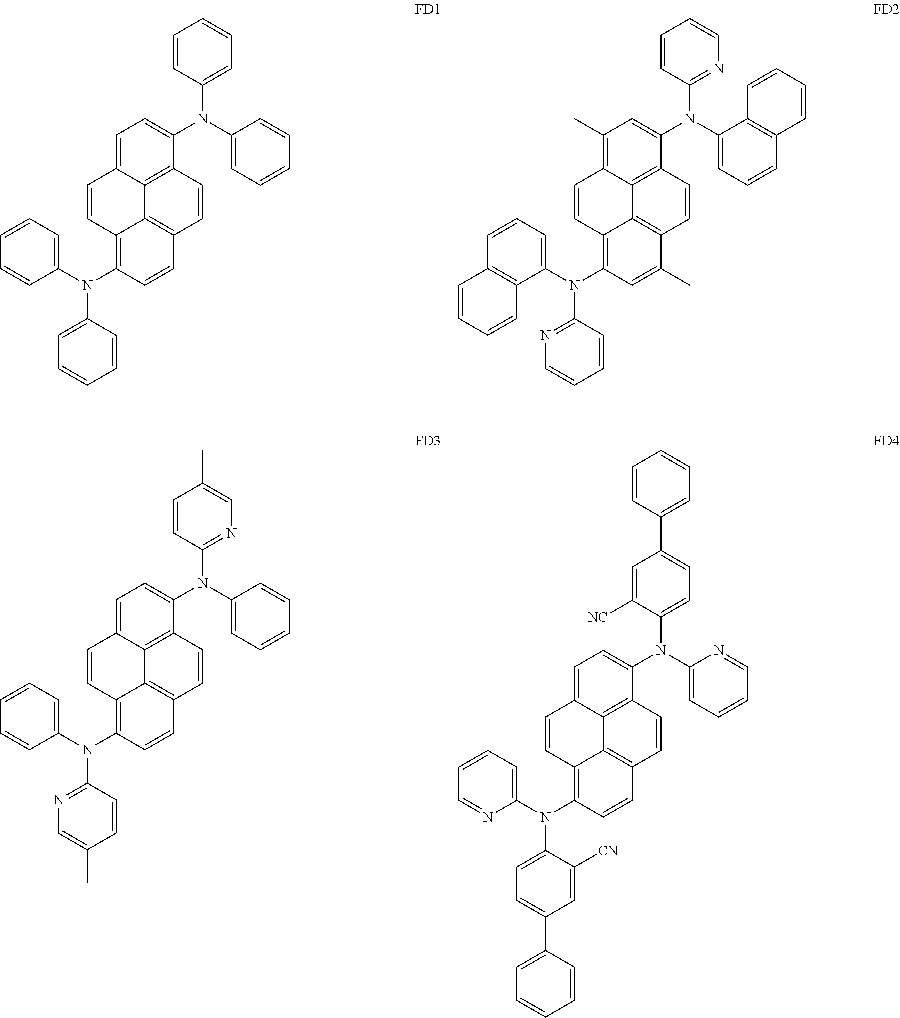 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1 ; N#Cc1cc(-c2ccccc2)ccc1N(c1ccccn1)c1ccc2ccc3c(N(c4ccccn4)c4ccc(-c5ccccc5)cc4C#N)ccc4ccc1c2c43]

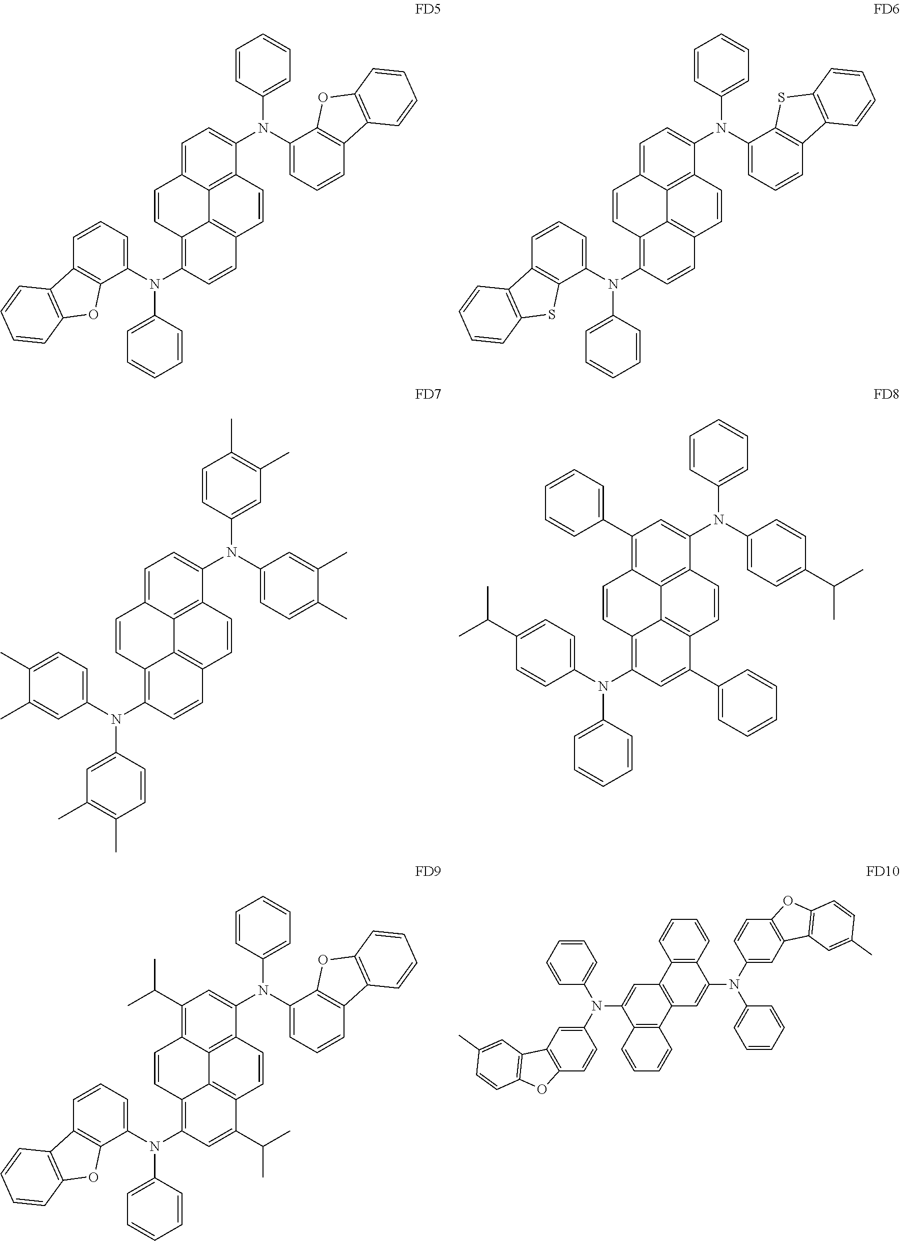

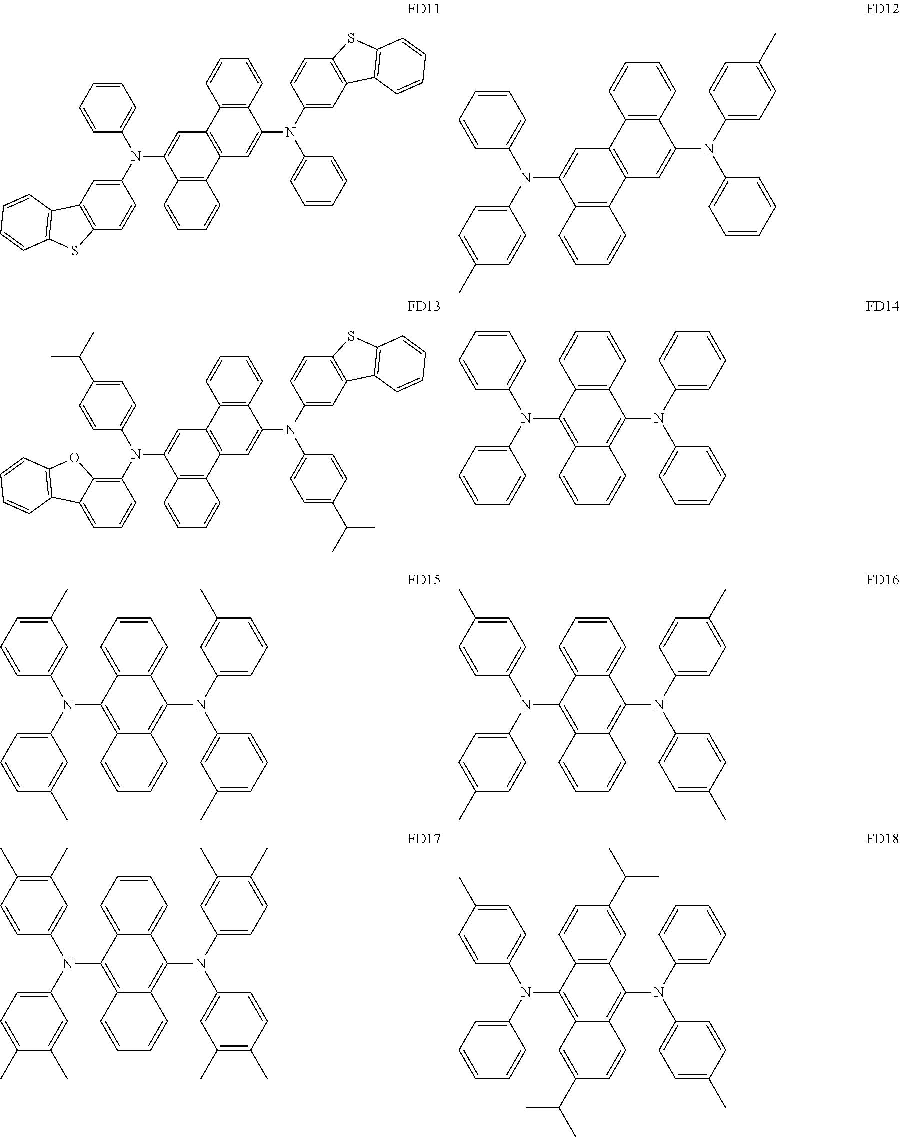

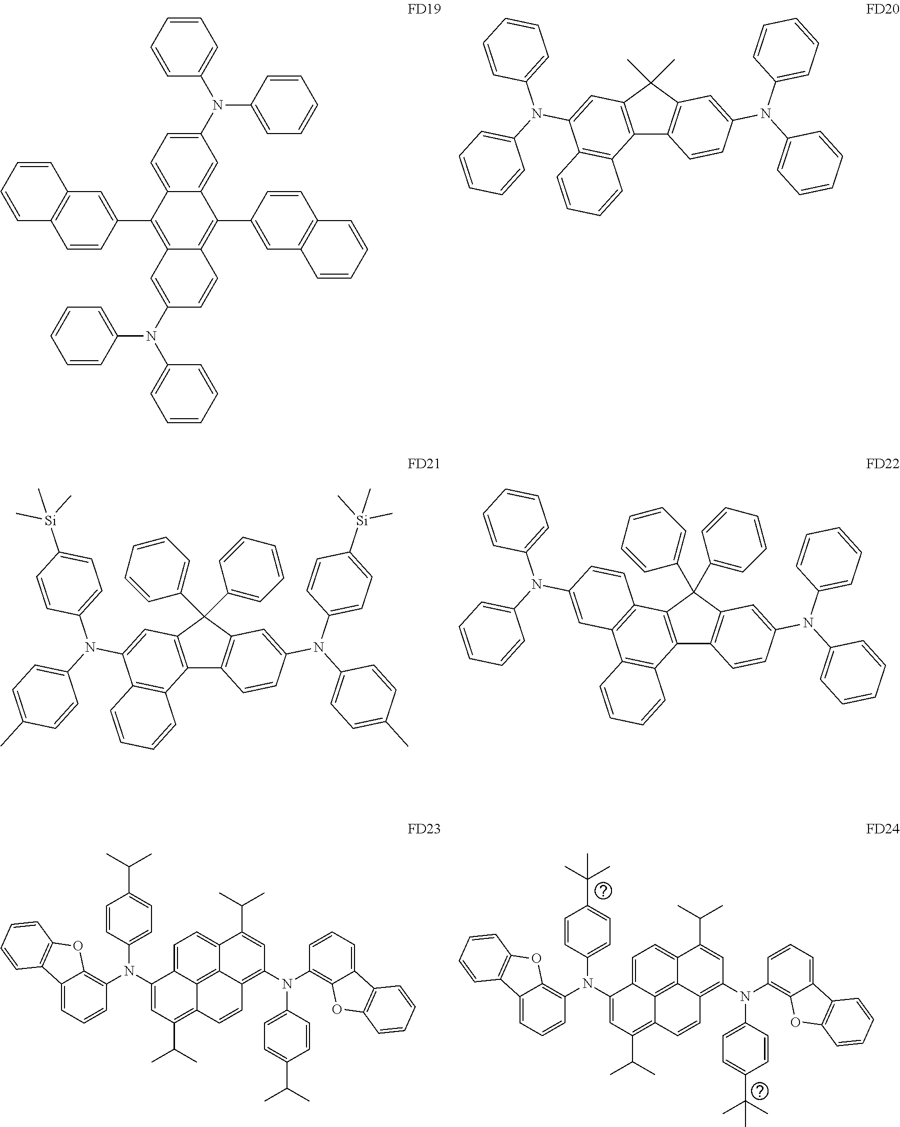

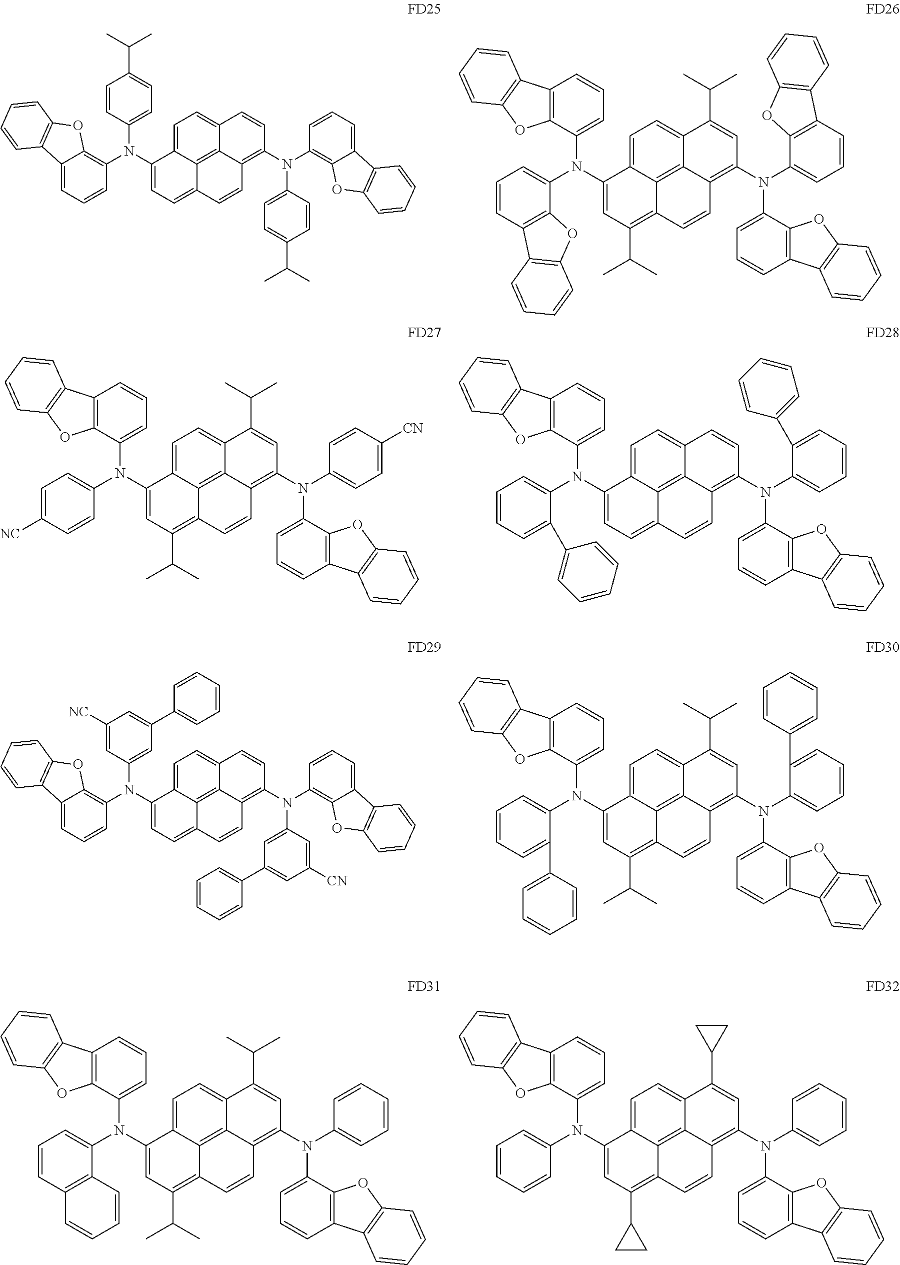

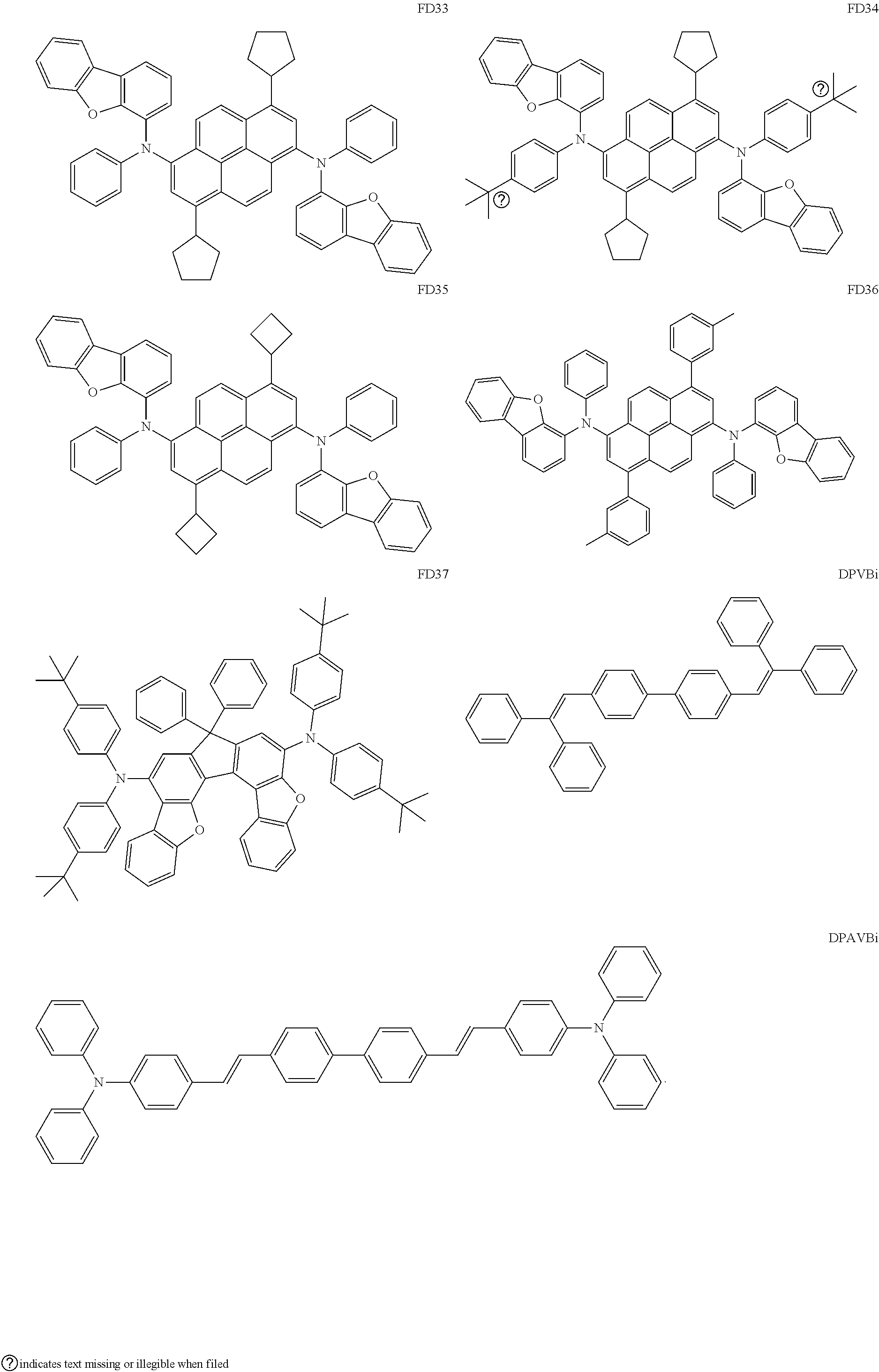

[Delayed Fluorescence Material]

The emission layer may include a delayed fluorescence material.

In the specification, the delayed fluorescence material may be selected from compounds capable of emitting delayed fluorescent light based on a delayed fluorescence emission mechanism.

The delayed fluorescence material included in the emission layer may serve as a host or as a dopant, depending on the types of other materials included in the emission layer.

In an embodiment, a difference between a triplet energy level (eV) of the delayed fluorescence material and a singlet energy level (eV) of the delayed fluorescence material may be in a range of about 0 eV to about 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively occur, and thus, the luminescence efficiency of the light-emitting device 10 may be improved.

In embodiments, the delayed fluorescence material may include: a material including at least one electron donor (for example, a Ti electron-rich C₃-C₆₀ cyclic group, such as a carbazole group) and at least one electron acceptor (for example, a sulfoxide group, a cyano group, or a Ti electron-deficient nitrogen-containing C₁-C₆₀ cyclic group); or a material including a C₈-C₆₀ polycyclic group in which two or more cyclic groups are condensed while sharing boron (B).

Examples of the delayed fluorescence material may include at least one of Compounds DF1 to DF14:

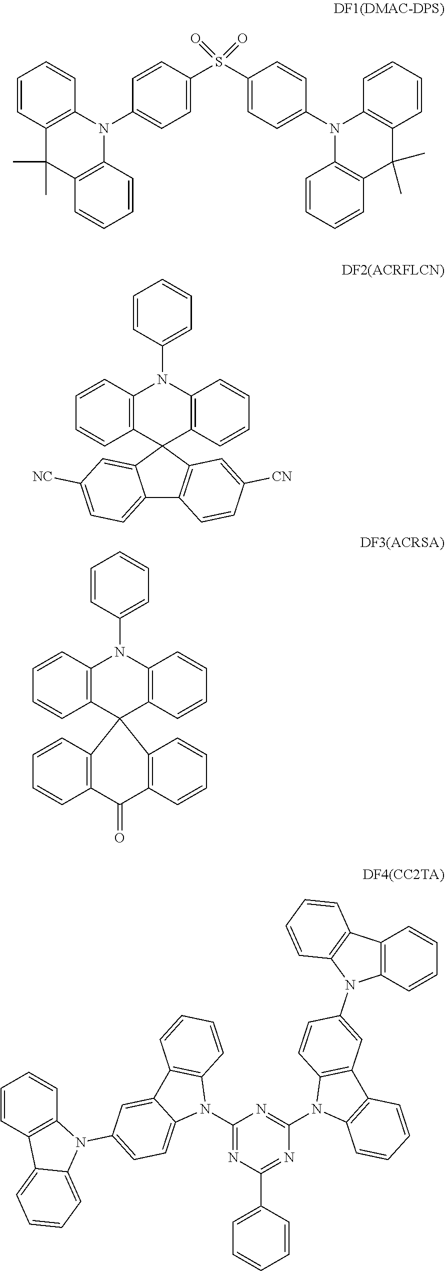

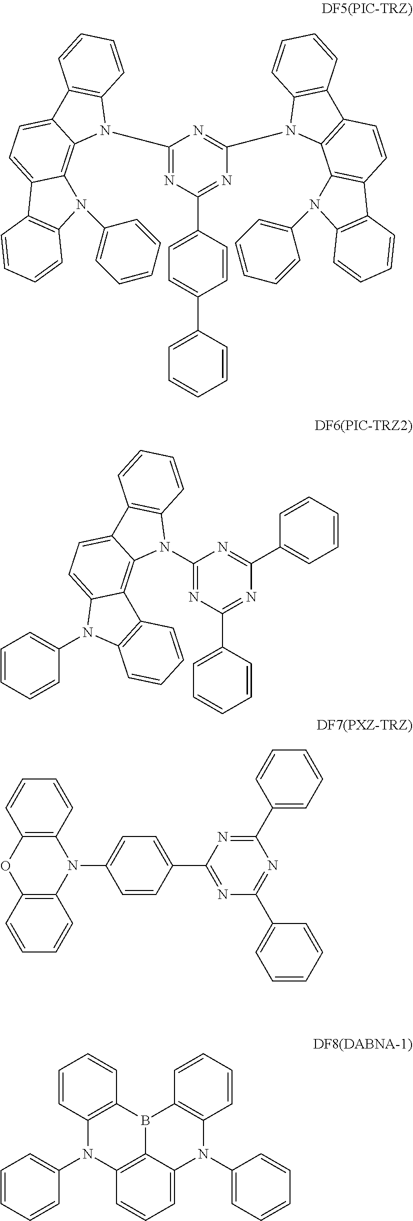 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1 ; c1ccc(N2c3ccccc3B3c4ccccc4N(c4ccccc4)c4cccc2c43)cc1]

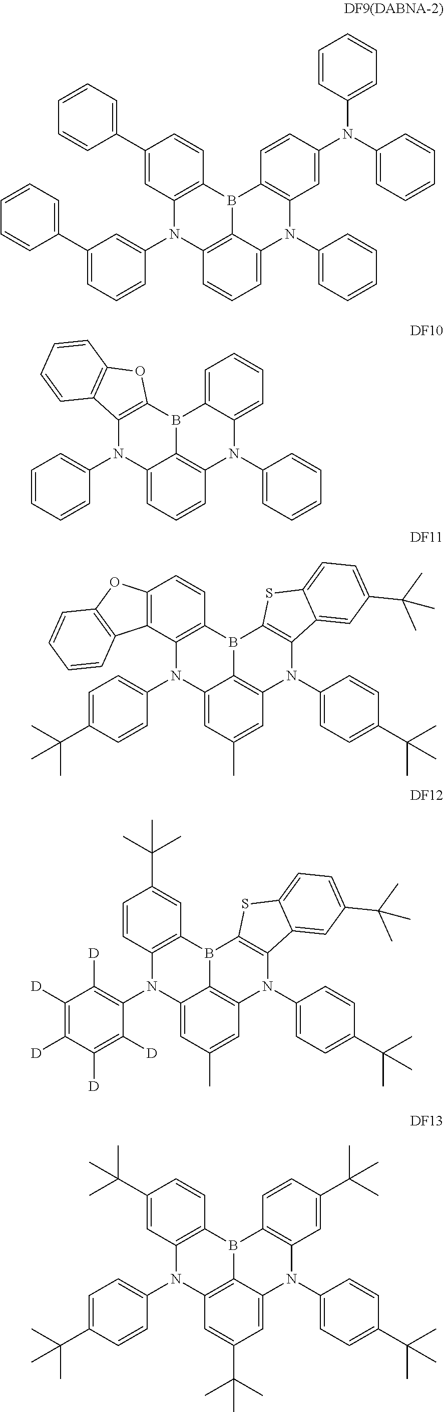

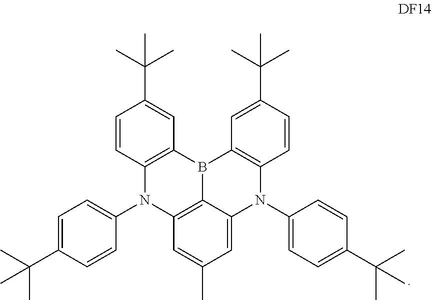 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

[Quantum Dot]

The emission layer may include a quantum dot.

In the specification, a quantum dot may be a crystal of a semiconductor compound, and may include any material capable of emitting light of various emission wavelengths according to a size of the crystal.

A diameter of the quantum dots may be, for example, in a range of about 1 nm to about 10 nm.

The quantum dot may be synthesized by a wet chemical process, a metal organic chemical vapor deposition process, a molecular beam epitaxy process, or any process similar thereto.

The wet chemical process is a method that may include mixing a precursor material with an organic solvent and growing a quantum dot particle crystal. When the crystal grows, the organic solvent naturally acts as a dispersant coordinated on the surface of the quantum dot crystal and controls the growth of the crystal so that the growth of quantum dot particles can be controlled through a process which costs less, and may be more readily performed than vapor deposition methods, such as metal organic chemical vapor deposition (MOCVD) or molecular beam epitaxy (MBE).

The quantum dot may include a Group II-VI semiconductor compound, a Group III-V semiconductor compound, a Group III-VI semiconductor compound, a Group I-III-VI semiconductor compound, a Group IV-VI semiconductor compound, a Group IV element or compound, or any combination thereof.

Examples of the Group II-VI semiconductor compound may include: a binary compound, such as CdS, CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any combination thereof.

Examples of the Group III-V semiconductor compound may include: a binary compound, such as GaN, GaP, GaAs, GaSb, AlN, AlP, AlAs, AISb, InN, InP, InAs, or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, or InPSb; a quaternary compound, such as GaAlNP, GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, or InAlPSb; or any combination thereof. In an embodiment, the Group III-V semiconductor compound may further include a Group II element. Examples of the Group III-V semiconductor compound further including a Group II element may include InZnP, InGaZnP, InAlZnP, etc.

Examples of the Group III-VI semiconductor compound may include: a binary compound, such as GaS, GaSe, Ga₂Se₃, GaTe, InS, InSe, In₂S₃, In₂Se₃, or InTe; a ternary compound, such as InGaS₃, or InGaSe₃; or any combination thereof.

Examples of the Group I-III-VI semiconductor compound may include: a ternary compound, such as AgInS, AgInS₂, CuInS, CuInS₂, CuGaO₂, AgGaO₂, or AgAlO₂; or any combination thereof.

Examples of the Group IV-VI semiconductor compound may include: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, or SnPbTe; a quaternary compound, such as SnPbSSe, SnPbSeTe, or SnPbSTe; or any combination thereof.

Examples of the Group IV element or compound may include: a single element material, such as Si or Ge; a binary compound, such as SiC or SiGe; or any combination thereof.

Each element included in a multi-element compound, such as a binary compound, a ternary compound, or a quaternary compound may be present in a particle at a uniform concentration or at a non-uniform concentration.

In embodiments, the quantum dot may have a single structure in which the concentration of each element in the quantum dot is uniform, or the quantum dot may have a core-shell structure. In an embodiment, in case that the quantum dot has a core-shell structure, a material included in the core and a material included in the shell may be different from each other.

The shell of the quantum dot may serve as a protective layer that prevents chemical degeneration of the core to maintain semiconductor characteristics, and/or may serve as a charging layer that imparts electrophoretic characteristics to the quantum dot. The shell may be a single layer or a multi-layer. An interface between the core and the shell may have a concentration gradient in which the concentration of a material that is present in the shell decreases toward the core.

Examples of the shell of the quantum dot may include a metal oxide, a metalloid oxide, a non-metal oxide, a semiconductor compound, or any combination thereof. Examples of the metal oxide, the metalloid oxide, or the non-metal oxide may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; or any combination thereof.

Examples of the semiconductor compound may include, as described herein, a Group II-VI semiconductor compound, a Group III-V semiconductor compound, a Group III-VI semiconductor compound, a Group I-III-VI semiconductor compound, a Group IV-VI semiconductor compound, or any combination thereof. Examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AISb, or any combination thereof.

A full width at half maximum (FWHM) of an emission wavelength spectrum of the quantum dot may be equal to or less than about 45 nm. For example, a FWHM of an emission wavelength spectrum of the quantum dot may be equal to or less than about 40 nm. For example, a FWHM of an emission wavelength spectrum of the quantum dot may be equal to or less than about 30 nm. Within these ranges, color purity or color reproducibility may be increased. Light emitted through the quantum dot may be emitted in all directions, so that a wide viewing angle may be improved.

In embodiments, the quantum dot may be in the form of a spherical particle, a pyramidal particle, a multi-arm particle, a cubic nanoparticle, a nanotube particle, a nanowire particle, a nanofiber particle, or a nanoplate particle.

Since the energy band gap may be adjusted by controlling the size of the quantum dot, light having various wavelength bands may be obtained from a quantum dot emission layer. Accordingly, by using quantum dots of different sizes, a light-emitting device that emits light of various wavelengths may be implemented. In embodiments, the size of the quantum dot may be selected to emit red, green, and/or blue light. In an embodiment, the size of the quantum dot may be configured to emit white light by combination of light of various colors.

[Electron Transport Region in Interlayer 130]

The electron transport region may have a structure consisting of a layer consisting of a single material, a structure consisting of a layer including different materials, or a including multiple layers including different materials.

The electron transport region may include a buffer layer, a hole blocking layer, an electron control layer, an electron transport layer, an electron injection layer, or any combination thereof.

In embodiments, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein the layers of each structure may be stacked from an emission layer in its respective stated order, but the structure of the electron transport region is not limited thereto.

In an embodiment, the electron transport region (for example, a buffer layer, a hole blocking layer, an electron control layer, or an electron transport layer in the electron transport region) may include a metal-free compound including at least one rr electron-deficient nitrogen-containing C₁-C₆₀ cyclic group.

In embodiments, the electron transport region may include a compound represented by Formula 601:

[Ar₆₀₁]ₓₑ₁₁-[(L₆₀₁)ₓₑ₁-R₆₀₁]ₓₑ₂₁ [Formula 601]

In Formula 601,
Ar₆₀₁ and L₆₀₁ may each independently be a C₃-C₆₀ carbocyclic group unsubstituted or substituted with at least one R₁₀ₐ or a C₁-C₆ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each independently be the same as described herein with respect to Q₁,
xe21 may be 1, 2, 3, 4, or 5,
at least one of Ar₆₀₁, L₆₀₁, and R₆₀₁ may each independently be a π electron-deficient nitrogen-containing C₁-C₆₀ cyclic group that is unsubstituted or substituted with at least one R₁₀ₐ.

In an embodiment, in Formula 601, when xe11 is 2 or more, two or more of Ar₆₀₁(s) may be linked to each other via a single bond.

In embodiments, in Formula 601, Ar₆₀₁ may be an anthracene group unsubstituted or substituted with at least one R₁₀ₐ.

In embodiments, the electron transport region may include a compound represented by Formula 601-1:

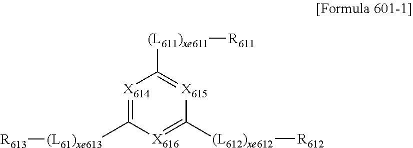

In Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may each be N,
L₆₁₁ to L₆₁₃ may each independently be the same as described herein with respect to L₆₀₁,
xe611 to xe613 may each independently be the same as described herein with respect to xe1,
R₆₁₁ to R₆₁₃ may each independently be the same as described herein with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

In an embodiment, in Formulae 601 and 601-1, xe1 and xe611 to xe613 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

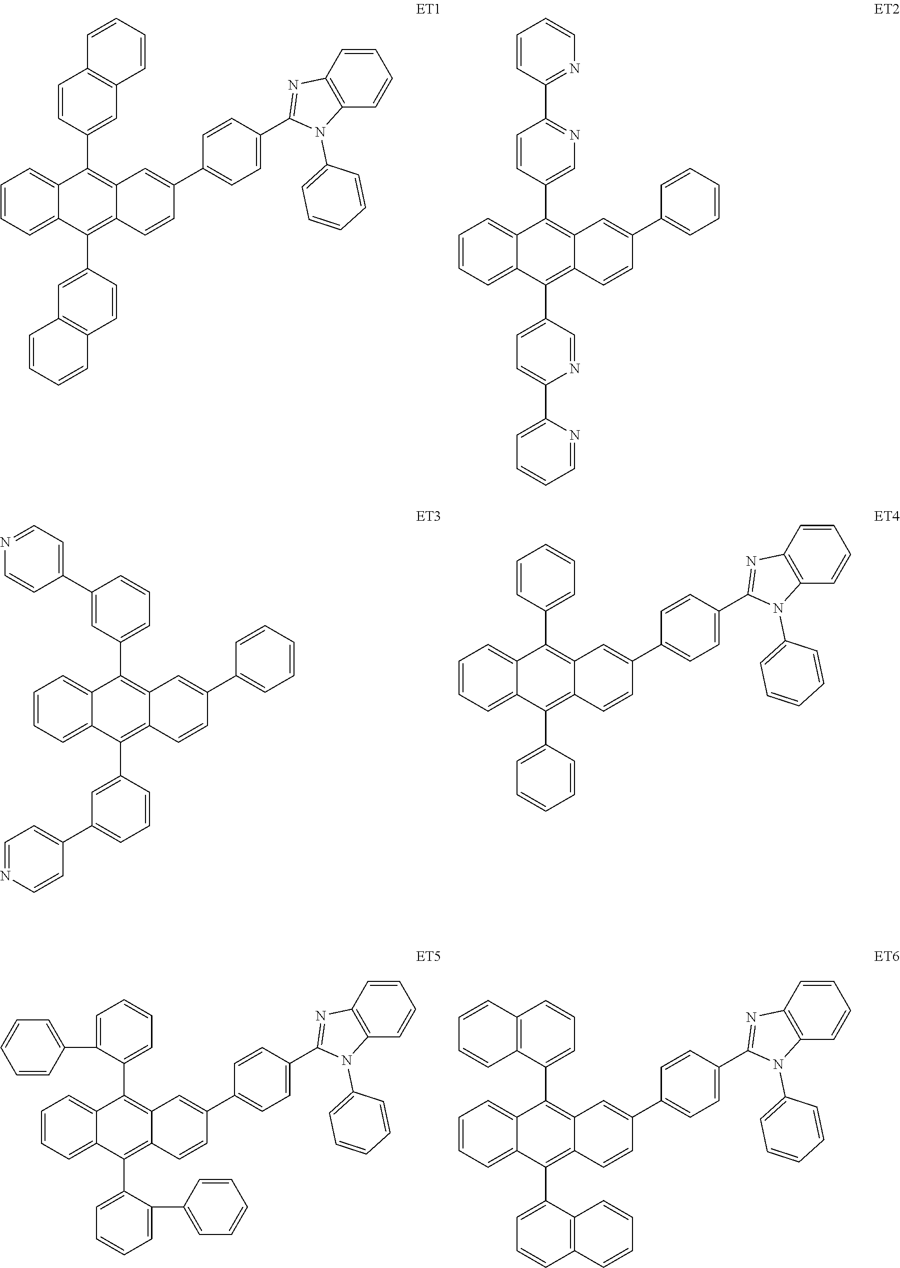

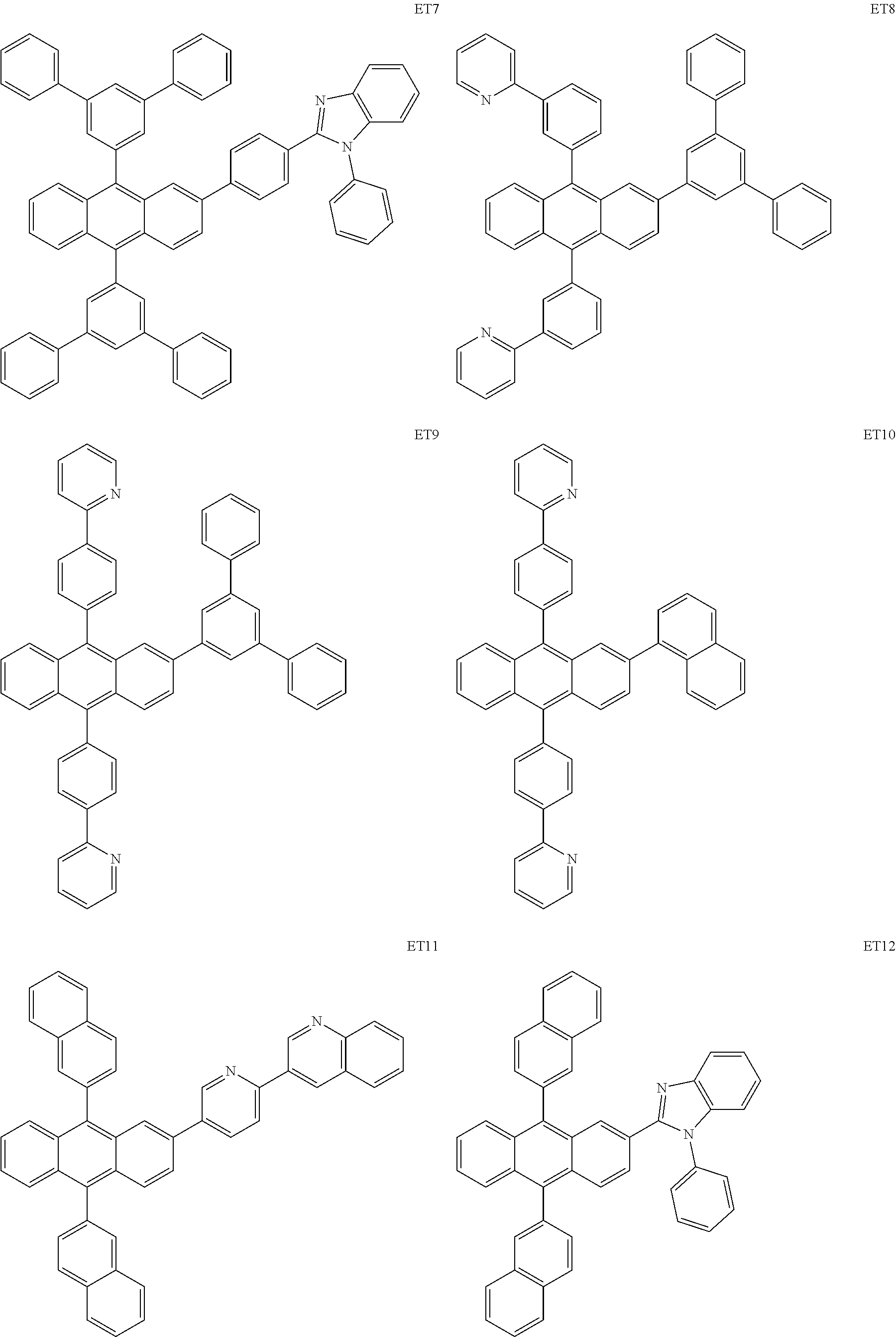

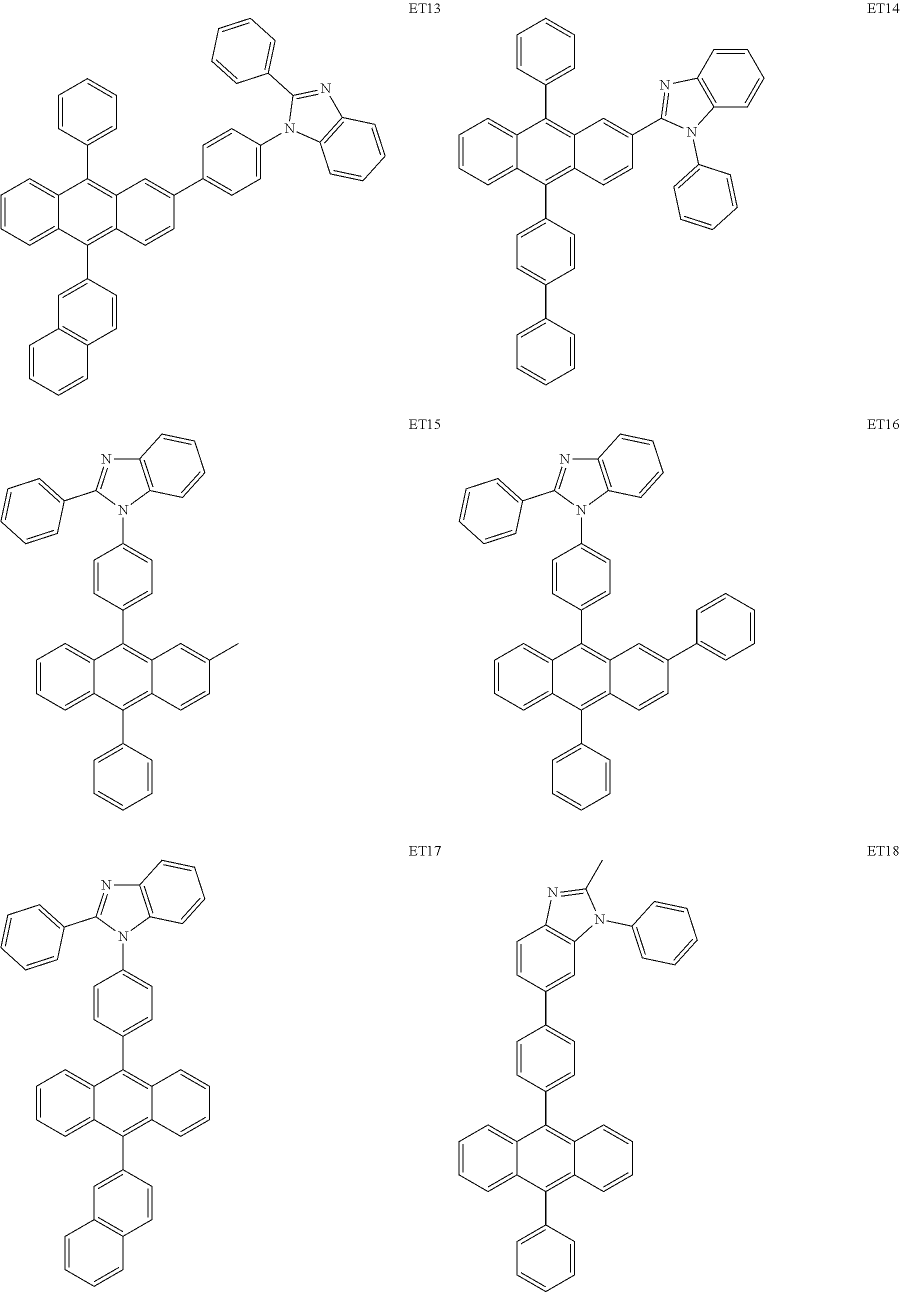 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

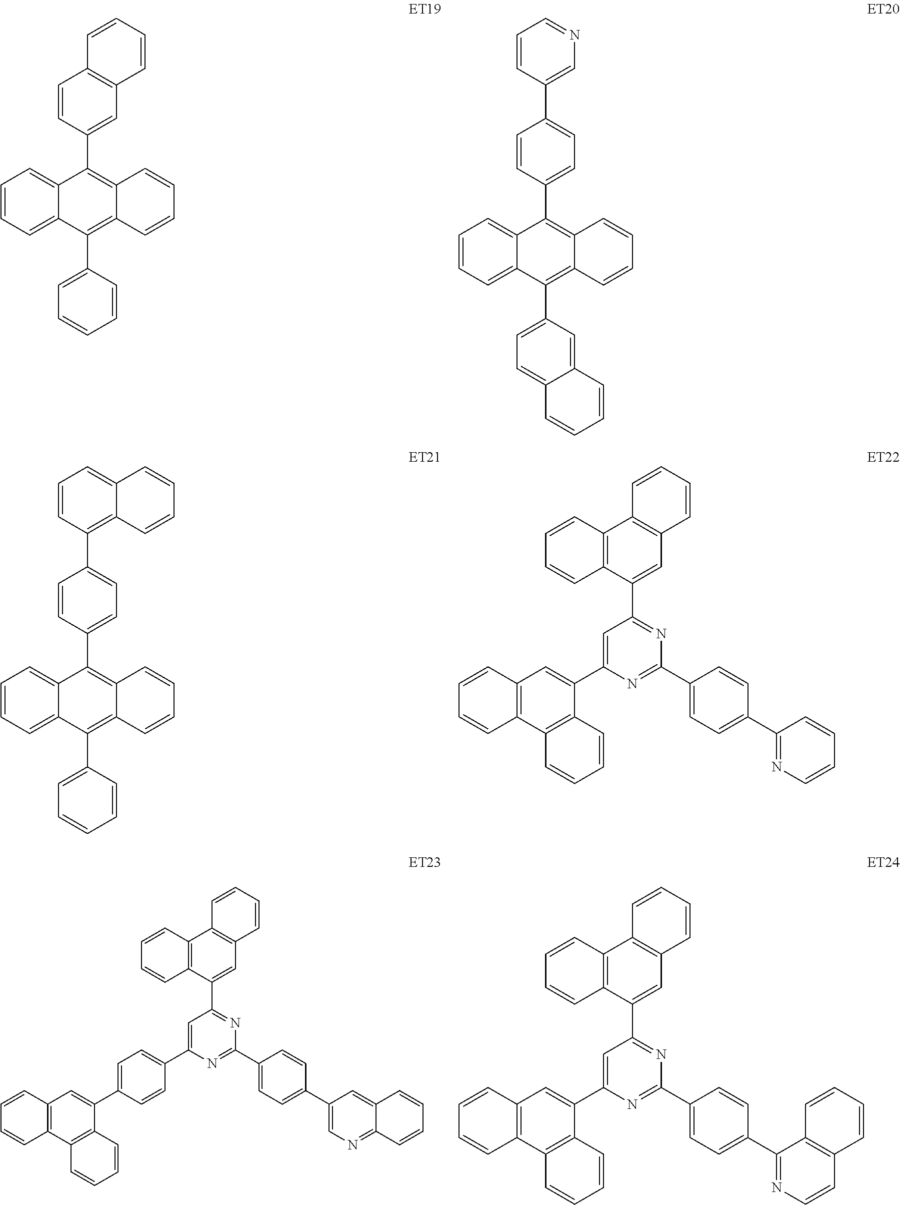

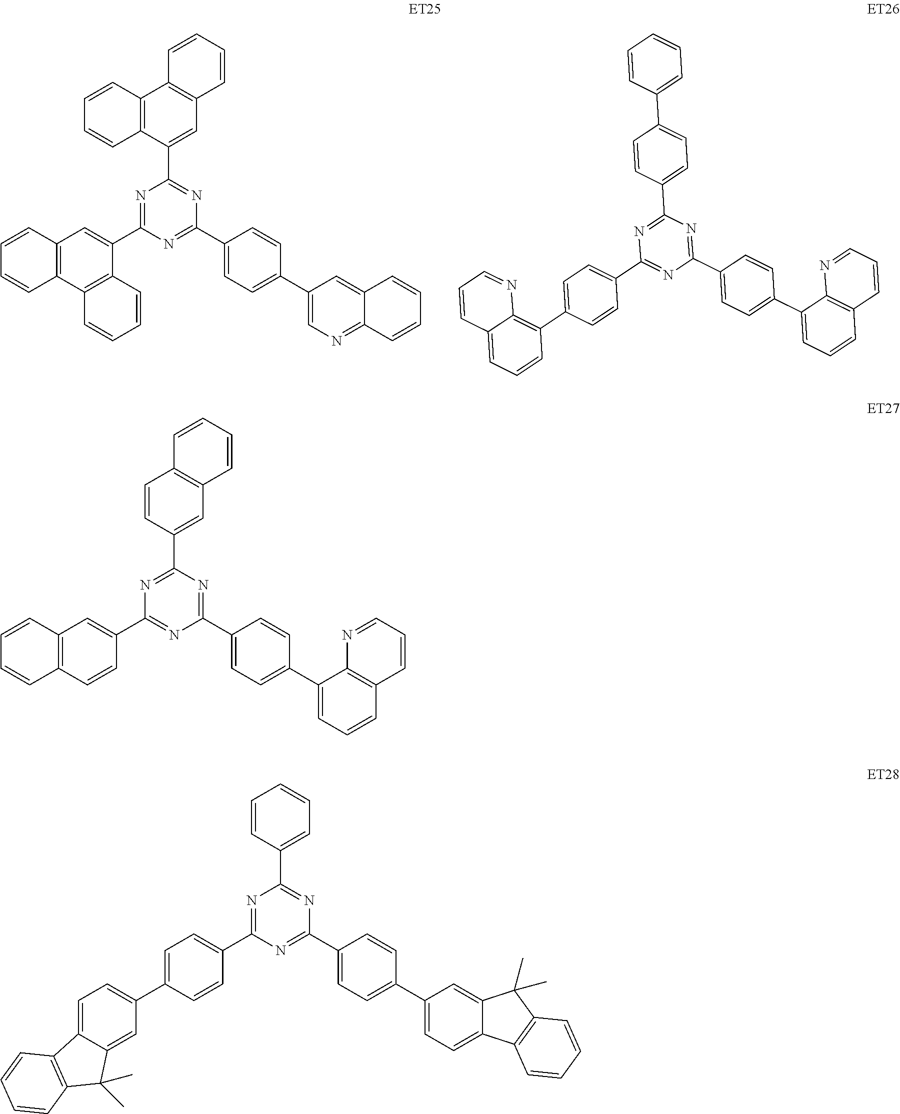

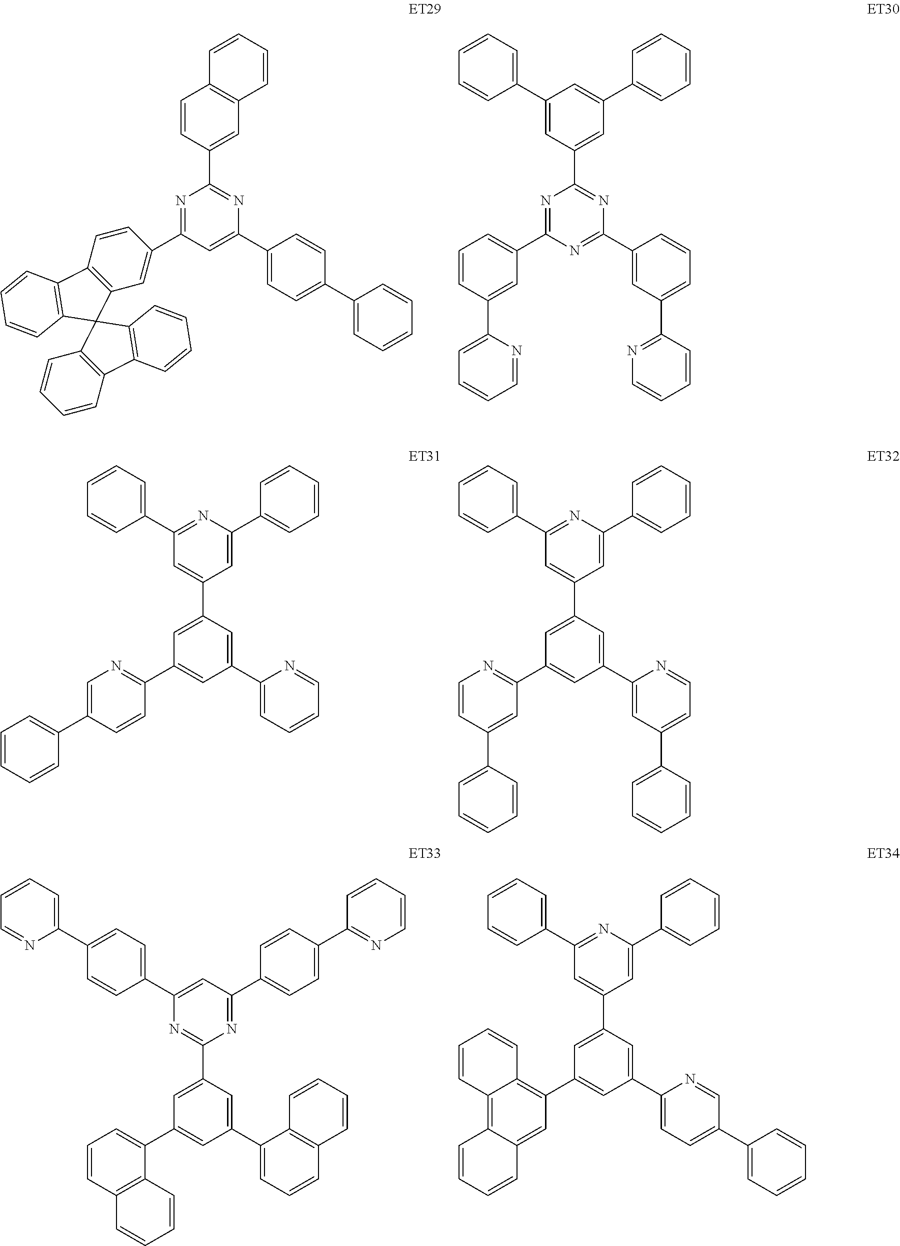

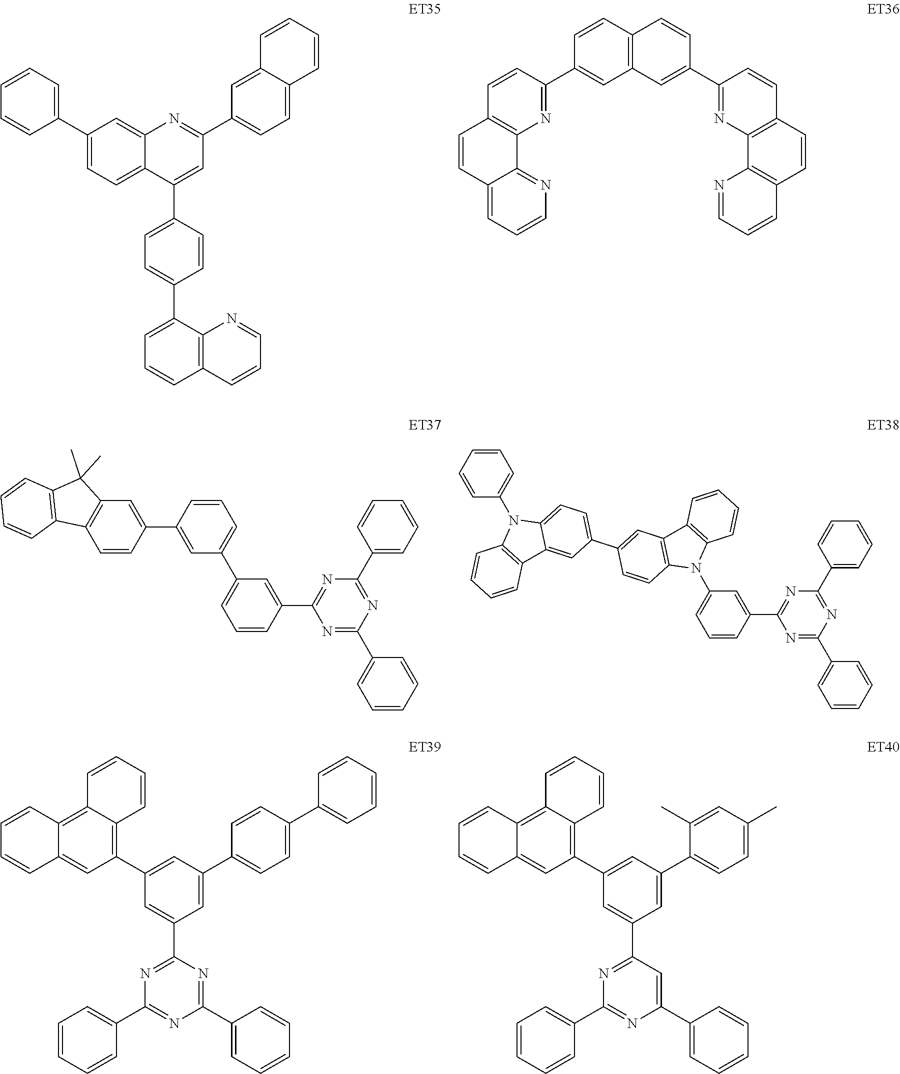 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

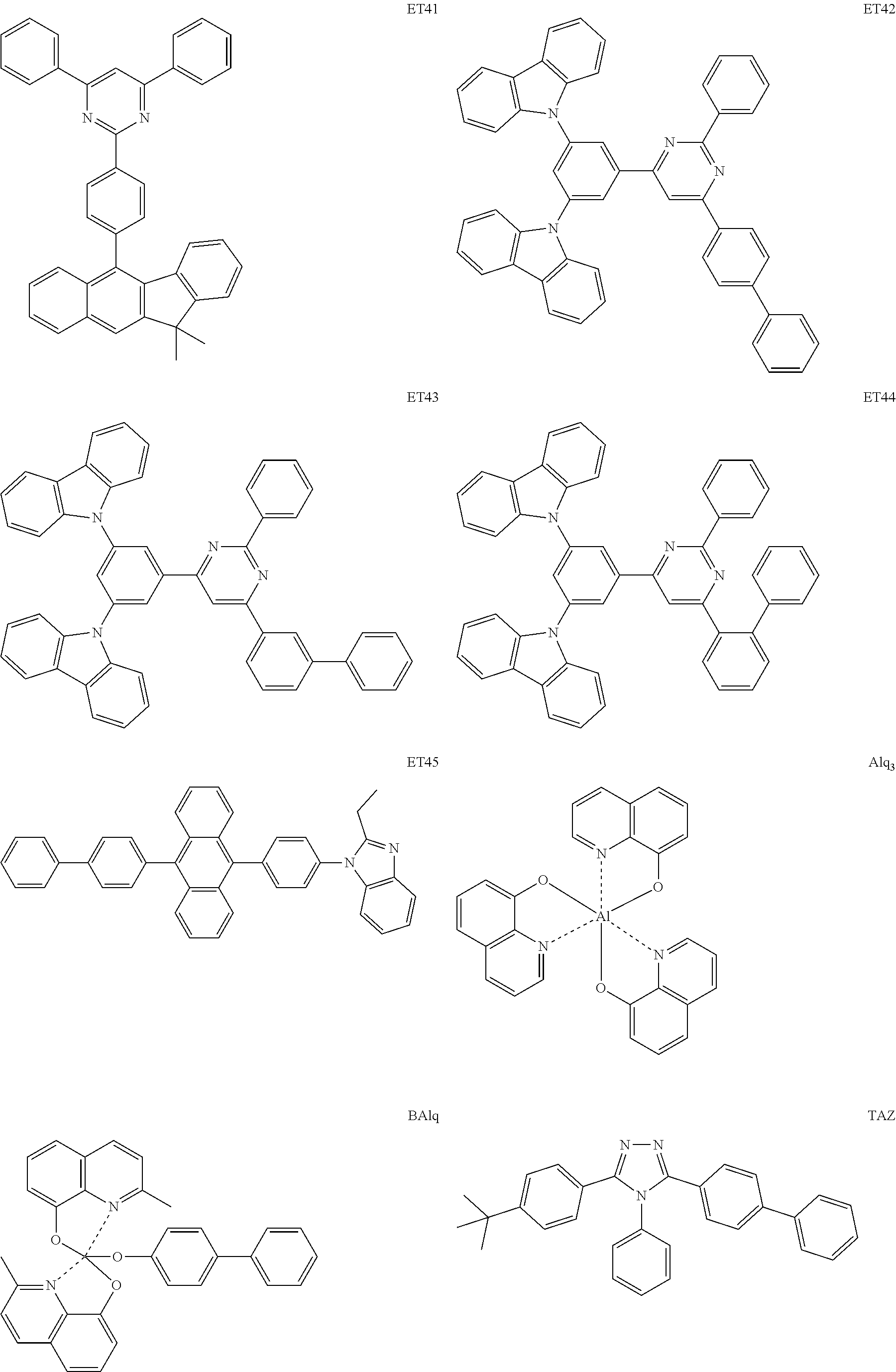

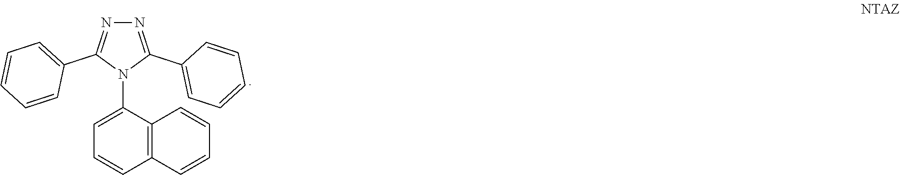 [SMILES: c1ccc(-c2nnc(-c3ccccc3)n2-c2cccc3ccccc23)cc1]

A thickness of the electron transport region may be in a range of about 100 Å to about 5,000 Å. For example, the thickness of the electron transport region may be in a range of about 160 Å to about 4,000 Å. When the electron transport region includes a buffer layer, a hole blocking layer, an electron control layer, an electron transport layer, or any combination thereof, a thickness of the buffer layer, the hole blocking layer, or the electron control layer may each independently be in a range of about 20 Å to about 1,000 Å, and a thickness of the electron transport layer may be in a range of about 100 Å to about 1,000 Å. For example, the thickness of the buffer layer, the hole blocking layer, or the electron control layer may each independently be in a range of about 30 Å to about 300 Å. For example, the thickness of the electron transport layer may be in a range of about 150 Å to about 500 Å. When the thickness of the buffer layer, the hole blocking layer, the electron control layer, the electron transport layer, and/or the electron transport region are within these ranges, satisfactory electron transporting characteristics may be obtained without a substantial increase in driving voltage.

The electron transport region (for example, an electron transport layer in the electron transport region) may further include, in addition to the materials described above, a metal-containing material.

The metal-containing material may include an alkali metal complex, an alkaline earth metal complex, or any combination thereof. A metal ion of an alkali metal complex may be a Li ion, a Na ion, a K ion, a Rb ion, or a Cs ion, and a metal ion of an alkaline earth metal complex may be a Be ion, a Mg ion, a Ca ion, a Sr ion, or a Ba ion.

A ligand coordinated with the metal ion of the alkali metal complex or with the metal ion of the alkaline earth-metal complex may each independently include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a lithium (Li) complex. The Li complex may include, for example, Compound ET-D1 (LiQ) or Compound ET-D2:

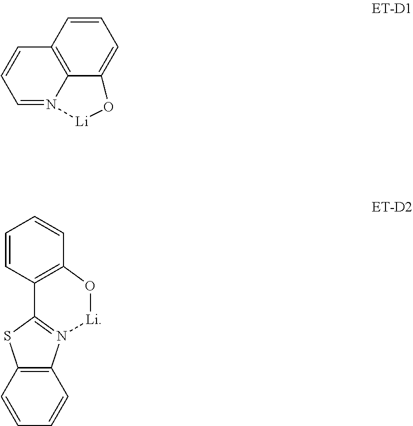

The electron transport region may include an electron injection layer that facilitates the injection of electrons from the second electrode 150. The electron injection layer may directly contact the second electrode 150.

The electron injection layer may have a structure consisting of a layer consisting of a single material, a structure consisting of a layer including different materials, or a structure including multiple layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, or iodides), or tellurides of the alkali metal, the alkaline earth metal, the rare earth metal, or any combination thereof.

The alkali metal-containing compound may include: alkali metal oxides, such as Li₂O, Cs₂O, or K₂O; alkali metal halides, such as LiF, NaF, CsF, KF, LiI, NaI, CsI, or KI; or any combination thereof. The alkaline earth metal-containing compound may include an alkaline earth metal compound, such as BaO, SrO, CaO, BaₓSr₁₋ₓO (wherein x is a real number satisfying the condition of 0<x<1), BaₓCa₁₋ₓO (wherein x is a real number satisfying the condition of 0<x<1), or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In embodiments, the rare earth metal-containing compound may include a lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include one of an alkali metal ion, an alkaline earth metal ion, and a rare earth metal ion, and a ligand bonded to the metal ion (for example, a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenyl benzimidazole, a hydroxyphenylbenzothiazole, a bipyridine, a phenanthroline, a cyclopentadiene, or any combination thereof).

The electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In embodiments, the electron injection layer may further include an organic material (for example, a compound represented by Formula 601).

In embodiments, the electron injection layer may consist of an alkali metal-containing compound (for example, an alkali metal halide); or the electron injection layer may consist of an alkali metal-containing compound (for example, an alkali metal halide), and an alkali metal, an alkaline earth metal, a rare earth metal, or any combination thereof. For example, the electron injection layer may be a KI:Yb co-deposited layer, an RbI:Yb co-deposited layer, a LiF:Yb co-deposited layer, or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å. For example, the thickness of the electron injection layer may be in a range of about 3 Å to about 90 Å. When the thickness of the electron injection layer is within the ranges described above, satisfactory electron injection characteristics may be obtained without a substantial increase in driving voltage.

[Second Electrode 150]

The second electrode 150 may be disposed on the interlayer 130. The second electrode 150 may be a cathode, which is an electron injection electrode. A material for forming the second electrode 150 may be a material with a low work function, such as a metal, an alloy, an electrically conductive compound, or any combination thereof.

The second electrode 150 may include Li, Ag, Mg, Al, Al—Li, Ca, Mg—In, Mg—Ag, Yb, Ag—Yb, ITO, IZO, or any combination thereof. The second electrode 150 may be a transmissive electrode, a semi-transmissive electrode, or a reflective electrode.

The second electrode 150 may have a single-layered structure or a multi-layered structure.

[Capping Layer]

The light-emitting device 10 may include a first capping layer located outside the first electrode 110, and/or a second capping layer located outside the second electrode 150. For example, the light-emitting device 10 may have a structure in which the first capping layer, the first electrode 110, the interlayer 130, and the second electrode 150 are stacked in this stated order, a structure in which the first electrode 110, the interlayer 130, the second electrode 150, and the second capping layer are stacked in this stated order, or a structure in which the first capping layer, the first electrode 110, the interlayer 130, the second electrode 150, and the second capping layer are stacked in this stated order.

For example, light generated by an emission layer in the interlayer 130 of the light-emitting device 10 may be extracted to the outside through the first electrode 110, which may be a semi-transmissive electrode or a transmissive electrode, and through the first capping layer. For example, light generated by an emission layer in the interlayer 130 of the light-emitting device 10 may be extracted to the outside through the second electrode 150, which may be a semi-transmissive electrode or a transmissive electrode, and through the second capping layer.

The first capping layer and the second capping layer may each increase external emission efficiency according to the principle of constructive interference. Accordingly, the light extraction efficiency of the light-emitting device 10 is increased, so that the luminescence efficiency of the light-emitting device 10 may be improved.

The first capping layer and the second capping layer may each include a material having a refractive index equal to or greater than about 1.6 (with respect to a wavelength of about 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and the second capping layer may each independently include carbocyclic compounds, heterocyclic compounds, amine group-containing compounds, porphine derivatives, phthalocyanine derivatives, naphthalocyanine derivatives, alkali metal complexes, alkaline earth metal complexes, or any combination thereof. In an embodiment, the carbocyclic compound, the heterocyclic compound, and the amine group-containing compound may be optionally substituted with a substituent including O, N, S, Se, Si, F, Cl, Br, I, or any combination thereof.

In embodiments, at least one of the first capping layer and the second capping layer may each independently include an amine group-containing compound.

For example, at least one of the first capping layer and the second capping layer may each independently include a compound represented by Formula 201, a compound represented by Formula 202, or any combination thereof.

In embodiments, at least one of the first capping layer and the second capping layer may each independently include one of Compounds HT28 to HT33, one of Compounds CP1 to CP6, β-NPB, or any combination thereof:

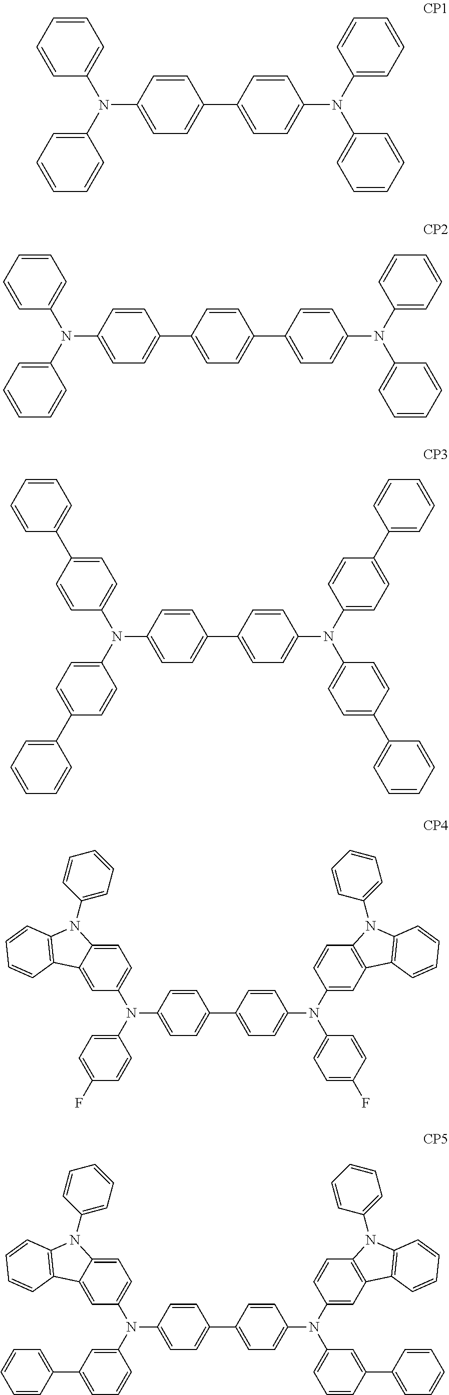 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

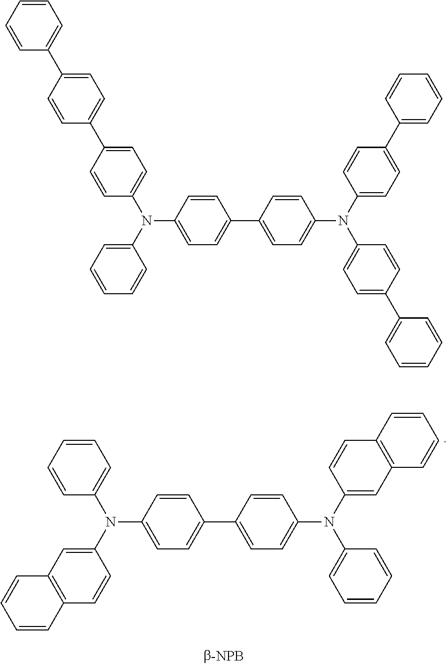 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

[Film]

At least one of the amine-containing compounds represented by Formulae 1c to 3 may be included in various films. Accordingly, another aspect provides a film which may include at least one of the amine-containing compounds represented by Formulae 1 to 3. The film may be, for example, an optical member (or a light control means) (for example, a color filter, a color conversion member, a capping layer, a light extraction efficiency enhancement layer, a selective light absorbing layer, a polarizing layer, a quantum dot-containing layer, or like), a light-blocking member (for example, a light reflective layer, a light absorbing layer, or the like), or a protective member (for example, an insulating layer, a dielectric layer, or the like).

[Electronic Apparatus]

The light-emitting device may be included in various electronic apparatuses. In an embodiment, an electronic apparatus including the light-emitting device may be a light-emitting apparatus, an authentication apparatus, or the like.

The electronic apparatus (for example, a light-emitting apparatus) may further include, in addition to the light-emitting device, a color filter, a color conversion layer, or a color filter and a color conversion layer. The color filter and/or the color conversion layer may be located in at least one direction in which light emitted from the light-emitting device travels. In an embodiment, light emitted from the light-emitting device may be blue light or white light. The light-emitting device may be the same as described herein. In embodiments, the color conversion layer may include a quantum dot. The quantum dot may be, for example, a quantum dot as described herein.

The electronic apparatus may include a first substrate. The first substrate may include subpixels, the color filter may include color filter areas respectively corresponding to the subpixels, and the color conversion layer may include color conversion areas respectively corresponding to the subpixels.

A pixel-defining film may be located between the subpixels to define each subpixel.

The color filter may further include color filter areas and light-shielding patterns located between the color filter areas, and the color conversion layer may further include color conversion areas and light-shielding patterns located between the color conversion areas.

The color filter areas (or the color conversion areas) may include a first area emitting first color light, a second area emitting second color light, and a third area emitting third color light. The first color light, the second color light, and/or the third color light may have different maximum emission wavelengths from one another. For example, the first color light may be red light, the second color light may be green light, and the third color light may be blue light. In an embodiment, the color filter areas (or the color conversion areas) may include quantum dots. For example, the first area may include a red quantum dot, the second area may include a green quantum dot, and the third area may not include a quantum dot. The quantum dot may be the same as described herein. The first area, the second area, and/or the third area may each include a scatterer.

In an embodiment, the light-emitting device may emit first light, the first area may absorb the first light to emit first-first color light, the second area may absorb the first light to emit second-first color light, and the third area may absorb the first light to emit third-first color light. The first-first color light, the second-first color light, and the third-first color light may have different maximum emission wavelengths from each other. For example, the first light may be blue light, the first-first color light may be red light, the second-first color light may be green light, and the third-first color light may be blue light.

The electronic apparatus may further include a thin-film transistor, in addition to the light-emitting device as described herein. The thin-film transistor may include a source electrode, a drain electrode, and an active layer, wherein one of the source electrode and the drain electrode may be electrically connected to one of the first electrode and the second electrode of the light-emitting device.

The thin-film transistor may further include a gate electrode, a gate insulating film, or the like.

The active layer may include crystalline silicon, amorphous silicon, an organic semiconductor, an oxide semiconductor, or the like.

The electronic apparatus may further include a sealing portion for sealing the light-emitting device. The sealing portion may be located between the color filter and/or the color conversion layer, and the light-emitting device. The sealing portion may allow light from the light-emitting device to be extracted to the outside, and may simultaneously prevent ambient air and moisture from penetrating into the light-emitting device. The sealing portion may be a sealing substrate including a transparent glass substrate or a plastic substrate. The sealing portion may be a thin-film encapsulation layer including an organic layer and/or an inorganic layer. When the sealing portion is a thin-film encapsulation layer, the electronic apparatus may be flexible.

Various functional layers may be further included on the sealing portion, in addition to the color filter and/or the color conversion layer, according to the use of the electronic apparatus. Examples of a functional layer may include a touch screen layer, a polarizing layer, an authentication apparatus, and the like. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, etc.).

The authentication apparatus may further include, in addition to the light-emitting device as described above, a biometric information collector.

The electronic apparatus may be applied to various displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, various measuring instruments, meters (for example, meters for a vehicle, an aircraft, and a vessel), projectors, and the like.

[Electronic Device]

The light-emitting device may be included in various electronic devices.

In embodiments, the electronic device including the light-emitting device may be a flat panel display, a curved display, a computer monitor, a medical monitor, a television, a billboard, an indoor light, an outdoor light, a signal light, a head-up display, a fully transparent display, a partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall with multiple displays tiled together, a theater screen, a stadium screen, a phototherapy device, or a signboard.

The light-emitting device has excellent luminescence efficiency and a long lifespan, such that the electronic device including the light-emitting device may have characteristics such as high luminance, high resolution, and low power consumption.

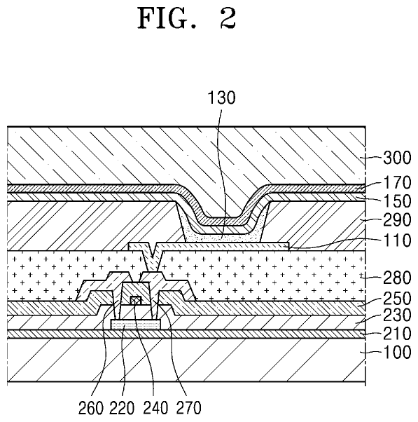

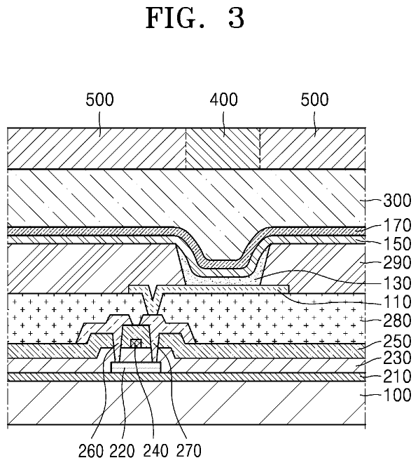

### DESCRIPTION OF FIGS. 2 AND 3

FIG. 2 is a schematic cross-sectional view of an electronic apparatus according to an embodiment.

The electronic apparatus of FIG. 2 may include a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be located on the substrate 100. The buffer layer 210 may prevent penetration of impurities through the substrate 100. The buffer layer 210 may provide a flat surface on the substrate 100.

A TFT may be located on the buffer layer 210. The TFT may include an active layer 220, a gate electrode 240, a source electrode 260, and a drain electrode 270.

The active layer 220 may include an inorganic semiconductor such as silicon or polysilicon, an organic semiconductor, or an oxide semiconductor. The active layer 220 may include a source region, a drain region, and a channel region.

A gate insulating film 230 for insulating the active layer 220 from the gate electrode 240 may be located on the active layer 220, and the gate electrode 240 may be located on the gate insulating film 230.

An interlayer insulating film 250 may be located on the gate electrode 240. The interlayer insulating film 250 may be located between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be located on the interlayer insulating film 250. The interlayer insulating film 250 and the gate insulating film 230 may be formed to expose the source region and the drain region of the active layer 220, and the source electrode 260 and the drain electrode 270 may respectively contact the exposed portions of the source region and the drain region of the active layer 220.

The TFT is electrically connected to a light-emitting device to drive the light-emitting device, and is covered and protected by a passivation layer 280. The passivation layer 280 may include an inorganic insulating film, an organic insulating film, or any combination thereof. A light-emitting device is provided on the passivation layer 280. The light-emitting device may include a first electrode 110, an interlayer 130, and a second electrode 150.

The first electrode 110 may be located on the passivation layer 280. The passivation layer 280 may not completely cover the drain electrode 270. The passivation layer 280 may expose a portion of the drain electrode 270. The first electrode 110 may be electrically connected to the exposed portion of the drain electrode 270.

A pixel defining layer 290 including an insulating material may be located on the first electrode 110. The pixel defining layer 290 may expose a portion of the first electrode 110. The interlayer 130 may be formed on the exposed portion of the first electrode 110. The pixel defining layer 290 may be a polyimide or polyacrylic organic film. Although not shown in FIG. 2, at least some layers of the interlayer 130 may extend beyond the upper portion of the pixel defining layer 290 to be provided in the form of a common layer.

The second electrode 150 may be located on the interlayer 130, and a capping layer 170 may be further included on the second electrode 150. The capping layer 170 may be formed to cover the second electrode 150.

The encapsulation portion 300 may be located on the capping layer 170. The encapsulation portion 300 may be located on a light-emitting device to protect the light-emitting device from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, or the like), an epoxy-based resin (for example, aliphatic glycidyl ether (AGE), or the like), or any combination thereof; or any combination of the inorganic films and the organic films.

FIG. 3 is a schematic cross-sectional view of an electronic apparatus according to another embodiment.

The electronic apparatus of FIG. 3 may differ from the electronic apparatus of FIG. 2, at least in that a light-shielding pattern 500 and a functional region 400 are further included on the encapsulation portion 300. The functional region 400 may be a color filter area, a color conversion area, or a combination of the color filter area and the color conversion area. In embodiments, the light-emitting device included in the electronic apparatus of FIG. 3 may be a tandem light-emitting device.

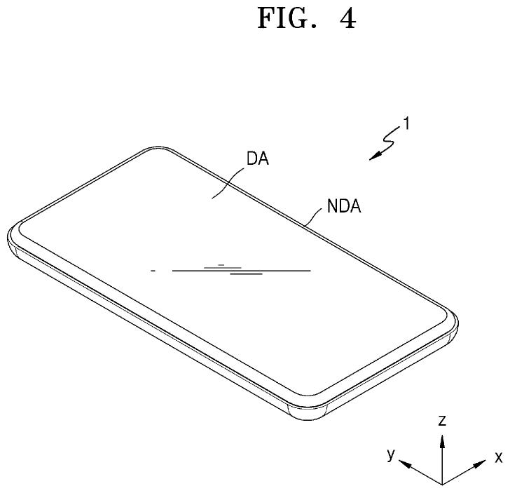

### DESCRIPTION OF FIG. 4

FIG. 4 is a schematic perspective view of an electronic device 1 including a light-emitting device according to an embodiment.

The electronic device 1, which may be a device displaying a video or a still image, may be a portable electronic device, or may be a portion of a portable electronic device, such as a mobile phone, a smart phone, a tablet personal computer (PC), a mobile telecommunication terminal, an electronic digital assistant, an electronic book, a portable multimedia player (PMP), a navigation system, an ultra-mobile PC (UMPC), as well as various products such as a television, a laptop, a monitor, an advertisement sign, or an internet of things (IOT).

In embodiments, the electronic device 1 may be a portable electronic device, or may be a portion of a portable electronic device, such as a smart watch, a watch phone, or a wearable device such as a glasses-type display or a head mounted display, (HMD). However, embodiments are not limited thereto.

For example, the electronic device 1 may be a dashboard of a vehicle, a center information display (CID) arranged on a center fascia or dashboard of a vehicle, a room mirror display that replaces the side mirror of a vehicle, a display arranged for entertainment in the back seat of a vehicle or arranged on the back of a front seat of a vehicle, a head-up display (HUD) installed on the front of a vehicle or projected onto the front window glass, or a computer generated hologram augmented reality head up display (CGH AR HUD). FIG. 4 shows an embodiment in which the electronic device 1 is a smartphone, for convenience of explanation.

The electronic device 1 may include a display area DA and a non-display area NDA outside the display area DA. The electronic device 1 may implement an image through a two-dimensional array of pixels in the display area DA.

The non-display area NDA, which is an area that does not display an image, may surround the display area DA. A driver for providing electrical signals or power to display elements arranged in the display area DA may be arranged in the non-display area NDA. A pad that is an area to which an electronic device or a printed circuit board may be electrically connected may be arranged in the non-display area NDA.

The electronic device 1 may have different lengths in an x-axis direction and a y-axis direction. In an embodiment, as shown in FIG. 4, the length in the x-axis direction may be less than the length in the y-axis direction. In another embodiment, the length in the x-axis direction and the length of the y-axis direction may be the same.

In still another embodiment, the length in the x-axis direction may be greater than the length in the y-axis direction.

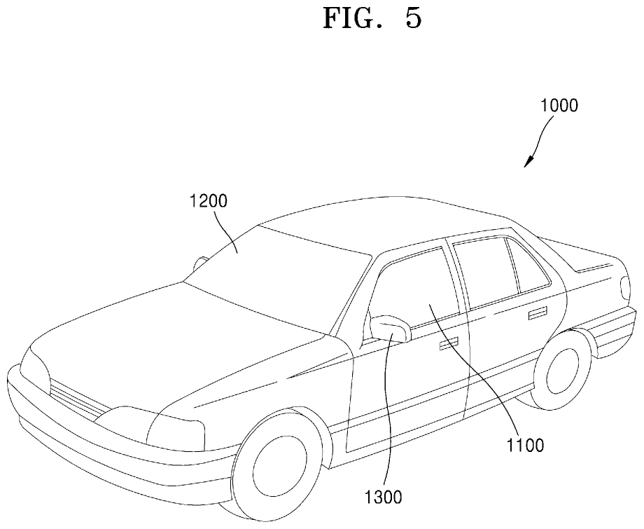

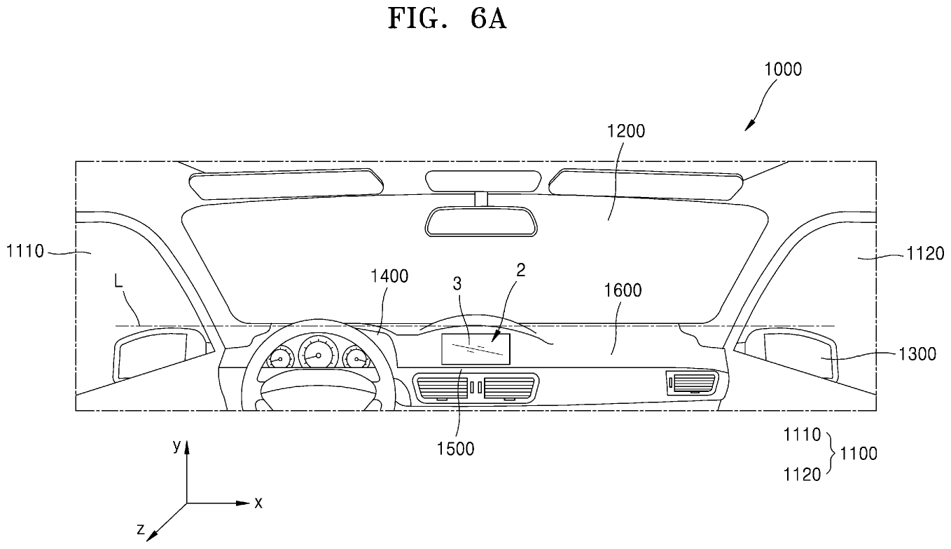

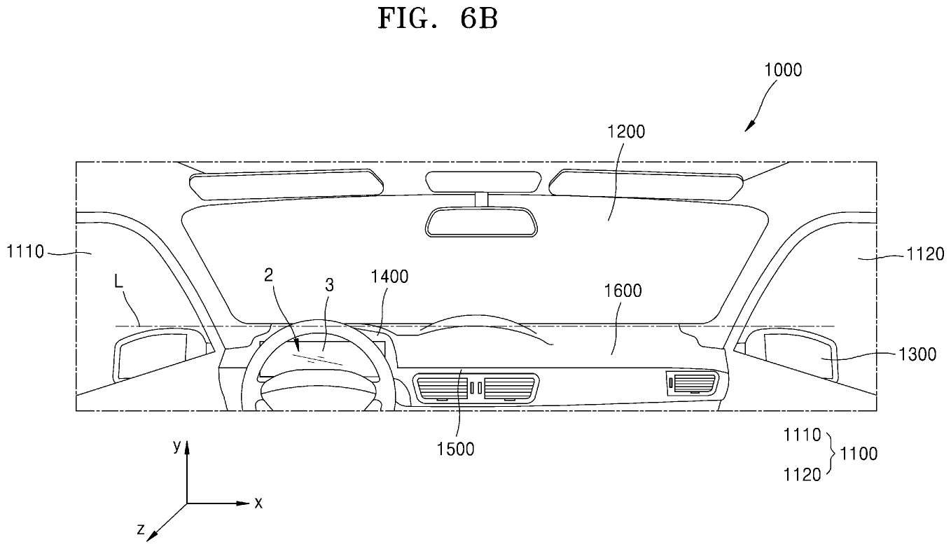

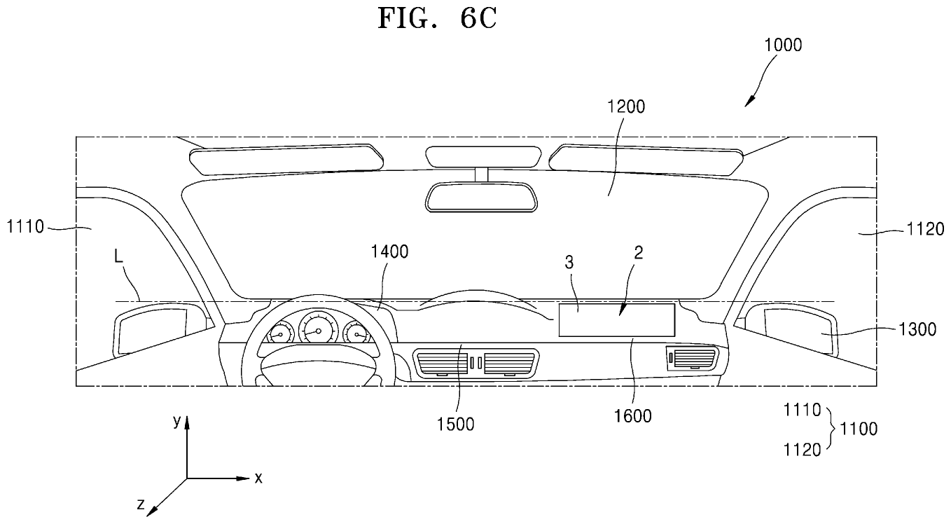

### DESCRIPTION OF FIGS. 5 AND 6A TO 6C

FIG. 5 is a schematic perspective view of the exterior of a vehicle 1000 as an electronic device including a light-emitting device according to an embodiment. FIGS. 6A to 6C are each a schematic diagram of the interior of a vehicle 1000 according to embodiments.

Referring to FIGS. 5, 6A, 6B, and 6C, a vehicle 1000 may refer to various devices that move a subject to be transported, such as a person, an object, or an animal, from a departure point to a destination. The vehicle 1000 may be a vehicle that drives on a road or a track, a ship that moves over a sea or a river, an airplane that flies in the air, or the like.

The vehicle 1000 may drive on a road or a track. The vehicle 1000 may move in a direction according to the rotation of at least one wheel. For example, the vehicle 1000 may be a three-wheeled or four-wheeled vehicle, a construction machine, a two-wheeled vehicle, an engine, a bicycle, or a train that runs on a track.

The vehicle 1000 may include a body having an interior and an exterior, and a chassis that is a portion excluding the body and includes mechanical devices installed therein that are necessary for driving the vehicle. The exterior of the body may include a front panel, a bonnet, a roof panel, the rear panel, a trunk, and a pillar provided at the boundaries between doors. The chassis of the vehicle 1000 may include a power generation device, a power transfer device, a driving device, a steering device, a braking device, a suspension, a transmission, a fuel device, and front, rear, left, and right wheels.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front windows glass 1200 may be partitioned by a pillar arranged between the side window glass 1100 and the front window glass 1200.

The side window glass 1100 may be installed on the side of the vehicle 1000. In an embodiment, the side window glass 1100 may be installed in the door of the vehicle 1000. Multiple side window glasses 1100 may be provided and may face each other. In an embodiment, the side window glass 1100 may include a first side window glass 1110 and a second side window glass 1120. In an embodiment, the first side window glass 1110 may be arranged adjacent to the cluster 1400. The second side window glass 1120 may be arranged adjacent to the passenger seat dashboard 1600.

In an embodiment, the side window glasses 1100 may be apart from each other in an x direction or in a −x direction. For example, the first side window glass 1110 and the second side window glass 1120 may be apart from each other in the x direction or in the −x direction. A virtual straight line L connecting the side window glasses 1100 may extend in the x direction or the −x direction. For example, a virtual straight line L connecting the first side window glass 1110 and the second side window glass 1120 to each other may extend in the x direction or in the −x direction.

The front window glass 1200 may be installed on the front of the vehicle 1000. The front window glass 1200 may be located between the side window glasses 1100 facing each other.

The side mirror 1300 may provide a rear view of the vehicle 1000. The side mirror 1300 may be installed on the exterior of the body. In an embodiment, multiple side mirrors 1300 may be provided. One of the side mirrors 1300 may be located on the outside of the first side window glass 1110. Another of the side mirrors 1300 may be located on the outside of the second side window glass 1120.

The cluster 1400 may be located in front of a steering wheel. The cluster 1400 may include a tachometer, a speedometer, a coolant thermometer, a fuel gauge, a turn signal indicator, a high beam indicator, a warning light, a seatbelt warning light, an odometer, a driving record system, an automatic transmission selection lever indicator, a door open warning light, an engine oil warning light, and/or fuel shortage warning light.

The center fascia 1500 may include a control panel having buttons for adjusting an audio device, an air conditioning device, and a seat heater. The center fascia 1500 may be located on a side of the cluster 1400.

The passenger seat dashboard 1600 may be spaced apart from the cluster 1400 with the center fascia 1500 therebetween. In an embodiment, the cluster 1400 may be arranged in correspondence to the driver's seat (not shown), and the passenger seat dashboard 1600 may be arranged in correspondence to the passenger seat (not shown). In an embodiment, the cluster 1400 may be adjacent to the first side window glass 1110, and the passenger seat dashboard 1600 may be adjacent to the second side window glass 1120.

In an embodiment, the display device 2 may include a display panel 3, and the display panel 3 may display an image. The display device 2 may be located inside the vehicle 1000. In an embodiment, the display device 2 may be located between the side window glasses 1100 facing each other. The display device 2 may be located in at least one of the cluster 1400, the center fascia 1500, and the passenger seat dashboard 1600.

The display device 2 may include an organic light-emitting display, an inorganic light-emitting display, a quantum dot display, or the like. Hereinafter, although the organic light-emitting display including the light-emitting device according to an embodiment is used as an example for the display device 2 according to an embodiment, various display devices as described above may be used as embodiments.

Referring to FIG. 6A, the display device 2 may be arranged in the center fascia 1500. In an embodiment, the display device 2 may display navigation information. In an embodiment, the display device 2 may display information about audio settings, video settings, or vehicle settings.

Referring to FIG. 6B, the display device 2 may be arranged in the cluster 1400. The cluster 1400 may show driving information and the like through the display device 2. Thus, the cluster 1400 may digitally implement driving information. The digital cluster 1400 may display vehicle information and driving information as images. For example, the needle and gauge of a tachometer and various warning lights may be displayed by a digital signal.

Referring to FIG. 6C, the display device 2 may be arranged in the passenger seat dashboard 1600. The display device 2 may be embedded in the passenger seat dashboard 1600 or located on the passenger seat dashboard 1600. In an embodiment, the display device 2 arranged in the passenger seat dashboard 1600 may display an image regarding information displayed on the cluster 1400 and/or information displayed on the center fascia 1500. In an embodiment, the display apparatus 2 arranged in the passenger seat dashboard 1600 may display an image related to information displayed on the cluster 1400 and/or related to information displayed on the center fascia 1500. In an embodiment, the display apparatus 2 arranged in the passenger seat dashboard 1600 may display an image that is different from the information displayed on the cluster 1400 and/or the information displayed on the center fascia 1500.

[Manufacturing Method]

Respective layers included in the hole transport region, the emission layer, and respective layers included in the electron transport region may be formed in a certain region by using one or more suitable methods selected from vacuum deposition, spin coating, casting, Langmuir-Blodgett (LB) deposition, ink-jet printing, laser-printing, and laser-induced thermal imaging.

When layers constituting the hole transport region, an emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definitions of Terms

The term “C₃-C₆₀ carbocyclic group” as used herein may be a cyclic group consisting of carbon atoms as the only ring-forming atoms and having 3 to 60 carbon atoms. The term “C₁-C₆₀ heterocyclic group” as used herein may be a cyclic group having 1 to 60 carbon atoms and further including, in addition to carbon atoms, at least one heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, a C₁-C₆₀ heterocyclic group may have 3 to 61 ring-forming atoms.

The term “cyclic group” as used herein may be a C₃-C₆₀ carbocyclic group or a C₁-C₆₀ heterocyclic group.

The term “π electron-rich C₃-C₆₀ cyclic group” as used herein may be a cyclic group having 3 to 60 carbon atoms and may not include *—N═*′ as a ring-forming moiety. The term “π electron-deficient nitrogen-containing C₁-C₆₀ cyclic group” as used herein may be a heterocyclic group having 1 to 60 carbon atoms and may include *—N═*′ as a ring-forming moiety.

In embodiments,
the C₃-C₆₀ carbocyclic group may be a T1 group or a group in which two or more T1 groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be a T2 group, a group in which at least two T2 groups are condensed with each other, or a group in which at least one T2 group and at least one T1 group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, or the like),
the π electron-rich C₃-C₆₀ cyclic group may be a T1 group, a group in which at least two T1 groups are condensed with each other, a T3 group, a group in which at least two T3 groups are condensed with each other, or a group in which at least one T3 group and at least one T1 group are condensed with each other (for example, a C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, or the like), and
the π electron-deficient nitrogen-containing C₁-C₆₀ cyclic group may be a T4 group, a group in which at least two T4 groups are condensed with each other, a group in which at least one T4 group and at least one T1 group are condensed with each other, a group in which at least one T4 group and at least one T3 group are condensed with each other, or a group in which at least one T4 group, at least one T1 group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and the like),
wherein the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,
the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the T3 group may be a furan group, a thiophene group, a 1H-pyrrole group, a silole group, or a borole group, and
the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group”, “C₃-C₆₀ carbocyclic group”, “C₁-C₆₀ heterocyclic group”, “π electron-rich C₃-C₆₀ cyclic group”, or “π electron-deficient nitrogen-containing C₁-C₆₀ cyclic group” as used herein may each be a group condensed to any cyclic group, a monovalent group, or a polyvalent group (for example, a divalent group, a trivalent group, a tetravalent group, etc.) according to the structure of a formula for which the corresponding term is used. For example, a “benzene group” may be a benzo group, a phenyl group, a phenylene group, or the like, which may be readily understood by one of ordinary skill in the art according to the structure of a formula including the “benzene group.”

Examples of a monovalent C₃-C₆₀ carbocyclic group and a monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Examples of a divalent C₃-C₆₀ carbocyclic group and a divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein may be a linear or branched aliphatic hydrocarbon monovalent group with 1 to 60 carbon atoms. Examples of a C₁-C₆₀ alkyl group may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group.

The term “C₁-C₆₀ alkylene group” as used herein may be a divalent group having a same structure as the C₁-C₆₀ alkyl group.

The term “C₂-C₆₀ alkenyl group” as used herein may be a monovalent hydrocarbon group having at least one carbon-carbon double bond in the middle or at a terminus of a C₂-C₆₀ alkyl group. Examples of a C₂-C₆₀ alkenyl group may include an ethenyl group, a propenyl group, and a butenyl group.

The term “C₂-C₆₀ alkenylene group” as used herein may be a divalent group having a same structure as the C₂-C₆₀ alkenyl group.

The term “C₂-C₆₀ alkynyl group” as used herein may be a monovalent hydrocarbon group having at least one carbon-carbon triple bond in the middle or at a terminus of a C₂-C₆₀ alkyl group. Examples of a C₂-C₆₀ alkynyl group may include an ethynyl group and a propynyl group.

The term “C₂-C₆₀ alkynylene group” as used herein may be a divalent group having a same structure as the C₂-C₆₀ alkynyl group.

The term “C₁-C₆₀ alkoxy group” as used herein may be a monovalent group represented by —O(A₁₀₁) (wherein A₁₀₁ may be a C₁-C₆₀ alkyl group). Examples of a C₁-C₆₀ alkoxy group may include a methoxy group, an ethoxy group, and an isopropyloxy group.

The term “C₃-C₁₀ cycloalkyl group” as used herein may be a monovalent saturated hydrocarbon cyclic group with 3 to 10 carbon atoms. Examples of a C₃-C₁₀ cycloalkyl group may include a cyclopropyl group, a cyclobutyl group, a cyclopentyl, cyclohexyl group, a cycloheptyl group, a cyclooctyl group, an adamantanyl group, a norbornanyl group (or a bicyclo[2.2.1]heptyl group), a bicyclo[1.1.1]pentyl group, a bicyclo[2.1.1]hexyl group, and a bicyclo[2.2.2]octyl group.

The term “C₃-C₁₀ cycloalkylene group” as used herein may be a divalent group having a same structure as the C₃-C₁₀ cycloalkyl group.

The term “C₁-C₁₀ heterocycloalkyl group” as used herein may be a monovalent cyclic group that has 1 to 10 carbon atoms, and at least one heteroatom as a ring-forming atom in addition to carbon atoms. Examples of a C₁-C₁₀ heterocycloalkyl group may include a 1,2,3,4-oxatriazolidinyl group, a tetrahydrofuranyl group, and a tetrahydrothiophenyl group.

The term “C₁-C₁₀ heterocycloalkylene group” as used herein may be a divalent group having a same structure as the C₁-C₁₀ heterocycloalkyl group.

The term “C₃-C₁₀ cycloalkenyl group” as used herein may be a monovalent cyclic group that has 3 to 10 carbon atoms and at least one carbon-carbon double bond in its ring, and is not aromatic. Examples of a C₃-C₁₀ cycloalkenyl group may include a cyclopentenyl group, a cyclohexenyl group, and a cycloheptenyl group.

The term “C₃-C₁₀ cycloalkenylene group” as used herein may be a divalent group having a same structure as the C₃-C₁₀ cycloalkenyl group.

The term “C₁-C₁₀ heterocycloalkenyl group” as used herein may be a monovalent cyclic group of 1 to 10 carbon atoms, further including, in addition to carbon atoms, at least one heteroatom, as ring-forming atoms, and having at least one carbon-carbon double bond in the cyclic structure thereof. Examples of a C₁-C₁₀ heterocycloalkenyl group may include a 4,5-dihydro-1,2,3,4-oxatriazolyl group, a 2,3-dihydrofuranyl group, and a 2,3-dihydrothiophenyl group.

The term “C₁-C₁₀ heterocycloalkenylene group” as used herein may be a divalent group having a same structure as the C₁-C₁₀ heterocycloalkenyl group.

The term “C₆-C₆₀ aryl group” as used herein may be a monovalent group having a carbocyclic aromatic system of 6 to 60 carbon atoms. Examples of a C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group.

The term “C₆-C₆₀ arylene group” as used herein may be a divalent group having a carbocyclic aromatic system of 6 to 60 carbon atoms.

When the C₆-C₆₀ aryl group and the C₆-C₆₀ arylene group each include two or more rings, the respective rings may be condensed with each other.

The term “C₁-C₆₀ heteroaryl group” as used herein may be a monovalent group that has a heterocyclic aromatic system of 1 to 60 carbon atoms and further including, in addition to a carbon atom, at least one heteroatom as a ring-forming atom. Examples of a C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group.

The term “C₁-C₆₀ heteroarylene group” as used herein may be a divalent group that has a heterocyclic aromatic system of 1 to 60 carbon atoms and further including, in addition to a carbon atom, at least one heteroatom as a ring-forming atom.

When the C₁-C₆₀ heteroaryl group and the C₁-C₆₀ heteroarylene group each include two or more rings, the respective rings may be condensed with each other.

The term “monovalent non-aromatic condensed polycyclic group” as used herein may be a monovalent group (for example, having 8 to 60 carbon atoms) having two or more rings condensed to each other, only carbon atoms as ring-forming atoms, and no aromaticity in its entire molecular structure. Examples of a monovalent non-aromatic condensed polycyclic group may include an indenyl group, a fluorenyl group, a spiro-bifluorenyl group, a benzofluorenyl group, an indenophenanthrenyl group, and an indenoanthracenyl group.

The term “divalent non-aromatic condensed polycyclic group” as used herein may be a divalent group having a same structure as the monovalent non-aromatic condensed polycyclic group described above.

The term “monovalent non-aromatic condensed heteropolycyclic group” as used herein may be a monovalent group (for example, having 1 to 60 carbon atoms) having two or more rings condensed to each other, further including, in addition to carbon atoms, at least one heteroatom, as ring-forming atoms, and having no aromaticity in its entire molecular structure. Examples of a monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group.

The term “divalent non-aromatic condensed heteropolycyclic group” as used herein may be a divalent group having a same structure as the monovalent non-aromatic condensed heteropolycyclic group described above.

The term “C₆-C₆₀ aryloxy group” as used herein may be a group represented by —O(A₁₀₂) (wherein A₁₀₂ may be a C₆-C₆₀ aryl group).

The term “C₆-C₆₀ arylthio group” as used herein may be a group represented by —S(A₁₀₃) (wherein A₁₀₃ may be a C₆-C₆₀ aryl group).

The term “C₇-C₆₀ arylalkyl group” as used herein may be a group represented by -(A₁₀₄)(A₁₀₅) (wherein A₁₀₄ may be a C₁-C₅₄ alkylene group and A₁₀₅ may be a C₆-C₅₉ aryl group).

The term “C₂-C₆₀ heteroarylalkyl group” as used herein may be a group represented by -(A₁₀₆)(A₁₀₇) (wherein A₁₀₆ may be a C₁-C₅₉ alkylene group and A₁₀₇ may be a C₁-C₅₉ heteroaryl group).

In the specification, the group “R₁₀ₐ” may be:
deuterium (˜D), —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

In the specification, Q₁ to Q₃, Q₁₁ to Q₁₃, Q₂₁ to Q₂₃, and Q₃₁ to Q₃₃ may each independently be:
hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom” as used herein may be any atom other than a carbon atom or a hydrogen atom. Examples of a heteroatom may include O, S, N, P, Si, B, Ge, Se, or any combination thereof.

The term “third-row transition metal” as used herein may include Hf, Ta, W, Re, Os, Ir, Pt, Au, or the like.

The term “Ph” as used herein refers to a phenyl group, the term “Me” as used herein refers to a methyl group, the term “Et” as used herein refers to an ethyl group, the terms “tert-Bu” or “Bu^{t}” as used herein each refer to a tert-butyl group, and the term “OMe” as used herein refers to a methoxy group.

The term “biphenyl group” as used herein may be a “phenyl group substituted with a phenyl group.” For example, the “biphenyl group” may be a substituted phenyl group having a C₆-C₆₀ aryl group as a substituent.

The term “terphenyl group” as used herein may be a “phenyl group substituted with a biphenyl group”. For example, the “terphenyl group” may be a substituted phenyl group having, as a substituent, a C₆-C₆₀ aryl group substituted with a C₆-C₆₀ aryl group.

The symbols * and *′ as used herein, unless defined otherwise, each refer to a binding site to a neighboring atom in a corresponding formula or moiety.

In the specification, the terms “x-axis,” “y-axis,” and “z-axis” are not limited to three axes in a Cartesian coordinate system or in an orthogonal coordinate system, and may have a broader meaning that includes these axes. For example, the x-axis, y-axis, and z-axis may describe axes that are orthogonal to each other, or may describe axes that are in different directions that are not orthogonal to each other.

Hereinafter, compounds according to embodiments and light-emitting devices according to embodiments will be described in detail with reference to the following Synthesis Examples and Examples. The wording “B was used instead of A” as used in describing Synthesis Examples means that an identical molar equivalent of B was used in place of A.

### EXAMPLES

### Synthesis Example 1: Synthesis of Compound 1

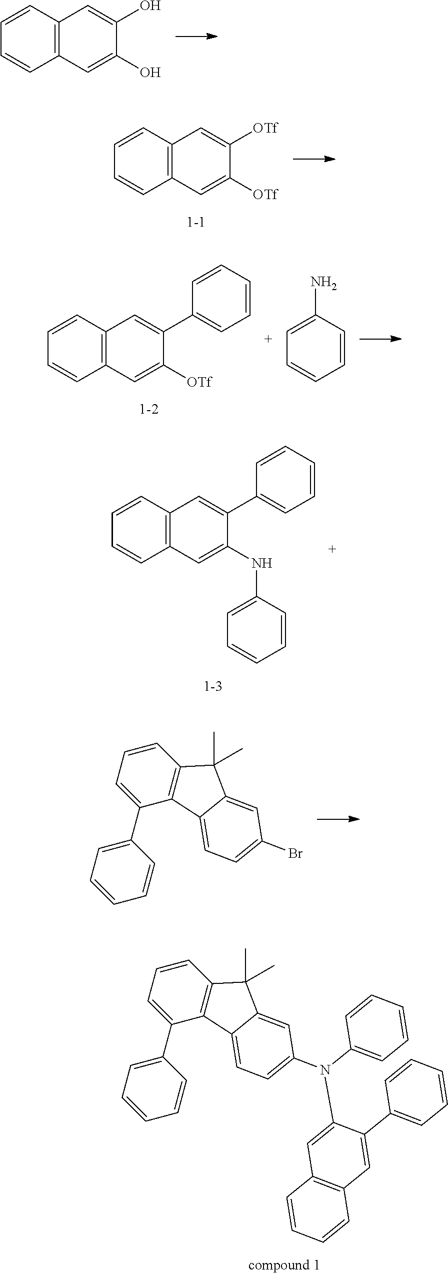

Synthesis of Intermediate 1-1

1.60 g (10 mmol) of naphthalene-2,3-diol, and 4.18 ml (30 mmol) of triethylamine were dissolved in 60 mL of dichloromethane (DCM) in a flask, and 3.36 ml (20 mmol) of trifluoromethanesulfonic anhydride was dissolved in 20 mL of DCM and slowly added to the flask at 0° C., followed by stirring for 5 hours at room temperature. 40 mL of water was added to the reaction solution, and the mixture was extracted three times with 50 mL of ethyl ether. The collected organic layer was dried using magnesium sulfate (MgSO₄), the residue obtained by evaporating the solvent was separated and purified by silica gel column chromatography, to thereby obtain 3.58 g (yield: 80%) of Intermediate 1-1. The resulting compound was identified by LC-MS. C₁₂H₆F₆O₆S₂: M⁺ 423.9

Synthesis of Intermediate 1-2

4.24 g (10.0 mmol) of Intermediate 1-1, 1.10 g (9.0 mmol) of phenylboronic acid, 0.58 g (0.5 mmol) of Pd(PPh₃)₄, and 4.14 g (30.0 mmol) of K₂CO₃ were dissolved in 60 mL of a tetrahydrofuran (THF)/H₂O (2/1) mixed solution, and stirred for 16 hours at 80° C. The reaction solution was cooled at room temperature, followed by three times of an extraction process using 60 ml of water and 60 ml of diethyl ether. The resulting organic layer was dried by using MgSO₄. A solvent was removed therefrom by evaporation. The resulting residue was separated and purified through silica gel chromatography to thereby obtain 1.76 g of Intermediate 1-2 (yield: 50%). The resulting compound was identified by LC-MS. C₁₇H₁₁F₃O₃S: M⁺352.3

Synthesis of Intermediate 1-3

3.52 g (10 mmol) of Intermediate 1-2, 1.40 g (15 mmol) of aniline, 0.46 g (0.5 mmol) of tris(dibenzylideneacetone)dipalladium(0) (Pd₂dba₃), 0.41 g (1 mmol) of s-phos, and 2.88 g (30 mmol) of sodium tert-butoxide were dissolved in 60 mL of toluene, followed by stirring at 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 1.77 g (yield: 60%) of Intermediate 1-3. The resulting compound was identified by LC-MS. C₂₂H₁₇N: M⁺ 295.1

Synthesis of Compound 1

2.95 g (10 mmol) of Intermediate 1-3, 3.49 g (10 mmol) of 2-bromo-9,9-dimethyl-5-phenyl-9H-fluorene, 0.46 g (0.5 mmol) of tris(dibenzylideneacetone)dipalladium(0)(Pd₂dba₃), 0.24 g (1 mmol) of P(t-Bu)3, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 3.95 g (yield: 70%) of Compound 1. The resulting compound was identified by MS/FAB and 1H NMR.

### Synthesis Example 2: Synthesis of Compound 2

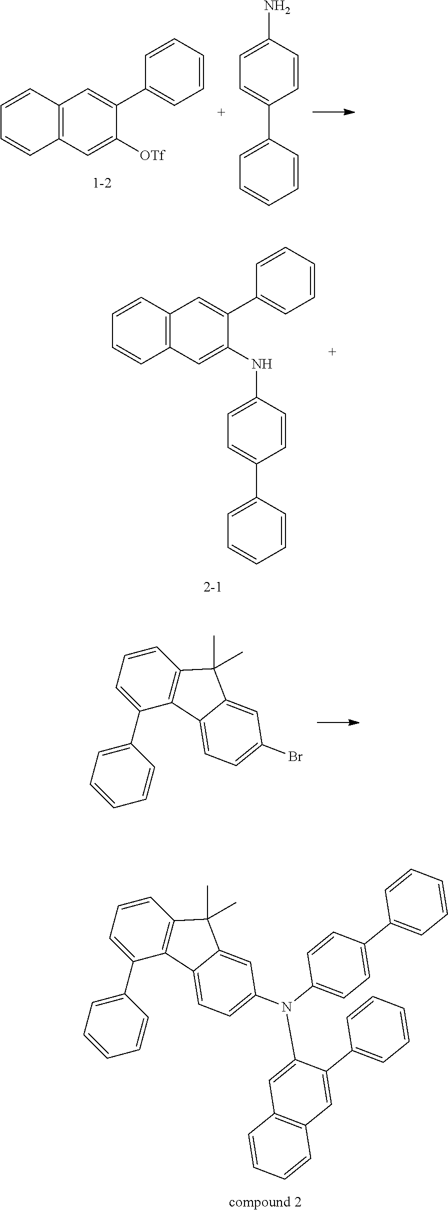

Compound 2 was synthesized in the same manner as used to synthesize Compound 1, except that 4-aminobiphenyl was used instead of aniline. The resulting compound was identified by MS/FAB and 1H NMR.

### Synthesis Example 3: Synthesis of Compound 9

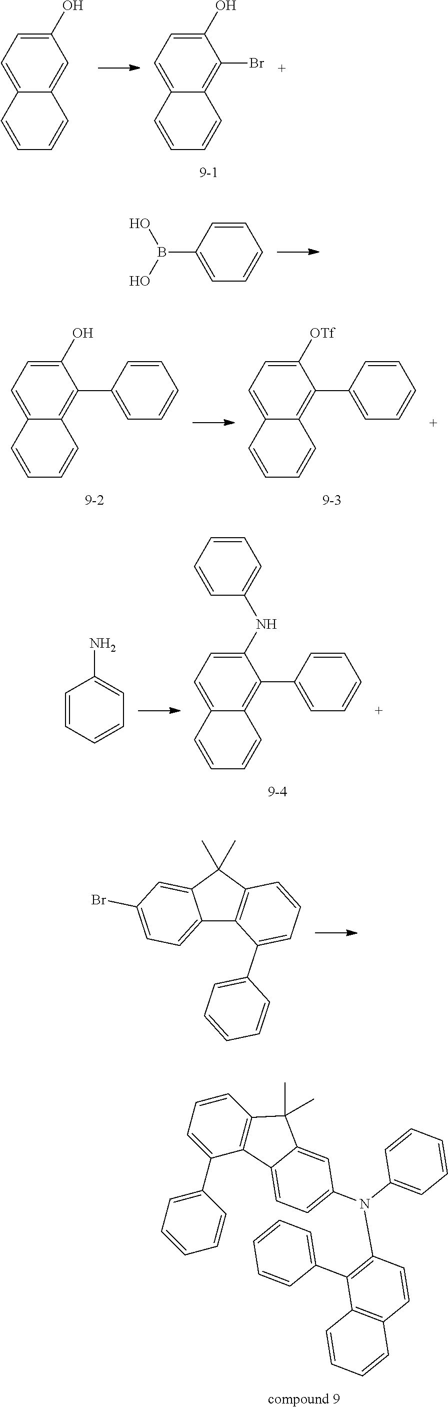

Synthesis of Intermediate 9-1

1.44 g (10 mmol) of 2-hydroxynaphthalene was dissolved in 20 mL of DCM and 1.78 g (10 mmol) of N-bromosuccinimide was dissolved in DCM 20 mL and added thereto at 0° C. The resultant was stirred for 5 hours at room temperature, and 3 g of Na₂S₂O₃ was dissolved in water and added thereto, followed by three times of washing using DCM (30 ml). The washed DCM layer was dried using MgSO₄ and dried under reduced pressure to obtain a product, which was separated and purified by silica gel column chromatography, to thereby obtain 1.78 g (yield: 80%) of Intermediate 9-1. The resulting compound was identified by LC-MS. C₁₀H₇BrO: M+ 235.9

Synthesis of Intermediate 9-2

2.23 g (10 mmol) of Intermediate 9-1, 1.46 g (12 mmol) of phenylboronic acid, 0.58 g (0.5 mmol) of Pd(PPh₃)₄, and 4.14 g (30 mmol) of K₂CO₃ were dissolved in 60 mL of a THF/H₂O (2/1) mixed solution, and stirred for 16 hours at 80° C. The reaction solution was cooled at room temperature, followed by three times of an extraction process using 60 ml of water and 60 ml of diethyl ether. The collected ethylether was dried using MgSO₄, and a solvent was evaporated therefrom to obtain a residue. The resulting residue was separated and purified through silica gel column chromatography to thereby obtain 1.54 g of Intermediate 9-2 (yield: 70%). The resulting compound was identified by LC-MS. C₁₆H₁₂O: M+ 234.1

Synthesis of Intermediate 9-3

2.34 g (10 mmol) of Intermediate 9-2 and 4.18 ml (30 mmol) of triethylamine were dissolved in 60 mL of DCM in a flask, and 1.68 ml (10 mmol) of trifluoromethanesulfonic anhydride was dissolved in 20 mL of DCM and slowly added to the flask at 0° C., followed by stirring for 5 hours at room temperature. 40 mL of water was added to the reaction solution, and the mixture was extracted three times with 50 mL of ethyl ether. The collected organic layer was dried using MgSO₄, and the residue obtained by evaporating the solvent was separated and purified by silica gel column chromatography to thereby obtain 2.64 g (yield: 75%) of Intermediate 9-3. The resulting compound was identified by LC-MS. C₁₇H₁₁F₃O₃S: M⁺ 352.0

Synthesis of Intermediate 9-4

3.52 g (10 mmol) of Intermediate 9-3, 1.40 g (15 mmol) of aniline, 0.46 g (0.5 mmol) of tris(dibenzylideneacetone)dipalladium(0) (Pd₂dba₃), 0.41 g (1 mmol) of s-phos, and 2.88 g (30 mmol) of sodium tert-butoxide were dissolved in 60 mL of toluene, followed by stirring at 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 1.77 g (yield: 60%) of Intermediate 9-4. The resulting compound was identified by LC-MS. C₂₂H₁₇N: M⁺ 295.1

Synthesis of Compound 9

2.95 g (10 mmol) of Intermediate 9-4, 3.49 g (10 mmol) of 2-bromo-9,9-dimethyl-5-phenyl-9H-fluorene, 0.46 g (0.5 mmol) of tris(dibenzylideneacetone)dipalladium(0)(Pd₂dba₃), 0.24 g (1 mmol) of P(t-Bu)₃, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 3.95 g (yield: 70%) of Compound 9. The resulting compound was identified by MS/FAB and 1H NMR.

### Synthesis Example 4: Synthesis of Compound 10

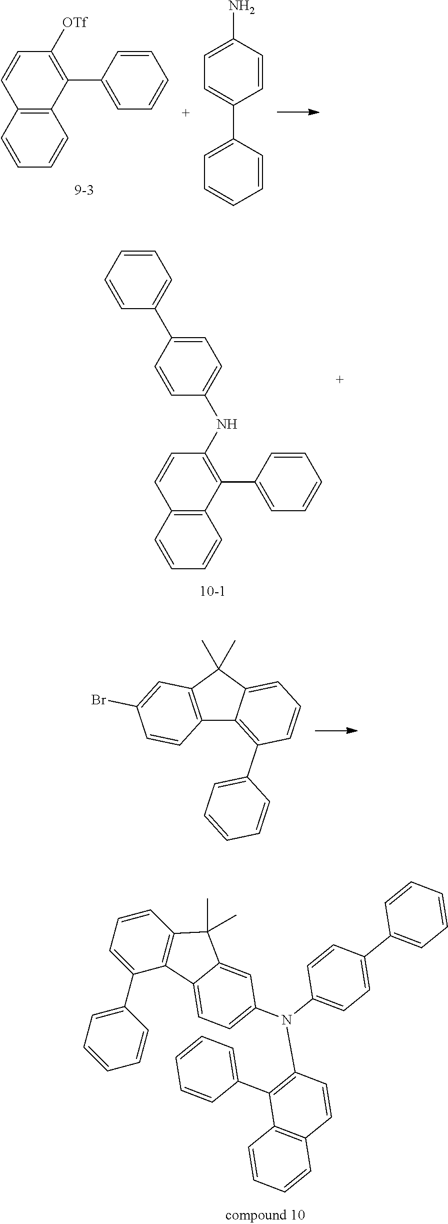

Compound 10 was synthesized in the same manner as used to synthesize Compound 9, except that 4-aminobiphenyl was used instead of aniline. The resulting compound was identified by MS/FAB and 1H NMR.

### Synthesis Example 5: Synthesis of Compound 18

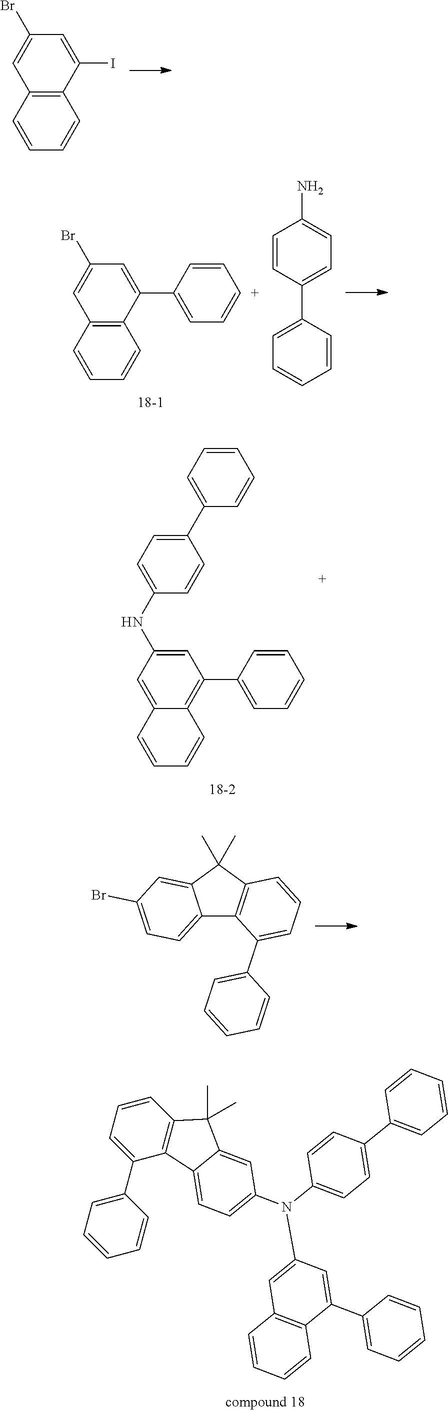 [SMILES: Brc1cc(I)c2ccccc2c1 ; Brc1cc(C2=CC=[C+]C=C2)c2ccccc2c1 ; Nc1ccc(-c2ccccc2)cc1 ; c1ccc(-c2ccc(Nc3cc(-c4ccccc4)c4ccccc4c3)cc2)cc1 ; CC1(C)c2cc(Br)ccc2-c2c(-c3ccccc3)cccc21 ; CC1(C)c2cc(N(c3ccc(-c4ccccc4)cc3)c3cc(-c4ccccc4)c4ccccc4c3)ccc2-c2c(-c3ccccc3)cccc21]

Synthesis of Intermediate 18-1

3.33 g (10 mmol) of 3-bromo-1-iodonaphthalene, 1.10 g (9 mmol) of phenylboronic acid, 0.58 g (0.5 mmol) of Pd(PPh₃)₄, and 4.14 g (30 mmol) of K₂CO₃ were dissolved in 60 mL of a THF/H₂O (2/1) mixed solution, and stirred for 16 hours at 80° C. The reaction solution was cooled at room temperature, followed by three times of an extraction process using 60 ml of water and 60 mL of diethyl ether. The resulting organic layer was dried by using MgSO₄. A solvent was removed therefrom by evaporation. The resulting residue was separated and purified through silica gel chromatography to thereby obtain 1.56 g of Intermediate 18-1 (yield: 55%). The resulting compound was identified by LC-MS. C₁₆H₁₁Br: M⁺ 282.0

Synthesis of Intermediate 18-2

2.82 g (10 mmol) of Intermediate 18-1, 2.54 g (15 mmol) of 4-aminobiphenyl, 0.46 g (0.5 mmol) of tris(dibenzylideneacetone)dipalladium(0) (Pd₂dba₃), 0.24 g (1 mmol) of P(t-Bu)3, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 2.41 g (yield: 65%) of Intermediate 18-2. The resulting compound was identified by LC-MS. C₂₈H₂₁N: M⁺ 371.1

Synthesis of Compound 18

3.71 g (10 mmol) of Intermediate 18-2, 3.49 g (10 mmol) of 2-bromo-9,9-dimethyl-5-phenyl-9H-fluorene, 0.46 g (0.5 mmol) of tris(dibenzylideneacetone)dipalladium(0)(Pd₂dba₃), 0.24 g (1 mmol) of P(t-Bu)3, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 4.16 g (yield: 65%) of Compound 18. The resulting compound was identified by MS/FAB and 1H NMR.

### Synthesis Example 6: Synthesis of Compound 45

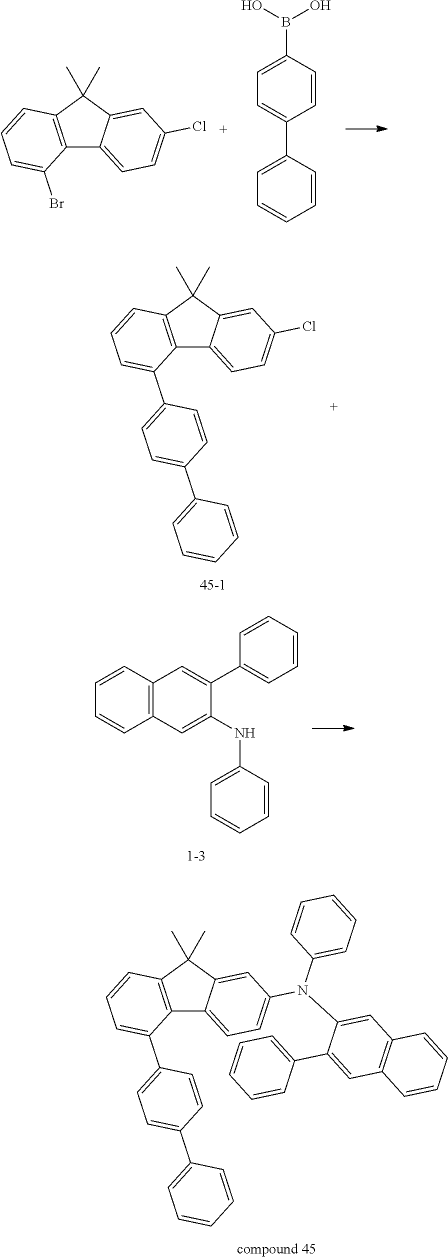 [SMILES: CC1(C)c2cc(Cl)ccc2-c2c(Br)cccc21 ; OB(O)c1ccc(-c2ccccc2)cc1 ; CC1(C)c2cc(Cl)ccc2-c2c(-c3ccc(-c4ccccc4)cc3)cccc21 ; c1ccc(Nc2cc3ccccc3cc2-c2ccccc2)cc1 ; CC1(C)c2cc(N(c3ccccc3)c3cc4ccccc4cc3-c3ccccc3)ccc2-c2c(-c3ccc(-c4ccccc4)cc3)cccc21]

Synthesis of Intermediate 45-1

3.08 g (10 mmol) of 5-bromo-2-chloro-9,9-dimethyl-9H-fluorene, 2.18 g (11 mmol) of [1,1′-biphenyl]-4-ylboronic acid, 0.58 g (0.5 mmol) of Pd(PPh₃)₄, and 4.14 g (30 mmol) of K₂CO₃ were dissolved in 60 mL of a THF/H₂O (2/1) mixed solution, and stirred for 16 hours at 80° C. The reaction solution was cooled at room temperature, followed by three times of an extraction process using 60 ml of water and 60 ml of diethyl ether. The resulting organic layer was dried by using MgSO₄. A solvent was removed therefrom by evaporation. The resulting residue was separated and purified through silica gel chromatography to thereby obtain 2.27 g of Intermediate 4-2 (yield: 70%). The resulting compound was identified by LC-MS. C₂₇H₂₁Cl: M⁺ 380.9

Synthesis of Compound 45

3.81 g (10 mmol) of Intermediate 45-1, 2.95 g (10 mmol) of Intermediate 1-3, 0.46 g (0.5 mmol) of tris(dibenzylideneacetone)dipalladium(0) (Pd₂dba₃), 0.24 g (1 mmol) of P(t-Bu)₃, and 2.88 g (30 mmol) of sodium tert-butoxide were dissolved in 60 mL of o-xylene, and stirred for 3 hours at 130° C. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 4.16 g (yield: 65%) of Compound 45. The resulting compound was identified by MS/FAB and 1H NMR.

### Synthesis Example 7: Synthesis of Compound 65

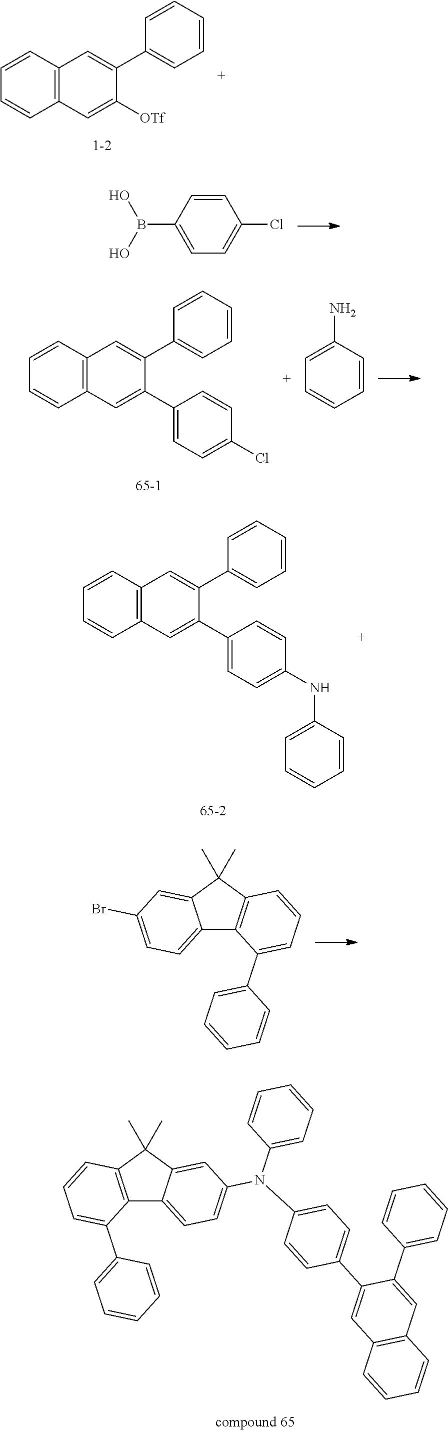

Synthesis of Intermediate 65-1

3.52 g (10 mmol) of Intermediate 1-2, 1.56 g (10 mmol) of (4-chlorophenyl)boronic acid, 0.58 g (0.5 mmol) of Pd(PPh₃)₄, and 4.14 g (30 mmol) of K₂CO₃ were dissolved in 60 mL of a THF/H₂O (2/1) mixed solution, and stirred for 16 hours at 80° C. The reaction solution was cooled at room temperature, followed by three times of an extraction process using 60 ml of water and 60 ml of diethyl ether. The resulting organic layer was dried by using MgSO₄. A solvent was removed therefrom by evaporation. The resulting residue was separated and purified through silica gel chromatography to thereby obtain 2.21 g of Intermediate 65-1 (yield: 70%). The resulting compound was identified by LC-MS. C₂₂H₁₅Cl: M⁺314.0

Synthesis of Intermediate 65-2

3.14 g (10 mmol) of Intermediate 65-1, 1.40 g (15 mmol) of aniline, 0.46 g (0.5 mmol) of tris(dibenzylideneacetone)dipalladium(0) (Pd₂dba₃), 0.41 g (1 mmol) of s-phos, and 2.88 g (30 mmol) of sodium tert-butoxide were dissolved in 60 mL of o-xylene, followed by stirring at 130° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 2.23 g (yield: 60%) of Intermediate 65-2. The resulting compound was identified by LC-MS. C₂₈H₂₁N: M⁺ 371.1

Synthesis of Compound 65

3.71 g (10 mmol) of Intermediate 65-2, 3.49 g (10 mmol) of 2-bromo-9,9-dimethyl-5-phenyl-9H-fluorene, 0.46 g (0.5 mmol) of tris(dibenzylideneacetone)dipalladium(0)(Pd₂dba₃), 0.24 g (1 mmol) of P(t-Bu)3, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 4.48 g (yield: 70%) of Compound 65. The resulting compound was identified by MS/FAB and 1H NMR.

### Synthesis Example 8: Synthesis of Compound 73

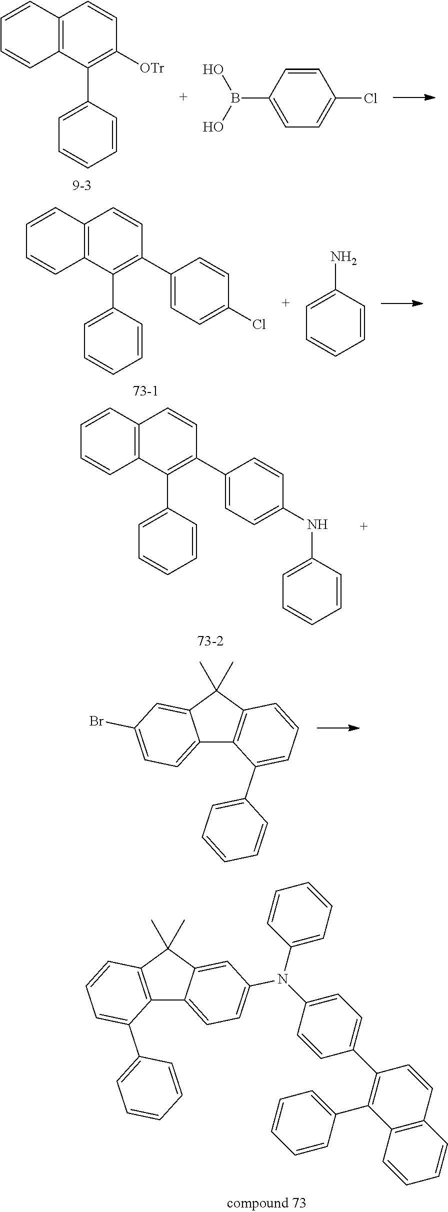

Compound 73 was synthesized in the same manner as used to synthesize Compound 65, except that Intermediate 9-3 was used instead of Intermediate 1-2. The resulting compound was identified by MS/FAB and 1H NMR.

### Synthesis Example 9: Synthesis of Compound 76

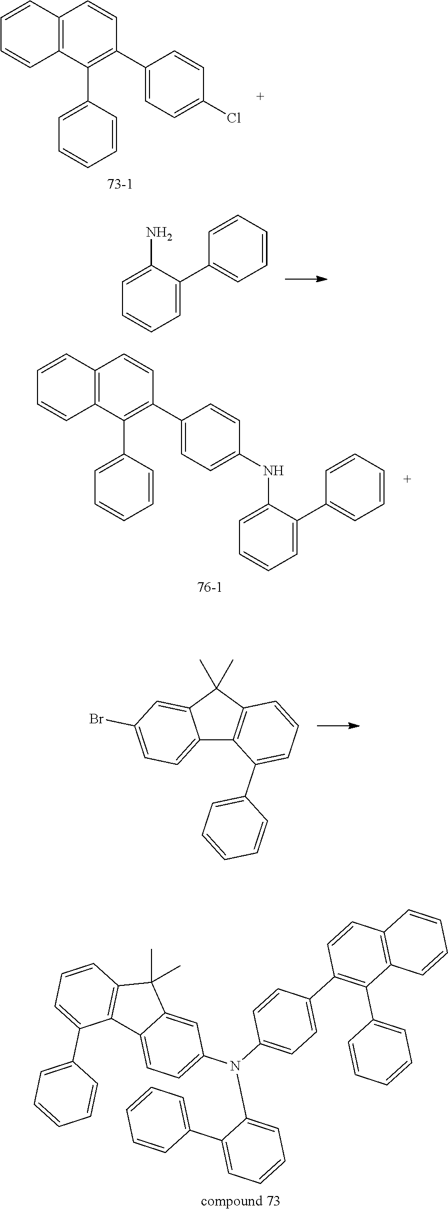

Compound 76 was synthesized in the same manner as used to synthesize Compound 73, except that 2-aminobiphenyl was used instead of aniline. The resulting compound was identified by MS/FAB and 1H NMR.

### [Synthesis Example 10: Synthesis of Compound 85]

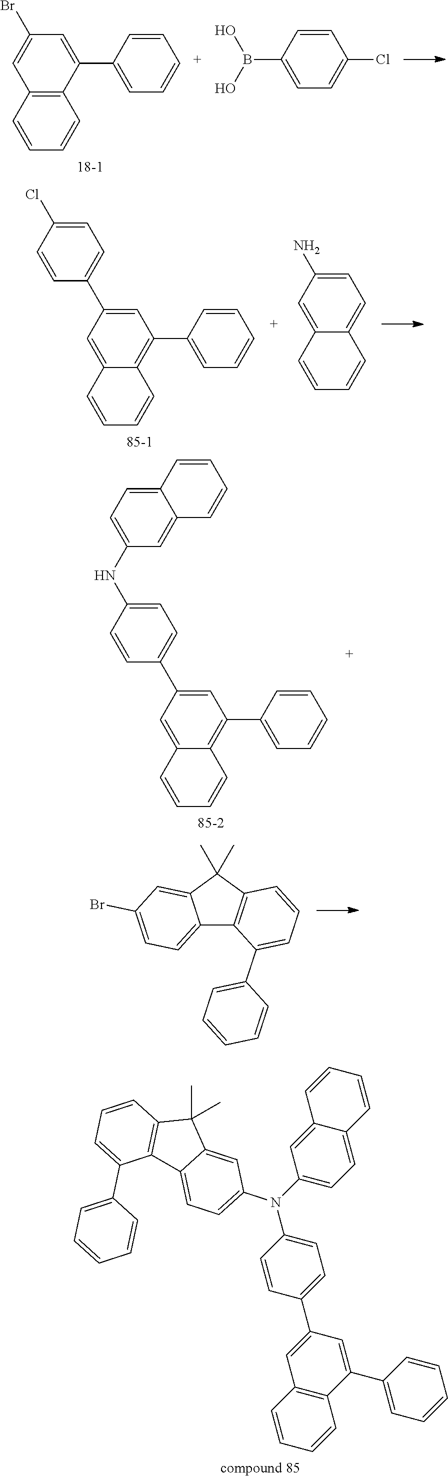

Synthesis of Intermediate 85-1

2.83 g (10 mmol) of Intermediate 18-1, 1.56 g (10 mmol) of (4-chlorophenyl)boronic acid, 0.58 g (0.5 mmol) of Pd(PPh₃)₄, and 4.14 g (30 mmol) of K₂CO₃ were dissolved in 60 mL of a THF/H₂O (2/1) mixed solution, and stirred for 16 hours at 80° C. The reaction solution was cooled at room temperature, followed by three times of an extraction process using 60 ml of water and 60 ml of diethyl ether. The resulting organic layer was dried by using MgSO₄. A solvent was removed therefrom by evaporation. The resulting residue was separated and purified through silica gel chromatography to thereby obtain 2.46 g of Intermediate 85-1 (yield: 78%). The resulting compound was identified by LC-MS. C₂₂H₁₅Cl: M⁺314.0

Synthesis of Intermediate 85-2

3.14 g (10 mmol) of Intermediate 65-1, 2.15 g (15 mmol) of naphthalen-2-amine, 0.46 g (0.5 mmol) of tris(dibenzylideneacetone)dipalladium(0) (Pd₂dba₃), 0.41 g (1 mmol) of s-phos, and 2.88 g (30 mmol) of sodium tert-butoxide were dissolved in 60 mL of o-xylene, followed by stirring at 130° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 2.79 g (yield: 66%) of Intermediate 85-2. The resulting compound was identified by LC-MS. C₃₂H₂₃N: M⁺ 421.1

Synthesis of Compound 85

4.21 g (10 mmol) of Intermediate 85-2, 3.49 g (10 mmol) of 2-bromo-9,9-dimethyl-5-phenyl-9H-fluorene, 0.46 g (0.5 mmol) of tris(dibenzylideneacetone)dipalladium(0)(Pd₂dba₃), 0.24 g (1 mmol) of P(t-Bu)3, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 4.83 g (yield: 70%) of Compound 85. The resulting compound was identified by MS/FAB and 1H NMR.

### Synthesis Example 11: Synthesis of Compound 166

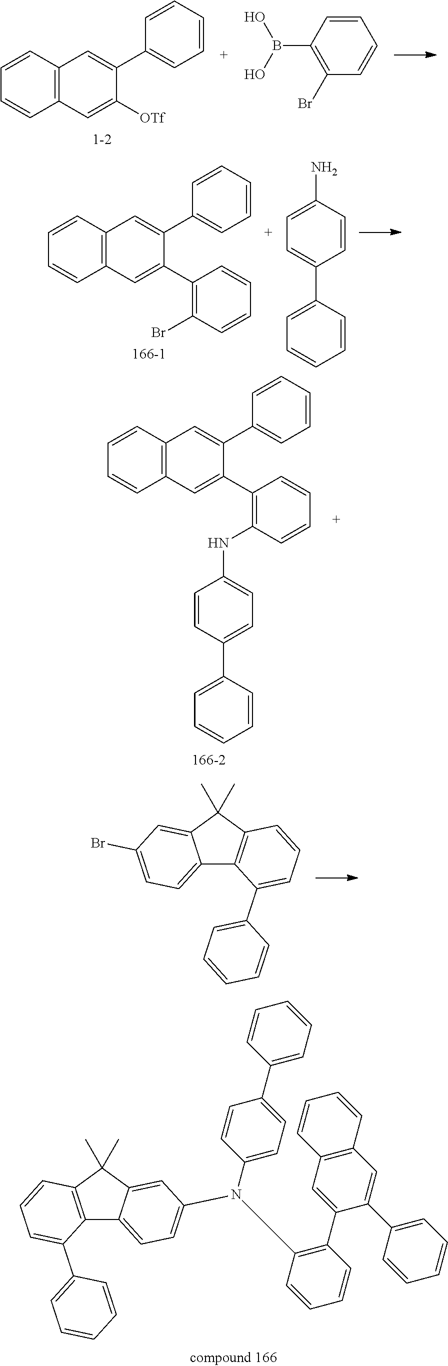

Synthesis of Intermediate 166-1

3.52 g (10 mmol) of Intermediate 1-2, 2.00 g (10 mmol) of (2-bromophenyl)boronic acid, 0.58 g (0.5 mmol) of Pd(PPh₃)₄, and 4.14 g (30 mmol) of K₂CO₃ were dissolved in 60 mL of a THF/H₂O (2/1) mixed solution, and stirred for 16 hours at 80° C. The reaction solution was cooled at room temperature, followed by three times of an extraction process using 60 ml of water and 60 ml of diethyl ether. The resulting organic layer was dried by using MgSO₄. A solvent was removed therefrom by evaporation. The resulting residue was separated and purified through silica gel chromatography to thereby obtain 2.23 g (yield: 62%) of Intermediate 166-1. The resulting compound was identified by LC-MS. C₂₂H₁₅Br: M⁺358.0

Synthesis of Intermediate 166-2

3.58 g (10 mmol) of Intermediate 166-1, 2.54 g (15 mmol) of 4-aminobiphenyl, 0.46 g (0.5 mmol) of tris(dibenzylideneacetone)dipalladium(0) (Pd₂dba₃), 0.24 g (1 mmol) of P(t-Bu)3, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 2.91 g (yield: 65%) of Intermediate 166-2. The resulting compound was identified by LC-MS. C₃₄H₂₅N: M⁺ 447.2

Synthesis of Compound 166

4.47 g (10 mmol) of Intermediate 166-2, 3.49 g (10 mmol) of 2-bromo-9,9-dimethyl-5-phenyl-9H-fluorene, 0.46 g (0.5 mmol) of tris(dibenzylideneacetone)dipalladium(0)(Pd₂dba₃), 0.24 g (1 mmol) of P(t-Bu)3, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 5.01 g (yield: 70%) of Compound 166. The resulting compound was identified by MS/FAB and 1H NMR.

### Synthesis Example 12: Synthesis of Compound 204

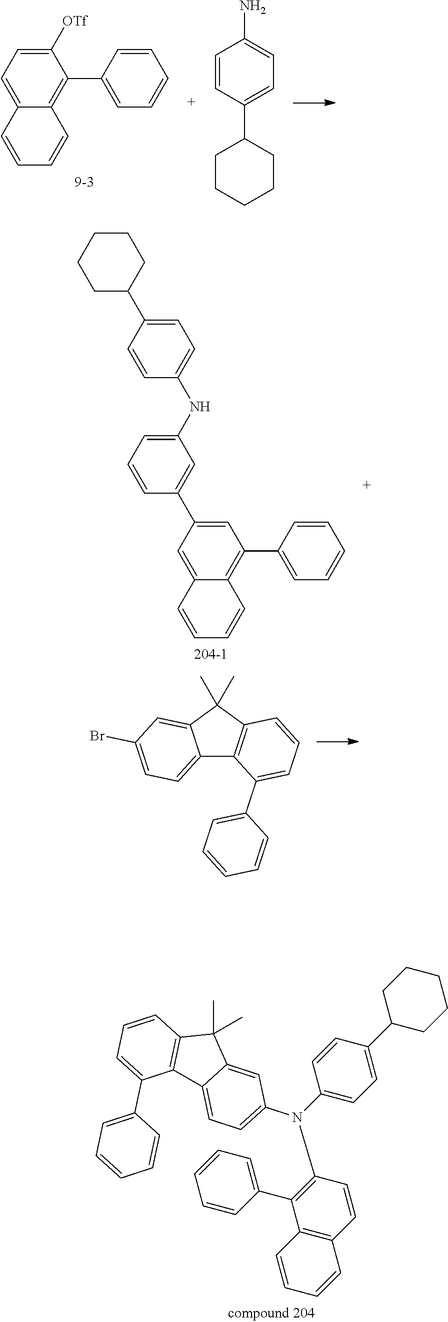

Synthesis of Intermediate 204-1

3.52 g (10 mmol) of Intermediate 9-3, 2.63 g (15 mmol) of 4-cyclohexylaniline, 0.46 g (0.5 mmol) of tris(dibenzylideneacetone)dipalladium(0) (Pd₂dba₃), 0.41 g (1 mmol) of s-phos, and 2.88 g (30 mmol) of sodium tert-butoxide were dissolved in 60 mL of toluene, followed by stirring at 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 2.27 g (yield: 60%) of Intermediate 204-1. The resulting compound was identified by LC-MS. C₂₈H₂₇N: M⁺ 377.2

Synthesis of Compound 204

3.77 g (10 mmol) of Intermediate 204-1, 3.49 g (10 mmol) of 2-bromo-9,9-dimethyl-5-phenyl-9H-fluorene, 0.46 g (0.5 mmol) of tris(dibenzylideneacetone)dipalladium(0)(Pd₂dba₃), 0.24 g (1 mmol) of P(t-Bu)3, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 4.52 g (yield: 70%) of Compound 204. The resulting compound was identified by MS/FAB and 1H NMR.

### Synthesis Example 13: Synthesis of Compound 209

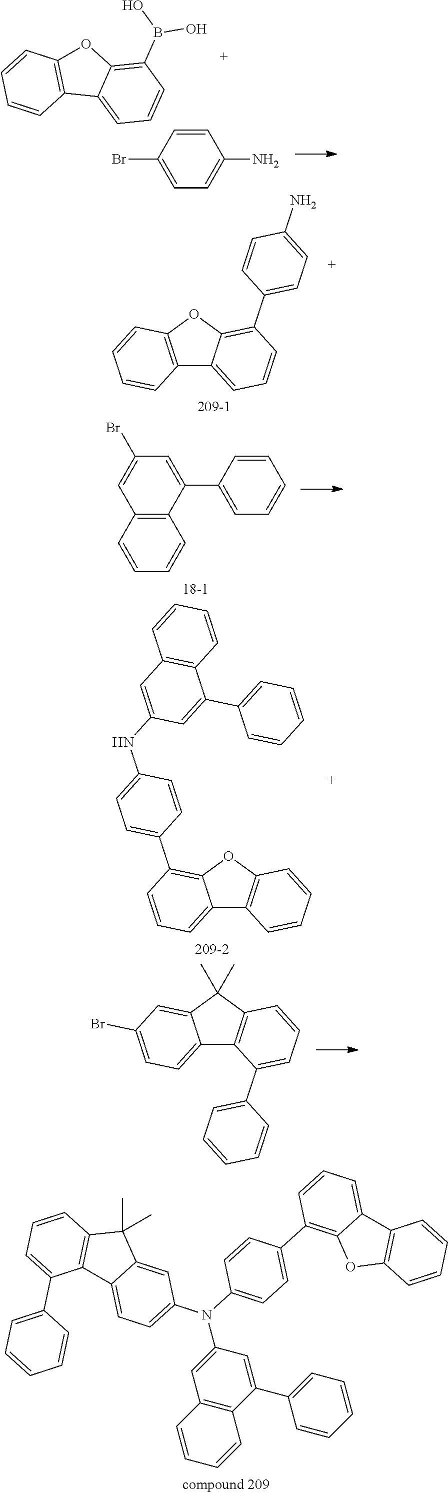

Synthesis of Intermediate 209-1

2.12 g (10 mmol) of dibenzo[b,d]furan-4-ylboronic acid, 1.72 g (10 mmol) of 4-bromoaniline, 0.58 g (0.5 mmol) of Pd(PPh₃)₄, and 4.14 g (30 mmol) of K₂CO₃ were dissolved in 60 mL of a mixture solution of THF/H₂O (2/1). The mixture was stirred at a temperature of 80° C. for 16 hours. The reaction solution was cooled at room temperature, followed by three times of an extraction process using 60 ml of water and 60 ml of diethyl ether. The resulting organic layer was dried by using MgSO₄. A solvent was removed therefrom by evaporation. The resulting residue was separated and purified through silica gel chromatography to thereby obtain 1.68 g of Intermediate 209-1 (yield: 65%). The resulting compound was identified by LC-MS. C1₈H₁₃NO: M⁺ 259.1

Synthesis of Intermediate 209-2

3.89 g (15 mmol) of Intermediate 209-1, 2.83 g (10 mmol) of Intermediate 18-1, 0.46 g (0.5 mmol) of tris(dibenzylideneacetone)dipalladium(0) (Pd₂dba₃), 0.41 g (1 mmol) of s-phos, and 2.88 g (30 mmol) of sodium tert-butoxide were dissolved in 60 mL of toluene, followed by stirring at 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 2.77 g (yield: 60%) of Intermediate 209-2. The resulting compound was identified by LC-MS. C₃₄H₂₃NO: M⁺ 461.1

Synthesis of Compound 209

4.61 g (10 mmol) of Intermediate 209-2, 3.49 g (10 mmol) of 2-bromo-9,9-dimethyl-5-phenyl-9H-fluorene, 0.46 g (0.5 mmol) of tris(dibenzylideneacetone)dipalladium(0)(Pd₂dba₃), 0.24 g (1 mmol) of P(t-Bu)3, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 4.75 g (yield: 65%) of Compound 209. The resulting compound was identified by MS/FAB and 1H NMR.

### Synthesis Example 14: Synthesis of Compound 210

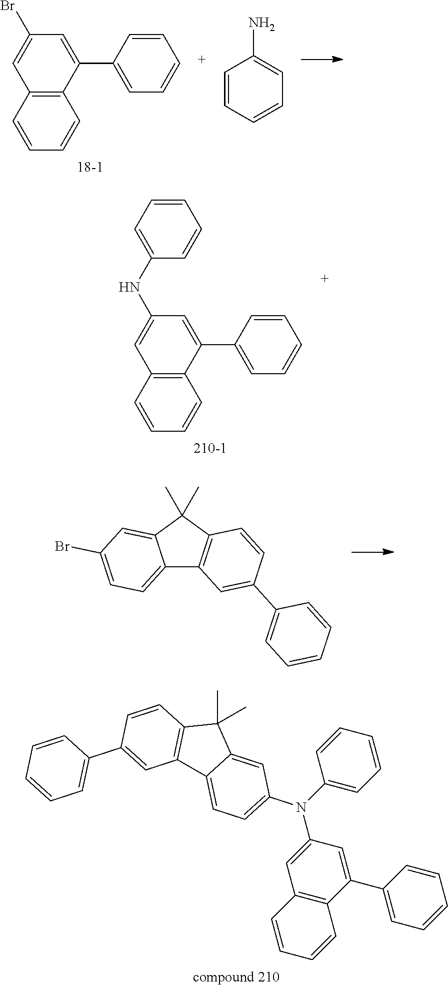

Synthesis of Intermediate 210-1

2.83 g (10 mmol) of Intermediate 18-1, 1.40 g (15 mmol) of aniline, 0.46 g (0.5 mmol) of tris(dibenzylideneacetone)dipalladium(0) (Pd₂dba₃), 0.41 g (1 mmol) of s-phos, and 2.88 g (30 mmol) of sodium tert-butoxide were dissolved in 60 mL of toluene, followed by stirring at 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 2.07 g (yield: 70%) of Intermediate 210-1. The resulting compound was identified by LC-MS. C₂₂H₁₇N: M⁺ 295.1

Synthesis of Compound 210

2.95 g (10 mmol) of Intermediate 210-1, 3.49 g (10 mmol) of 2-bromo-9,9-dimethyl-5-phenyl-9H-fluorene, 0.46 g (0.5 mmol) of tris(dibenzylideneacetone)dipalladium(0)(Pd₂dba₃), 0.24 g (1 mmol) of P(t-Bu)3, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 3.67 g (yield: 65%) of Compound 209. The resulting compound was identified by MS/FAB and 1H NMR.

### Synthesis Example 15: Synthesis of Compound 211

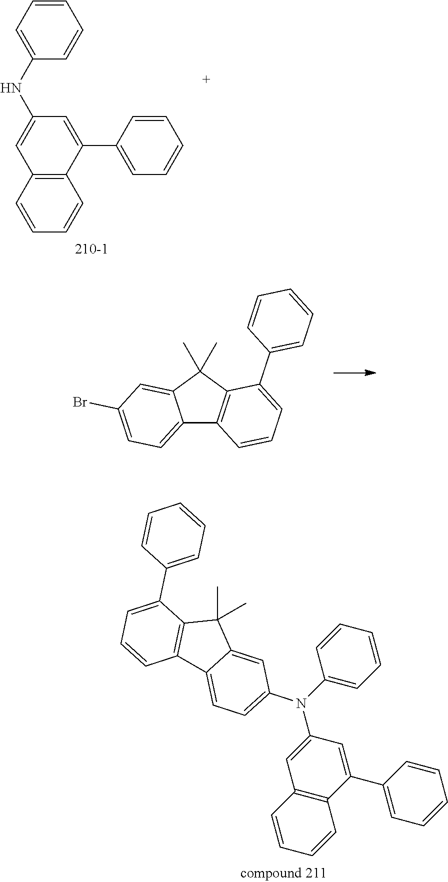 [SMILES: c1ccc(Nc2cc(-c3ccccc3)c3ccccc3c2)cc1 ; CC1(C)c2cc(Br)ccc2-c2cccc(-c3ccccc3)c21 ; CC1(C)c2cc(N(c3ccccc3)c3cc(-c4ccccc4)c4ccccc4c3)ccc2-c2cccc(-c3ccccc3)c21]

Compound 211 was synthesized in the same manner as used to synthesize Compound 210, except that 2-bromo-9,9-dimethyl-8-phenyl-9H-fluorene was used instead of 2-bromo-9,9-dimethyl-6-phenyl-9H-fluorene. The resulting compound was identified by MS/FAB and 1H NMR.

### Synthesis Example 16: Synthesis of Compound 212

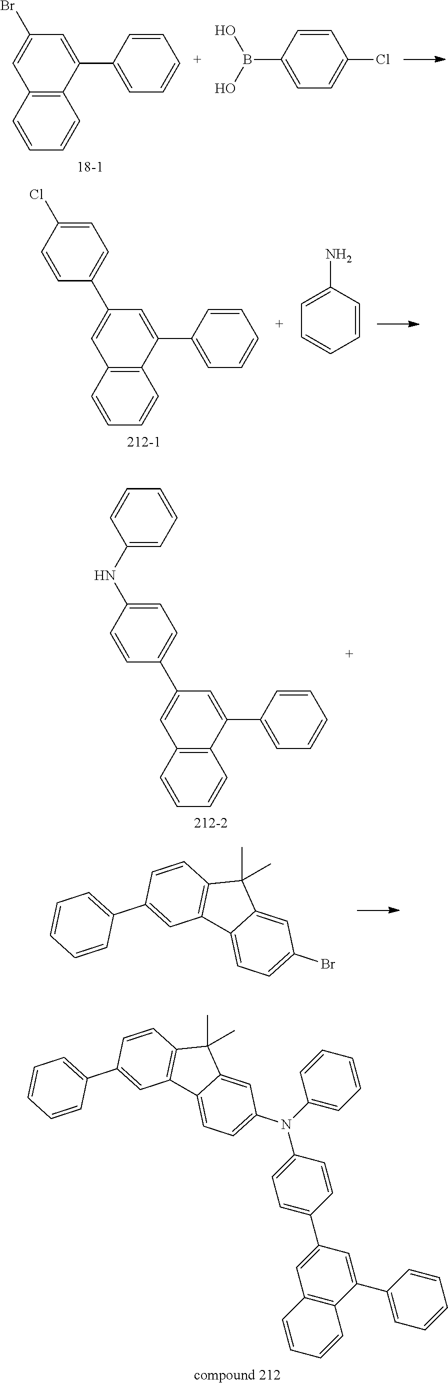

Synthesis of Intermediate 212-1

2.83 g (10 mmol) of Intermediate 18-1, 1.56 g (10 mmol) of (4-chlorophenyl)boronic acid, 0.58 g (0.5 mmol) of Pd(PPh₃)₄, and 4.14 g (30 mmol) of K₂CO₃ were dissolved in 60 mL of a THF/H₂O (2/1) mixed solution, and stirred for 16 hours at 80° C. The reaction solution was cooled at room temperature, followed by three times of an extraction process using 60 ml of water and 60 ml of diethyl ether. The resulting organic layer was dried by using MgSO₄. A solvent was removed therefrom by evaporation. The resulting residue was separated and purified through silica gel chromatography to thereby obtain 2.21 g of Intermediate 212-1 (yield: 70%). The resulting compound was identified by LC-MS. C₂₂H₁₅Cl: M⁺ 314.0

Synthesis of Intermediate 212-2

3.14 g (10 mmol) of Intermediate 212-1, 1.40 g (15 mmol) of aniline, 0.46 g (0.5 mmol) of tris(dibenzylideneacetone)dipalladium(0) (Pd₂dba₃), 0.41 g (1 mmol) of s-phos, and 2.88 g (30 mmol) of sodium tert-butoxide were dissolved in 60 mL of o-xylene, followed by stirring at 130° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 2.23 g (yield: 60%) of Intermediate 212-2. The resulting compound was identified by LC-MS. C₂₈H₂₁N: M⁺ 371.1

Synthesis of Compound 212

3.71 g (10 mmol) of Intermediate 212-2, 3.49 g (10 mmol) of 2-bromo-9,9-dimethyl-6-phenyl-9H-fluorene, 0.46 g (0.5 mmol) of tris(dibenzylideneacetone)dipalladium(0)(Pd₂dba₃), 0.24 g (1 mmol) of P(t-Bu)3, and 2.88 g (30 mmol) of sodium tert-butoxide were dissolved in 60 mL of toluene, followed by stirring at a temperature of 80° C. for 3 hours. The reaction solution was cooled at room temperature, and 40 ml of water was added thereto, followed by three times of an extraction process using 50 ml of ethyl ether. The collected organic layer was dried using MgSO₄, and the residue obtained by evaporating the solvent was separated and purified by silica gel column chromatography to thereby obtain 4.16 g (yield: 65%) of Compound 212. The resulting compound was identified by MS/FAB and 1H NMR.

### Synthesis Example 17: Synthesis of Compound 213

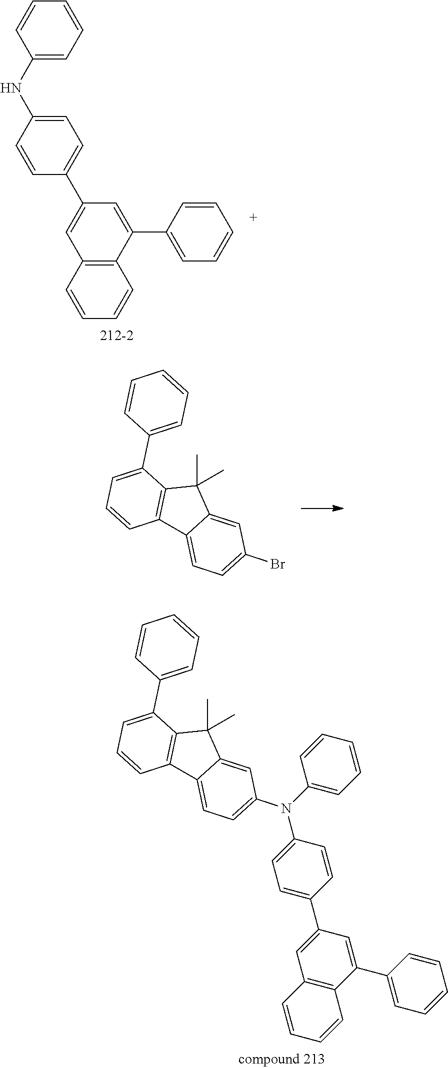 [SMILES: c1ccc(Nc2ccc(-c3cc(-c4ccccc4)c4ccccc4c3)cc2)cc1 ; CC1(C)c2cc(Br)ccc2-c2cccc(-c3ccccc3)c21 ; CC1(C)c2cc(N(c3ccccc3)c3ccc(-c4cc(-c5ccccc5)c5ccccc5c4)cc3)ccc2-c2cccc(-c3ccccc3)c21]

Compound 213 was synthesized in the same manner as used to synthesize Compound 212, except that 2-bromo-9,9-dimethyl-8-phenyl-9H-fluorene was used instead of 2-bromo-9,9-dimethyl-6-phenyl-9H-fluorene. The resulting compound was identified by MS/FAB and ¹H NMR.

### Synthesis Example 18: Synthesis of Compound 214

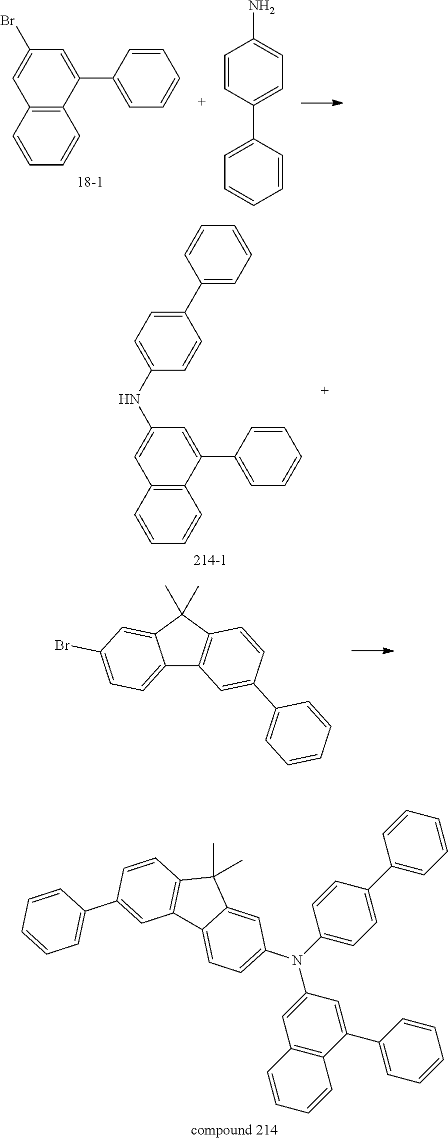

Compound 214 was synthesized in the same manner as used to synthesize Compound 210, except that 4-aminobiphenyl was used instead of aniline. The resulting compound was identified by MS/FAB and ¹H NMR.

### Synthesis Example 19: Synthesis of Compound 215

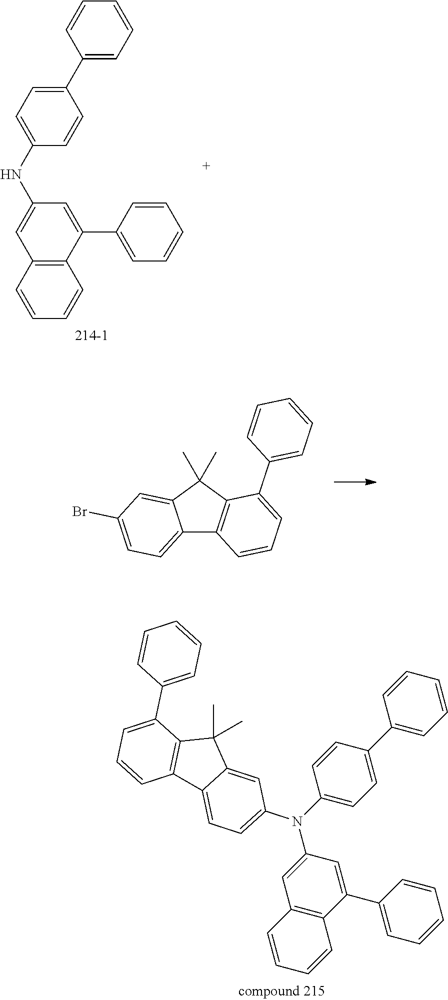 [SMILES: c1ccc(-c2ccc(Nc3cc(-c4ccccc4)c4ccccc4c3)cc2)cc1 ; CC1(C)c2cc(Br)ccc2-c2cccc(-c3ccccc3)c21 ; CC1(C)c2cc(N(c3ccc(-c4ccccc4)cc3)c3cc(-c4ccccc4)c4ccccc4c3)ccc2-c2cccc(-c3ccccc3)c21]

Compound 2 was synthesized in the same manner as used to synthesize Compound 214, except that 2-bromo-9,9-dimethyl-8-phenyl-9H-fluorene was used instead of 2-bromo-9,9-dimethyl-6-phenyl-9H-fluorene. The resulting compound was identified by MS/FAB and ¹H NMR.

### Synthesis Example 20: Synthesis of Compound 216

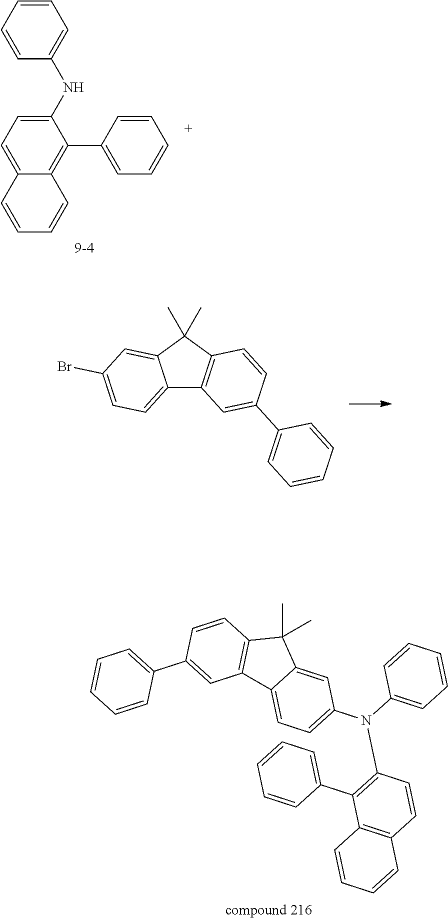 [SMILES: c1ccc(Nc2ccc3ccccc3c2-c2ccccc2)cc1 ; CC1(C)c2ccc(-c3ccccc3)cc2-c2ccc(Br)cc21 ; CC1(C)c2ccc(-c3ccccc3)cc2-c2ccc(N(c3ccccc3)c3ccc4ccccc4c3-c3ccccc3)cc21]

Compound 216 was synthesized in the same manner as used to synthesize Compound 9, except that 2-bromo-9,9-dimethyl-6-phenyl-9H-fluorene was used instead of 2-bromo-9,9-dimethyl-5-phenyl-9H-fluorene. The resulting compound was identified by MS/FAB and ¹H NMR.

### Synthesis Example 21: Synthesis of Compound 217

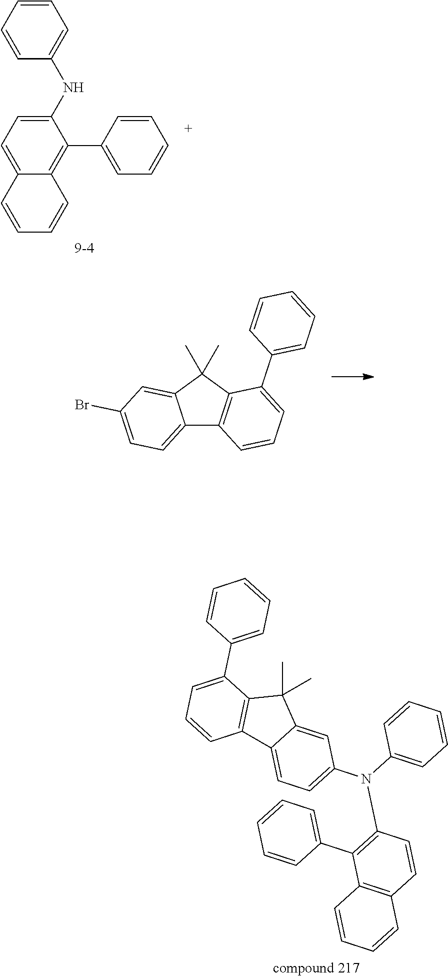 [SMILES: c1ccc(Nc2ccc3ccccc3c2-c2ccccc2)cc1 ; CC1(C)c2cc(Br)ccc2-c2cccc(-c3ccccc3)c21 ; CC1(C)c2cc(N(c3ccccc3)c3ccc4ccccc4c3-c3ccccc3)ccc2-c2cccc(-c3ccccc3)c21]

Compound 217 was synthesized in the same manner as used to synthesize Compound 9, except that 2-bromo-9,9-dimethyl-8-phenyl-9H-fluorene was used instead of 2-bromo-9,9-dimethyl-6-phenyl-9H-fluorene. The resulting compound was identified by MS/FAB and 1H NMR.

The results of MS/FAB and ¹H NMR of Example Compounds are shown in [Table 1]

TABLE 1

| | | MS/FAB | |
|---|---|---|---|
| Compound | ¹H NMR (CDCl₃, 400 MHz) | found | calc. |
| Compound 1 | 7.86-7.77(m, 6H), 7.70-7.65(m, 2H), 7.47-7.33(m, 11H), | 563.15 | 563.26 |
| | 7.24-7.08(m, 8H), 1.69(s, 6H) | | |
| Compound 2 | 7.86-7.65(m, 10H), 7.55-7.33(m, 18H), 7.16(d, 1H), | 639.18 | 639.29 |
| | 7.08(d, 2H), 1.69(s, 6H) | | |
| Compound 9 | 8.80(d, 1H), 7.97-7.78(m, 7H), 7.65(d, 1H), 7.51-7.33(m, | 563.15 | 563.26 |
| | 10H), 7.25-7.16(m, 5H), 7.08-7.00(m, 3H), 1.69(s, 6H) | | |
| Compound 10 | 8.80(d, 1H), 7.97-7.75(m, 9H), 7.65(d, 1H), 7.51-7.33(m, | 639.18 | 639.29 |
| | 17H), 7.25-7.16(m, 3H), 1.69(s, 6H) | | |
| Compound 18 | 8.72(d, 1H), 8.05(d, 1H), 7.86-7.75(m, 8H), 7.65(d, 1H), | 639.18 | 639.29 |
| | 7.55-7.29(m, 17H), 7.16-7.12(m, 2H), 6.77(s, 1H), 1.69(s, | | |
| | 6H) | | |
| Compound 45 | 7.86(m, 2H), 7.78-7.65(m, 6H), 7.49-7.33(m, 11H), 7.25- | 639.18 | 639.29 |
| | 7.00(m, 12H), 1.69(s, 6H) | | |
| Compound 65 | 8.57(s, 2H), 8.15(d, 2H), 7.86-7.78(m, 6H), 7.65-7.64(m, | 639.18 | 639.29 |
| | 3H), 7.55-7.33(m, 12H), 7.24-7.16(m, 3H), 7.08-7.00(m, | | |
| | 3H), 1.69(s, 6H) | | |
| Compound 73 | 8.93(d, 1H), 8.31-8.26(m, 2H), 7.86-7.65(m, 8H), 7.55- | 639.18 | 639.29 |
| | 7.33(m, 14H), 7.24-7.16(m, 3H), 7.08-7.00(m, 3H), 1.69(s, | | |
| | 6H) | | |
| Compound 76 | 8.93(d, 1H), 8.31-8.26(m, 2H), 8.10(d, 1H), 7.86-7.33(m, | 715.21 | 715.32 |
| | 27H), 7.16-7.08(m, 4H), 1.69(s, 6H) | | |
| Compound 85 | 9.01(d, 1H), 8.18(d, 1H), 7.98(s, 1H), 7.86-7.65(m, 9H), | 689.20 | 689.31 |
| | 7.55-7.33(m, 19H), 7.16-7.11(m, 2H), 1.69(s, 6H) | | |
| Compound 166 | 8.57(s, 2H), 8.15-8.10(m, 3H), 7.86-7.75(m, 8H), 7.65- | 715.21 | 715.32 |
| | 7.64(m, 3H), 7.55-7.33(m, 17H), 7.16-7.14(m, 2H), 1.69(s, | | |
| | 6H) | | |
| Compound 204 | 8.80(d, 1H), 7.97-7.78(m, 7H), 7.65(d, 1H), 7.51-7.33(m, | 645.23 | 645.34 |
| | 10H), 7.25-7.16(m, 5H), 7.06(d, 2H), 2.72(m, 1H), 1.86- | | |
| | 8.80(d, 1H), 7.97-7.78(m, 7H), 7.65(d, 1H), 7.51-7.33(m, | | |
| | 1.43(m, 16H) | | |
| Compound 209 | 8.72(d, 1H), 8.08-7.98(m, 4H), 7.88-7.78(m, 6H), 7.65(d, | 739.20 | 739.30 |
| | 1H), 7.55-7.29(m, 18H), 7.16-7.12(m, 2H), 6.77(s, 1H), | | |
| | 1.69(s, 6H) | | |
| Compound 210 | 8.72(d, 1H), 8.18(s, 1H), 8.05(d, 1H), 7.86-7.68(m, 7H), | 563.15 | 563.26 |
| | 7.49-7.00(m, 16H), 6.77(s, 1H), 1.69(s, 6H) | | |
| Compound 211 | 8.72(d, 1H), 8.05-8.00(m, 2H), 7.86-7.79(m, 3H), 7.68(d, | 563.15 | 563.26 |
| | 1H), 7.57(t, 1H), 7.46-7.00(m, 18H), 6.77(s, 1H), 1.69(s, | | |
| | 6H) | | |
| Compound 212 | 9.01(s, 1H), 8.18-8.17(m, 2H), 7.98(s, 1H), 7.86-7.68(m, | 639.18 | \|639.29 |
| | 7H), 7.55-7.33(m, 14H), 7.24-7.16(m, 3H), 7.08-7.00(m, | | |
| | 3H), 1.69(s, 6H) | | |
| Compound 213 | 9.01(s, 1H), 8.18(d, 1H), 8.00-7.98(m, 2H), 7.86-7.79(m, | 639.18 | 639.29 |
| | 3H), 7.68(d, 1H), 7.57-7.33(m, 17H), 7.24-7.16(m, 3H), | | |
| | 7.08-7.00(m, 3H), 1.69(s, 6H) | | |
| Compound 214 | 8.72(d, 1H), 8.18(s, 1H), 8.05(d, 1H), 7.86-7.68(m, 9H), | 639.18 | 639.29 |
| | 7.55-7.29(m, 16H), 7.16-7.12(m, 2H), 6.77(s, 1H), 1.69(s, | | |
| | 6H) | | |
| Compound 215 | 8.72(d, 1H), 8.05-8.00(m, 2H), 7.86-7.68(m, 6H), 7.57- | 639.18 | 639.29 |
| | 2.29(m, 19H), 7.16-7.12(m, 2H), 6.77(s, 1H), 1.69(s, 6H) | | |
| Compound 216 | 8.80(d, 1H), 8.18(s, 1H), 7.97(d, 1H), 7.89-7.86(m, 2H), | 563.15 | 563.26 |
| | 7.76-7.68(m, 5H), 7.51-7.33(m, 9H), 7.25-7.16(m, 5H), | | |
| | 7.08-7.00(m, 3H), 1.69(s, 6H) | | |
| Compound 217 | 8.80(d, 1H), 8.00-7.97(m, 2H), 7.89-7.86(m, 2H), 7.76(d, | 563.15 | 563.26 |
| | 1H), 7.68(d, 1H), 7.57-7.33(m, 12H), 7.25-7.16(m, 5H), | | |
| | 7.08-7.00(m, 3H), 1.69(s, 6H) | | |

### Comparative Example 1

A glass substrate (available from Corning Co., Ltd) on which an ITO anode (15 Ohms per square centimeter (Q/cm²)) having a thickness of 1,200 Å was formed was cut to a size of 50 millimeters (mm)×50 mm×0.7 mm, sonicated in isopropyl alcohol and pure water for 5 minutes in each solvent, cleaned by irradiation of ultraviolet rays and exposure to ozone for 30 minutes, and was mounted on a vacuum deposition apparatus.

2-TNATA was vacuum-deposited on the anode to form a hole injection layer having a thickness of 600 Å. 4,4′-bis[N-(1-naphthyl)-N-phenylamino]biphenyl (NPB) was vacuum-deposited on the hole injection layer to form a hole transport layer having a thickness of 300 Å.

9,10-di(naphthalen-2-yl)anthracene (DNA) and 4,4′-bis[2-(4-(N,N-diphenylamino)phenyl)vinyl]biphenyl (DPAVBi) were co-deposited on the hole transport layer to a weight ratio of 98:2 to form an emission layer having a thickness of 300 Å.

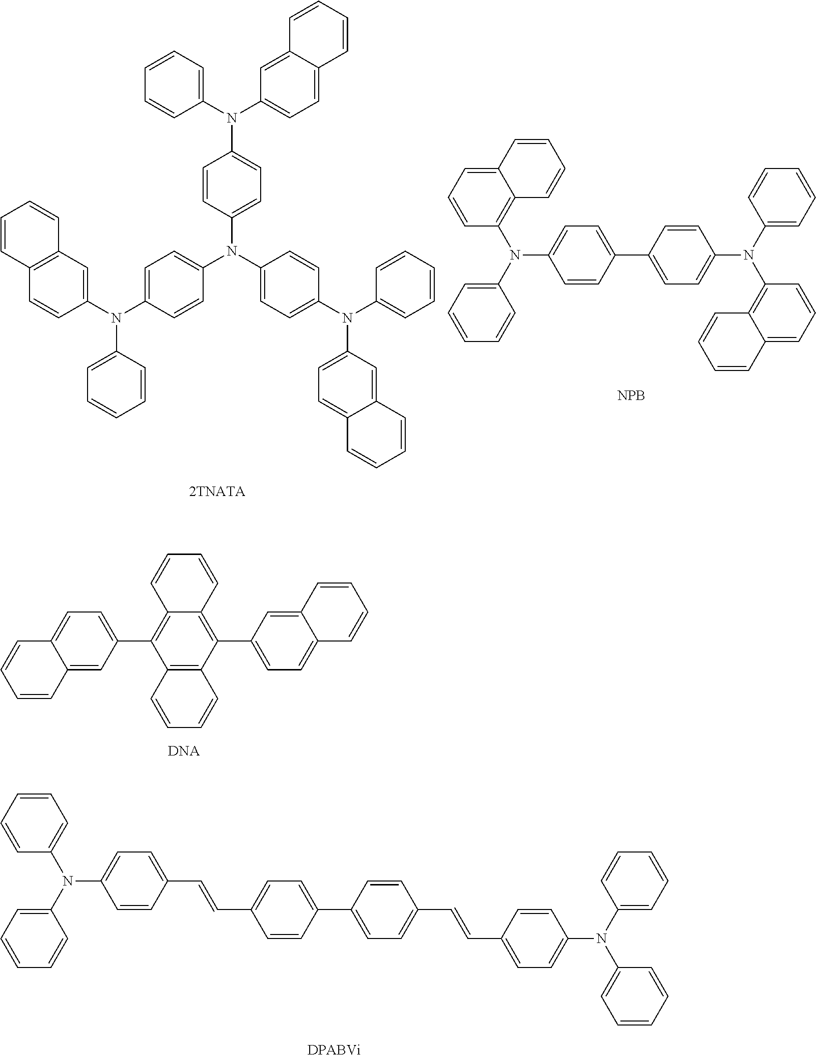

Alq₃ was vacuum-deposited on the emission layer to form an electron transport layer having a thickness of 300 Å. LiF was vacuum-deposited on the electron transport layer to form an electron injection layer having a thickness of 10 Å. Al was vacuum-deposited on the electron injection layer to form a cathode having a thickness of 3,000 Å, thereby completing the formation of an organic light-emitting device.

### Comparative Examples 2 to 7 and Examples 1 to 12

Organic light-emitting devices were manufactured in the same manner as in Comparative Example 1, except that the compounds shown in Table 1 were used instead of NPB in forming the hole transport layer.

### Example 13

A organic light-emitting device was manufactured in the same manner as in Comparative Example 1, except that, when forming a hole transport layer, Compound 204 was vacuum-deposited on the hole injection layer to form a first hole transport layer having a thickness of 100 Å, Compound HT1 was vacuum-deposited on the first hole transport layer to form a second hole transport layer having a thickness of 100 Å, and Compound 204 was vacuum-deposited on the second hole transport layer to form a third hole transport layer having a thickness of 100 Å, instead of using NPB for forming a hole transport layer having a thickness of 300 Å.

### Example 14

A organic light-emitting device was manufactured in the same manner as in Comparative Example 1, except that, when forming a hole transport layer, Compound 204 was vacuum-deposited on the hole injection layer to form a first hole transport layer having a thickness of 100 Å, Compound 2 was vacuum-deposited on the first hole transport layer to form a second hole transport layer having a thickness of 100 Å, and Compound 204 was vacuum-deposited on the second hole transport layer to form a third hole transport layer having a thickness of 100 Å, instead of using NPB for forming a hole transport layer having a thickness of 300 Å.

### Evaluation Example 1

To evaluate the characteristics of the organic light-emitting devices manufactured according to Comparative Examples 1 to 7 and Examples 1 to 14, the driving voltage, luminance, luminescence efficiency, and lifespan thereof were measured, and the results thereof are shown in Table 1.

The driving voltage at a current density of 50 mA/cm² was measured using a source meter (Keithley Instrument Inc., 2400 series).

For evaluating luminance and luminescence efficiency, power was supplied from a current-voltmeter (Kethley SMU 236), and measurements were made using a luminance meter PR650.

TABLE 2

| | | Driving | Current | | Luminescence | | Lifespan |
|---|---|---|---|---|---|---|---|
| | | voltage | density | Luminance | efficiency | Emission | (hr @ |
| | Compound | (V) | (mA/cm²) | (cd/m²) | (cd/A) | color | 100 mA/cm²) |
| Comparative | NPB | 7.01 | 50 | 2645 | 5.29 | Blue | 258 |
| Example 1 | | | | | | | |
| Comparative | Compound A | 5.43 | 50 | 2995 | 5.99 | Blue | 510 |
| Example 2 | | | | | | | |
| Comparative | Compound B | 5.21 | 50 | 2925 | 5.85 | Blue | 505 |
| Example 3 | | | | | | | |
| Comparative | Compound C | 5.25 | 50 | 3010 | 6.02 | Blue | 520 |
| Example 4 | | | | | | | |
| Comparative | Compound D | 5.44 | 50 | 2950 | 5.90 | Blue | 490 |
| Example 5 | | | | | | | |
| Comparative | Compound E | 5.30 | 50 | 2760 | 5.52 | Blue | 450 |
| Example 6 | | | | | | | |
| Comparative | Compound F | 6.50 | 50 | 2475 | 4.95 | Blue | 200 |
| Example 7 | | | | | | | |
| Example 1 | Compound 1 | 4.85 | 50 | 3155 | 6.31 | Blue | 580 |
| Example 2 | Compound 2 | 4.88 | 50 | 3175 | 6.35 | Blue | 560 |
| Example 3 | Compound 9 | 4.81 | 50 | 3165 | 6.33 | Blue | 555 |
| Example 4 | Compound10 | 4.84 | 50 | 3200 | 6.40 | Blue | 550 |
| Example 5 | Compound 18 | 4.88 | 50 | 3300 | 6.40 | Blue | 585 |
| Example 6 | Compound 45 | 4.82 | 50 | 3180 | 6.36 | Blue | 570 |
| Example 7 | Compound 65 | 4.92 | 50 | 3200 | 6.40 | Blue | 552 |
| Example 8 | Compound 73 | 4.92 | 50 | 3230 | 6.46 | Blue | 580 |
| Example 9 | Compound 76 | 4.91 | 50 | 3230 | 6.46 | Blue | 565 |
| Example 10 | Compound 85 | 4.89 | 50 | 3200 | 6.40 | Blue | 580 |
| Example 11 | Compound166 | 4.85 | 50 | 3215 | 6.43 | Blue | 590 |
| Example 12 | Compound 204 | 5.05 | 50 | 3245 | 6.49 | Blue | 620 |
| Example 13 | Compound 204/HT1/ | 5.30 | 50 | 3440 | 6.88 | Blue | 630 |
| | Compound 204 | | | | | | |
| Example 14 | Compound 204/ | 5.25 | 50 | 3425 | 6.85 | Blue | 665 |
| | Compound 2/ | | | | | | |
| | Compound 204 | | | | | | |
| Example 15 | Compound 209 | 4.92 | 50 | 3165 | 6.33 | Blue | 560 |
| Example 16 | Compound 210 | 4.95 | 50 | 3150 | 6.30 | Blue | 580 |
| Example 17 | Compound 211 | 4.95 | 50 | 3165 | 6.33 | Blue | 570 |
| Example 18 | Compound 212 | 5.00 | 50 | 3210 | 6.42 | Blue | 600 |
| Example 19 | Compound 213 | 5.00 | 50 | 3215 | 6.43 | Blue | 620 |
| Example 20 | Compound 214 | 4.89 | 50 | 3200 | 6.40 | Blue | 600 |
| Example 21 | Compound 215 | 4.89 | 50 | 3205 | 6.41 | Blue | 615 |
| Example 22 | Compound 216 | 4.85 | 50 | 3175 | 6.35 | Blue | 590 |
| Example 23 | Compound 217 | 4.88 | 50 | 3190 | 6.38 | Blue | 602 |

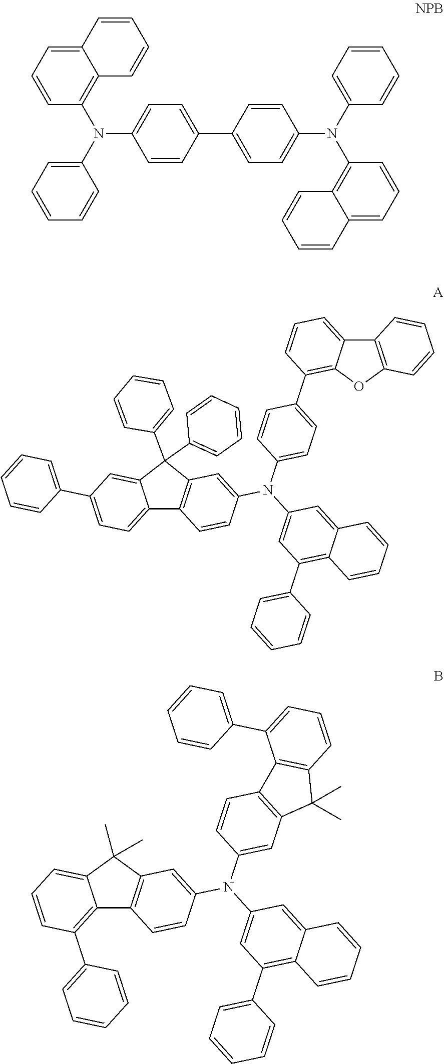 [SMILES: c1ccc(N(c2ccc(-c3ccc(N(c4ccccc4)c4cccc5ccccc45)cc3)cc2)c2cccc3ccccc23)cc1 ; c1ccc(-c2ccc3c(c2)C(c2ccccc2)(c2ccccc2)c2cc(N(c4ccc(-c5cccc6c5oc5ccccc56)cc4)c4cc(-c5ccccc5)c5ccccc5c4)ccc2-3)cc1 ; CC1(C)c2cc(N(c3ccc4c(c3)C(C)(C)c3cccc(-c5ccccc5)c3-4)c3cc(-c4ccccc4)c4ccccc4c3)ccc2-c2c(-c3ccccc3)cccc21]

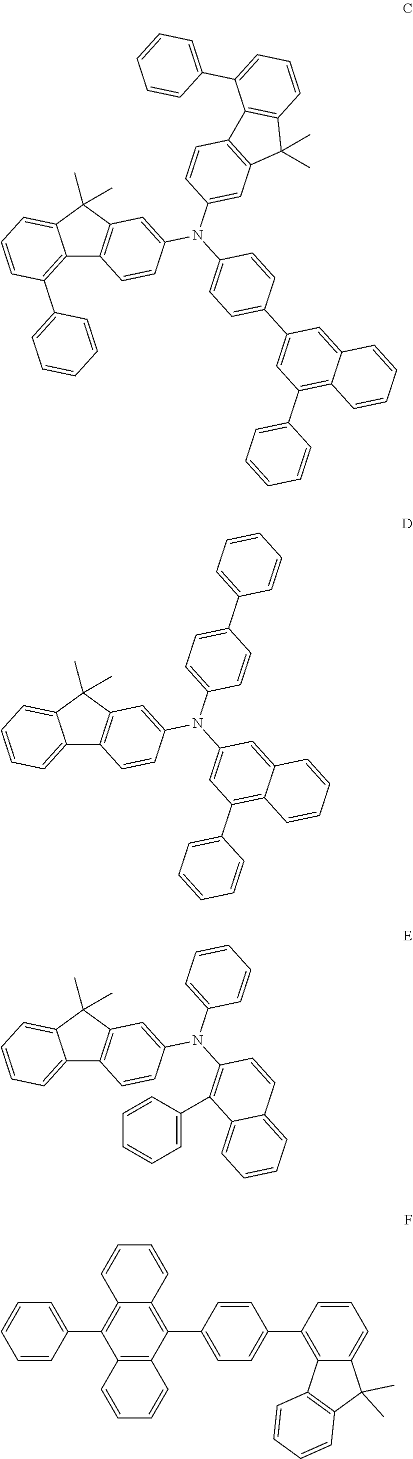

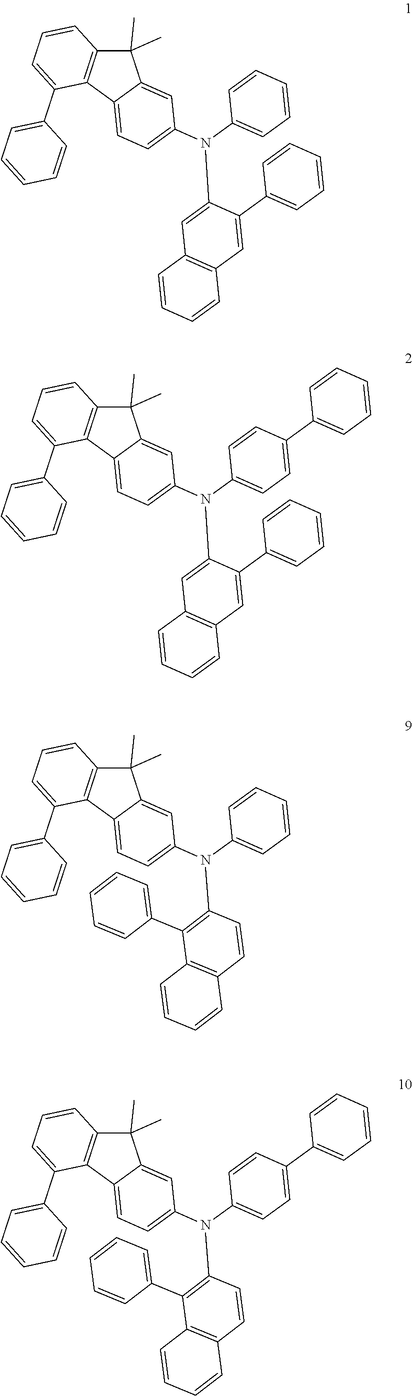 [SMILES: CC1(C)c2cc(N(c3ccccc3)c3cc4ccccc4cc3-c3ccccc3)ccc2-c2c(-c3ccccc3)cccc21 ; CC1(C)c2cc(N(c3ccc(-c4ccccc4)cc3)c3cc4ccccc4cc3-c3ccccc3)ccc2-c2c(-c3ccccc3)cccc21 ; CC1(C)c2cc(N(c3ccccc3)c3ccc4ccccc4c3-c3ccccc3)ccc2-c2c(-c3ccccc3)cccc21 ; CC1(C)c2cc(N(c3ccc(-c4ccccc4)cc3)c3ccc4ccccc4c3-c3ccccc3)ccc2-c2c(-c3ccccc3)cccc21]

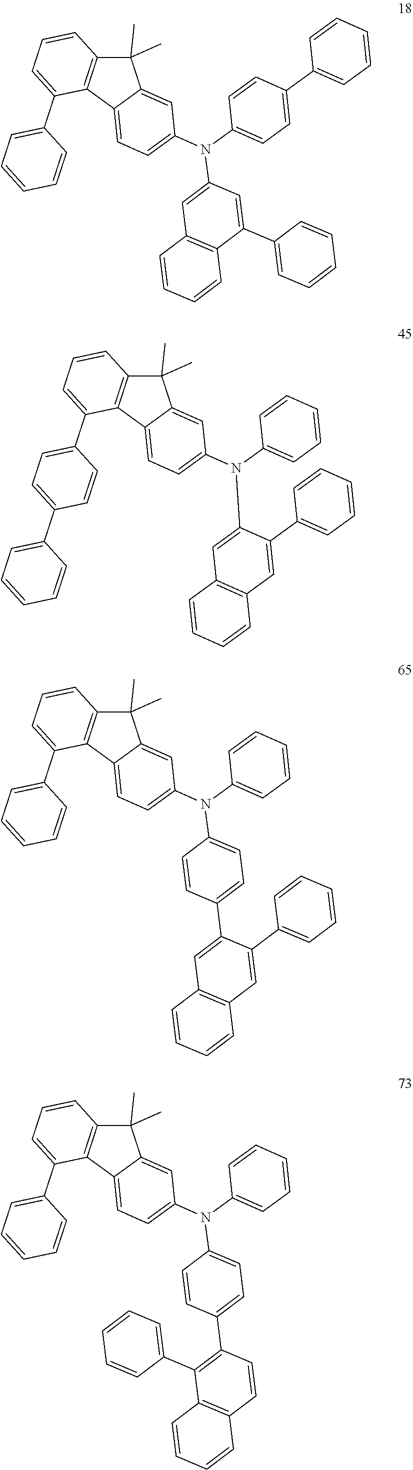 [SMILES: CC1(C)c2cc(N(c3ccc(-c4ccccc4)cc3)c3cc(-c4ccccc4)c4ccccc4c3)ccc2-c2c(-c3ccccc3)cccc21 ; CC1(C)c2cc(N(c3ccccc3)c3cc4ccccc4cc3-c3ccccc3)ccc2-c2c(-c3ccc(-c4ccccc4)cc3)cccc21 ; CC1(C)c2cc(N(c3ccccc3)c3ccc(-c4cc5ccccc5cc4-c4ccccc4)cc3)ccc2-c2c(-c3ccccc3)cccc21 ; CC1(C)c2cc(N(c3ccccc3)c3ccc(-c4ccc5ccccc5c4-c4ccccc4)cc3)ccc2-c2c(-c3ccccc3)cccc21]

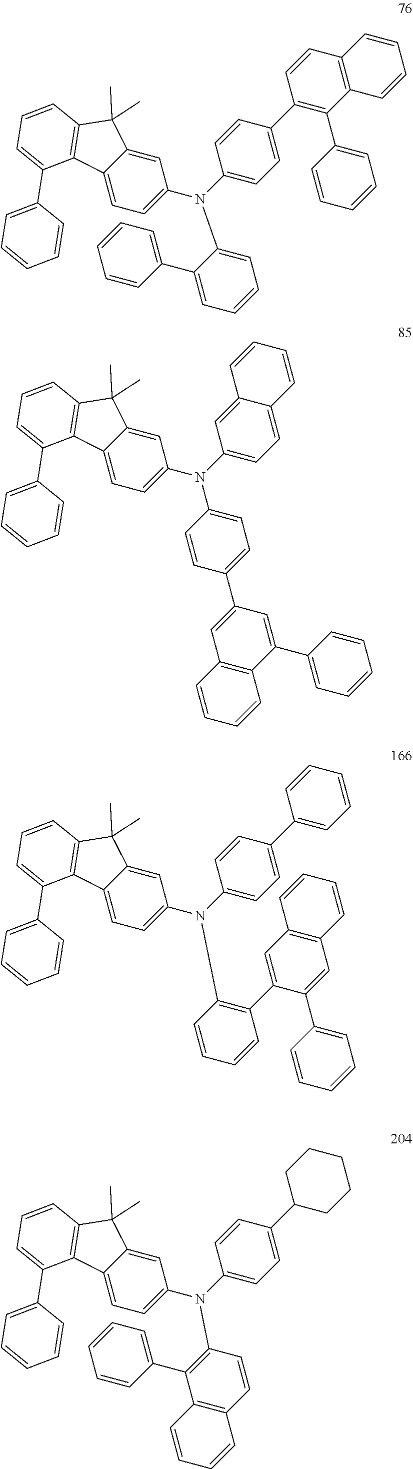

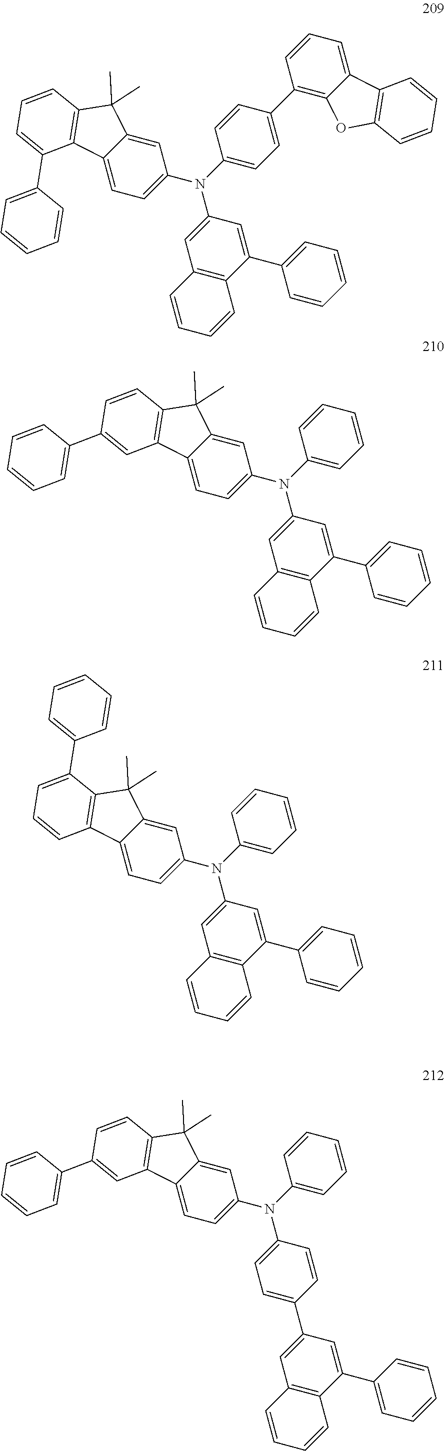

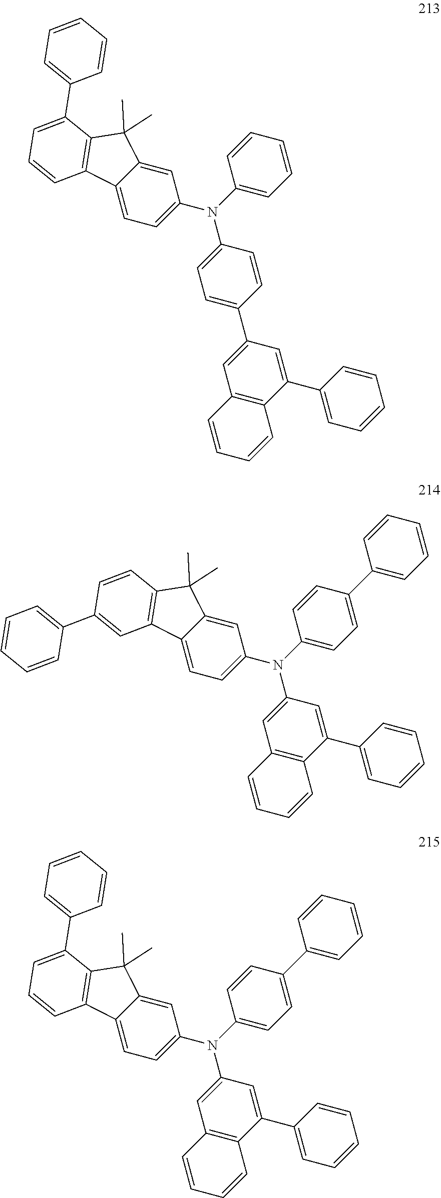 [SMILES: CC1(C)c2cc(N(c3ccccc3)c3ccc(-c4cc(-c5ccccc5)c5ccccc5c4)cc3)ccc2-c2cccc(-c3ccccc3)c21 ; CC1(C)c2ccc(-c3ccccc3)cc2-c2ccc(N(c3ccc(-c4ccccc4)cc3)c3cc(-c4ccccc4)c4ccccc4c3)cc21 ; CC1(C)c2cc(N(c3ccc(-c4ccccc4)cc3)c3cc(-c4ccccc4)c4ccccc4c3)ccc2-c2cccc(-c3ccccc3)c21]

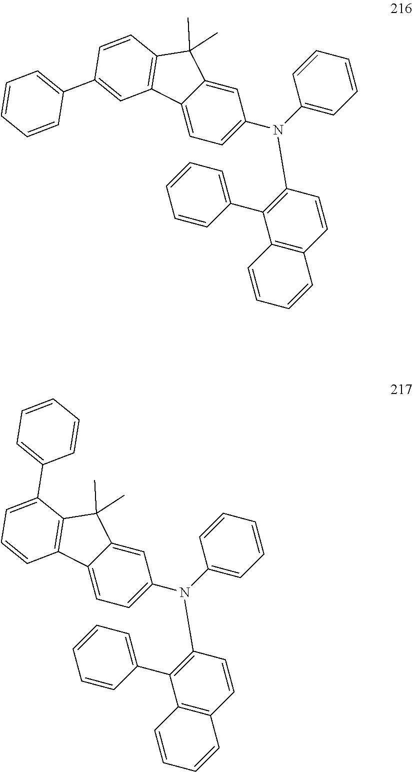 [SMILES: CC1(C)c2ccc(-c3ccccc3)cc2-c2ccc(N(c3ccccc3)c3ccc4ccccc4c3-c3ccccc3)cc21 ; CC1(C)c2cc(N(c3ccccc3)c3ccc4ccccc4c3-c3ccccc3)ccc2-c2cccc(-c3ccccc3)c21]

From Table 1, it was confirmed that the organic light-emitting device according to Examples 1 to 14 have relatively low driving voltage, high luminance, high luminescence efficiency, and long lifespan compared to the organic light-emitting devices according to Comparative Examples 1 to 7.

The amine-containing compound represented by one of Formulae 1 to 3 may have excellent hole transporting characteristics. The light-emitting device including the amine-containing compound may have a low driving voltage, high luminance, high efficiency, and long lifespan. The display quality of the electronic apparatus including the light-emitting device and the electronic device using the electronic apparatus may be improved.

Embodiments have been disclosed herein, and although terms are employed, they are used and are to be interpreted in a generic and descriptive sense only and not for purpose of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. A light-emitting device comprising:
a first electrode;
a second electrode facing the first electrode;
an interlayer between the first electrode and the second electrode and including an emission layer; and
an amine-containing compound represented by Formula 1, an amine-containing compound represented by Formula 2, an amine-containing compound represented by Formula 3, or a combination thereof: 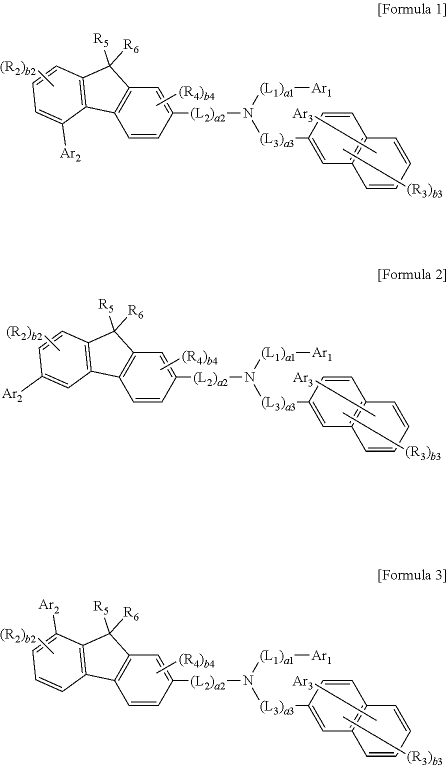
wherein in Formulae 1 to 3,
L₁ to L₃ and Ar₁ to Ar₃ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
except that each of L₁ and Ar₁ is not a fluorene group unsubstituted or substituted with at least one R₁₀ₐ,
a1 to a3 are each independently an integer from 0 to 5,
when a1 is 0, a group represented by *-(L₁)ₐ₁-*′ is a single bond,
when a2 is 0, a group represented by *-(L₂)ₐ₂-*′ is a single bond,
when a3 is 0, a group represented by *-(L₃)ₐ₃-*′ is a single bond,
when a1 is 2 to 5, a plurality of L₁(s) are identical to or different from each other,
when a2 is 2 to 5, a plurality of L₂(s) are identical to or different from each other,
when a3 is 2 to 5, a plurality of L₃(s) are identical to or different from each other,
R₂ to R₆ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₅ and R₆ are optionally bound to each other to form a C₃-C₁₀ carbocyclic group unsubstituted or substituted with at least one R₁₀ₐ or a C₁-C₆₀ heterocyclic group unsubstituted or substituted with at least one R₁₀ₐ,
b2 and b4 are each independently an integer from 0 to 3,
b3 is an integer from 0 to 6,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or a combination thereof, and
* and *′ each indicate a binding site to an adjacent atom.

2. The light-emitting device of claim 1, wherein
the interlayer further comprises:
a hole transport region between the first electrode and the emission layer, and
an electron transport region between the emission layer and the second electrode,
the hole transport region comprises a hole injection layer, a hole transport layer, an emission auxiliary layer, an electron blocking layer, or a combination thereof, and
the electron transport region comprises a buffer layer, a hole blocking layer, an electron control layer, an electron transport layer, an electron injection layer, or a combination thereof.

3. The light-emitting device of claim 1, wherein the interlayer comprises the amine-containing compound represented by Formula 1, the amine-containing compound represented by Formula 2, the amine-containing compound represented by Formula 3, or a combination thereof.

4. The light-emitting device of claim 2, wherein the hole transport region comprises the amine-containing compound represented by Formula 1, the amine-containing compound represented by Formula 2, the amine-containing compound represented by Formula 3, or a combination thereof.

5. The light-emitting device of claim 2, wherein
the hole transport layer comprises the amine-containing compound represented by Formula 1, the amine-containing compound represented by Formula 2, the amine-containing compound represented by Formula 3, or a combination thereof, and
the hole transport layer directly contacts the emission layer.

6. The light-emitting device of claim 1, further comprising:
a capping layer outside the first electrode, wherein
the capping layer comprises the amine-containing compound represented by Formula 1, the amine-containing compound represented by Formula 2, the amine-containing compound represented by Formula 3, or a combination thereof.

7. The light-emitting device of claim 1, further comprising:
a first capping layer outside the first electrode; and
a second capping layer outside the second electrode, wherein
the first capping layer or the second capping layer comprises the amine-containing compound represented by Formula 1, the amine-containing compound represented by Formula 2, the amine-containing compound represented by Formula 3, or a combination thereof.

8. An electronic apparatus comprising the light-emitting device of claim 1

9. The electronic apparatus of claim 8, further comprising:
a thin-film transistor electrically connected to the light-emitting device; and
a color filter, a color conversion layer, a touch screen layer, a polarizing layer, or a combination thereof.

10. An electronic device comprising the electronic apparatus of claim 8, wherein
the electronic device is a flat panel display, a curved display, a computer monitor, a medical monitor, a television, a billboard, an indoor light, an outdoor light, a signal light, a head-up display, a fully transparent display, a partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall with multiple displays tiled together, a theater screen, a stadium screen, a phototherapy device, or a signboard.

11. An amine-containing compound represented by one of Formulae 1 to 3: 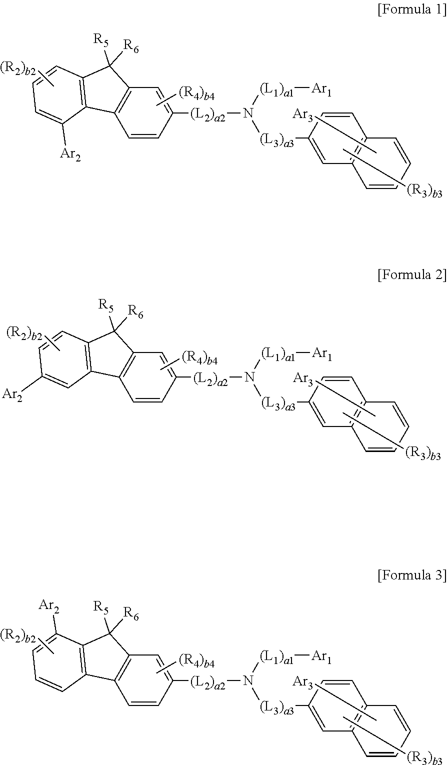
wherein in Formulae 1 to 3,
L₁ to L₃ and Ar₁ to Ar₃ are each independently a C₃-C₆₀ carbocyclic group unsubstituted or substituted with at least one R₁₀ₐ, or a C₁-C₆₀ heterocyclic group unsubstituted or substituted with at least one R₁₀ₐ,
except that each of L₁ and Ar₁ are not a fluorene group unsubstituted or substituted with at least one R₁₀ₐ,
a1 to a3 are each independently an integer from 0 to 5,
when a1 is 0, a group represented by *-(L₁)ₐ₁-*′ is a single bond,
when a2 is 0, a group represented by *-(L₂)ₐ₂-*′ is a single bond,
when a3 is 0, a group represented by *-(L₃)ₐ₃-*′ is a single bond,
when a1 is 2 to 5, a plurality of L₁(s) are identical to or different from each other,
when a2 is 2 to 5, a plurality of L₂(s) are identical to or different from each other,
when a3 is 2 to 5, a plurality of L₃(s) are identical to or different from each other,
R₂ to R₆ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₅ and R₆ are optionally bound to each other to form a C₃-C₀ carbocyclic group unsubstituted or substituted with at least one R₁₀ₐ or a C₁-C₆₀ heterocyclic group unsubstituted or substituted with at least one R₁₀ₐ,
b2 and b4 are each independently an integer from 0 to 3,
b3 is an integer from 0 to 6,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or a combination thereof, and
* and *′ each indicate a binding site to an adjacent atom.

12. The amine-containing compound of claim 11, wherein the amine-containing compound does not comprise a carbazole group and a fluorene group.

13. The amine-containing compound of claim 11, wherein R₁₀ₐ is deuterium, a C₁-C₂₀ alkyl group, a cyclohexyl group, a cycloheptyl group, a cyclooctyl group, an adamantanyl group, a norbornanyl group, a phenyl group, a naphthyl group, a phenanthrenyl group, a dibenzofuranyl group, or a dibenzothiophenyl group.

14. The amine-containing compound of claim 11, wherein L₁ to L₃ and Ar₁ to Ar₃ are each independently a cyclohexane group, a cycloheptane group, a cyclooctane group, an adamantane group, a norbornane group, a benzene group, a naphthalene group, a phenanthrene group, a dibenzofuran group, or a dibenzothiophene group, each unsubstituted or substituted with deuterium, a C₁-C₂₀ alkyl group, a cyclohexyl group, a cycloheptyl group, a cyclooctyl group, an adamantanyl group, a norbornanyl group, a phenyl group, a naphthyl group, a phenanthrenyl group, a dibenzofuranyl group, a dibenzothiophenyl group, or a combination thereof.

15. The amine-containing compound of claim 11, wherein L₁ to L₃ are each independently a group represented by one of Formulae 4-1 to 4-3: 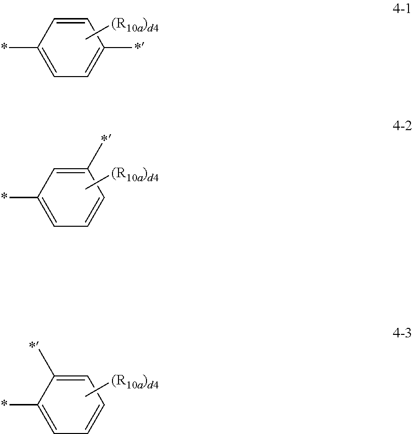
wherein in Formulae 4-1 to 4-3
R₁₀ₐ is as described in Formulae 1 to 3,
d4 is an integer from 0 to 4, and
* and *′ each indicate a binding site to an adjacent atom.

16. The amine-containing compound of claim 11, wherein Ar to Ar₃ are each independently a group represented by one of Formulae 5-1 to 5-7: 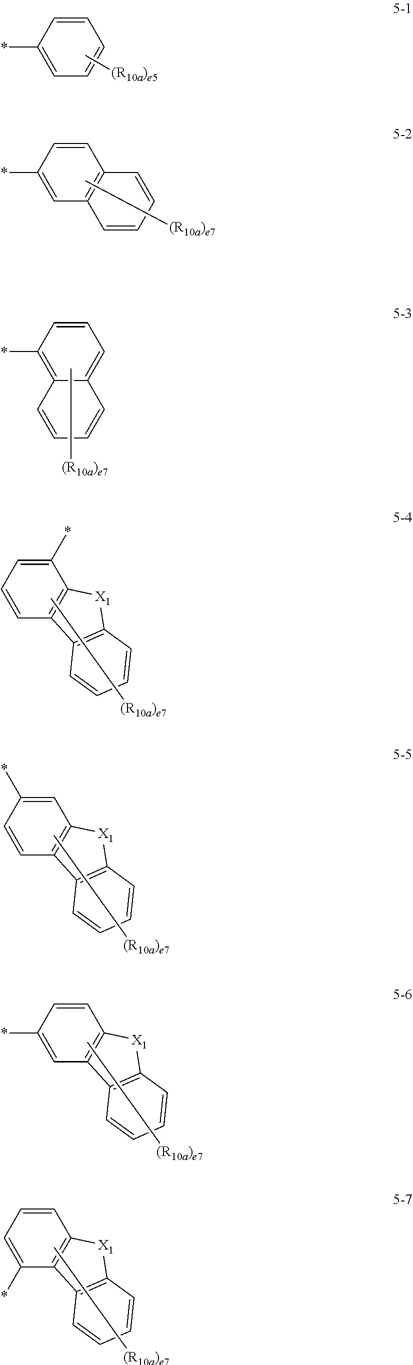
wherein in Formulae 5-1 to 5-7,
R₁₀ₐ is as described in Formulae 1 to 3,
X₁ is O or S,
e5 is an integer from 0 to 5,
e7 is an integer from 0 to 7, and
* indicates a binding site to an adjacent atom.

17. The amine-containing compound of claim 11, wherein Ar to Ar₃ are each independently a group represented by one of Formulae 6-1 to 6-12: 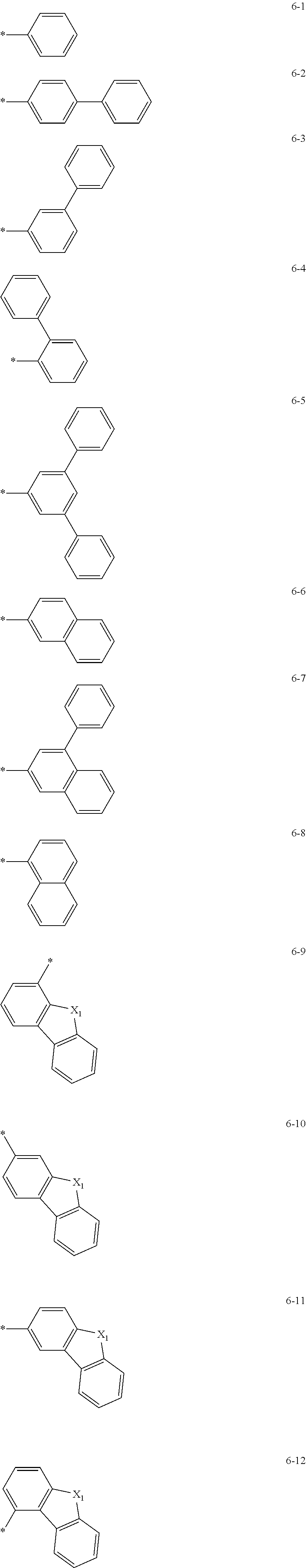
wherein in Formulae 6-1 to 6-12,
X₁ is O or S, and
* indicates a binding site to an adjacent atom.

18. The amine-containing compound of claim 11, wherein in Formulae 1 to 3, a group represented by 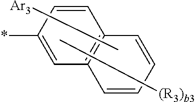
is a group represented by one of Formulae A-1 to A-3: 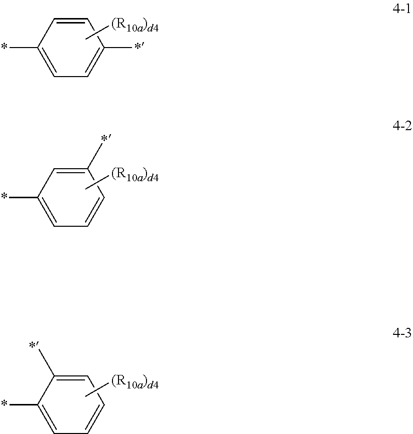
wherein in Formulae A-1 to A-3,
Ar₃, R₃, and b3 are each as described in Formulae 1 to 3, and
* indicates a binding site to an adjacent atom.

19. The amine-containing compound of claim 11, wherein R₅ and R₆ are linked to each other to form a cyclopentane group or a cyclohexane group, each unsubstituted or substituted with at least one R₁₀ₐ.

20. The amine-containing compound of claim 11, wherein the amine-containing compound is one of Compounds 1 to 217: 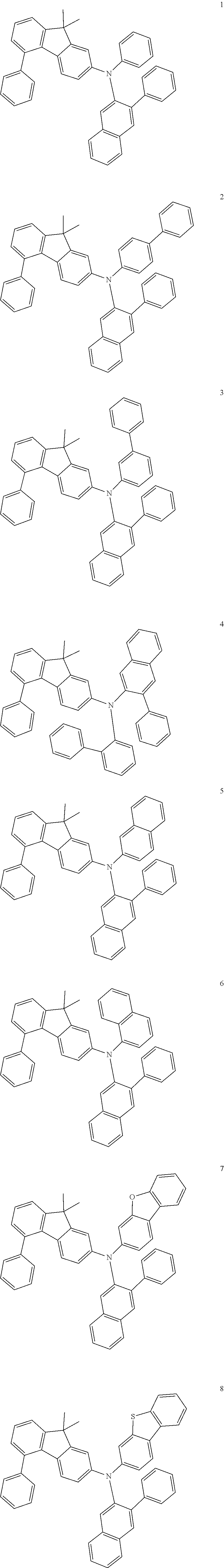 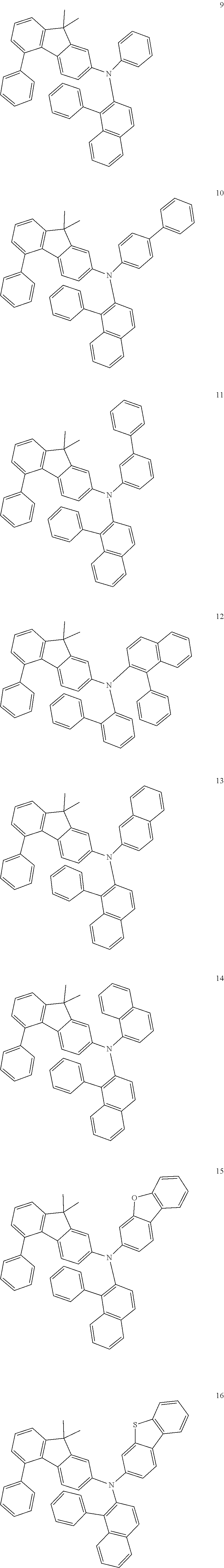 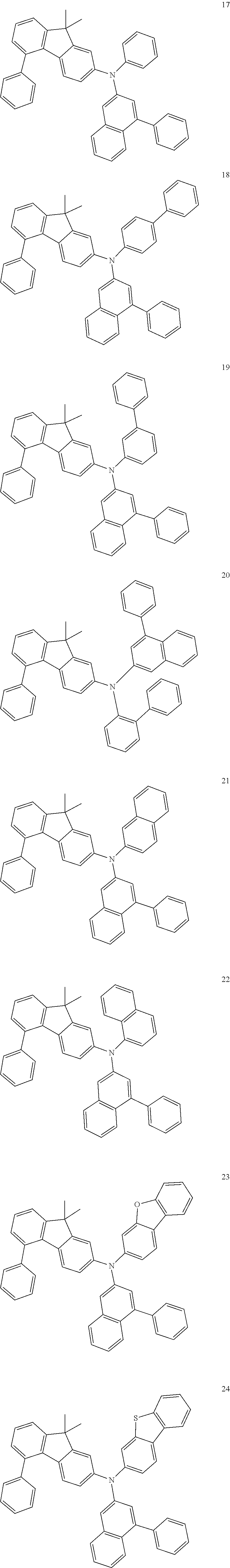 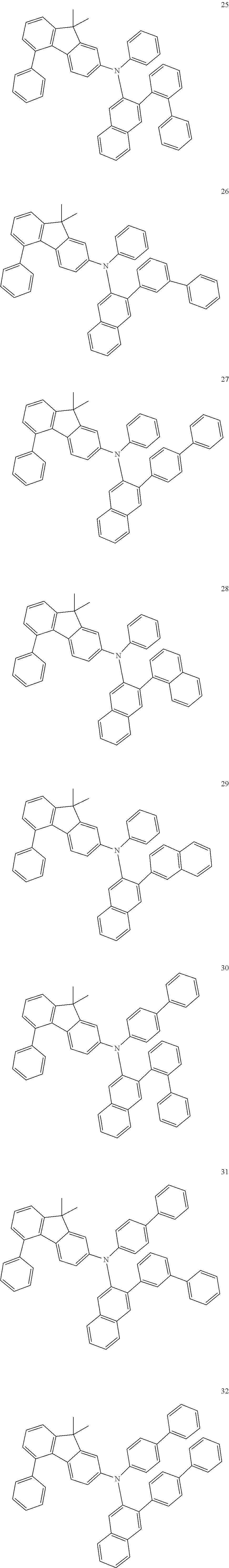 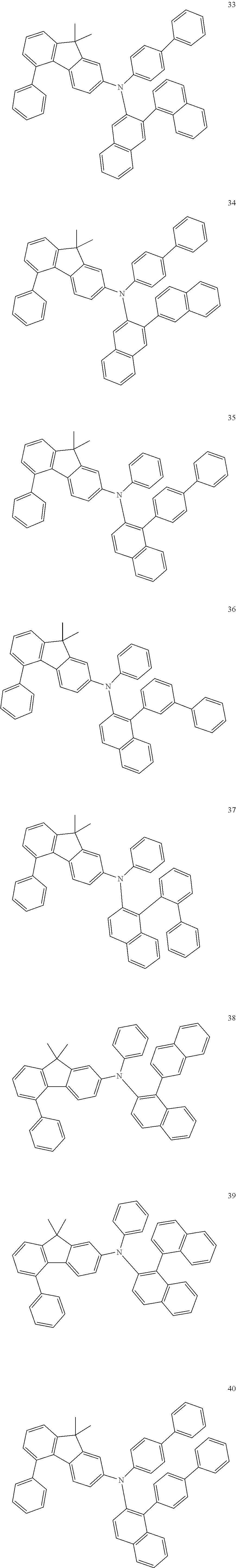 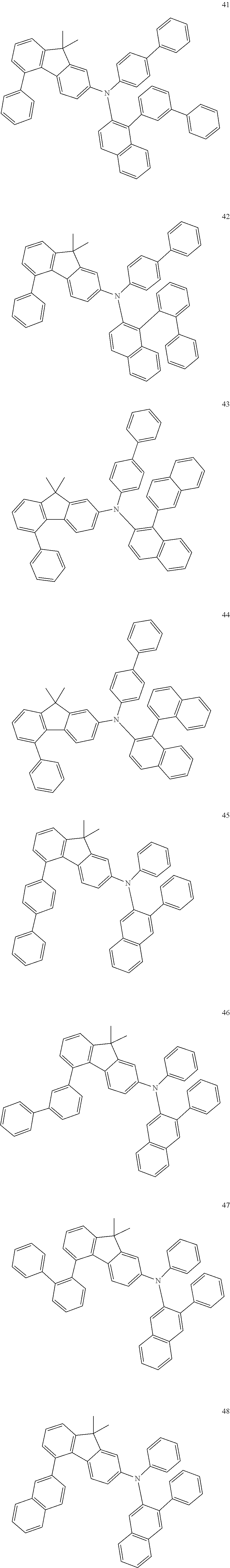 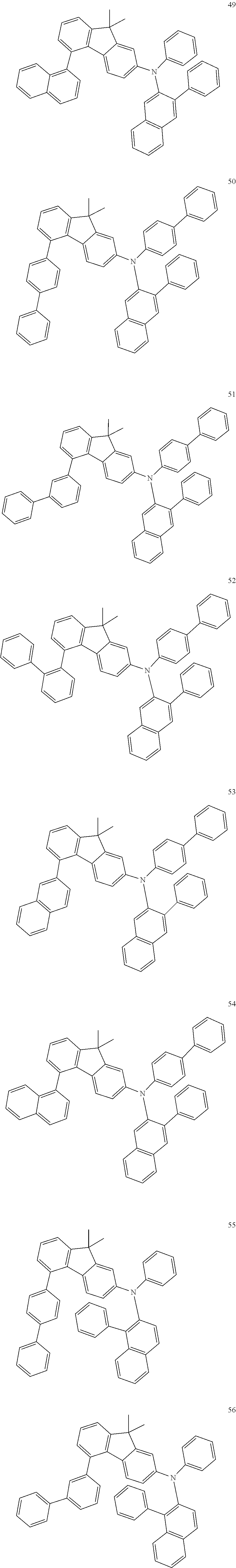 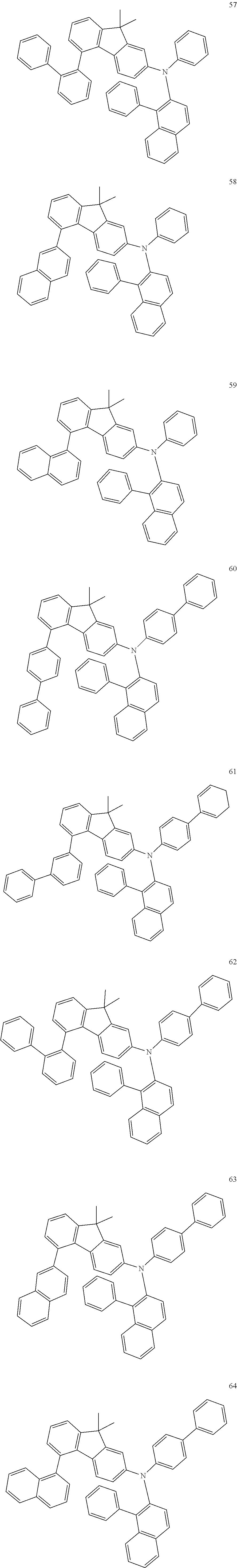 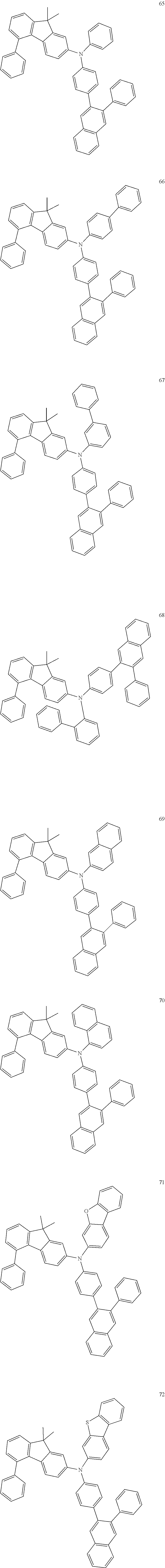 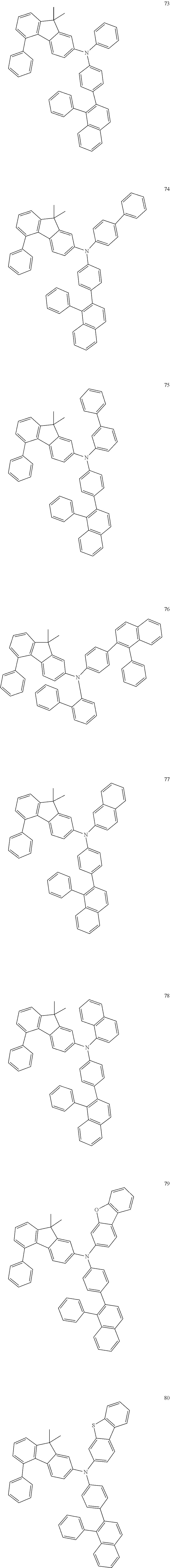 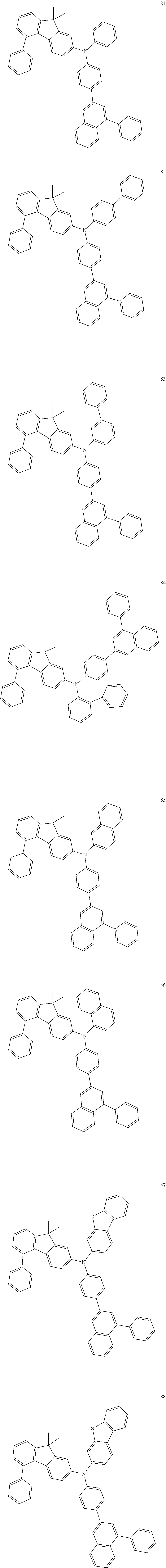 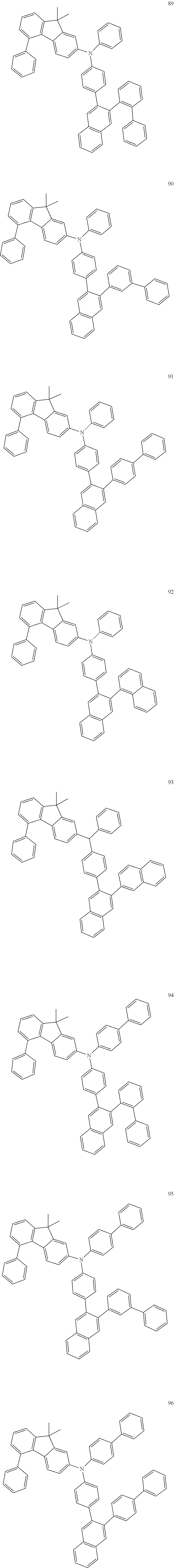 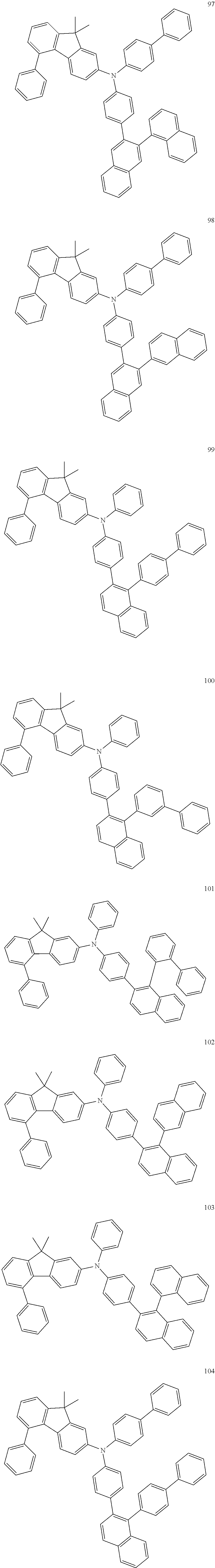 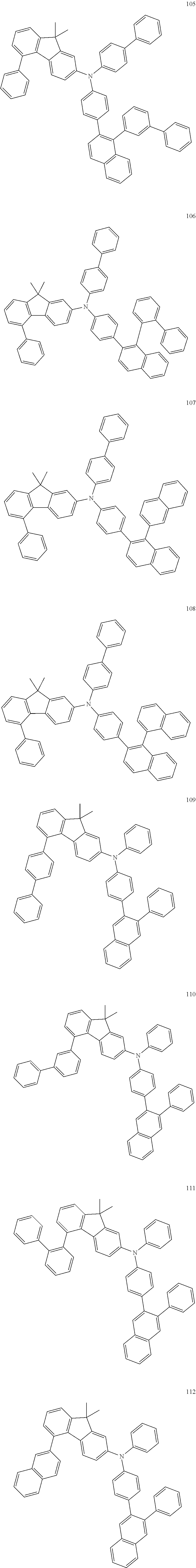 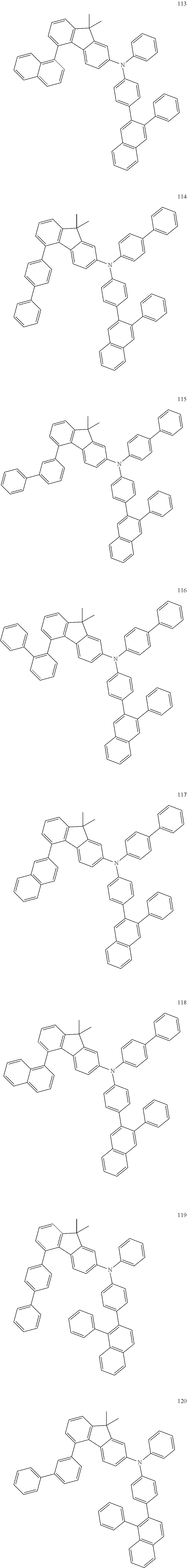 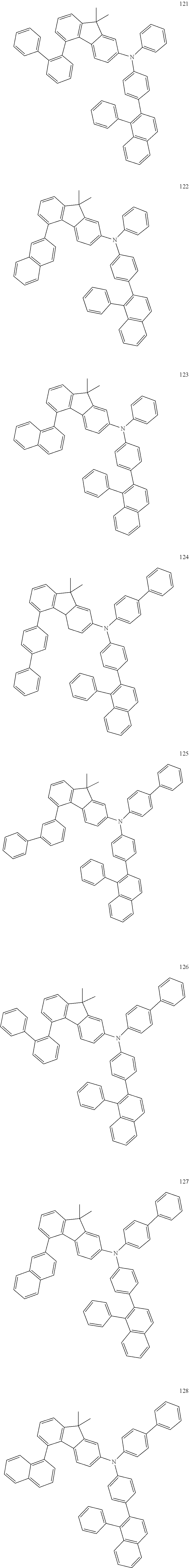 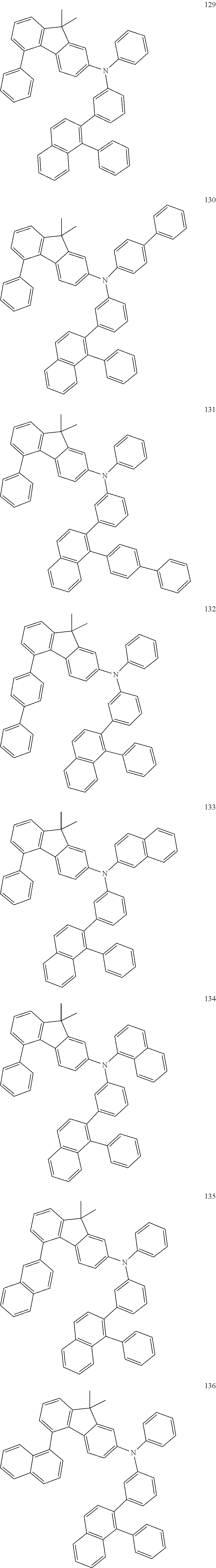 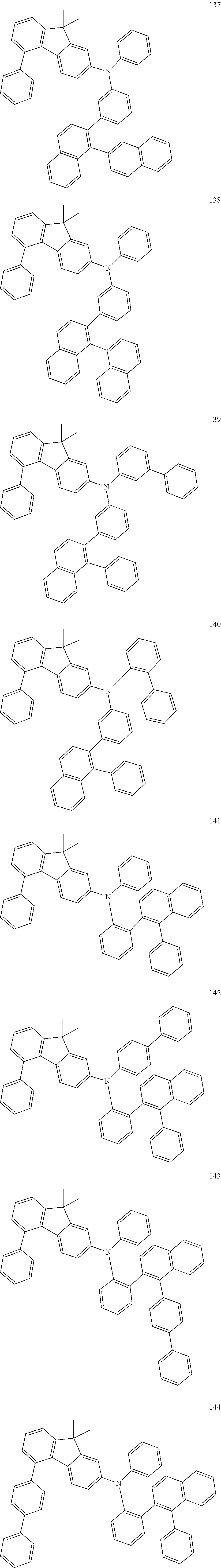 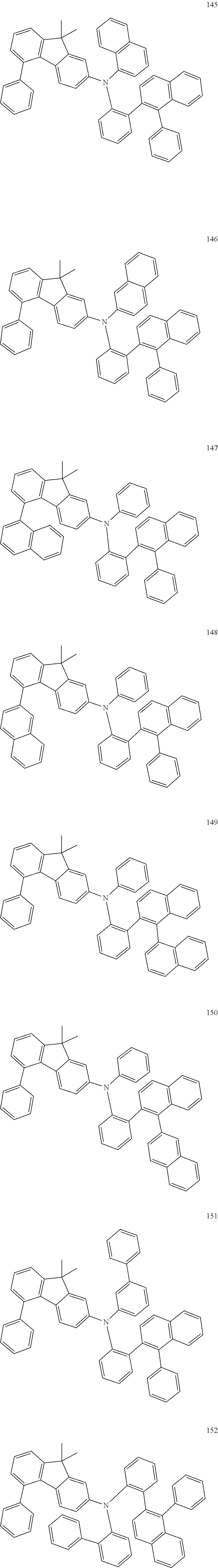 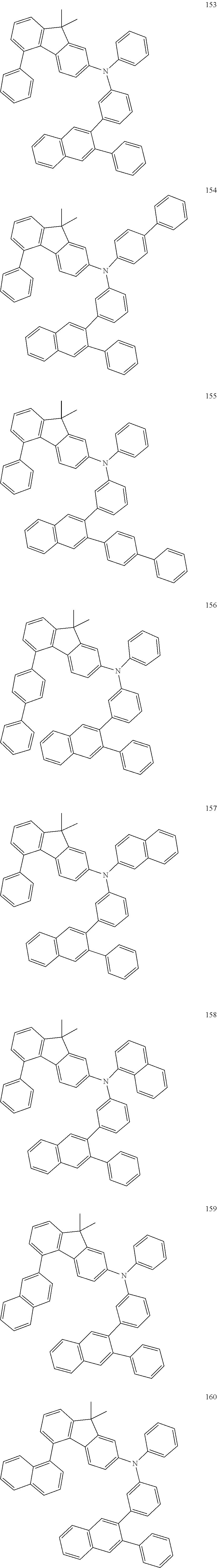 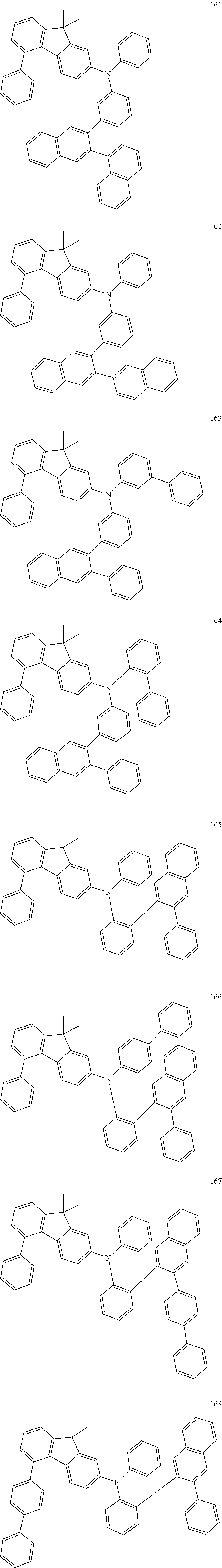 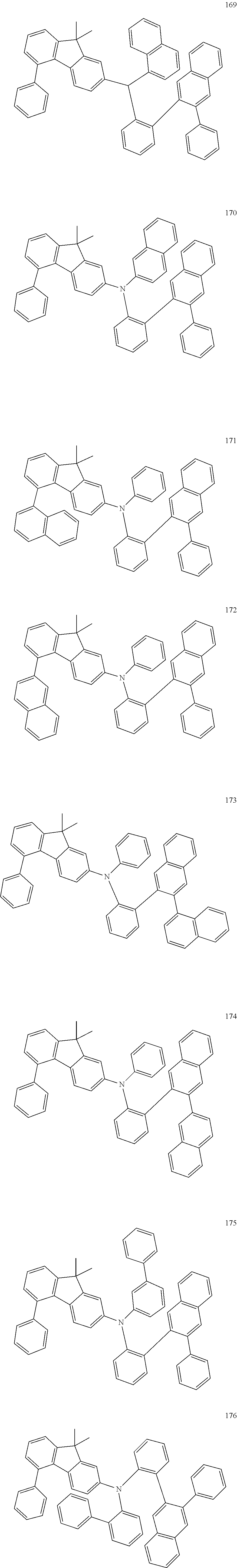 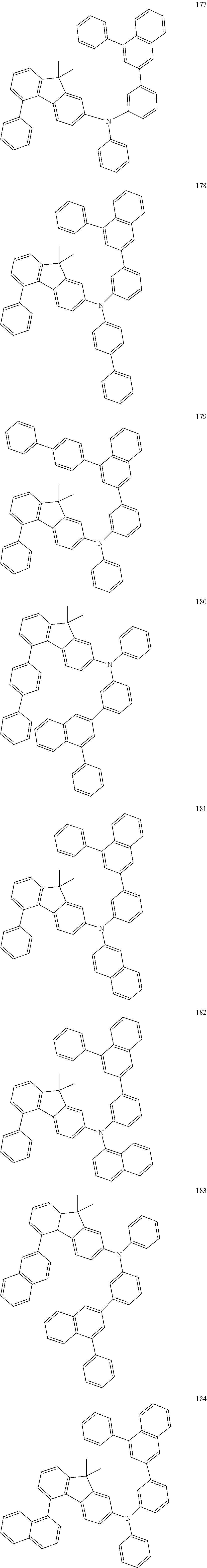 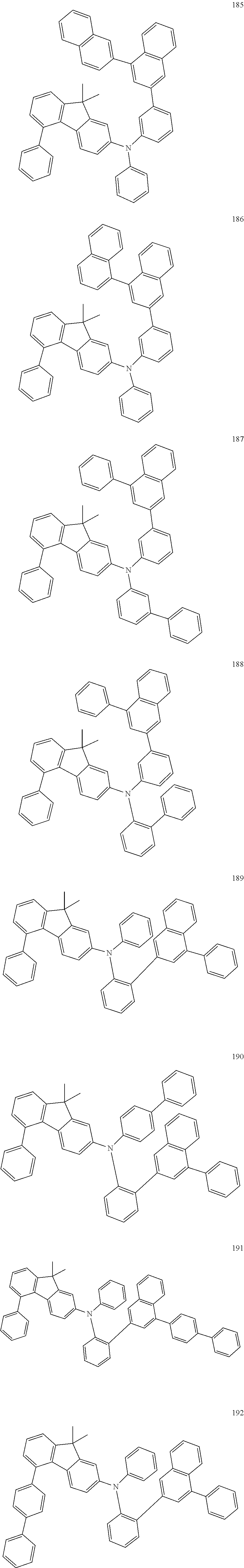 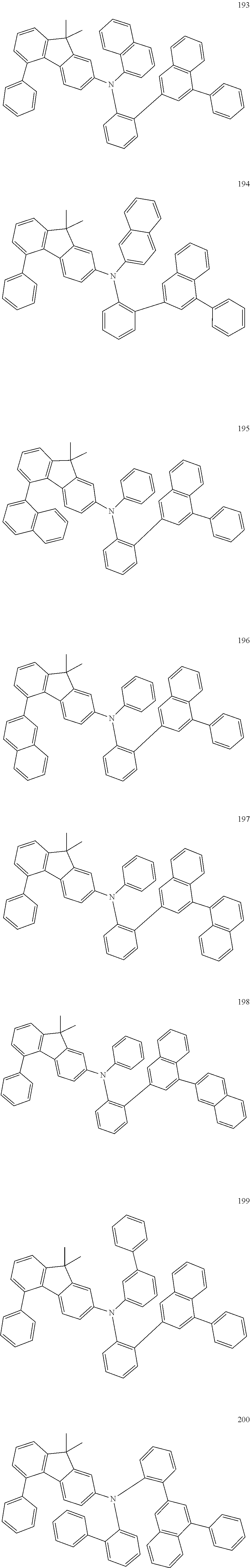 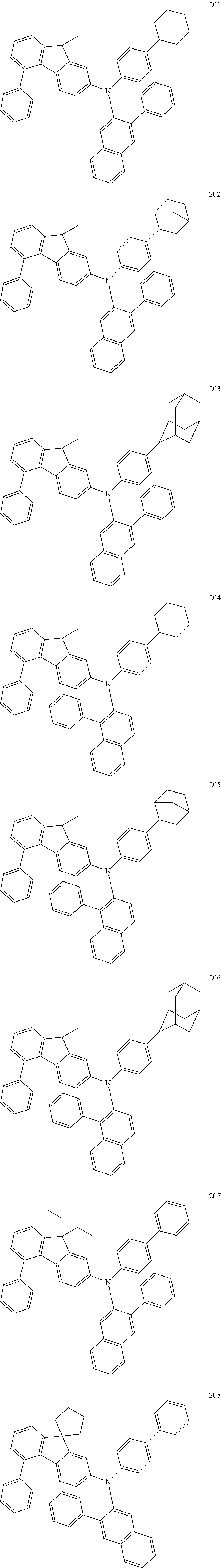 [chemical structure]
